(19) United States
(12) Patent Application Publication
(10) Pub. No.: US 2023/0374549 A1
(43) Pub. Date: Nov. 23, 2023

(54) COMPOSITIONS AND METHODS FOR INHIBITING THE EXPRESSION OF MULTIPLE GENES

(71) Applicant: FLAGSHIP PIONEERING INNOVATIONS V, INC., Cambridge, MA (US)

(72) Inventors: Lauren Marie BEECH, Plymouth, MA (US); Jesse Jerome SMITH, Waltham, MA (US); Rahul KARNIK, Cambridge, MA (US); Kendrick Alan GOSS, Lexington, MA (US); Adam Walter SCHEIDEGGER, Somerville, MA (US); Jodi Michelle KENNEDY, Dedham, MA (US); Jeremiah Dale FARELLI, Marblehead, MA (US); Houda BELAGHZAL, Cambridge, MA (US); Laura Anh NGUYEN, Medford, MA (US); Charles W. O'DONNELL, Winchester, MA (US)

(21) Appl. No.: 18/028,975

(22) PCT Filed: Sep. 29, 2021
(86) PCT No.: PCT/US2021/052720

§ 371 Date: Mar. 28, 2023

Related U.S. Application Data
(60) Provisional application No. 63/085,013, filed on Sep. 29, 2020.
(60) Provisional application No. 63/216,487, filed on Jun. 29, 2021.

(51) Int. Cl. C12N 15/90; C12N 9/22; C12N 15/11; C12N 9/10; C12N 9/80; A61P 37/06; A61K 35/12; A61K 38/46; A61K 31/7088; C07K 14/47

(52) U.S. Cl. CPC C12N 15/907; C12N 9/22; C12N 15/11; C12N 9/1007; C12Y 201/01043; C12N 9/80; C12Y 305/01098; A61P 37/06; A61K 35/12; A61K 38/465; A61K 31/7088; C07K 14/4703; C12Y 201/01037; C12Y 201/01072; C12N 2310/20; A61K 2035/122; C07K 2319/00

(57) ABSTRACT

The present disclosure relates to site-specific disrupting agents for modulating, e.g., decreasing, expression of a target plurality of genes in a cell. In some embodiments, the target plurality of genes comprises pro-inflammatory genes, e.g., CXCL1, CXCL2, CXCL3, CXCL4, CXCL5, CXCL6, CXCL7, and IL-8. In some embodiments, the method comprises using a first site-specific disrupting agent that targets a first anchor sequence and a second site-specific disrupting agents that disrupts a second anchor sequence.

### CROSS-REFERENCE TO RELATED APPLICATIONS

The present application claims the benefit of U.S. provisional applications 63/085,013 filed Sep. 29, 2020, and 63/216,487 filed Jun. 29, 2021. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 29, 2021, is named 02057-7021WO_SL.txt and is 666,818 bytes in size.

### BACKGROUND

Mis-regulation of gene expression is the underlying cause of many diseases (e.g., in mammals, e.g., humans). A number of diseases and conditions are associated with pluralities of related genes. There is a need for novel tools, systems, and methods to alter, e.g., decrease, expression of pluralities of associated genes.

### SUMMARY

The disclosure provides, among other things, site-specific disrupting agents or systems comprising site-specific disrupting agents that may be used to modulate, e.g., decrease, expression of a plurality of target genes, e.g., a first gene and a second gene, that are within an anchor sequence-mediated conjunction (ASMC) comprising a first anchor sequence and a second anchor sequence. In one aspect, a site-specific disrupting agent comprises a targeting moiety that binds specifically to a first anchor sequence or proximal to the first anchor sequence in an ASMC. In some embodiments, binding of the site-specific disrupting agent occurs in an amount sufficient to modulate, e.g., decrease, expression of the plurality of target genes, e.g., the first gene and second gene. In some embodiments, the site-specific disrupting agent further comprises an effector moiety. Generally, modulation of expression of a target plurality of genes by a site-specific disrupting agent involves the binding of the site-specific disrupting agent to or proximal to the first anchor sequence. In some embodiments, binding of the site-specific disrupting agent to the first anchor sequence may disrupt binding of a nucleating polypeptide, e.g., CTCF, to the first anchor sequence, e.g., thereby disrupting formation and/or maintenance of the ASMC, e.g., and thereby modulating, e.g., decreasing, expression of the plurality of genes. In some embodiments, binding of the site-specific disrupting agent to or proximal to the first anchor sequence may localize a functionality of an effector moiety to the first anchor sequence and/or ASMC, e.g., thereby disrupting formation and/or maintenance of the ASMC, e.g., and thereby modulating, e.g., decreasing, expression of the plurality of genes. In some embodiments, binding of the site-specific disrupting agent to or proximal to the first anchor sequence may localize a functionality of an effector moiety to the first anchor sequence and/or ASMC, e.g., thereby modulating, e.g., decreasing, expression of the plurality of genes. Without wishing to be bound by theory, in some embodiments it is thought that targeting a plurality of genes that are within the same ASMC may more effectively modulate, e.g., decrease, expression of the plurality of genes and/or more effectively achieve a therapeutic effect relating to the functionality of the plurality of genes. For example, in some embodiments a targeted plurality of genes may all be pro-inflammatory genes; targeting the plurality of pro-inflammatory genes for modulation, e.g., reduction, in expression as taught herein may more effectively decrease inflammation than targeting individual genes. Targeting a plurality of genes comprised within the same genomic complex, e.g., ASMC, (e.g., by targeting the ASMC or an anchor sequence of the ASMC) may have an additive or synergistic effect (e.g., with regard to expression modulation or stability/duration of modulation) that is greater than the effect of targeting individual genes of the plurality.

In some aspects, the disclosure provides a method of decreasing expression of a first gene and a second gene in a cell, comprising: contacting the cell with a site-specific disrupting agent comprising a targeting moiety that binds specifically to a first anchor sequence or a site proximal to a first anchor sequence, in an amount sufficient to decrease expression of the first and second genes, the first and second genes being within an anchor sequence-mediated conjunction that comprises the first anchor sequence and a second anchor sequence. In some embodiments, the first gene and the second gene are proinflammatory genes.

In some aspects, the disclosure is directed to a site-specific disrupting agent, comprising: a DNA-binding, e.g., a targeting moiety that binds specifically to or proximal to a first anchor sequence within a cell. In some embodiments, the first anchor sequence is part of an anchor sequence-mediated conjunction that further comprises a second anchor sequence, a first gene, and a second gene. In some embodiments, the first gene and the second gene are proinflammatory genes.

In some aspects, the disclosure is directed to a site-specific disrupting agent, comprising: a targeting moiety that binds specifically to or proximal to a first anchor sequence within a cell, wherein the first anchor sequence is part of an anchor sequence-mediated conjunction that further comprises a second anchor sequence, a first gene, and a second gene, wherein the first gene and the second gene are proinflammatory genes.

In another aspect, the disclosure provides a system comprising a first site-specific disrupting agent comprising a first targeting moiety and optionally a first effector moiety, wherein the first site-specific disrupting agent binds specifically to a first anchor sequence (or proximal to the first anchor sequence) of an anchor sequence mediated conjunction (ASMC), comprising a first gene and a second gene, and a second site-specific disrupting agent comprising a second targeting moiety and optionally a second effector moiety, wherein the second site-specific disrupting agent binds to a second anchor sequence (or proximal to the second anchor sequence) of the ASMC.

In another aspect, the disclosure is directed to a method of decreasing expression of a first gene and a second gene in a cell, comprising: contacting the cell with a site-specific disrupting agent that comprises a targeting moiety that binds specifically to a first anchor sequence or a site proximal to a first anchor sequence, in an amount sufficient to decrease expression of the first and second genes, the first and second genes being within an anchor sequence-mediated conjunction that comprises the first anchor sequence and a second anchor sequence, wherein the first gene and the second gene are proinflammatory genes; thereby decreasing expression of the first and second genes.

In another aspect, the disclosure is directed to a method of decreasing expression of a first gene and a second gene in a cell, comprising:
contacting the cell with a system comprising a first site-specific disrupting agent comprising a first targeting moiety and optionally a first effector moiety that binds specifically to a first anchor sequence or a site proximal to a first anchor sequence and a second site-specific disrupting agent comprising a second targeting moiety and optionally a second effector moiety wherein the second site-specific disrupting agent binds to a second anchor sequence (or proximal to the second anchor sequence) of the ASMC, in an amount sufficient to decrease expression of the first and second genes, the first and second genes being within the ASMC, wherein the first gene and the second gene are proinflammatory genes.

In another aspect, the disclosure is directed to a reaction mixture comprising a cell (e.g., a human cell, e.g., a primary human cell) and a site-specific disrupting agent or a system as described herein.

In another aspect, the disclosure is directed to a method of treating a subject having an inflammatory disorder, comprising administering to the subject a site-specific disrupting agent or a system as described herein in an amount sufficient to treat the inflammatory disorder.

In another aspect, the disclosure is directed to a method of treating inflammation, e.g., local inflammation, in a subject having an infection, e.g., viral infection, e.g., COVID-19, comprising, administering to the subject a site-specific disrupting agent or a system as described herein in an amount sufficient to treat the inflammation.

In another aspect, the disclosure is directed to a human cell having decreased expression of a first gene and a second gene, wherein the first gene and the second gene are proinflammatory genes, wherein the cell comprises a disrupted (e.g., fully disrupted) anchor sequence-mediated conjunction that comprises the first and second genes. In some embodiments, the human cell was previously contacted with a site-specific disrupting agent or system described herein. In some embodiments, the human cell no longer comprises a site-specific disrupting agent or system described herein.

In another aspect, the disclosure is directed to a human cell comprising a mutation at genomic coordinates chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, or chr4:74570526-74570548, or within 5, 10, 15, 20, 30, 40, or 50 nucleotides of said region.

Those skilled in the art will recognize or be able to ascertain using no more than routine experimentation, many equivalents to the specific embodiments of the disclosure described herein.

All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Sep. 29, 2020. When one gene or protein references a plurality of sequence accession numbers, all of the sequence variants are encompassed.

### Definitions

Anchor Sequence: The term “anchor sequence” as used herein, refers to a nucleic acid sequence recognized by a nucleating agent that binds sufficiently to form an anchor sequence-mediated conjunction, e.g., a complex. In some embodiments, an anchor sequence comprises one or more CTCF binding motifs. In some embodiments, an anchor sequence is not located within a gene coding region. In some embodiments, an anchor sequence is located within an intergenic region. In some embodiments, an anchor sequence is not located within either of an enhancer or a promoter. In some embodiments, an anchor sequence is located at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1 kb away from any transcription start site. In some embodiments, an anchor sequence is located within a region that is not associated with genomic imprinting, monoallelic expression, and/or monoallelic epigenetic marks. In some embodiments, the anchor sequence has one or more functions selected from binding an endogenous nucleating polypeptide (e.g., CTCF), interacting with a second anchor sequence to form an anchor sequence mediated conjunction, or insulating against an enhancer that is outside the anchor sequence mediated conjunction. In some embodiments of the present disclosure, technologies are provided that may specifically target a particular anchor sequence or anchor sequences, without targeting other anchor sequences (e.g., sequences that may contain a nucleating agent (e.g., CTCF) binding motif in a different context); such targeted anchor sequences may be referred to as the “target anchor sequence”. In some embodiments, sequence and/or activity of a target anchor sequence is modulated while sequence and/or activity of one or more other anchor sequences that may be present in the same system (e.g., in the same cell and/or in some embodiments on the same nucleic acid molecule—e.g., the same chromosome) as the targeted anchor sequence is not modulated. In some embodiments, the anchor sequence comprises or is a nucleating polypeptide binding motif. In some embodiments, the anchor sequence is adjacent to a nucleating polypeptide binding motif.

Anchor Sequence-Mediated Conjunction: The term “anchor sequence-mediated conjunction” as used herein, refers to a DNA structure, in some cases, a complex, that occurs and/or is maintained via physical interaction or binding of at least two anchor sequences in the DNA by one or more polypeptides, such as nucleating polypeptides, or one or more proteins and/or a nucleic acid entity (such as RNA or DNA), that bind the anchor sequences to enable spatial proximity and functional linkage between the anchor sequences (see, e.g. FIG. 1).

Associated with: Two events or entities are “associated” with one another, as that term is used herein, if presence, level, form and/or function of one is correlated with that of the other. For example, in some embodiments, a particular entity (e.g., polypeptide, genetic signature, metabolite, microbe, etc.) is considered to be associated with a particular disease, disorder, or condition, if its presence, level, form and/or function correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof. In some embodiments, a DNA sequence is “associated with” a target genomic or transcription complex when the nucleic acid is at least partially within the target genomic or transcription complex, and expression of a gene in the DNA sequence is affected by formation or disruption of the target genomic or transcription complex.

Site-specific disrupting agent: As used herein, the term “site-specific disrupting agent” refers to an agent or entity that specifically inhibits, dissociates, degrades, and/or modifies one or more components of a genomic complex, e.g., ASMC, thereby modulating, e.g., decreasing, expression of a target plurality of genes as described herein. In some embodiments, a site-specific disrupting agent interacts with one or more components of a genomic complex. In some embodiments, a site-specific disrupting agent binds (e.g., directly or, in some embodiments, indirectly) to one or more genomic complex components. In some embodiments, a site-specific disrupting agent binds to an anchor sequence, e.g., a first and/or second anchor sequence, that may be part of an ASMC comprising a target plurality of genes. In some embodiments, a site-specific disrupting agent binds to a site proximal to an anchor sequence, e.g., a first and/or second anchor sequence, that may be part of an ASMC comprising a target plurality of genes. In some embodiments, a site-specific disrupting agent modifies one or more genomic complex components. In some embodiments, a site-specific disrupting agent comprises an oligonucleotide. In some embodiments, a site-specific disrupting agent comprises a polypeptide. In some embodiments, a site-specific disrupting agent comprises an antibody (e.g., a monospecific or multi-specific antibody construct) or antibody fragment. In some embodiments, a site-specific disrupting agent is directed to a particular genomic location and/or to a genomic complex by a targeting moiety, as described herein. In some embodiments, a site-specific disrupting agent comprises a genomic complex component or variant thereof. In some embodiments, a site-specific disrupting agent comprises a targeting moiety. In some embodiments, a site-specific disrupting agent comprises an effector moiety. In some embodiments, a site-specific disrupting agent comprises a plurality of effector moieties. In some embodiments, a site-specific disrupting agent comprises a targeting moiety and one or more effector moieties. In some embodiments, the site-specific disrupting agent specifically binds a first site in the genome with higher affinity than a second site in the genome (e.g., relative to any other site in the genome). In some embodiments, the site-specific disrupting agent preferentially inhibits, dissociates, degrades, and/or modifies one or more components of a first genomic complex relative to a second genomic complex (e.g., relative to any other genomic complex).

Domain: As used herein, the term “domain” refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively, or additionally, in some embodiments, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is or comprises a section or portion of a molecule (e.g., a small molecule, carbohydrate, lipid, nucleic acid, polypeptide, etc.). In some embodiments, a domain is or comprises a section of a polypeptide. In some such embodiments, a domain is characterized by a particular structural element (e.g., a particular amino acid sequence or sequence motif, alpha-helix character, beta-sheet character, coiled-coil character, random coil character, etc.), and/or by a particular functional feature (e.g., binding activity, enzymatic activity, folding activity, signaling activity, etc.).

Effector moiety: As used herein, the term “effector moiety” refers to a domain with one or more functionalities that modulate, e.g., decrease, expression of a target plurality of genes in a cell when appropriately localized in the nucleus of a cell. In some embodiments, an effector moiety comprises a polypeptide. In some embodiments, an effector moiety comprises a polypeptide and a nucleic acid. A functionality associated with an effector moiety may directly affect expression of a target plurality of genes, e.g., blocking recruitment of a transcription factor that would stimulate expression of the gene. A functionality associated with an effector moiety may indirectly affect expression of a target plurality of genes, e.g., introducing epigenetic modifications or recruiting other factors that introduce epigenetic modifications that induce a change in chromosomal topology that inhibits expression of a target plurality of genes.

Genomic complex: As used herein, the term “genomic complex” is a complex that brings together two genomic sequence elements that are spaced apart from one another on one or more chromosomes, via interactions between and among a plurality of protein and/or other components (potentially including, the genomic sequence elements). In some embodiments, the genomic sequence elements are anchor sequences to which one or more protein components of the complex binds. In some embodiments, a genomic complex may comprise an anchor sequence-mediated conjunction. In some embodiments, a genomic sequence element may be or comprise a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s). As will be understood by those skilled in the art, in some embodiments, co-localization (e.g., conjunction) of the genomic sites via formation of the complex alters DNA topology at or near the genomic sequence element(s), including, in some embodiments, between them. In some embodiments, a genomic complex comprises an anchor sequence-mediated conjunction, which comprises one or more loops. In some embodiments, a genomic complex as described herein is nucleated by a nucleating polypeptide such as, for example, CTCF and/or Cohesin. In some embodiments, a genomic complex as described herein may include, for example, one or more of CTCF, Cohesin, non-coding RNA (e.g., eRNA), transcriptional machinery proteins (e.g., RNA polymerase, one or more transcription factors, for example selected from the group consisting of TFIIA, TFIIB, TFIID, TFIIE, TFIIF, TFIIH, etc.), transcriptional regulators (e.g., Mediator, P300, enhancer-binding proteins, repressor-binding proteins, histone modifiers, etc.), etc. In some embodiments, a genomic complex as described herein includes one or more polypeptide components and/or one or more nucleic acid components (e.g., one or more RNA components), which may, in some embodiments, be interacting with one another and/or with one or more genomic sequence elements (e.g., anchor sequences, promoter sequences, regulatory sequences (e.g., enhancer sequences)) so as to constrain a stretch of genomic DNA into a topological configuration (e.g., a loop) that it does not adopt when the complex is not formed.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, the phrase “operably linked” refers to a juxtaposition wherein the components described are in a relationship permitting them to function in their intended manner. A transcription control element “operably linked” to a functional element, e.g., gene, is associated in such a way that expression and/or activity of the functional element, e.g., gene, is achieved under conditions compatible with the transcription control element. In some embodiments, “operably linked” transcription control elements are contiguous (e.g., covalently linked) with coding elements, e.g., genes, of interest; in some embodiments, operably linked transcription control elements act in trans to or otherwise at a distance from the functional element, e.g., gene, of interest. In some embodiments, operably linked means two nucleic acid sequences are comprised on the same nucleic acid molecule. In a further embodiment, operably linked may further mean that the two nucleic acid sequences are proximal to one another on the same nucleic acid molecule, e.g., within 1000, 500, 100, 50, or 10 base pairs of each other or directly adjacent to each other.

Peptide, Polypeptide, Protein: As used herein, the terms “peptide,” “polypeptide,” and “protein” refer to a compound comprised of amino acid residues covalently linked by peptide bonds, or by means other than peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds or by means other than peptide bonds. As used herein, the term refers to both short chains, which also commonly are referred to in the art as peptides, oligopeptides and oligomers, for example, and to longer chains, which generally are referred to in the art as proteins, of which there are many types.

Proximal: As used herein, “proximal” refers to a closeness of two sites, e.g., nucleic acid sites, such that binding of a site-specific disrupting agent at the first site and/or modification of the first site by a site-specific disrupting agent will produce the same or substantially the same effect as binding and/or modification of the other site. For example, a DNA-targeting moiety may bind to a first site that is proximal to an anchor sequence (the second site), and the effector moiety associated with said DNA-targeting moiety may epigenetically modify the first site such that the binding of the anchor sequence to an endogenous nucleating polypeptide modified, substantially the same as if the second site (the anchor sequence) had been bound and/or modified. In some embodiments, sites that are proximal to one another are less than 5000, 4000, 3000, 2000, 1000, 900, 800, 700, 600, 500, 400, 300, 200, 100, 50, 40, 30, 20, 10, or 5 base pairs from one another.

Sequence targeting polypeptide: As used herein, the term “sequence targeting polypeptide” as used herein, refers to a protein, such as an enzyme, e.g., Cas9 or a TALEN, that recognizes or specifically binds to a target nucleic acid sequence. In some embodiments, the sequence targeting polypeptide is a catalytically inactive protein, such as dCas9, a TAL effector molecule, or a Zn Finger molecule, that lacks endonuclease activity.

Specific binding: As used herein, the term “specific binding” refers to an ability to discriminate between possible binding partners in the environment in which binding is to occur. In some embodiments, a binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex. In some embodiments, specific binding is assessed by detecting or determining ability of the binding agent to compete with an alternative interaction between its partner and another entity. In some embodiments, specific binding is assessed by performing such detections or determinations across a range of concentrations.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” may therefore be used in some embodiments herein to capture potential lack of completeness inherent in many biological and chemical phenomena.

Symptoms are reduced: As used herein, the phrase “symptoms are reduced” may be used when one or more symptoms of a particular disease, disorder or condition is reduced in magnitude (e.g., intensity, severity, etc.) and/or frequency. In some embodiments, a delay in the onset of a particular symptom is considered one form of reducing the frequency of that symptom.

Target: An agent or entity is considered to “target” another agent or entity, in accordance with the present disclosure, if it binds specifically to the targeted agent or entity under conditions in which they come into contact with one another. In some embodiments, for example, an antibody (or antigen-binding fragment thereof) targets its cognate epitope or antigen. In some embodiments, a nucleic acid having a particular sequence targets a nucleic acid of substantially complementary sequence. In some embodiments, a targeting moiety that specifically binds an anchor sequence targets the anchor sequence, the ASMC comprising the anchor sequence, and/or the plurality of genes within the ASMC.

Target plurality of genes: As used herein, the term “target plurality of genes” means a group of more than one gene (e.g., 2, 3, 4, 5, 6, 7, 8, 9, or more genes) that is targeted for modulation, e.g., of expression. In some embodiments, a target plurality of genes is part of a targeted genomic complex. In some embodiments, each gene of a target plurality of genes has at least part (e.g., part or all) of its genomic sequence as part of a target genomic complex, e.g., at least partly inside an ASMC, which genomic complex is targeted by a site-specific disrupting agent as described herein. In some embodiments, modulation comprises inhibition of expression of the target plurality of genes. In some embodiments, a target plurality of genes is modulated by contacting the target plurality of genes or a transcription control element operably linked to one or more of the target plurality of genes with a site-specific disrupting agent described herein. In some embodiments, one or more of a target plurality of genes is aberrantly expressed (e.g., over-expressed) in a cell, e.g., a cell in a subject (e.g., patient). In some embodiments, the target plurality of genes has related functionalities. For example, the genes of a target plurality of genes may all have a pro-inflammatory effect when expressed; the genes of such a target plurality of genes may be referred to herein as pro-inflammatory genes or target pro-inflammatory genes. In some embodiments, a gene of a target plurality of genes encodes a protein. In some embodiments, a gene of a target plurality of genes encodes a functional RNA.

Targeting moiety: As used herein, the term “targeting moiety” means an agent or entity that specifically interacts (e.g., targets) with a component or set of components, e.g., a component or components that participate in a genomic complex as described herein (e.g., an anchor sequence-mediated conjunction). In some embodiments, a targeting moiety in accordance with the present disclosure targets one or more target component(s) of a genomic complex as described herein. In some embodiments, a targeting moiety targets a genomic complex component that comprises a genomic sequence element (e.g., an anchor sequence). In some embodiments, a targeting moiety targets a genomic complex component other than a genomic sequence element. In some embodiments, a targeting moiety targets a plurality or combination of genomic complex components, which plurality in some embodiments may include a genomic sequence element. In some aspects, contributions of the present disclosure include the insight that inhibition, dissociation, degradation, and/or modification of one or more genomic complexes, e.g., comprising a target anchor sequence proximal to a target gene (e.g., fusion gene, e.g., fusion oncogene) and/or breakpoint, as described herein, can be achieved by targeting genomic complex component(s), including genomic sequence element(s), with site-specific disrupting agents. In some aspects, effective inhibition, dissociation, degradation, and/or modification of one or more genomic complexes, as described herein, can be achieved by targeting complex component(s) comprising genomic sequence element(s). In some embodiments, the present disclosure contemplates that improved (e.g., with respect to, for example, degree of specificity for a particular genomic complex as compared with other genomic complexes that may form or be present in a given system, effectiveness of the inhibition, dissociation, degradation, or modification [e.g., in terms of impact on number of complexes detected in a population]) inhibition, dissociation, degradation, or modification may be achieved by targeting one or more complex components that is not a genomic sequence element and, optionally, may alternatively or additionally include targeting a genomic sequence element, wherein improved inhibition, dissociation, degradation, or modification is relative to that typically achieved through targeting genomic sequence element(s) alone. In some embodiments, a site-specific disrupting agent as described herein promotes inhibition, dissociation, degradation, or modification of a target genomic complex. For example, by way of non-limiting example, in some embodiments, a site-specific disrupting agent as described herein inhibits, dissociates, degrades (e.g., a component of), and/or modifies (e.g., a component of) an anchor sequence-mediated conjunction by targeting at least one component of a given genomic complex (e.g., comprising the anchor sequence-mediated conjunction). In some embodiments, a site-specific disrupting agent as described herein inhibits, dissociates, degrades (e.g., a component of), and/or modifies (e.g., a component of) a particular genomic complex (i.e., a target genomic complex) and does not inhibit, dissociate, degrade (e.g., a component of), and/or modify (e.g., a component of) at least one other particular genomic complex (i.e., a non-target genomic complex) that, for example, may be present in other cells (e.g., in non-target cells) and/or that may be present at a different site in the same cell (i.e., within a target cell). A site-specific disrupting agent as described herein may comprise a targeting moiety. In some embodiments, a targeting moiety also acts as an effector moiety (e.g., disrupting moiety); in some such embodiments a provided site-specific disrupting agent may lack any effector moiety (e.g., disrupting, modifying, or other effector moiety) separate (or meaningfully distinct) from the targeting moiety.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, an effective amount of a substance may vary depending on such factors as desired biological endpoint(s), substance to be delivered, target cell(s) or tissue(s), etc. For example, in some embodiments, an effective amount of compound in a formulation to treat a disease, disorder, and/or condition is an amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Transcriptional control sequence: As used herein, the term “transcriptional control sequence” refers to a nucleic acid sequence that increases or decreases transcription of a gene. An “enhancing sequence” increases the likelihood of gene transcription. A “silencing or repressor sequence” decreases the likelihood of gene transcription. Examples of transcriptional control sequences include promoters and enhancers. In some embodiments, an ASMC comprises a transcriptional control sequence. Such a transcriptional control sequence is referred to as an internal transcriptional control sequence (e.g., an enhancing sequence that is comprised within an ASMC is referred to as an internal enhancing sequence).

### BRIEF DESCRIPTION OF THE DRAWINGS

The following detailed description of the embodiments of the disclosure will be better understood when read in conjunction with the appended drawings. For the purpose of illustrating the disclosure, there are shown in the drawings embodiments, which are presently exemplified. It should be understood, however, that the disclosure is not limited to the precise arrangement and instrumentalities of the embodiments shown in the drawings.

FIG. 1 shows a diagram showing exemplary positioning of gRNA sequences in an anchor sequence. Figure discloses SEQ ID NOS 244-245, respectively, in order of appearance.

FIG. 2 shows a diagram showing exemplary positioning of gRNA sequences in an anchor sequence and restriction site information. Figure discloses SEQ ID NOS 246-247, respectively, in order of appearance.

FIG. 3 shows a graph of expression (mRNA) of various chemokines in TNF-treated cells with and without treatment with a site-specific disrupting agent comprising a CRISPR/Cas molecule and first exemplary gRNA.

FIG. 4 shows a graph of expression (mRNA) of various chemokines in TNF-treated cells with and without treatment with a site-specific disrupting agent comprising a CRISPR/Cas molecule and a second exemplary gRNA.

FIG. 5 shows a diagram depicting different types of genomic complex, e.g., ASMCs, e.g., loops, and models for how to alter expression of genes contained within.

FIG. 6 shows a graph of cytokine expression measured by RNA levels of CXCL1, CXCL2, CXCL3, and IL-8 in THP-1 cells treated with a site-specific disrupting agent comprising a CRISPR/Cas molecule and sgRNAs targeted to the anchor sequences of a genomic complex (e.g., ASMC) comprising cytokine-encoding genes.

FIG. 7 shows a graph of cytokine secretion (CXCL1 and IL-8) of THP-1 cells treated with site-specific disrupting agent comprising a CRISPR/Cas molecule and different sgRNAs targeted to the anchor sequences of a genomic complex (e.g., ASMC) comprising cytokine-encoding genes.

FIG. 8 shows a graph (top) of cytokine expression (CXCL3) measured by RNA level in THP-1 cells treated with a site-specific disrupting agent comprising a CRISPR/Cas molecule and sgRNAs targeted to the anchor sequences of a genomic complex (e.g., ASMC) comprising cytokine-encoding genes, and a flow chart (bottom) showing how cells were processed in the experiment.

FIG. 9A shows a graph (top) of cytokine expression (CXCL1) measured by RNA level in THP-1 cells 3 weeks after treatment with a site-specific disrupting agent comprising a CRISPR/Cas molecule and sgRNAs targeted to the anchor sequences of a genomic complex (e.g., ASMC) comprising cytokine-encoding genes, and a flow chart (bottom) showing how cells were processed in the experiment. FIG. 9B shows a graph of cytokine expression (CXCL3) measured by RNA level in THP-1 cells 3 weeks after treatment with a site-specific disrupting agent comprising a CRISPR/Cas molecule and sgRNAs targeted to the anchor sequences of a genomic complex (e.g., ASMC) comprising cytokine-encoding genes.

FIG. 10 shows a graph of cytokine expression (CXCL1) measured by RNA level in THP-1 cells after treatment with a site-specific disrupting agent comprising a catalytically inactive CRISPR/Cas molecule and a transcriptional repressor (KRAB) and sgRNAs targeted to the anchor sequences of a genomic complex (e.g., ASMC) comprising cytokine-encoding genes.

FIG. 11 shows a graph of cytokine expression (CXCL1) measured by RNA level in THP-1 cells after treatment with a site-specific disrupting agent comprising a catalytically inactive CRISPR/Cas molecule and a histone methyltransferase (EZH2) and sgRNAs targeted to the anchor sequences of a genomic complex (e.g., ASMC) comprising cytokine-encoding genes.

FIG. 12 shows a graph of cytokine expression (CXCL1) measured by RNA level in THP-1 cells after treatment with a site-specific disrupting agent comprising a catalytically inactive CRISPR/Cas molecule and a DNA methyltransferase (MQ1) and sgRNAs targeted to the anchor sequences of a genomic complex (e.g., ASMC) comprising cytokine-encoding genes.

FIG. 13 shows a graph (top) of cytokine expression (CXCL1) measured by RNA level in THP-1 cells after treatment with different site-specific disrupting agents for 72 hours, 3 weeks, or 4 weeks, and a flow chart (bottom) showing how cells were processed in the experiment.

FIG. 14 shows a graph (top) of cytokine expression (CXCL3) measured by RNA level in THP-1 cells after treatment with different site-specific disrupting agents and sgRNAs targeted to the anchor sequences of a genomic complex (e.g., ASMC) comprising cytokine-encoding genes, and a flow chart (bottom) showing how cells were processed in the experiment.

FIG. 15 shows a graph (top) of cytokine expression (CXCL1) measured by RNA level in THP-1 cells after treatment with different site-specific disrupting agents and sgRNAs targeted to the anchor sequences of a genomic complex (e.g., ASMC) comprising cytokine-encoding genes.

FIG. 16 shows human CXCL IGD and gene cluster organization. FIG. 16A shows a schematic Insulated Genomic Domain (IGD) illustrating the two loops within CXLC1-8 gene cluster. CXCL8, CXCL6, and CXCL 1 genes reside on the left loop of the IGD. CXCL2-5 and CXCL7 genes reside on the right loop of the IGD. Investigation of the IGD data from different cell lines suggested that middle CTCF is only present in cells that secrete CXCL (e.g., not in lymphocytes).

FIG. 16B shows guides were designed to the four different CTCF targets: Left CTCF-2, Left CTCF, Middle CTCF, and Right CTCF.

FIG. 17 shows CXCL1-8 genes were downregulated when dCas9-EZH2 guide 30183 targeted Middle CTCF motif located within the CXCL1-8 cluster in TNF-alpha treated Human A549 lung cancer epithelial cells. Cells stimulated with TNF alpha were treated as control.

FIG. 18 shows CXCL1, 2, 3, 8 genes were downregulated when dCas9-EZH2 guide 30183 targeted Middle CTCF motif located within the CXCL1-8 cluster in TNF-alpha treated Human IMR-90 normal lung fibroblast cells. Cells stimulated with TNF alpha were treated as control.

FIG. 19 shows that CXCL1, 2, 3, 8 genes were downregulated when Controller A targeted Left CTCF motif located within the CXCL1-8 cluster in TNF-alpha treated Human monocytes. Cells stimulated with TNF alpha were treated as control.

FIG. 20 shows mouse CXCL IGD and gene cluster organization. FIG. 20A shows a schematic Insulated Genomic Domain (IGD) illustrating the two loops within CXCL gene cluster. FIG. 20B illustrates the two loops within the CXLC1-5, 7 and 15 gene cluster. CXCL4, CXCL5, and CXCL7 genes reside on the left loop of the IGD. CXCL1-3 and CXCL15 genes reside on the right loop of the IGD guides were designed to the four different CTCF targets: Left (L), Middle 1 (M1), Middle 2 (M2), and Right (R) CTCF.

FIG. 21A shows IGD guides were designed to the four different CTCF targets: Middle 1 (M1), Middle 2 (M2), and Right (R) CTCF.

FIG. 21B shows in vitro downregulation of mouse CXCL IGD in Hep 1.6 using dCas9-MQ1. dCas9-MQ1 was transfected using guides targeting the right, or one of the two middle CTCF motifs in the CXCL gene cluster, which showed no down regulation in any of the seven CXCL genes after TNF alpha stimulation (orange). When dCas9-MQ1 was transfected using combination guides targeting both middle CTCF and right, the entire gene cluster was down regulated (blue).

FIG. 22A shows schematic experimental design to determine the effect of dCas9-MQ1 on decreasing leukocyte filtration in inflamed lungs. Each mouse was treated with either LNP alone or with dCas9-MQ1 at 3 mg/kg targeting the two middle and right CTCF at −2 hour time point. The mice were simulated with 5 mg/kg LPS at zero hours followed by a second dose of LNP alone or a dCas9-MQ1 at 3 mg/kg targeting the two middle and right CTCF at the +8 hour time point. Dexamethasone was administered intraperitoneal at 10 mg/kg dose at time 0, 24, and 48 hours. The animals were terminated at 72 hours and bronchiolar lavage fluid were collected from the lungs for flow staining.

FIG. 22B shows systemic administration of an dCas9-MQ1 decreased leukocyte infiltration in the inflamed lungs. Total leukocyte count/mL in the bronchiolar lavage fluid obtained from dCas9-MQ1 treated mice showed significant differences compared to LPS+disease animals.

FIG. 23A shows the composition of infiltrating cells found in the bronchiolar lavage fluid obtained from an inflamed lung of a mice. The leukocyte cell types that make up the majority of the infiltrating cells are neutrophils, followed by B cells, T cells, macrophages and other types of hematopoietic cells.

FIG. 23B shows dCas9-MQ1 decreased the count of neutrophils infiltrating the lungs with significant difference compared to the +LPS disease group.

FIG. 24 shows the decrease of leukocyte cells in the BALF was lung specific and not due to the decrease of white blood cells in the peripheral blood. This graph illustrated that the effect of decreasing leukocyte count in the BALF with the dCas9-MQ1 treatment was lung specific and was not because the mouse itself had a decrease in leukocyte population. The hematopoietic cell population in the peripheral blood was similar across all groups.

FIGS. 25A-G show CXCL1-5, CXCL7, and CXCL15 gene expression was decreased in the lung tissue. After treating the animals with LNP alone or with dCas9-MQ1, the lung tissues were processed to check for CXCL gene expression by qPCR methods. All CXCL genes show downregulation when treated with dCA9-MQ1. CXCL2 expression was most downregulated.

FIG. 26 shows decreasing CXCL expression and cellular recruitment to the site of inflammation had a beneficial downstream effect of decreasing the presence of other cytokines. The chemokine protein levels secreted in the BALF showed decrease in CXCL 1 and 2 protein levels. Decreasing CXCL expression and cellular recruitment to the site of inflammation had beneficial downstream effects of decreasing the presence of GM-CSF (FIG. 26C) and IL6 (FIG. 26D).

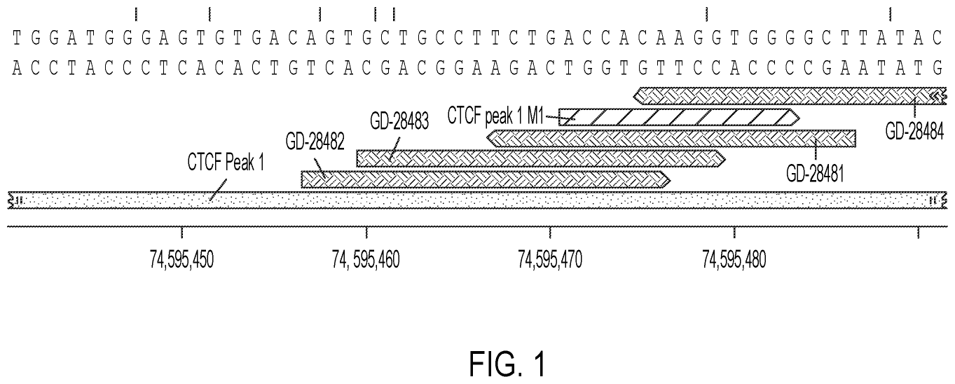

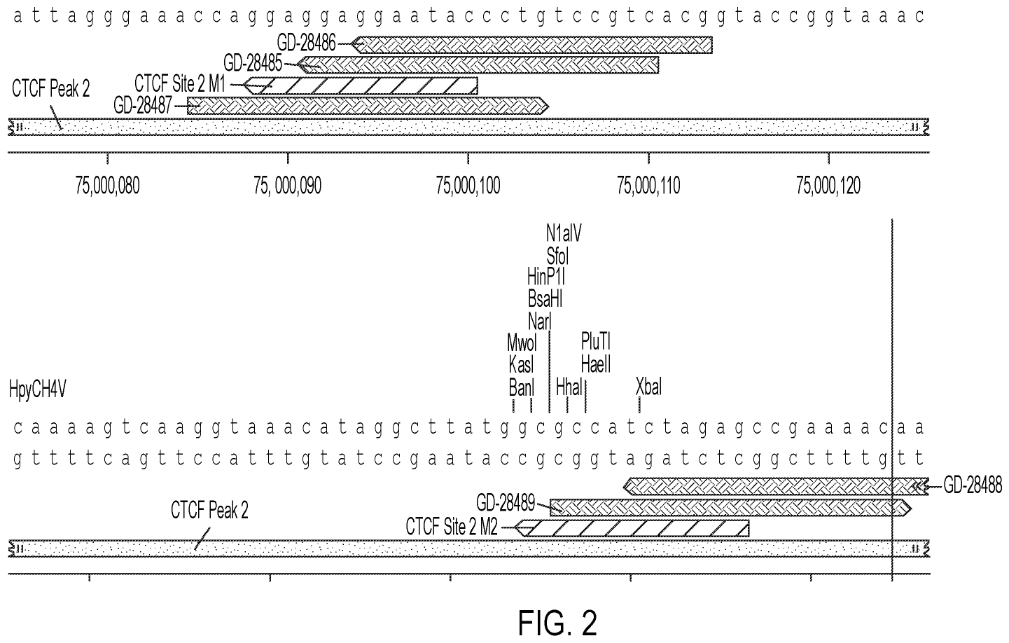

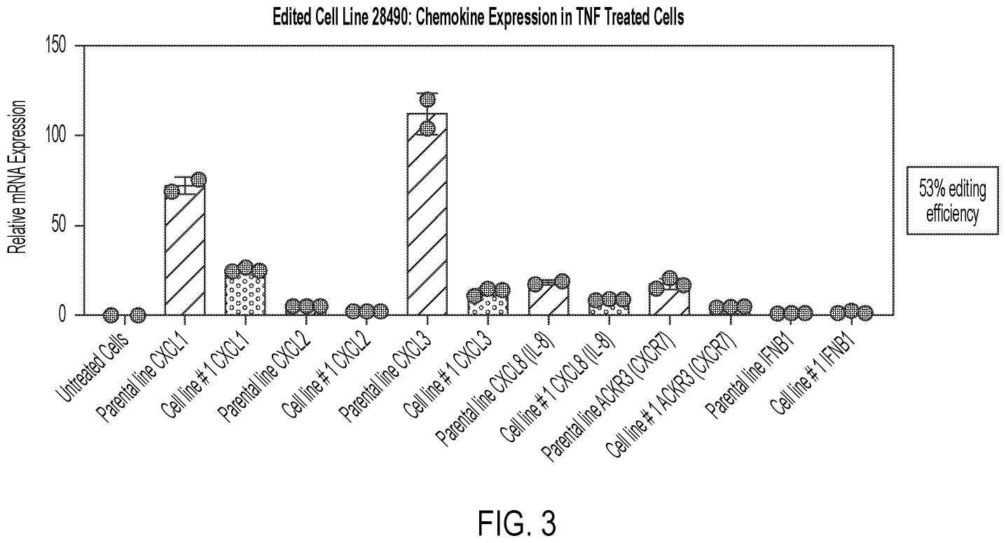

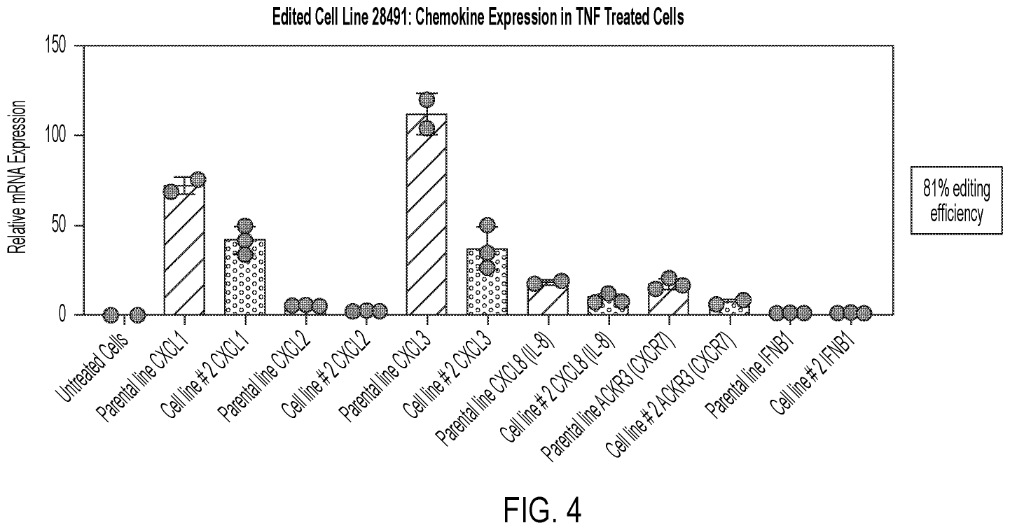

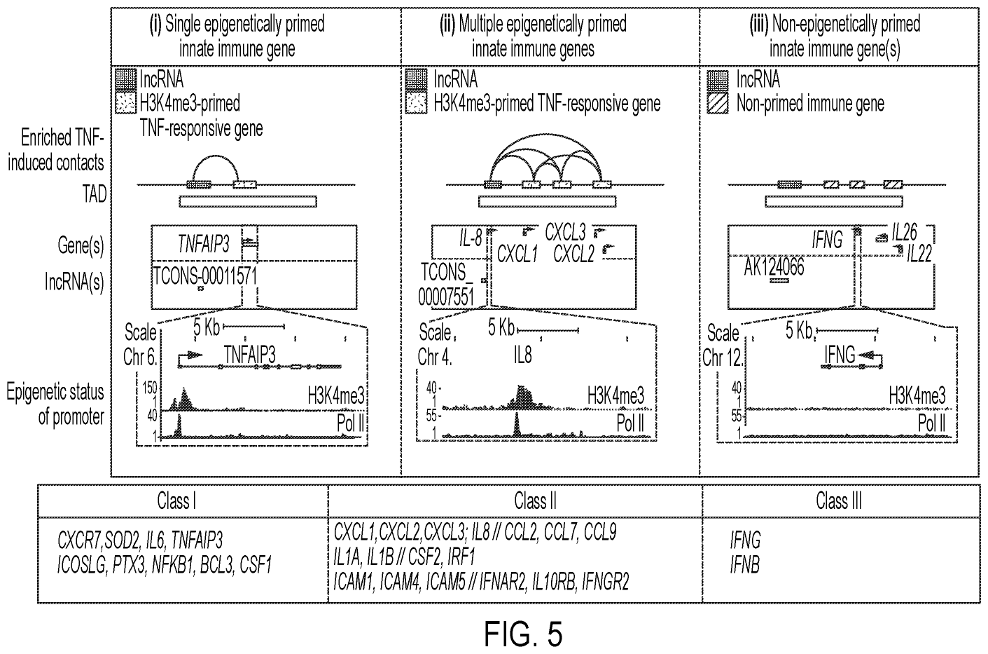

### DETAILED DESCRIPTION OF CERTAIN EMBODIMENTS

The present disclosure provides technologies for modulating, e.g., decreasing, expression of a target plurality of genes in a cell, e.g., in a subject or patient, through the use of site-specific disrupting agent or a system comprising two or more site-specific disrupting agents. In some embodiments, a site-specific disrupting agent comprises a targeting moiety. In some embodiments, a site-specific disrupting agent comprises a targeting moiety and an effector moiety. Without wishing to be bound by theory, a number of diseases and conditions are associated with groups of genes with related functionalities that are associated with a common genomic complex, e.g., ASMC. Modulation, e.g., disruption, of a genomic complex, e.g., ASMC, comprising (wholly or in part) a target plurality of genes may be an improved approach to altering (e.g., decreasing) expression of the target plurality of genes (e.g., with respect to improved efficiency, efficacy, and/or stability of alteration) over modulation of individual target genes. Said improvements may translate to corresponding improvements in the treatment of diseases and conditions associated with the target plurality of genes. For example, a plurality of genes may be associated with a pro-inflammatory effect and a site-specific disrupting agent can target a genomic complex, e.g., ASMC, comprising (wholly or in part) the plurality of genes to modulate, e.g., decrease, expression of the plurality of genes and thereby achieve an anti-inflammatory effect (e.g., a superior anti-inflammatory effect relative to individually targeting the genes of the plurality). Examples of site-specific disrupting agents, targeting moieties, effector moieties, and target pluralities of genes are provided herein.

A site-specific disrupting agent may modulate, e.g., decrease, expression of a target plurality of genes by one or more modalities. In some embodiments, a site-specific disrupting agent binds to a target site, e.g., anchor sequence, and physically or sterically competes for binding with other genomic complex components, e.g., a nucleating polypeptide. Without wishing to be bound by theory, physical or steric blockage of an anchor sequence, e.g., such that binding of a genomic complex component (e.g., a nucleating polypeptide) to the anchor sequence is inhibited (e.g., prevented), is one mechanism by which a site-specific disrupting agent may modulate, e.g., decrease, expression of a target plurality of genes. A site-specific disrupting agent may destabilize the interaction of a genomic complex component (e.g., nucleating polypeptide) with an anchor sequence, e.g., by altering (e.g., decreasing) the affinity and/or avidity at which the genomic complex component binds the anchor sequence. Blocking or destabilizing binding of a genomic complex component (e.g., nucleating polypeptide) to an anchor sequence may be accomplished by one or more means, including: epigenetic modification of the anchor sequence or a sequence proximal thereto, genetic modification of the anchor sequence or a sequence proximal thereto, or binding of the site-specific disrupting agent to the anchor sequence or a sequence proximal thereto. Inhibiting (e.g., preventing) binding of a genomic complex component (e.g., a nucleating polypeptide) to an anchor sequence may inhibit (e.g., disrupt or prevent formation of) a genomic complex, e.g., ASMC. Inhibition of a genomic complex, e.g., ASMC, comprising, wholly or partly, a target plurality of genes may modulate, e.g., decrease, expression of the genes of the target plurality of genes. In some embodiments, a site-specific disrupting agent comprises a targeting moiety, a first effector moiety, and a second effector moiety. In some embodiments, the first effector moiety has a sequence that is different from the sequence of the second effector moiety. In some embodiments, the first effector moiety has a sequence that is identical to the sequence of the second effector moiety.

The disclosure further provides in part, a system comprising two or more site-specific disrupting agents, each comprising a targeting moiety and optionally an effector moiety. In some embodiments, the targeting moieties target two or more different sequences (e.g., each site-specific disrupting agent may target a different sequence). In some embodiments, the first site-specific disrupting agent binds to a transcription regulatory element (e.g., a promoter or transcription start site (TSS)) operably linked to a target plurality of genes, e.g., human CXCL1-8 and the second site-specific disrupting agent binds to an anchor sequence of an anchor sequence mediated conjunction (ASMC) comprising a target plurality of genes, e.g., human CXCL1-8. In some embodiments, modulation of expression of a target plurality of genes, e.g., human CXCL 1-8 by a system involves the binding of the first site-specific disrupting agent and second site-specific disrupting agent to the first and second DNA sequences, respectively. Binding of the first and second DNA sequences localizes the functionalities of the first and second effector moieties to those sites. Without wishing to be bound by theory, in some embodiments employing the functionalities of both the first and second site-specific disrupting agent effector moieties stably represses expression of a target plurality of gene associated with or comprising the first and/or second DNA sequences, e.g., wherein the first and/or second DNA sequences are or comprise sequences of the target plurality of gene or one or more operably linked transcription control elements.

Site-Specific Disrupting Agents

In some embodiments, a site-specific disrupting agent comprises a targeting moiety. In some embodiments, the targeting moiety specifically binds a DNA sequence, e.g., an anchor sequence, and thereby modulates, e.g., disrupts, a genomic complex (e.g., ASMC) comprising said DNA sequence. In some embodiments, a site-specific disrupting agent comprises a targeting moiety and an effector moiety. In some embodiments, the targeting moiety specifically binds a DNA sequence, thereby localizing the effector moiety's functionality to the DNA sequence, thereby modulating, e.g., disrupting, a genomic complex (e.g., ASMC) comprising said DNA sequence. In some embodiments, a site-specific disrupting agent comprises one targeting moiety and one effector moiety. In some embodiments, a site-specific disrupting agent comprises one targeting moiety and more than one effector moiety, e.g., two, three, four, or five effector moieties, each of which may be the same or different from another of the more than one effector moieties. In some embodiments, a site-specific disrupting agent may comprise two effector moieties where the first effector moiety comprises a different functionality than the second effector moiety. For example, a site-specific disrupting agent may comprise two effector moieties, where the first effector moiety comprises DNA methyltransferase functionality (e.g., comprises G9A or EZH2 or a functional fragment or variant thereof) and the second effector moiety comprises a transcriptional repressor functionality (e.g., comprises KRAB or a functional fragment or variant thereof). In some embodiments, a site-specific disrupting agent comprises effector moieties whose functionalities are complementary to one another with regard to decreasing expression of a target plurality of gene, where the functionalities together inhibit expression and, optionally, do not inhibit or negligibly inhibit expression when present individually. In some embodiments, a site-specific disrupting agent comprises a plurality of effector moieties, wherein each effector moiety complements each other effector moiety, each effector moiety decreases expression of a target plurality of gene.

In some embodiments, a site-specific disrupting agent comprises a combination of effector moieties whose functionalities synergize with one another with regard to decreasing expression of a target plurality of gene. Without wishing to be bound by theory, in some embodiments, epigenetic modifications to a genomic locus are cumulative, in that multiple transcription activating epigenetic markers (e.g., multiple different types of epigenetic markers and/or more extensive marking of a given type) individually together inhibit expression more effectively than individual modifications alone (e.g., producing a greater decrease in expression and/or a longer-lasting decrease in expression). In some embodiments, a site-specific disrupting agent comprises a plurality of effector moieties, wherein each effector moiety synergizes with each other effector moiety, e.g., each effector moiety decreases expression of a target plurality of gene. In some embodiments, a site-specific disrupting agent (comprising a plurality of effector moieties which synergize with one another) is more effective at inhibiting expression of a target plurality of gene, than a site-specific disrupting agent comprising an individual effector moiety. In some embodiments, a site-specific disrupting agent comprising said plurality of effector moieties is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at decreasing expression of a target plurality of gene, than a site-specific disrupting agent comprising an individual effector moiety.

In some embodiments, a site-specific disrupting agent comprises one or more targeting moieties e.g., a Cas domain, TAL effector domain, or Zn Finger domain. In an embodiment, when system comprises two or more targeting moieties of the same type, e.g., two or more Cas domains, the targeting moieties specifically bind two or more different sequences. For example, in a site-specific disrupting agent system comprising two or more Cas domains, the two or more Cas domains may be chosen or altered such that they only appreciably bind the gRNA corresponding to their target sequence (e.g., and do not appreciably bind the gRNA corresponding to the target of another Cas domain).

In some embodiments, a site-specific disrupting agent comprises a targeting moiety and an effector moiety that are covalently linked, e.g., by a peptide bond. In some embodiments, the targeting moiety and effector moiety are situated on the same polypeptide chain, e.g., connected by one or more peptide bonds and/or a linker. In some embodiments, a site-specific disrupting agent comprises a fusion molecule, e.g., comprising the targeting moiety and effector moiety linked by a peptide bond and/or a linker. In some embodiments, a site-specific disrupting agent comprises a targeting moiety that is disposed N-terminal of an effector moiety on the same polypeptide chain. In some embodiments, a site-specific disrupting agent comprises a targeting moiety that is disposed C-terminal of an effector moiety on the same polypeptide chain. In some embodiments, a site-specific disrupting agent comprises a targeting moiety and an effector moiety that are covalently linked by a non-peptide bond. In some embodiments, a targeting moiety is conjugated to an effector moiety by a non-peptide bond. In some embodiments, a site-specific disrupting agent comprises a targeting moiety and a plurality of effector moieties, wherein the targeting moiety and the plurality of effector moieties are covalently linked, e.g., by peptide bonds (e.g., the targeting moiety and plurality of effector moieties are all connected by a series of covalent bonds, although each individual moiety may not share a covalent bond with every other moiety).

In other embodiments, a site-specific disrupting agent comprises a targeting moiety and an effector moiety that are not covalently linked, e.g., that are non-covalently associated with one another. In some embodiments, a site-specific disrupting agent comprises a targeting moiety that non-covalently binds to an effector moiety or vice versa. In some embodiments, a site-specific disrupting agent comprises a targeting moiety and a plurality of effector moieties, wherein the targeting moiety and at least one effector moiety are not covalently linked, e.g., are non-covalently associated with one another, and wherein the targeting moiety and at least one other effector moiety are covalently linked, e.g., by a peptide bond.

In some embodiments, a site-specific disrupting agent comprises a first effector moiety comprising G9A and a second effector moiety comprising KRAB. In some embodiments, a site-specific disrupting agent comprises a first effector moiety comprising G9A and a second effector moiety comprising EZH2. In some embodiments, a site-specific disrupting agent comprises a first effector moiety comprising EZH2 and a second effector moiety comprising KRAB.

In some embodiments, a site-specific disrupting agent comprises a targeting moiety and an effector moiety, wherein the C-terminal end of the effector moiety, e.g., an effector moiety chosen from, EZH2, or G9A or a functional variant or fragment thereof and the N-terminal end of the targeting moiety are covalently linked. In some embodiments, a site-specific disrupting agent comprises a targeting moiety and an effector moiety wherein the N-terminal end of the effector moiety, e.g., an effector moiety chosen from HDAC8, MQ1, DNMT3a/3L, KRAB, or a functional variant or fragment thereof and the C-terminal end of the targeting moiety are covalently linked. In some embodiments, a site-specific disrupting agent comprises a targeting moiety, a first effector moiety and a second effector moiety, wherein, the C-terminal end of the first effector moiety, e.g., a effector moiety chosen from EZH2, G9A, or a functional variant or fragment thereof, and the N-terminal end of the targeting moiety are covalently linked and the C-terminal end of the targeting moiety and the N-terminal end of the second effector moiety, e.g., a effector moiety chosen from HDAC8, MQ1, DNMT3a/3L, KRAB or a functional variant or fragment thereof are covalently linked. The covalent linkage may be, e.g., via a linker sequence.

In some embodiments, a site-specific disrupting agent comprises a targeting moiety, a first effector moiety and a second effector moiety, wherein the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is KRAB, or a functional variant or fragment thereof, e.g., wherein the second effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is EZH2, or a functional variant or fragment thereof, e.g., wherein the first effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is HDAC8, or a functional variant or fragment thereof, e.g., wherein the second effector moiety comprises an amino acid sequence of SEQ ID NO: 19 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is KRAB, or a functional variant or fragment thereof, e.g., wherein the second effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, a site-specific disrupting agent comprises a targeting moiety, a first effector moiety and a second effector moiety, wherein the first effector moiety is G9A, or a functional variant or fragment thereof, e.g., wherein the first effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the first effector moiety is N-terminal of the targeting moiety; and the second effector moiety is EZH2, or a functional variant or fragment thereof, e.g., wherein the second effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, and the second effector moiety is C-terminal of the targeting moiety.

In some embodiments, the first effector moiety comprises a histone methyltransferase activity and the second effector moiety comprises a different histone methyltransferase activity. In some embodiments, the first effector moiety comprises a histone methyltransferase activity and the second effector moiety comprises the same histone methyltransferase activity. In some embodiments, the first effector moiety comprises a histone demethylase activity and the second effector moiety comprises a histone deacetylase activity. In some embodiments, the first effector moiety comprises a histone demethylase activity and the second effector moiety comprises a DNA methyltransferase activity. In some embodiments, the first effector moiety comprises a histone demethylase activity and the second effector moiety comprises a DNA demethylase activity. In some embodiments, the first effector moiety comprises a histone demethylase activity and the second effector moiety comprises a transcription repressor activity. In some embodiments, the first effector moiety comprises a histone demethylase activity and the second effector moiety comprises a different histone demethylase activity. In some embodiments, the first effector moiety comprises a histone demethylase activity and the second effector moiety comprises the same histone demethylase activity. In some embodiments, the first effector moiety comprises a histone deacetylase activity and the second effector moiety comprises a DNA methyltransferase activity. In some embodiments, the first effector moiety comprises a histone deacetylase activity and the second effector moiety comprises a DNA demethylase activity. In some embodiments, the first effector moiety comprises a histone deacetylase activity and the second effector moiety comprises a transcription repressor activity. In some embodiments, the first effector moiety comprises a histone deacetylase activity and the second effector moiety comprises a different histone deacetylase activity. In some embodiments, the first effector moiety comprises a histone deacetylase activity and the second effector moiety comprises the same histone deacetylase activity. In some embodiments, the first effector moiety comprises a DNA methyltransferase activity and the second effector moiety comprises a DNA demethylase activity. In some embodiments, the first effector moiety comprises a DNA methyltransferase activity and the second effector moiety comprises a transcription repressor activity. In some embodiments, the first effector moiety comprises a DNA methyltransferase activity and the second effector moiety comprises a different DNA methyltransferase activity. In some embodiments, the first effector moiety comprises a DNA methyltransferase activity and the second effector moiety comprises the same DNA methyltransferase activity. In some embodiments, the first effector moiety comprises a DNA demethylase activity and the second effector moiety comprises a transcription repressor activity. In some embodiments, the first effector moiety comprises a DNA demethylase activity and the second effector moiety comprises a different DNA demethylase activity. In some embodiments, the first effector moiety comprises a DNA demethylase activity and the second effector moiety comprises the same DNA demethylase activity. In some embodiments, the first effector moiety comprises a transcription repressor activity and the second effector moiety comprises a different transcription repressor activity. In some embodiments, the first effector moiety comprises a transcription repressor activity and the second effector moiety comprises the same transcription repressor activity.

In some embodiments, the first effector moiety comprises, DNMT3a/31, MQ1, KRAB, G9A, HDAC8, or EZH2 and the second effector moiety comprises DNMT3a/31, MQ1, KRAB, G9A, HDAC8, or EZH2.

### Linkers

A site-specific disrupting agent may comprise one or more linkers. A linker may connect a targeting moiety to an effector moiety, an effector moiety to another effector moiety, or a targeting moiety to another targeting moiety. A linker may be a chemical bond, e.g., one or more covalent bonds or non-covalent bonds. In some embodiments, a linker is covalent. In some embodiments, a linker is non-covalent. In some embodiments, a linker is a peptide linker. Such a linker may be between 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, 5-10, or 2-5 amino acids in length, or greater than or equal to 2, 5, 10, 15, 20, 25, or 30 amino acids in length (and optionally up to 50, 40, 30, 25, 20, 15, 10, or 5 amino acids in length). In some embodiments, a linker can be used to space a first moiety from a second moiety, e.g., a targeting moiety from an effector moiety. In some embodiments, for example, a linker can be positioned between a targeting moiety and an effector moiety, e.g., to provide molecular flexibility of secondary and tertiary structures. In some embodiments, a site-specific disrupting agent may comprise a first effector moiety linked to the targeting moiety via a first linker and a second effector moiety linked to the targeting moiety via a second linker. In some embodiments, the first linker has a sequence that is identical to the sequence of the second linker. In some embodiments, the first linker has a sequence that is not identical to the sequence of the second linker. In some embodiments, the first effector moiety is N-terminal of the targeting moiety. In some embodiments, the C-terminal of the targeting moiety. In some embodiments, the C-terminal end of the first effector moiety is linked to the N-terminal end of the targeting moiety via the first linker and the N-terminal end of the second effector moiety is linked to the C-terminal end of the targeting moiety via the second linker.

A linker may comprise flexible, rigid, and/or cleavable linkers described herein. In some embodiments, a linker includes at least one glycine, alanine, and serine amino acids to provide for flexibility. In some embodiments, a linker is a hydrophobic linker, such as including a negatively charged sulfonate group, polyethylene glycol (PEG) group, or pyrophosphate diester group. In some embodiments, a linker is cleavable to selectively release a moiety (e.g., polypeptide) from a modulating agent, but sufficiently stable to prevent premature cleavage.

In some embodiments, one or more moieties of a site-specific disrupting agent described herein are linked with one or more linkers.

As will be known by one of skill in the art, commonly used flexible linkers have sequences consisting primarily of stretches of Gly and Ser residues (“GS” linker). Flexible linkers may be useful for joining domains/moieties that require a certain degree of movement or interaction and may include small, non-polar (e.g., Gly) or polar (e.g., Ser or Thr) amino acids. Incorporation of Ser or Thr can also maintain the stability of a linker in aqueous solutions by forming hydrogen bonds with water molecules, and therefore reduce unfavorable interactions between a linker and moieties/domains.

Rigid linkers are useful to keep a fixed distance between domains/moieties and to maintain their independent functions. Rigid linkers may also be useful when a spatial separation of domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains/moieties in vivo. In some embodiments, linkers may be cleaved under specific conditions, such as presence of reducing reagents or proteases. In vivo cleavable linkers may utilize reversible nature of a disulfide bond. One example includes a thrombin-sensitive sequence (e.g., PRS) between the two Cys residues. In vitro thrombin treatment of CPRSC (SEQ ID NO: 243) results in the cleavage of a thrombin-sensitive sequence, while a reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under certain conditions, in specific cells or tissues, or constrained within certain cellular compartments. Specificity of many proteases offers slower cleavage of the linker in constrained compartments.

Examples of molecules suitable for use in linkers described herein include a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof; noncarbon linkers; carbohydrate linkers; phosphodiester linkers, or other molecule capable of covalently linking two or more components of a site-specific disrupting agent. Non-covalent linkers are also included, such as hydrophobic lipid globules to which the polypeptide is linked, for example through a hydrophobic region of a polypeptide or a hydrophobic extension of a polypeptide, such as a series of residues rich in leucine, isoleucine, valine, or perhaps also alanine, phenylalanine, or even tyrosine, methionine, glycine, or other hydrophobic residue. Components of a site-specific disrupting agent may be linked using charge-based chemistry, such that a positively charged component of a site-specific disrupting agent is linked to a negative charge of another component.

### Nucleic Acids

In one aspect, the disclosure provides nucleic acid sequences encoding a site-specific disrupting agent, a system, a targeting moiety and/or an effector moiety as described herein. A skilled artisan is aware that the nucleic acid sequences of RNA are identical to the corresponding DNA sequences, except that typically thymine (T) is replaced by uracil (U). It will be understood that when a nucleotide sequence is represented by a DNA sequence (e.g., comprising, A, T, G, C), this disclosure also provides the corresponding RNA sequence (e.g., comprising, A, U, G, C) in which “U” replaces “T.” Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

It will be appreciated by those skilled in the art that due to the degeneracy of the genetic code, a multitude of nucleotide sequences encoding a site-specific disrupting agent comprising DNA-targeting moiety and/or an effector moiety as described herein may be produced, some of which have similarity, e.g., 90%, 95%, 96%, 97%, 98%, or 99% identity to the nucleic acid sequences disclosed herein. For instance, codons AGA, AGG, CGA, CGC, CGG, and CGU all encode the amino acid arginine. Thus, at every position in the nucleic acid molecules of the disclosure where an arginine is specified by a codon, the codon can be altered to any of the corresponding codons described above without altering the encoded polypeptide.

In some embodiments a nucleic acid sequence encoding a site-specific disrupting agent comprising a targeting moiety and/or one or more effector moieties may be part or all of a codon-optimized coding region, optimized according to codon usage in mammals, e.g., humans. In some embodiments, a nucleic acid sequence encoding a targeting moiety and/or one or more effector moieties is codon optimized for increasing the protein expression and/or increasing the duration of protein expression. In some embodiments, a protein produced by the codon optimized nucleic acid sequence is at least 1%, at least 2%, at least 5%, at least 10%, at least 15%, at least 20%, at least 30%, at least 40%, or at least 50% higher compared to levels of the protein when encoded by a nucleic acid sequence that is not codon optimized.

In some embodiments, a system described herein comprises, or a method described herein comprises the use of, a polypeptide comprising one or more (e.g., one) DNA-targeting moiety and one or more effector moiety, e.g., wherein the effector moiety is or comprises MQ1, e.g., bacterial MQ1, or a functional variant or fragment thereof. In some embodiments, MQ1 is Spiroplasma monobiae MQ1, e.g., MQ1 from strain ATCC 33825 and/or corresponding to Uniprot ID P15840. In some embodiments, MQ1 effector moiety is encoded by a nucleotide sequence of SEQ ID NO: 10. In some embodiments, a nucleotide sequence described herein comprises a sequence of SEQ ID NO: 10 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

MQ1
(SEQ ID NO: 10)
AGCAAGGTGGAGAACAAGACCAAGAAGCTGCGGGTGTTCGA
GGCCTTCGCCGGCATCGGCGCCCAGCGGAAGGCCCTGGAG
AAGGTGCGGAAGGACGAGTACGAGATCGTGGGCCTGGCCG
AGTGGTACGTGCCCGCCATCGTGATGTACCAGGCCATCCA
CAACAACTTCCACACCAAGCTGGAGTACAAGAGCGTGAGC
CGGGAGGAGATGATCGACTACCTGGAGAACAAGACCCTGA
GCTGGAACAGCAAGAACCCCGTGAGCAACGGCTACTGGAA
GCGGAAGAAGGACGACGAGCTGAAGATCATCTACAACGCC
ATCAAGCTGAGCGAGAAGGAGGGCAACATCTTCGACATCC
GGGACCTGTACAAGCGGACCCTGAAGAACATCGACCTGCT
GACCTACAGCTTCCCCTGCCAGGACCTGAGCCAGCAGGGC
ATCCAGAAGGGCATGAAGCGGGGCAGCGGCACCCGGAGCG
GCCTGCTGTGGGAGATCGAGCGGGCCCTGGACAGCACCGA
GAAGAACGACCTGCCCAAGTACCTGCTGATGGAGAACGTG
GGCGCCCTGCTGCACAAGAAGAACGAGGAGGAGCTGAACC
AGTGGAAGCAGAAGCTGGAGAGCCTGGGCTACCAGAACAG
CATCGAGGTGCTGAACGCCGCCGACTTCGGCAGCAGCCAG
GCCCGGCGGCGGGTGTTCATGATCAGCACCCTGAACGAGT
TCGTGGAGCTGCCCAAGGGCGACAAGAAGCCCAAGAGCAT
CAAGAAGGTGCTGAACAAGATCGTGAGCGAGAAGGACATC
CTGAACAACCTGCTGAAGTACAACCTGACCGAGTTCAAGA
AAACCAAGAGCAACATCAACAAGGCCAGCCTGATCGGCTA
CAGCAAGTTCAACAGCGAGGGCTACGTGTACGACCCCGAG
TTCACCGGCCCCACCCTGACCGCCAGCGGCGCCAACAGCC
GGATCAAGATCAAGGACGGCAGCAACATCCGGAAGATGAA
CAGCGACGAGACCTTCCTGTACATCGGCTTCGACAGCCAG
GACGGCAAGCGGGTGAACGAGATCGAGTTCCTGACCGAGA
ACCAGAAGATCTTCGTGTGCGGCAACAGCATCAGCGTGGA
GGTGCTGGAGGCCATCATCGACAAGATCGGCGGC

In some embodiments, MQ1 comprises an amino acid sequence of SEQ ID NO: 11. In some embodiments, MQ1 comprises an amino acid sequence of SEQ ID NO: 12. In some embodiments, an effector domain described herein comprises SEQ ID NO: 11 or 12, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

MQ1
(SEQ ID NO: 11)
MSKVENKTKKLRVFEAFAGIGAQRKALEKVRKDEYEIVGL
AEWYVPAIVMYQAIHNNFHTKLEYKSVSREEMIDYLENKT
LSWNSKNPVSNGYWKRKKDDELKIIYNAIKLSEKEGNIFD
IRDLYKRTLKNIDLLTYSFPCQDLSQQGIQKGMKRGSGTR
SGLLWEIERALDSTEKNDLPKYLLMENVGALLHKKNEEEL
NQWKQKLESLGYQNSIEVLNAADFGSSQARRRVFMISTLN
EFVELPKGDKKPKSIKKVLNKIVSEKDILNNLLKYNLTEF
KKTKSNINKASLIGYSKFNSEGYVYDPEFTGPTLTASGAN
SRIKIKDGSNIRKMNSDETFLYIGFDSQDGKRVNEIEFLT
ENQKIFVCGNSISVEVLEAIIDKIGG
(SEQ ID NO: 12)
SKVENKTKKLRVFEAFAGIGAQRKALEKVRKDEYEIVGLA
EWYVPAIVMYQAIHNNFHTKLEYKSVSREEMIDYLENKTL
SWNSKNPVSNGYWKRKKDDELKIIYNAIKLSEKEGNIFDI
RDLYKRTLKNIDLLTYSFPCQDLSQQGIQKGMKRGSGTRS
GLLWEIERALDSTEKNDLPKYLLMENVGALLHKKNEEELN
QWKQKLESLGYQNSIEVLNAADFGSSQARRRVFMISTLNE
FVELPKGDKKPKSIKKVLNKIVSEKDILNNLLKYNLTEFK
KTKSNINKASLIGYSKFNSEGYVYDPEFTGPTLTASGANS
RIKIKDGSNIRKMNSDETFLYIGFDSQDGKRVNEIEFLTE
NQKIFVCGNSISVEVLEAIIDKIGG

In some embodiments, MQ1 for use in a site-specific disrupting agent described herein is a variant, e.g., comprising one or more mutations, relative to wildtype MQ1 (e.g., SEQ ID NO: 11 or SEQ ID NO: 12). In some embodiments, an MQ1 variant comprises one or more amino acid substitutions, deletions, or insertions relative to wildtype MQ1. In some embodiments, an MQ1 variant comprises a K297P substitution. In some embodiments, an MQ1 variant comprises a N299C substitution. In some embodiments, an MQ1 variant comprises a E301Y substitution. In some embodiments, an MQ1 variant comprises a Q147L substitution (e.g., and has reduced DNA methyltransferase activity relative to wildtype MQ1). In some embodiments, an MQ1 variant comprises K297P, N299C, and E301Y substitutions (e.g., and has reduced DNA binding affinity relative to wildtype MQ1). In some embodiments, an MQ1 variant comprises Q147L, K297P, N299C, and E301Y substitutions (e.g., and has reduced DNA methyltransferase activity and DNA binding affinity relative to wildtype MQ1). In some embodiments, the site-specific disrupting agent comprises one or more linkers described herein, e.g., connecting a moiety/domain to another moiety/domain. In some embodiments, the site-specific disrupting agent comprises a targeting moiety that is or comprises a CRISPR/Cas molecule, e.g., comprising a CRISPR/Cas protein, e.g., a dCas9 protein. In some embodiments, the site-specific disrupting agent is a fusion protein comprising an effector moiety that is or comprises MQ1 and a DNA-targeting moiety that is or comprises a CRISPR/Cas molecule, e.g., comprising a CRISPR/Cas protein, e.g., a dCas9 protein; e.g., a dCas9m4. In some embodiments, the site-specific disrupting agent comprises an additional moiety described herein. In some embodiments, the site-specific disrupting agent decreases expression of a target gene or a plurality of target genes (e.g., a target gene or a plurality of target gene described herein). In some embodiments, the site-specific disrupting agent may be used in methods of modulating, e.g., decreasing, gene expression, methods of treating a condition, or methods of epigenetically modifying a target gene or transcription control element described herein. In some embodiments, a system comprises two or more site-specific disrupting agents.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises Krueppel-associated box (KRAB) domain of Zinc Finger protein 10 e.g., as according to NP_056209.2 or the protein encoded by NM_015394.5 or a functional variant or fragment thereof. In some embodiments, KRAB is a synthetic KRAB construct. In some embodiments, KRAB for use in a site-specific disrupting agent described herein is a variant, e.g., comprising one or more mutations, relative to wildtype KRAB (e.g., e.g., as according to NP_056209.2 or the protein encoded by NM_015394.5). In some embodiments, a KRAB variant comprises one or more amino acid substitutions, deletions, or insertions relative to wildtype KRAB. In some embodiments, a KRAB variant comprises a L37P substitution. In some embodiments, KRAB comprises an amino acid sequence of SEQ ID NO: 13:

(SEQ ID NO: 13)
KRAB
DAKSLTAWSRTLVTFKDVFVDFTREEWKLLDTAQQILYRN
VMLENYKNLVSLGYQLTKPDVILRLEKGEEPWLVEREIHQ
ETHPDSETAFEIKSSV

In some embodiments, the KRAB effector moiety is encoded by a nucleotide sequence of SEQ ID NO: 14. In some embodiments, a nucleotide sequence described herein comprises a sequence of SEQ ID NO: 14 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 14)
KRAB
GACGCCAAGAGCCTGACCGCCTGGAGCCGGACCCTGGTGA
CCTTCAAGGACGTGTTCGTGGACTTCACCCGGGAGGAGTG
GAAGCTGCTGGACACCGCCCAGCAGATCCTGTACCGGAAC
GTGATGCTGGAGAACTACAAGAACCTGGTGAGCCTGGGCT
ACCAGCTGACCAAGCCCGACGTGATCCTGCGGCTGGAGAA
GGGCGAGGAGCCCTGGCTGGTGGAGCGGGAGATCCACCAG
GAGACCCACCCCGACAGCGAGACCGCCTTCGAGATCAAGA
GCAGCGTG

In some embodiments, KRAB for use in a polypeptide or a site-specific disrupting agent described herein is a variant, e.g., comprising one or more mutations, relative to the KRAB sequence of SEQ ID NO: 13. In some embodiments, an KRAB variant comprises one or more amino acid substitutions, deletions, or insertions relative to SEQ ID NO: 13.

In some embodiments, the polypeptide or the site-specific disrupting agent is a fusion protein comprising an effector moiety that is or comprises KRAB and a targeting moiety, e.g., a Crisper/Cas protein. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a transcription control element described herein.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises DNMT3a/3L complex, or a functional variant or fragment thereof. In some embodiments, the DNMT3a/3L complex is a fusion construct. In some embodiments the DNMT3a/3L complex comprises DNMT3A, e.g., human DNMT3A, e.g., as according to NP_072046.2 or the protein encoded by NM_022552.4) or the protein encoded by NM_022552.4 or a functional variant or fragment thereof, e.g., aa 679-912 of human DNMT3A, e.g., as according to NP_072046.2 or the protein encoded by NM_022552.4). In some embodiments the DNMT3a/3L complex comprises human DNMT3L or a functional fragment or variant thereof (e.g., as according to NP_787063.1 or the protein encoded by NM_175867.3 or a functional variant or fragment thereof, e.g., aa 274-386 of human DNMT3L as according to NP_787063.1 or the protein encoded by NM_175867.3). In some embodiments, DNMT3a/3L comprises an amino acid sequence of SEQ ID NO:15. In some embodiments, an effector moiety described herein comprises SEQ ID NO: 15, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

(SEQ ID NO: 15)
DNMT3A/31 (h)
NHDQEFDPPKVYPPVPAEKRKPIRVLSLFDGIATGLLVLK
DLGIQVDRYIASEVCEDSITVGMVRHQGKIMYVGDVRSVT
QKHIQEWGPFDLVIGGSPCNDLSIVNPARKGLYEGTGRLF
FEFYRLLHDARPKEGDDRPFFWLFENVVAMGVSDKRDISR
FLESNPVMIDAKEVSAAHRARYFWGNLPGMNRPLASTVND
KLELQECLEHGRIAKFSKVRTITTRSNSIKQGKDQHFPVF
MNEKEDILWCTEMERVFGFPVHYTDVSNMSRLARQRLLGR
SWSVPVIRHLFAPLKEYFACVSSGNSNANSRGPSFSSGLV
PLSLRGSHMNPLEMFETVPVWRRQPVRVLSLFEDIKKELT
SLGFLESGSDPGQLKHVVDVTDTVRKDVEEWGPFDLVYGA
TPPLGHTCDRPPSWYLFQFHRLLQYARPKPGSPRPFFWMF
VDNLVLNKEDLDVASRFLEMEPVTIPDVHGGSLQNAVRVW
SNIPAIRSRHWALVSEEELSLLAQNKQSSKLAAKWPTKLV
KNCFLPLREYFKYFSTELTSSL

In some embodiments, DNMT3a/3L is encoded by a nucleotide sequence of SEQ ID NO: 16. In some embodiments, a nucleic acid described herein comprises a sequence of SEQ ID NO: 16 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

DNMT3A/31 (h)
(SEQ ID NO: 16)
AACCACGACCAGGAGTTCGACCCCCCCAAGGTGTACCCCC
CCGTGCCCGCCGAGAAGCGGAAGCCCATCCGGGTGCTGAG
CCTGTTCGACGGCATCGCCACCGGCCTGCTGGTGCTGAAG
GACCTGGGCATCCAGGTGGACCGGTACATCGCCAGCGAGG
TGTGCGAGGACAGCATCACCGTGGGCATGGTGCGGCACCA
GGGCAAGATCATGTACGTGGGCGACGTGCGGAGCGTGACC
CAGAAGCACATCCAGGAGTGGGGCCCCTTCGACCTGGTGA
TCGGCGGCAGCCCCTGCAACGACCTGAGCATCGTGAACCC
CGCCCGGAAGGGCCTGTACGAGGGCACCGGCCGGCTGTTC
TTCGAGTTCTACCGGCTGCTGCACGACGCCCGGCCCAAGG
AGGGCGACGACCGGCCCTTCTTCTGGCTGTTCGAGAACGT
GGTGGCCATGGGCGTGAGCGACAAGCGGGACATCAGCCGG
TTCCTGGAGAGCAACCCCGTGATGATCGACGCCAAGGAGG
TGAGCGCCGCCCACCGGGCCCGGTACTTCTGGGGCAACCT
GCCCGGCATGAACCGGCCCCTGGCCAGCACCGTGAACGAC
AAGCTGGAGCTGCAGGAGTGCCTGGAGCACGGCCGGATCG
CCAAGTTCAGCAAGGTGCGGACCATCACCACCCGGAGCAA
CAGCATCAAGCAGGGCAAGGACCAGCACTTCCCCGTGTTC
ATGAACGAGAAGGAGGACATCCTGTGGTGCACCGAGATGG
AGCGGGTGTTCGGCTTCCCCGTGCACTACACCGACGTGAG
CAACATGAGCCGGCTGGCCCGGCAGCGGCTGCTGGGCCGG
AGCTGGAGCGTGCCCGTGATCCGGCACCTGTTCGCCCCCC
TGAAGGAGTACTTCGCCTGCGTGAGCAGCGGCAACAGCAA
CGCCAACAGCCGGGGCCCCAGCTTCAGCAGCGGCCTGGTG
CCCCTGAGCCTGCGGGGCAGCCACATGAATCCTCTGGAGA
TGTTCGAGACAGTGCCCGTGTGGAGAAGGCAACCCGTGAG
GGTGCTGAGCCTCTTCGAGGACATTAAGAAGGAGCTGACC
TCTCTGGGCTTTCTGGAATCCGGCAGCGACCCCGGCCAGC
TGAAACACGTGGTGGACGTGACCGACACAGTGAGGAAGGA
CGTGGAAGAGTGGGGCCCCTTTGACCTCGTGTATGGAGCC
ACACCTCCTCTCGGCCACACATGCGATAGGCCTCCCAGCT
GGTATCTCTTCCAGTTCCACAGACTGCTCCAGTACGCCAG
ACCTAAGCCCGGCAGCCCCAGACCCTTCTTCTGGATGTTC
GTGGACAATCTGGTGCTGAACAAGGAGGATCTGGATGTGG
CCAGCAGATTTCTGGAGATGGAACCCGTGACAATCCCCGA
CGTGCATGGCGGCTCTCTGCAGAACGCCGTGAGAGTGTGG
TCCAACATCCCCGCCATTAGAAGCAGACACTGGGCTCTGG
TGAGCGAGGAGGAACTGTCTCTGCTGGCCCAGAATAAGCA
GTCCTCCAAGCTGGCCGCCAAGTGGCCCACCAAGCTGGTG
AAGAACTGCTTTCTGCCTCTGAGGGAGTATTTCAAGTATT
TCAGCACCGAACTGACCAGCAGCCTG

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises EZH2, e.g., as according to NP-004447.2 or NP_001190176.1 2 or the protein encoded by NM_004456.5 or NM_001203247.2 or a functional variant or fragment thereof. In some embodiments, MQ1 for use in a site-specific disrupting agent described herein is a variant, e.g., comprising one or more mutations, relative to EZH2, e.g., as according to NP-004447.2 or NP_001190176.1 2 or the protein encoded by NM_004456.5 or NM_001203247.2. In some embodiments, an EZH2 variant comprises one or more amino acid substitutions, deletions, or insertions relative to wildtype EZH2. In some embodiments, EZH2 comprises an amino acid sequence of SEQ ID NO: 17:

EZH2
(SEQ ID NO: 17)
GQTGKKSEKGPVCWRKRVKSEYMRLRQLKRFRRADEVKSM
FSSNRQKILERTEILNQEWKQRRIQPVHILTSVSSLRGTR
ECSVTSDLDFPTQVIPLKTLNAVASVPIMYSWSPLQQNFM
VEDETVLHNIPYMGDEVLDQDGTFIEELIKNYDGKVHGDR
ECGFINDEIFVELVNALGQYNDDDDDDDGDDPEEREEKQK
DLEDHRDDKESRPPRKFPSDKIFEAISSMFPDKGTAEELK
EKYKELTEQQLPGALPPECTPNIDGPNAKSVQREQSLHSF
HTLFCRRCFKYDCFLHPFHATPNTYKRKNTETALDNKPCG
PQCYQHLEGAKEFAAALTAERIKTPPKRPGGRRRGRLPNN
SSRPSTPTINVLESKDTDSDREAGTETGGENNDKEEEEKK
DETSSSSEANSRCQTPIKMKPNIEPPENVEWSGAEASMFR
VLIGTYYDNFCAIARLIGTKTCRQVYEFRVKESSIIAPAP
AEDVDTPPRKKKRKHRLWAAHCRKIQLKKDGSSNHVYNYQ
PCDHPRQPCDSSCPCVIAQNFCEKFCQCSSECQNRFPGCR
CKAQCNTKQCPCYLAVRECDPDLCLTCGAADHWDSKNVSC
KNCSIQRGSKKHLLLAPSDVAGWGIFIKDPVQKNEFISEY
CGEIISQDEADRRGKVYDKYMCSFLFNLNNDFVVDATRKG
NKIRFANHSVNPNCYAKVMMVNGDHRIGIFAKRAIQTGEE
LFFDYRYSQADALKYVGIEREMEIP

In some embodiments, the EZH2 effector moiety is encoded by a nucleotide sequence of SEQ ID NO: 18. In some embodiments, a nucleotide sequence described herein comprises a sequence of SEQ ID NO: 18 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2
(SEQ ID NO: 18)
GGCCAGACCGGCAAGAAGAGCGAGAAGGGCCCCGTGTGCT
GGCGGAAGCGGGTGAAGAGCGAGTACATGCGGCTGCGGCA
GCTGAAGCGGTTCCGGCGGGCCGACGAGGTGAAGAGCATG
TTCAGCAGCAACCGGCAGAAGATCCTGGAGCGGACCGAGA
TCCTGAACCAGGAGTGGAAGCAGCGGCGAATCCAGCCCGT
GCACATCCTGACCAGCGTGAGCAGCCTGCGGGGCACCCGG
GAGTGCAGCGTGACCAGCGACCTGGACTTCCCCACCCAGG
TGATCCCCCTAAAGACCCTGAACGCCGTGGCCAGCGTGCC
CATCATGTACAGCTGGAGCCCCCTGCAGCAGAACTTCATG
GTGGAGGACGAGACCGTGCTGCACAACATCCCCTACATGG
GCGACGAGGTGCTGGACCAGGACGGCACCTTCATCGAGGA
GCTGATCAAGAACTACGACGGCAAGGTGCACGGCGACCGG
GAGTGCGGCTTCATCAACGACGAGATCTTCGTGGAGCTGG
TGAACGCCCTGGGCCAGTACAACGACGACGACGACGACGA
CGACGGCGACGACCCCGAGGAGCGGGAGGAGAAGCAGAAG
GACCTGGAGGACCACCGGGACGACAAGGAGAGCCGGCCCC
CCCGGAAGTTCCCCAGCGACAAGATCTTCGAGGCCATCAG
CAGCATGTTCCCCGACAAGGGCACCGCCGAGGAGCTGAAG
GAGAAGTACAAGGAGCTGACCGAGCAGCAGCTGCCCGGCG
CCCTGCCCCCCGAGTGCACCCCCAACATCGACGGCCCCAA
CGCCAAGAGCGTGCAGCGGGAGCAGAGCCTGCACAGCTTC
CACACCCTGTTCTGCCGGCGGTGCTTCAAGTACGACTGCT
TCCTGCACCCCTTCCACGCCACCCCCAACACCTACAAGCG
GAAGAACACCGAGACCGCCCTGGACAACAAGCCCTGCGGC
CCCCAGTGCTACCAGCACCTGGAGGGCGCCAAGGAGTTCG
CCGCCGCCCTGACCGCCGAGCGGATCAAGACCCCCCCCAA
GCGGCCCGGCGGCCGGCGGCGGGGCCGGCTGCCCAACAAC
AGCAGCCGGCCCAGCACCCCCACCATCAACGTGCTGGAGA
GCAAGGACACCGACAGCGACCGGGAGGCCGGCACCGAGAC
CGGCGGCGAGAACAACGACAAGGAGGAGGAGGAGAAGAAG
GACGAGACCAGCAGCAGCAGCGAGGCCAACAGCCGGTGCC
AGACCCCCATCAAGATGAAGCCCAACATCGAGCCCCCCGA
GAACGTGGAGTGGAGCGGCGCCGAGGCCAGCATGTTCCGG
GTGCTGATCGGCACCTACTACGACAACTTCTGCGCCATCG
CCCGGCTGATCGGCACCAAGACCTGCCGGCAGGTGTACGA
GTTCCGGGTGAAGGAGAGCAGCATCATCGCCCCCGCCCCC
GCCGAGGACGTGGACACCCCCCCCCGGAAGAAGAAGCGGA
AGCACCGGCTGTGGGCCGCCCACTGCCGGAAGATCCAGCT
GAAGAAGGACGGCAGCAGCAACCACGTGTACAACTACCAG
CCCTGCGACCACCCCCGGCAGCCCTGCGACAGCAGCTGCC
CCTGCGTGATCGCCCAGAACTTCTGCGAGAAGTTCTGCCA
GTGCAGCAGCGAGTGCCAGAACCGGTTCCCCGGCTGCCGG
TGCAAGGCCCAGTGCAACACCAAGCAGTGCCCCTGCTACC
TGGCCGTGCGGGAGTGCGACCCCGACCTGTGCCTGACCTG
CGGCGCCGCCGACCACTGGGACAGCAAGAACGTGAGCTGC
AAGAACTGCAGCATCCAGCGGGGCAGCAAGAAGCACCTGC
TGCTGGCCCCCAGCGACGTGGCCGGCTGGGGCATCTTCAT
CAAGGACCCCGTGCAGAAGAACGAGTTCATCAGCGAGTAC
TGCGGCGAGATCATCAGCCAGGACGAGGCCGACCGGCGGG
GCAAGGTGTACGACAAGTACATGTGCAGCTTCCTGTTCAA
CCTGAACAACGACTTCGTGGTGGACGCCACCCGGAAGGGC
AACAAGATCCGGTTCGCCAACCACAGCGTGAACCCCAACT
GCTACGCCAAGGTGATGATGGTGAACGGCGACCACCGGAT
CGGCATCTTCGCCAAGCGGGCCATCCAGACCGGCGAGGAG
CTGTTCTTCGACTACCGGTACAGCCAGGCCGACGCCCTGA
AGTACGTGGGCATCGAGCGGGAGATGGAGATCCCC

In some embodiments, EZH2 for use in a polypeptide or a site-specific disrupting agent described herein is a variant, e.g., comprising one or more mutations, relative to the EZH2 sequence of SEQ ID NO: 17. In some embodiments, an EZH2 variant comprises one or more amino acid substitutions, deletions, or insertions relative to SEQ ID NO: 17.

In some embodiments, the polypeptide or the site-specific disrupting agent is a fusion protein comprising an effector moiety that is or comprises EZH2 and a targeting moiety. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a transcription control element described herein.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises HDAC8, e.g., as according to NP_001159890 or NP_060956.1 or the protein encoded by NM_001166418 or NM_018486.3 or a functional variant or fragment thereof. In some embodiments, HDAC8 comprises an amino acid sequence of SEQ ID NO: 19:

HDAC8
(SEQ ID NO: 19)
EEPEEPADSGQSLVPVYIYSPEYVSMCDSLAKIPKRASMV
HSLIEAYALHKQMRIVKPKVASMEEMATFHTDAYLQHLQK
VSQEGDDDHPDSIEYGLGYDCPATEGIFDYAAAIGGATIT
AAQCLIDGMCKVAINWSGGWHHAKKDEASGFCYLNDAVLG
ILRLRRKFERILYVDLDLHHGDGVEDAFSFTSKVMTVSLH
KFSPGFFPGTGDVSDVGLGKGRYYSVNVPIQDGIQDEKYY
QICESVLKEVYQAFNPKAVVLQLGADTIAGDPMCSFNMTP
VGIGKCLKYILQWQLATLILGGGGYNLANTARCWTYLTGV
ILGKTLSSEIPDHEFFTAYGPDYVLEITPSCRPDRNEPHR
IQQILNYIKGNLKHVV

In some embodiments, the HDAC8 effector moiety is encoded by a nucleotide sequence of SEQ ID NO: 66. In some embodiments, a nucleotide sequence described herein comprises a sequence of SEQ ID NO: 66 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

HDAC8
(SEQ ID NO: 66)
GAGGAGCCCGAGGAGCCCGCCGATAGCGGACAATCTCTGG
TGCCCGTCTACATCTACAGCCCCGAATATGTGAGCATGTG
TGATTCCCTCGCCAAGATCCCTAAGAGAGCCAGCATGGTG
CATTCTCTGATCGAGGCCTACGCTCTGCATAAGCAAATGA
GGATCGTGAAGCCCAAGGTCGCCAGCATGGAAGAGATGGC
CACCTTTCACACCGATGCCTACCTCCAACATCTCCAGAAG
GTGTCCCAAGAGGGCGACGACGACCACCCCGACTCCATTG
AGTACGGACTGGGCTATGATTGCCCCGCCACCGAGGGCAT
CTTTGACTATGCCGCCGCTATCGGCGGAGCTACCATCACA
GCCGCCCAGTGTCTGATTGATGGCATGTGCAAGGTCGCCA
TCAACTGGTCCGGAGGCTGGCATCATGCCAAGAAGGATGA
GGCCTCCGGCTTCTGTTATCTGAATGACGCCGTGCTGGGC
ATTCTGAGACTGAGGAGGAAATTCGAGAGGATTCTGTACG
TGGATCTGGATCTGCATCACGGAGATGGAGTCGAAGATGC
CTTCAGCTTCACCAGCAAGGTGATGACAGTCTCTCTGCAC
AAGTTCTCCCCCGGCTTCTTTCCCGGAACCGGCGACGTGT
CCGACGTGGGACTGGGCAAGGGAAGGTACTACAGCGTGAA
CGTGCCCATTCAAGACGGCATCCAAGACGAGAAGTACTAC
CAGATCTGCGAGTCCGTGCTCAAGGAGGTCTACCAAGCCT
TCAATCCTAAGGCTGTCGTGCTCCAACTGGGAGCTGATAC
CATTGCTGGCGATCCCATGTGCAGCTTCAATATGACACCC
GTCGGAATCGGCAAGTGCCTCAAGTACATCCTCCAGTGGC
AGCTCGCCACCCTCATTCTCGGAGGAGGCGGATACAATCT
GGCTAATACCGCCAGATGCTGGACCTATCTGACCGGCGTG
ATTCTGGGCAAAACACTGAGCAGCGAAATCCCCGACCACG
AGTTTTTCACCGCTTACGGCCCCGACTACGTGCTGGAGAT
CACCCCCAGCTGCAGACCCGATAGAAACGAACCCCATAGA
ATCCAGCAAATTCTGAACTATATCAAGGGCAACCTCAAGC
ACGTCGTG

In some embodiments, the HDAC8 for use in a polypeptide or a site-specific disrupting agent described herein is a variant, e.g., comprising one or more mutations, relative to the HDAC8 sequence of SEQ ID NO: 19. In some embodiments, an HDAC8 variant comprises one or more amino acid substitutions, deletions, or insertions relative to SEQ ID NO: 19.

In some embodiments, the polypeptide or the site-specific disrupting agent is a fusion protein comprising an effector moiety that is or comprises HDAC8 and a targeting moiety. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a transcription control element described herein.

In some embodiments, a system described herein comprises, or a method described herein comprises the use of, a site-specific disrupting agent or a polypeptide comprising one or more (e.g., one) targeting moiety and one or more effector moiety, wherein the effector moiety is or comprises G9A e.g., as according to NP_001350618.1 or the protein encoded by NM_001363689.1 or a functional variant or fragment thereof, e.g., aa967-1250 of comprises G9A e.g., as according to NP_001350618.1 or the protein encoded by NM_001363689.1. In some embodiments, G9A comprises an amino acid sequence of SEQ ID NO: 67:

G9A
(SEQ ID NO: 67)
GNRAIRTEKIICRDVARGYENVPIPCVNGVDGEPCPEDYK
YISENCETSTMNIDRNITHLQHCTCVDDCSSSNCLCGQLS
IRCWYDKDGRLLQEFNKIEPPLIFECNQACSCWRNCKNRV
VQSGIKVRLQLYRTAKMGWGVRALQTIPQGTFICEYVGEL
ISDAEADVREDDSYLFDLDNKDGEVYCIDARYYGNISRFI
NHLCDPNIIPVRVFMLHQDLRFPRIAFFSSRDIRTGEELG
FDYGDRFWDIKSKYFTCQCGSEKCKHSAEAIALEQSRLAR
LD

In some embodiments, the G9A effector moiety is encoded by a nucleotide sequence of SEQ ID NO: 68. In some embodiments, a nucleotide sequence described herein comprises a sequence of SEQ ID NO: 68 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A
(SEQ ID NO: 68)
GGAAATAGGGCTATCAGAACCGAGAAGATCATCTGTAGGG
ACGTGGCTAGAGGCTACGAGAACGTGCCCATTCCTTGCGT
GAATGGCGTGGATGGCGAACCTTGCCCCGAGGACTACAAA
TACATCTCCGAGAACTGCGAAACCAGCACAATGAACATCG
ACAGAAACATCACCCACCTCCAGCACTGCACATGTGTGGA
TGACTGCTCCTCCAGCAACTGTCTGTGCGGCCAGCTCTCC
ATCAGATGCTGGTACGACAAGGACGGCAGACTGCTGCAAG
AGTTCAACAAGATCGAACCCCCTCTCATCTTCGAGTGTAA
CCAAGCTTGCAGCTGCTGGAGAAACTGCAAGAATAGAGTG
GTCCAGAGCGGCATCAAGGTGAGACTGCAACTGTACAGAA
CCGCCAAGATGGGATGGGGAGTGAGGGCTCTGCAAACCAT
TCCCCAAGGCACCTTCATCTGCGAATACGTGGGCGAACTG
ATCTCCGACGCCGAAGCTGACGTGAGAGAGGACGACAGCT
ATCTCTTCGATCTGGACAATAAGGACGGCGAGGTGTACTG
CATCGACGCTAGATATTACGGCAACATCTCTAGATTCATC
AACCACCTCTGCGATCCCAACATCATTCCCGTGAGGGTGT
TCATGCTGCACCAAGATCTGAGGTTCCCTAGAATCGCCTT
CTTCAGCTCTAGAGACATCAGAACCGGCGAGGAGCTGGGC
TTCGATTACGGCGATAGATTCTGGGACATCAAGTCCAAGT
ACTTCACATGCCAGTGCGGCAGCGAGAAGTGTAAGCACAG
CGCTGAGGCCATTGCTCTGGAGCAGTCTAGACTGGCCAGA
CTGGAT

In some embodiments, G9A for use in a polypeptide or a site-specific disrupting agent described herein is a variant, e.g., comprising one or more mutations, relative to the G9A sequence of SEQ ID NO: 67. In some embodiments, an G9A variant comprises one or more amino acid substitutions, deletions, or insertions relative to SEQ ID NO: 67.

In some embodiments, the polypeptide or the site-specific disrupting agent is a fusion protein comprising an effector moiety that is or comprises G9A and a targeting moiety. In some embodiments, the polypeptide or the site-specific disrupting agent comprises an additional moiety described herein. In some embodiments, the polypeptide or the site-specific disrupting agent decreases expression of a target gene or a plurality of target genes. In some embodiments, the polypeptide or the site-specific disrupting agent may be used in methods of modulating, e.g., decreasing, gene expression, methods of treating a condition, or methods of epigenetically modifying a target gene or a plurality of target genes, e.g., a transcription control element described herein.

### Systems

Systems of the present disclosure may comprise two or more site-specific disrupting agents. In some embodiments, a site-specific disrupting agent system comprises 2, 3, 4, 5, 6, 7, 8, 9, 10, 11, 12, or more, site-specific disrupting agents (and optionally no more than 15, 14, 13, 12, 11, 10, 9, 8, 7, 6, 5, 4, 3, or 2). In some embodiments, system targets two or more different sequences (e.g., a 1st and 2nd, 3rd, 4th, 5th, 6th, 7th, 8th, 9th, 10th, 11th, 12th, and/or further DNA sequence, and optionally no more than a 20th, 19th, 18th, 17th, 16th, 15th, 14th, 13th, 12th, 11th, 10th, 9th, 8th, 6th, 5th, 4th, 3rd, or 2nd sequence). In some embodiments, system comprises a plurality of site-specific disrupting agents, wherein each member of the plurality of site-specific disrupting agents does not detectably bind, e.g., does not bind, to another member of the plurality of site-specific disrupting agents. In some embodiments, system comprises a first site-specific disrupting agent and a second site-specific disrupting agent, wherein the first site-specific disrupting agent does not detectably bind, e.g., does not bind, to the second site-specific disrupting agent.

In some embodiments, a system of the present disclosure comprises two or more site-specific disrupting agents, wherein the site-specific disrupting agents are present together in a composition, pharmaceutical composition, or mixture. In some embodiments, a system of the present disclosure comprises two or more site-specific disrupting agents, wherein one or more site-specific disrupting agents is not admixed with at least one other site-specific disrupting agent. In some embodiments, a system may comprise a first site-specific disrupting agent and a second site-specific disrupting agent, wherein the presence of the first site-specific disrupting agent in the nucleus of a cell does not overlap with the presence of the second site-specific disrupting agent in the nucleus of the same cell, wherein the system achieves a decrease in expression of a plurality of genes via the non-overlapping presences of the first and second site-specific disrupting agents. In some embodiments, the first site-specific disrupting agent and a second site-specific disrupting agent may act simultaneously or sequentially.

In some embodiments, the site-specific disrupting agents of a system each comprise a different targeting moiety (e.g., the first, second, third, or further site-specific disrupting agents each comprise different targeting moieties from one another). For example, a system may comprise a first site-specific disrupting agent and a second site-specific disrupting agent wherein the first site-specific disrupting agent comprises a first targeting moiety (e.g., a Cas9 domain, TAL effector domain, or Zn Finger domain), and the second site-specific disrupting agent comprises a second targeting moiety (e.g., a Cas9 domain, TAL effector domain, or Zn Finger domain) different from the first targeting moiety. In some embodiments, different can mean comprising distinct types of targeting moiety, e.g., the first targeting moiety comprises a Cas9 domain, and the second DNA-targeting moiety comprises a Zn finger domain. In other embodiments, different can mean comprising distinct variants of the same type of targeting moiety, e.g., the first targeting moiety comprises a first Cas9 domain (e.g., from a first species) and the second targeting moiety comprises a second Cas9 domain (e.g., from a second species).

In an embodiment, when a system comprises two or more targeting moieties of the same type, e.g., two or more Cas9 or Zn finger domains, the targeting moieties specifically bind two or more different sequences. For example, in a system comprising two or more Cas9 domains, the two or more Cas9 domains may be chosen or altered such that they only appreciably bind the gRNA corresponding to their target sequence (e.g., and do not appreciably bind the gRNA corresponding to the target of another Cas9 domain). In a further example, in a system comprising two or more effector moieties, the two or more effector moieties may be chosen or altered such that they only appreciably bind to their target sequence (e.g., and do not appreciably bind the target sequence of another effector moiety).

In some embodiments, a system comprises three or more site-specific disrupting agents and two or more site-specific disrupting agents comprise the same targeting moiety. For example, a system may comprise three site-specific disrupting agents, wherein the first and second site-specific disrupting agents both comprise a first targeting moiety and the third site-specific disrupting agent comprises a second different targeting moiety. For a further example, a system may comprise four site-specific disrupting agents, wherein the first and second site-specific disrupting agents both comprise a first targeting moiety and the third and fourth site-specific disrupting agents comprises a second different targeting moiety. For a further example, a system may comprise five site-specific disrupting agents, wherein the first and second site-specific disrupting agents both comprise a first targeting moiety, the third and fourth site-specific disrupting agents both comprise a second different targeting moiety, and the fifth site-specific disrupting agent comprises a third different targeting moiety. As described above, different can mean comprising different types of -targeting moieties or comprising distinct variants of the same type of targeting moiety.

In some embodiments, the site-specific disrupting agents of a system each bind to a different DNA sequence (e.g., the first, second, third, or further site-specific disrupting agents each bind DNA sequences that are different from one another). For example, a system may comprise a first site-specific disrupting agent and a second site-specific disrupting agent wherein the first site-specific disrupting agent binds to a first DNA sequence, and the second site-specific disrupting agent binds to a second DNA sequence. In some embodiments involving different DNA sequences, there is at least one position that is not identical between the DNA sequence bound by one site-specific disrupting agent and the DNA sequence bound by another site-specific disrupting agent, or there is at least one position present in the DNA sequence bound by one site-specific disrupting agent that is not present in the DNA sequence bound by another site-specific disrupting agent.

In some embodiments, the first DNA sequence may be situated on a first genomic DNA strand and the second DNA sequence may be situated on a second genomic DNA strand. In some embodiments, the first DNA sequence may be situated on the same genomic DNA strand as the second DNA sequence.

In some embodiments, a system comprises three or more site-specific disrupting agents and two or more site-specific disrupting agents bind the same DNA sequence. For example, a system may comprise three site-specific disrupting agents, wherein the first and site-specific disrupting agents both bind a first DNA sequence, and the third site-specific disrupting agent binds a second different DNA sequence. For a further example, a system may comprise four site-specific disrupting agents, wherein the first and second site-specific disrupting agents both bind a first DNA sequence and the third and fourth site-specific disrupting agents both bind a second DNA sequence. For a further example, a system may comprise five site-specific disrupting agents, wherein the first and second site-specific disrupting agents both bind a first DNA sequence, the third and fourth site-specific disrupting agents both bind a second DNA sequence, and the fifth site-specific disrupting agent binds a third DNA sequence. As described above, different can mean that there is at least one position that is not identical between the DNA sequence bound by one site-specific disrupting agent and the DNA sequence bound by another site-specific disrupting agent, or that there is at least one position present in the DNA sequence bound by one site-specific disrupting agent that is not present in the DNA sequence bound by another site-specific disrupting agent.

In some embodiments, a system comprises two or more (e.g., two) site-specific disrupting agents and a plurality (e.g., two) of the site-specific disrupting agents comprise targeting moieties that bind to different DNA sequences. In such embodiments, a first targeting moiety may bind to a first DNA sequence and a second DNA-targeting moiety may bind to a second DNA sequence, wherein the first and the second DNA sequences are different and do not overlap. In some such embodiments, the first DNA sequence is separated from the second DNA sequence by at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no more than 500, 400, 300, 200, 100, 95, 90, 85, 80, 75, 70, 65, 60, 55, or 50 base pairs). In some such embodiments, the first DNA sequence is separated from the second DNA sequence by no more than 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 base pairs (and optionally, no base pairs, e.g., the first and second sequence are directly adjacent one another).

In some embodiments, the site-specific disrupting agents of a system each comprise a different effector moiety (e.g., the first, second, third, or further site-specific disrupting agents each comprise a different effector moiety from one another). For example, a system may comprise a first site-specific disrupting agent and a second site-specific disrupting agent wherein the first site-specific disrupting agent comprises a first effector moiety, and the second site-specific disrupting agent comprises a second effector moiety different from the first effector moiety. In some embodiments, the different effector moieties comprise distinct types of effector moiety. In other embodiments, the different effector moieties comprise distinct variants of the same type of effector moiety.

Targeting Moieties

Targeting moieties may specifically bind a DNA sequence, e.g., a DNA sequence associated with a target plurality of genes, e.g., an anchor sequence of an ASMC comprising the target plurality of genes. Any molecule or compound that specifically binds a DNA sequence may be used as a targeting moiety. In some embodiments, a targeting moiety comprises a nucleic acid, e.g., comprising a sequence that is complementary to an anchor sequence, e.g., an anchor sequence of an ASMC comprising the target plurality of genes. In some embodiments, the nucleic acid is an oligonucleotide that physically/sterically blocks binding a genomic complex component (e.g., a nucleating polypeptide, e.g., CTCF) to an anchor sequence. In some embodiments, the nucleic acid comprises a guide RNA (gRNA), e.g., compatible with a CRISPR/Cas molecule. In some embodiments, a targeting moiety comprises a CRISPR/Cas molecule, a TAL effector molecule, a Zn finger molecule, a tetR domain, a meganuclease, a peptide nucleic acid (PNA) or a nucleic acid molecule.

In some embodiments, a targeting moiety binds to its target sequence with a K_{D} of less than or equal to 500, 450, 400, 350, 300, 250, 200, 150, 100, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM (and optionally, a K_{D} of at least 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.002, or 0.001 nM). In some embodiments, a targeting moiety binds to its target sequence with a K_{D} of 0.001 nM to 500 nM, e.g., 0.1 nM to 5 nM, e.g., about 0.5 nM. In some embodiments, a targeting moiety binds to a non-target sequence with a K_{D} of at least 500, 600, 700, 800, 900, 1000, 2000, 5000, 10,000, or 100,000 nM (and optionally, does not appreciably bind to a non-target sequence). In some embodiments, a targeting moiety does not bind to a non-target sequence.

In some embodiments, a targeting moiety comprises a nucleic acid comprising a sequence selected from Table 7 or a sequence having at least 70, 75, 80, 85, 90, 91, 92, 93, 94, 95, 96, 97, 98, or 99% identity thereto, or differing at no more than 1, 2, 3, 4, or 5 positions relative thereto.

TABLE 7
Exemplary sequences

| Name | Sequence | SEQ ID NO. |
|---|---|---|
| GD-28481 | AGCCCCACCTTGTGGTCAGA | 21 |
| GD-28482 | AGTGCTGCCTTCTGACCACA | 22 |
| GD-28483 | GCTGCCTTCTGACCACAAGG | 23 |
| GD-28484 | CCAGTATAAGCCCCACCTTG | 24 |
| GD-28485 | CTGCCTGTCCCATAAGGAGG | 25 |
| GD-28486 | GCACTGCCTGTCCCATAAGG | 26 |
| GD-28487 | GGTCCTCCTCCTTATGGGAC | 27 |
| GD-28488 | GCCTTGTTTTCGGCTCTAGA | 28 |
| GD-28489 | GCCATCTAGAGCCGAAAACA | 29 |
| GD-29251 | CCAATGAAGATGAAACTGGG | 30 |
| GD-29252 | AACGTGCTTGCCTAAGATTC | 31 |
| GD-29253 | AGCCCTTAATCATATCTAGT | 32 |
| GD-29254 | CAGAGCTTAAGACCTGTACT | 33 |
| GD-29255 | GCCCACCTTGACCTTCACAA | 34 |

In some embodiments, a targeting moiety comprises a nucleic acid comprising a sequence selected from Table 6 or a sequence having at least 70, 75, 80, 85, 90, 91, 92, 93, 94, 95, 96, 97, 98, or 99% identity thereto, or differing at no more than 1, 2, 3, 4, or 5 positions relative thereto.

TABLE 6
Exemplary guide sequence

| | | | Genomic |
|---|---|---|---|
| Guide | Sequence | Strand | Coordinate |
| GD-30178 | GTTACTGCGTAATTACCAGG | + | GRCh37: |
| | (SEQ ID NO: 35) | | chr4: |
| | | | 74528428- |
| | | | 74528450 |
| GD-30179 | TATTACATCCTACCTATAAG | + | GRCh37: |
| | (SEQ ID NO: 36) | | chr4: |
| | | | 74528567- |
| | | | 74528589 |
| GD-30180 | TGGGCTCTGGACTTAGATCG | + | GRCh37: |
| | (SEQ ID NO: 37) | | chr4: |
| | | | 74528609- |
| | | | 74528631 |
| GD-30181 | TAAGTGGGCTATGTATACAC | + | GRCh37: |
| | (SEQ ID NO: 38) | | chr4: |
| | | | 74789132- |
| | | | 74789154 |
| GD-30182 | TTTCTAAGTCTGTCACAAGG | − | GRCh37: |
| | (SEQ ID NO: 39) | | chr4: |
| | | | 74789250- |
| | | | 74789272 |
| GD-30183 | AAAGTAATATGATCTAGGAA | − | GRCh37: |
| | (SEQ ID NO: 40) | | chr4: |
| | | | 74789312- |
| | | | 74789334 |
| GD-30184 | GTTCGAGCGGCTGTGCGAGG | + | GRCh37: |
| | (SEQ ID NO: 41) | | chr4: |
| | | | 74964853- |
| | | | 74964875 |
| GD-30185 | GCTCTGTGGCTCTCCGAGAA | + | GRCh37: |
| | (SEQ ID NO: 42) | | chr4: |
| | | | 74964906- |
| | | | 74964928 |
| GD-30186 | GTGTGTGTGTTTCAACGTAG | + | GRCh37: |
| | (SEQ ID NO: 43) | | chr4: |
| | | | 74965538- |
| | | | 74965560 |
| GD-30187 | GGAAGTCACTGGGAGCTGCG | + | GRCh37: |
| | (SEQ ID NO: 44) | | chr4: |
| | | | 74965737- |
| | | | 74965759 |
| GD-30200 | GGCCACGGGTGTGTTCCCAG | − | GRCh37: |
| | (SEQ ID NO: 45) | | chr4: |
| | | | 75000031- |
| | | | 75000053 |
| GD-30201 | ATGGCCATTTGCAAAAGTCA | + | GRCh37: |
| | (SEQ ID NO: 46) | | chr4: |
| | | | 75000115- |
| | | | 75000137 |
| GD-30202 | CCAAACTAGACAGATAAAGC | + | GRCh37: |
| | (SEQ ID NO: 47) | | chr4: |
| | | | 75000231- |
| | | | 75000253 |
| GD-30203 | CCAGCATGACTCTAGCATGC | − | GRCh37: |
| | (SEQ ID NO: 48) | | chr4: |
| | | | 74975146- |
| | | | 74975168 |
| GD-30204 | TGGCCAAGGTCTGATATGCA | + | GRCh37: |
| | (SEQ ID NO: 49) | | chr4: |
| | | | 74975369- |
| | | | 74975391 |
| GD-30205 | TCATGAGTCCCAGAACATGT | − | GRCh37: |
| | (SEQ ID NO: 50) | | chr4: |
| | | | 74976318- |
| | | | 74976340 |
| GD-30206 | GCGAAAGAAGTAGTAGCTAA | − | GRCh37: |
| | (SEQ ID NO: 51) | | chr4: |
| | | | 74570348- |
| | | | 74570370 |
| GD-30207 | GACTAAGACTGGCAAATCTG | − | GRCh37: |
| | (SEQ ID NO: 52) | | chr4: |
| | | | 74570503- |
| | | | 74570525 |
| GD-30208 | GACTAAGAGGAGCCGACATG | + | GRCh37: |
| | (SEQ ID NO: 53) | | chr4: |
| | | | 74570526- |
| | | | 74570548 |
| GD-30238 | GAAAAACGGGTGTTGTGACG | + | GRCm38: |
| | (SEQ ID NO: 54) | | chr5: |
| | | | 90661492- |
| | | | 90661514 |
| GD-30239 | TTTGTGAACTAAGGATTCTG | − | GRCm38: |
| | (SEQ ID NO: 55) | | chr5: |
| | | | 90661646- |
| | | | 90661668 |
| GD-30240 | GTCCGTGTAGAGTTACCATG | + | GRCm38: |
| | (SEQ ID NO: 56) | | chr5: |
| | | | 90661744- |
| | | | 90661766 |
| GD-30241 | GATGTATTCACAAGAGGACT | + | GRCm38: |
| | | | chr5: |
| | | | 90785610- |
| | (SEQ ID NO: 57) | | 90785632 |
| GD-30242 | AATTACTACCTCATAGCTAG | − | GRCm38: |
| | (SEQ ID NO: 58) | | chr5: |
| | | | 90909047- |
| | | | 90909069 |
| GD-30592 | GAAGGTAGAAATCCGCCACT | | GRCm38: |
| | (SEQ ID NO: 59) | | chr5: |
| | | | 90785724- |
| | | | 90785746 |
| GD-30593 | GAAACGCCGAGGTAACTCAT | | GRCm38: |
| | (SEQ ID NO: 60) | | chr5: |
| | | | 90788137- |
| | | | 90788159 |
| GD-30594 | CAACTAAAATTTCTAGCCCT | | GRCm38: |
| | (SEQ ID NO: 61) | | chr5: |
| | | | 90908926- |
| | | | 90908948 |
| GD-28044 | GACTCCAGTCTTTCTAGAAGA | | GRCm38: |
| | (SEQ ID NO: 62) | | chr6: |
| | | | 113076028- |
| | | | 113076047 |

In some embodiments, a targeting moiety comprises a nucleic acid comprising a sequence that is complementary to the sequence of an anchor sequence, e.g., of an ASMC comprising the target plurality of genes, or having no more than 1, 2, 3, 4, 5, 6, 7, 8, 9, or 10 positions of non-complementarity thereto.

In some embodiments, a targeting moiety comprises a nucleic acid comprising a sequence that at least partially overlapping with the region having genomic coordinates chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, chr4:74570526-74570548, chr5:90785724-90785746, chr5:90788137-90788159, chr5:90908926-90908948, chr5:90661492-90661514, chr5:90661646-90661668, chr5:90661744-90661766, chr5:90785610-90785632, chr5:90909047-90909069, or chr6:113076028-113076047 or a sequence that is within 5, 10, 15, 20, 30, 40, or 50 nucleotides of said region, or comprises a sequence having at least 70, 75, 80, 85, 90, 91, 92, 93, 94, 95, 96, 97, 98, or 99% identity to a sequence at said genomic region, or differing at no more than 1, 2, 3, 4, or 5 positions relative thereto.

In some embodiments, a targeting moiety binds to a sequence at genomic positions chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, chr4:74570526-74570548, chr5:90661492-90661514, chr5:90661646-90661668, chr5:90661744-90661766, chr5:90785610-90785632, chr5:90909047-90909069, chr5:90785724-90785746, chr5:90788137-90788159, chr5:90908926-90908948, or chr6:113076028-113076047.

In some embodiments, a targeting moiety binds to an anchor sequence or to a site proximal to an anchor sequence, e.g., an anchor sequence that is part of an ASMC comprising, wholly or in part, a target plurality of genes.

In some embodiments, a targeting moiety comprises a CRISPR/Cas molecule. In some embodiments, an effector moiety comprises a CRISPR/Cas molecule. A CRISPR/Cas molecule comprises a protein involved in the clustered regulatory interspaced short palindromic repeat (CRISPR) system, e.g., a Cas protein, and optionally a guide RNA, e.g., single guide RNA (sgRNA).

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. For example, in a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a partially double-stranded structure which is cleaved by RNase III, resulting in a crRNA/tracrRNA hybrid. A crRNA/tracrRNA hybrid then directs Cas9 endonuclease to recognize and cleave a target DNA sequence. A target DNA sequence must generally be adjacent to a “protospacer adjacent motif” (“PAM”) that is specific for a given Cas endonuclease; however, PAM sequences appear throughout a given genome. CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningitidis). Some endonucleases, e.g., Cas9 endonucleases, are associated with G-rich PAM sites, e. g., 5′-NGG (e.g., TGG, e.g., CGG, e.g., AGG), and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpfl (from Lachnospiraceae sp.). Cpf1-associated CRISPR arrays are processed into mature crRNAs without the requirement of a tracrRNA; in other words, a Cpf1 system requires only Cpf1 nuclease and a crRNA to cleave a target DNA sequence. Cpf1 endonucleases, are associated with T-rich PAM sites, e. g., 5′-TTN. Cpf1 can also recognize a 5′-CTA PAM motif. Cpf1 cleaves a target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from a PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e.g., Zetsche et al. (2015) Cell, 163:759-771.

A variety of CRISPR associated (Cas) genes or proteins can be used in the technologies provided by the present disclosure and the choice of Cas protein will depend upon the particular conditions of the method. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, a targeting moiety includes a sequence targeting polypeptide, such as a Cas protein, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus (e.g., a S. pyogenes, or a S. thermophilus), a Francisella (e.g., an F. novicida), a Staphylococcus (e.g., an S. aureus), an Acidaminococcus (e.g., an Acidaminococcus sp. BV3L6), a Neisseria (e.g., an N. meningitidis), a Cryptococcus, a Corynebacterium, a Haemophilus, a Eubacterium, a Pasteurella, a Prevotella, a Veillonella, or a Marinobacter.

In some embodiments, a Cas protein requires a protospacer adjacent motif (PAM) to be present in or adjacent to a target DNA sequence for the Cas protein to bind and/or function. In some embodiments, the PAM is or comprises, from 5′ to 3′, NGG, YG, NNGRRT, NNNRRT, NGA, TYCV, TATV, NTTN, or NNNGATT, where N stands for any nucleotide, Y stands for C or T, R stands for A or G, and V stands for A or C or G. In some embodiments, a Cas protein is a protein listed in Table 1. In some embodiments, a Cas protein comprises one or more mutations altering its PAM. In some embodiments, a Cas protein comprises E1369R, E1449H, and R1556A mutations or analogous substitutions to the amino acids corresponding to said positions. In some embodiments, a Cas protein comprises E782K, N968K, and R1015H mutations or analogous substitutions to the amino acids corresponding to said positions. In some embodiments, a Cas protein comprises D1135V, R1335Q, and T1337R mutations or analogous substitutions to the amino acids corresponding to said positions. In some embodiments, a Cas protein comprises S542R and K607R mutations or analogous substitutions to the amino acids corresponding to said positions. In some embodiments, a Cas protein comprises S542R, K548V, and N552R mutations or analogous substitutions to the amino acids corresponding to said positions.

TABLE 1

| | | | # of | | Mutations to alter | Mutations to make |
|---|---|---|---|---|---|---|
| Name | Enzyme | Species | AAs | PAM | PAM recognition | catalytically dead |
| FnCas9 | Cas9 | Francisella | 1629 | 5′-NGG-3′ | Wt | D11A/H969A/ |
| | | novicida | | | | N995A |
| FnCas9 | Cas9 | Francisella | 1629 | 5′-YG-3′ | E1369R/E1449H/R1556A | D11A/H969A/ |
| RHA | | novicida | | | | N995A |
| SaCas9 | Cas9 | Staphylococcus | 1053 | 5′-NNGRRT-3′ | Wt | D10A/H557A |
| | | aureus | | | | |
| SaCas9 | Cas9 | Staphylococcus | 1053 | 5′-NNNRRT-3′ | E782K/N968K/R1015H | D10A/H557A |
| KKH | | aureus | | | | |
| SpCas9 | Cas9 | Streptococcus | 1368 | 5′-NGG-3′ | Wt | D10A/D839A/ |
| | | pyogenes | | | | H840A/N863A |
| SpCas9 | Cas9 | Streptococcus | 1368 | 5′-NGA-3′ | D1135V/R1335Q/T1337R | D10A/D839A/ |
| VQR | | pyogenes | | | | H840A/N863A |
| AsCpf1 | Cpf1 | Acidaminococcus | 1307 | 5′-TYCV-3′ | S542R/K607R | E993A |
| RR | | sp. BV3L6 | | | | |
| AsCpf1 | Cpf1 | Acidaminococcus | 1307 | 5′-TATV-3′ | S542R/K548V/N552R | E993A |
| RVR | | sp. BV3L6 | | | | |
| FnCpf1 | Cpf1 | Francisella | 1300 | 5′-NTTN-3′ | Wt | D917A/E1006A/ |
| | | novicida | | | | D1255A |
| NmCas9 | Cas9 | Neisseria | 1082 | 5′-NNNGATT-3′ | Wt | D16A/D587A/ |
| | | meningitidis | | | | H588A/N611A |

In some embodiments, the Cas protein is catalytically active and cuts one or both strands of the target DNA site. In some embodiments, cutting the target DNA site is followed by formation of an alteration, e.g., an insertion or deletion, e.g., by the cellular repair machinery.

In some embodiments, the Cas protein is modified to deactivate the nuclease, e.g., nuclease-deficient Cas9. Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut target DNA. In some embodiments, dCas9 binding to a DNA sequence may interfere with transcription at that site by steric hindrance. In some embodiments, dCas9 binding to an anchor sequence may interfere with (e.g., decrease or prevent) genomic complex (e.g., ASMC) formation and/or maintenance. In some embodiments, a targeting moiety comprises a catalytically inactive Cas9, e.g., dCas9, e.g., Cas9m4. Many catalytically inactive Cas9 proteins are known in the art. In some embodiments, dCas9 comprises mutations in each endonuclease domain of the Cas protein, e.g., D10A and H840A mutations.

In some embodiments, a catalytically inactive Cas9 protein, e.g., dCas9, comprises a D11A mutation or an analogous substitution to the amino acid corresponding to said position. In some embodiments, a catalytically inactive Cas9 protein, e.g., dCas9, comprises a H969A mutation or an analogous substitution to the amino acid corresponding to said position. In some embodiments, a catalytically inactive Cas9 protein, e.g., dCas9, comprises a N995A mutation or an analogous substitution to the amino acid corresponding to said position. In some embodiments, a catalytically inactive Cas9 protein, e.g., dCas9, comprises D11A, H969A, and N995A mutations or analogous substitutions to the amino acids corresponding to said positions.

In some embodiments, a catalytically inactive Cas9 protein, e.g., dCas9, comprises a D10A mutation or an analogous substitution to the amino acid corresponding to said position. In some embodiments, a catalytically inactive Cas9 protein, e.g., dCas9, comprises a H557A mutation or an analogous substitution to the amino acid corresponding to said position. In some embodiments, a catalytically inactive Cas9 protein, e.g., dCas9, comprises D10A and H557A mutations or analogous substitutions to the amino acids corresponding to said positions.

In some embodiments, a catalytically inactive Cas9 protein, e.g., dCas9, comprises a D839A mutation or an analogous substitution to the amino acid corresponding to said position. In some embodiments, a catalytically inactive Cas9 protein, e.g., dCas9, comprises a H840A mutation or an analogous substitution to the amino acid corresponding to said position. In some embodiments, a catalytically inactive Cas9 protein, e.g., dCas9, comprises a N863A mutation or an analogous substitution to the amino acid corresponding to said position. In some embodiments, a catalytically inactive Cas9 protein, e.g., dCas9, comprises D10A, D839A, H840A, and N863A mutations or analogous substitutions to the amino acids corresponding to said positions.

In some embodiments, a catalytically inactive Cas9 protein, e.g., dCas9, comprises a E993A mutation or an analogous substitution to the amino acid corresponding to said position.

In some embodiments, a catalytically inactive Cas9 protein, e.g., dCas9, comprises a D917A mutation or an analogous substitution to the amino acid corresponding to said position. In some embodiments, a catalytically inactive Cas9 protein, e.g., dCas9, comprises a E1006A mutation or an analogous substitution to the amino acid corresponding to said position. In some embodiments, a catalytically inactive Cas9 protein, e.g., dCas9, comprises a D1255A mutation or an analogous substitution to the amino acid corresponding to said position. In some embodiments, a catalytically inactive Cas9 protein, e.g., dCas9, comprises D917A, E1006A, and D1255A mutations or analogous substitutions to the amino acids corresponding to said positions.

In some embodiments, a catalytically inactive Cas9 protein, e.g., dCas9, comprises a D16A mutation or an analogous substitution to the amino acid corresponding to said position. In some embodiments, a catalytically inactive Cas9 protein, e.g., dCas9, comprises a D587A mutation or an analogous substitution to the amino acid corresponding to said position. In some embodiments, a catalytically inactive Cas9 protein, e.g., dCas9, comprises a H588A mutation or an analogous substitution to the amino acid corresponding to said position. In some embodiments, a catalytically inactive Cas9 protein, e.g., dCas9, comprises a N611A mutation or an analogous substitution to the amino acid corresponding to said position. In some embodiments, a catalytically inactive Cas9 protein, e.g., dCas9, comprises D16A, D587A, H588A, and N611A mutations or analogous substitutions to the amino acids corresponding to said positions.

In some aspects, a system described herein comprises, or a method described herein comprises the use of, a site-specific disrupting agent or a polypeptide comprising one or more (e.g., one) targeting moiety and one or more effector moieties (e.g., one or two effector moieties), wherein the one or more targeting moiety is or comprises a CRISPR/Cas molecule comprising a Cas protein, e.g., catalytically inactive Cas9 protein, e.g., sadCas9, dCas9, e.g., dCas9m4, or a functional variant or fragment thereof. In some embodiments, dCas9 comprises an amino acid sequence of SEQ ID NO: 5, 6, or 7:

Cas9
(SEQ ID NO: 5)
DKKYSIGLDIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFLVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNFDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNFDKNLPNEKVLPKHSLLYEYFTVY
NELTKVKYVTEGMRKPAFLSGEQKKAIVDLLFKTNRKVTV
KQLKEDYFKKIECFDSVEISGVEDRFNASLGTYHDLLKII
KDKDFLDNEENEDILEDIVLTLTLFEDREMIEERLKTYAH
LFDDKVMKQLKRRRYTGWGRLSRKLINGIRDKQSGKTILD
FLKSDGFANRNFMQLIHDDSLTFKEDIQKAQVSGQGDSLH
EHIANLAGSPAIKKGILQTVKVVDELVKVMGRHKPENIVI
EMARENQTTQKGQKNSRERMKRIEEGIKELGSQILKEHPV
ENTQLQNEKLYLYYLQNGRDMYVDQELDINRLSDYDVDHI
VPQSFLKDDSIDNKVLTRSDKNRGKSDNVPSEEVVKKMKN
YWRQLLNAKLITQRKFDNLTKAERGGLSELDKAGFIKRQL
VETRQITKHVAQILDSRMNTKYDENDKLIREVKVITLKSK
LVSDFRKDFQFYKVREINNYHHAHDAYLNAVVGTALIKKY
PKLESEFVYGDYKVYDVRKMIAKSEQEIGKATAKYFFYSN
IMNFFKTEITLANGEIRKRPLIETNGETGEIVWDKGRDFA
TVRKVLSMPQVNIVKKTEVQTGGFSKESILPKRNSDKLIA
RKKDWDPKKYGGFDSPTVAYSVLVVAKVEKGKSKKLKSVK
ELLGITIMERSSFEKNPIDFLEAKGYKEVKKDLIIKLPKY
SLFELENGRKRMLASAGELQKGNELALPSKYVNFLYLASH
YEKLKGSPEDNEQKQLFVEQHKHYLDEIIEQISEFSKRVI
LADANLDKVLSAYNKHRDKPIREQAENIIHLFTLTNLGAP
AAFKYFDTTIDRKRYTSTKEVLDATLIHQSITGLYETRID
LSQLGGD
dCas9 (Cas9m4)
(SEQ ID NO: 6)
DKKYSIGLAIGTNSVGWAVITDEYKVPSKKFKVLGNTDRH
SIKKNLIGALLFDSGETAEATRLKRTARRRYTRRKNRICY
LQEIFSNEMAKVDDSFFHRLEESFLVEEDKKHERHPIFGN
IVDEVAYHEKYPTIYHLRKKLVDSTDKADLRLIYLALAHM
IKFRGHFLIEGDLNPDNSDVDKLFIQLVQTYNQLFEENPI
NASGVDAKAILSARLSKSRRLENLIAQLPGEKKNGLFGNL
IALSLGLTPNFKSNFDLAEDAKLQLSKDTYDDDLDNLLAQ
IGDQYADLFLAAKNLSDAILLSDILRVNTEITKAPLSASM
IKRYDEHHQDLTLLKALVRQQLPEKYKEIFFDQSKNGYAG
YIDGGASQEEFYKFIKPILEKMDGTEELLVKLNREDLLRK
QRTFDNGSIPHQIHLGELHAILRRQEDFYPFLKDNREKIE
KILTFRIPYYVGPLARGNSRFAWMTRKSEETITPWNFEEV
VDKGASAQSFIERMTNFDKNLPNEKVLPKHSLLYEYFTVY
NELTKVKYVTEGMRKPAFLSGEQKKAIVDLLFKTNRKVTV
KQLKEDYFKKIECFDSVEISGVEDRFNASLGTYHDLLKII
KDKDFLDNEENEDILEDIVLTLTLFEDREMIEERLKTYAH
LFDDKVMKQLKRRRYTGWGRLSRKLINGIRDKQSGKTILD
FLKSDGFANRNFMQLIHDDSLTFKEDIQKAQVSGQGDSLH
EHIANLAGSPAIKKGILQTVKVVDELVKVMGRHKPENIVI
EMARENQTTQKGQKNSRERMKRIEEGIKELGSQILKEHPV
ENTQLQNEKLYLYYLQNGRDMYVDQELDINRLSDYDVAAI
VPQSFLKDDSIDNKVLTRSDKARGKSDNVPSEEVVKKMKN
YWRQLLNAKLITQRKFDNLTKAERGGLSELDKAGFIKRQL
VETRQITKHVAQILDSRMNTKYDENDKLIREVKVITLKSK
LVSDFRKDFQFYKVREINNYHHAHDAYLNAVVGTALIKKY
PKLESEFVYGDYKVYDVRKMIAKSEQEIGKATAKYFFYSN
IMNFFKTEITLANGEIRKRPLIETNGETGEIVWDKGRDFA
TVRKVLSMPQVNIVKKTEVQTGGFSKESILPKRNSDKLIA
RKKDWDPKKYGGFDSPTVAYSVLVVAKVEKGKSKKLKSVK
ELLGITIMERSSFEKNPIDFLEAKGYKEVKKDLIIKLPKY
SLFELENGRKRMLASAGELQKGNELALPSKYVNFLYLASH
YEKLKGSPEDNEQKQLFVEQHKHYLDEIIEQISEFSKRVI
LADANLDKVLSAYNKHRDKPIREQAENIIHLFTLTNLGAP
AAFKYFDTTIDRKRYTSTKEVLDATLIHQSITGLYETRID
LSQLGGD
Sa-dCas9
(SEQ ID NO: 7)
AKRNYILGLAIGITSVGYGIIDYETRDVIDAGVRLFKEAN
VENNEGRRSKRGARRLKRRRRHRIQRVKKLLFDYNLLTDH
SELSGINPYEARVKGLSQKLSEEEFSAALLHLAKRRGVHN
VNEVEEDTGNELSTKEQISRNSKALEEKYVAELQLERLKK
DGEVRGSINRFKTSDYVKEAKQLLKVQKAYHQLALNDLNN
LVITRDENEKLEYYEKFQUIENVFKQKKKPTLKQIAKEIL
VNEEDIKGYRVTSTGKPEFTNLKVYHDIKDITARKEIIEN
AELLDQIAKILTIYQSSEDIQEELTNLNSELTQEEIEQIS
NLKGYTGTHNLSLKAINLILDELWHTNDNQIAIFNRLKLV
PKKVDLSQQKEIPTTLVDDFILSPVVKRSFIQSIKVINAI
IKKYGLPNDIIIELAREKNSKDAQKMINEMQKRNRQTNER
IEEIIRTTGKENAKYLIEKIKLHDMQEGKCLYSLEAIPLE
DLLNNPFNYEVDAIIPRSVSFDNSFNNKVLVKQEENSKKG
NRTPFQYLSSSDSKISYETFKKHILNLAKGKGRISKTKKE
YLLEERDINRFSVQKDFINRNLVDTRYATRGLMNLLRSYF
RVNNLDVKVKSINGGFTSFLRRKWKFKKERNKGYKHHAED
ALIIANADFIFKEWKKLDKAKKVMENQMFEEKQAESMPEI
ETEQEYKEIFITPHQIKHIKDFKDYKYSHRVDKKPNRKLI
NDTLYSTRKDDKGNTLIVNNLNGLYDKDNDKLKKLINKSP
EKLLMYHHDPQTYQKLKLIMEQYGDEKNPLYKYYEETGNY
LTKYSKKDNGPVIKKIKYYGNKLNAHLDITDDYPNSRNKV
VKLSLKPYRFDVYLDNGVYKFVTVKNLDVIKKENYYEVNS
KCYEEAKKLKKISNQAEFIASFYKNDLIKINGELYRVIGV
NNDLLNRIEVNMIDITYREYLENMNDKRPPHIIKTIASKT
QSIKKYSTDILGNLYEVKSKKHPQIIKKG

In some embodiments, the dCas9 is encoded by a nucleic acid sequence of SEQ ID NO: 8, or 9:

(SEQ ID NO: 8)
GACAAGAAGTACAGCATCGGCCTGGCCATCGGCACCAACAGCGTGGGCTGGGCCGTGATCA
CCGACGAGTACAAGGTGCCCAGCAAGAAGTTCAAGGTGCTGGGCAACACCGACCGGCACAG
CATCAAGAAGAACCTGATCGGCGCCCTGCTGTTCGACAGCGGCGAGACCGCCGAGGCCACC
CGGCTGAAGCGGACCGCCCGGCGGCGGTACACCCGGCGGAAGAACCGGATCTGCTACCTGC
AGGAGATCTTCAGCAACGAGATGGCCAAGGTGGACGACAGCTTCTTCCACCGGCTGGAGGA
GAGCTTCCTGGTGGAGGAGGACAAGAAGCACGAGCGGCACCCCATCTTCGGCAACATCGTG
GACGAGGTGGCCTACCACGAGAAGTACCCCACCATCTACCACCTGCGGAAGAAGCTGGTGG
ACAGCACCGACAAGGCCGACCTGCGGCTGATCTACCTGGCCCTGGCCCACATGATCAAGTTC
CGGGGCCACTTCCTGATCGAGGGCGACCTGAACCCCGACAACAGCGACGTGGACAAGCTGT
TCATCCAGCTGGTGCAGACCTACAACCAGCTGTTCGAGGAGAACCCCATCAACGCCAGCGG
CGTGGACGCCAAGGCCATCCTGAGCGCCCGGCTGAGCAAGAGCCGGCGGCTGGAGAACCTG
ATCGCCCAGCTGCCCGGCGAGAAGAAGAACGGCCTGTTCGGCAACCTGATCGCCCTGAGCC
TGGGCCTGACCCCCAACTTCAAGAGCAACTTCGACCTGGCCGAGGACGCCAAGCTGCAGCT
GAGCAAGGACACCTACGACGACGACCTGGACAACCTGCTGGCCCAGATCGGCGACCAGTAC
GCCGACCTGTTCCTGGCCGCCAAGAACCTGAGCGACGCCATCCTGCTGAGCGACATCCTGCG
GGTGAACACCGAGATCACCAAGGCCCCCCTGAGCGCCAGCATGATCAAGCGGTACGACGAG
CACCACCAGGACCTGACCCTGCTGAAGGCCCTGGTGCGGCAGCAGCTGCCCGAGAAGTACA
AGGAGATCTTCTTCGACCAGAGCAAGAACGGCTACGCCGGCTACATCGACGGCGGCGCCAG
CCAGGAGGAGTTCTACAAGTTCATCAAGCCCATCCTGGAGAAGATGGACGGCACCGAGGAG
CTGCTGGTGAAGCTGAACCGGGAGGACCTGCTGCGGAAGCAGCGGACCTTCGACAACGGCA
GCATCCCCCACCAGATCCACCTGGGCGAGCTGCACGCCATCCTGCGGCGGCAGGAGGACTTC
TACCCCTTCCTGAAGGACAACCGGGAGAAGATCGAGAAGATCCTGACCTTCCGGATCCCCTA
CTACGTGGGCCCCCTGGCCCGGGGCAACAGCCGGTTCGCCTGGATGACCCGGAAATCCGAG
GAGACCATCACCCCCTGGAACTTCGAGGAGGTGGTGGACAAGGGCGCCAGCGCCCAGAGCT
TCATCGAGCGGATGACCAACTTCGACAAGAACCTGCCCAACGAGAAGGTGCTGCCCAAGCA
CAGCCTGCTGTACGAGTACTTCACCGTGTACAACGAGCTGACCAAGGTGAAGTACGTGACCG
AGGGCATGCGGAAGCCCGCCTTCCTGAGCGGCGAGCAGAAGAAGGCCATCGTGGACCTGCT
GTTCAAGACCAACCGGAAGGTGACCGTGAAGCAGCTGAAGGAGGACTACTTCAAGAAGATC
GAGTGCTTCGACAGCGTGGAGATCAGCGGCGTGGAGGACCGGTTCAACGCCAGCCTGGGCA
CCTACCACGACCTGCTGAAGATCATCAAGGACAAGGACTTCCTGGACAACGAGGAGAACGA
GGACATCCTGGAGGACATCGTGCTGACCCTGACCCTGTTCGAGGACCGGGAGATGATCGAG
GAGCGGCTGAAAACCTACGCCCACCTGTTCGACGACAAGGTGATGAAGCAGCTGAAGCGGC
GGCGGTACACCGGCTGGGGCCGGCTGAGCCGGAAGCTGATCAACGGCATCCGGGACAAGCA
GAGCGGCAAGACCATCCTGGACTTCCTGAAATCCGACGGCTTCGCCAACCGGAACTTCATGC
AGCTGATCCACGACGACAGCCTGACCTTCAAGGAGGACATCCAGAAGGCCCAGGTGAGCGG
CCAGGGCGACAGCCTGCACGAGCACATCGCCAACCTGGCCGGCAGCCCCGCCATCAAGAAG
GGCATCCTGCAGACCGTGAAGGTGGTGGACGAGCTGGTGAAGGTGATGGGCCGGCACAAGC
CCGAGAACATCGTGATCGAGATGGCCCGGGAGAACCAGACCACCCAGAAGGGCCAGAAGA
ACAGCCGGGAGCGGATGAAGCGGATCGAGGAGGGCATCAAGGAGCTGGGCAGCCAGATCC
TGAAGGAGCACCCCGTGGAGAACACCCAGCTGCAGAACGAGAAGCTGTACCTGTACTACCT
GCAGAACGGCCGGGACATGTACGTGGACCAGGAGCTGGACATCAACCGGCTGAGCGACTAC
GACGTGGCCGCCATCGTGCCCCAGAGCTTCCTGAAGGACGACAGCATCGACAACAAGGTGC
TGACCCGGAGCGACAAGGCCCGGGGCAAGAGCGACAACGTGCCCAGCGAGGAGGTGGTGA
AGAAGATGAAGAACTACTGGCGGCAGCTGCTGAACGCCAAGCTGATCACCCAGCGGAAGTT
CGACAACCTGACCAAGGCCGAGCGGGGCGGCCTGAGCGAGCTGGACAAGGCCGGCTTCATC
AAGCGGCAGCTGGTGGAGACCCGGCAGATCACCAAGCACGTGGCCCAGATCCTGGACAGCC
GGATGAACACCAAGTACGACGAGAACGACAAGCTGATCCGGGAGGTGAAGGTGATCACCCT
GAAATCCAAGCTGGTGAGCGACTTCCGGAAGGACTTCCAGTTCTACAAGGTGCGGGAGATC
AACAACTACCACCACGCCCACGACGCCTACCTGAACGCCGTGGTGGGCACCGCCCTGATCA
AGAAGTACCCCAAGCTGGAGAGCGAGTTCGTGTACGGCGACTACAAGGTGTACGACGTGCG
GAAGATGATCGCCAAGAGCGAGCAGGAGATCGGCAAGGCCACCGCCAAGTACTTCTTCTAC
AGCAACATCATGAACTTCTTCAAGACCGAGATCACCCTGGCCAACGGCGAGATCCGGAAGC
GGCCCCTGATCGAGACCAACGGCGAGACCGGCGAGATCGTGTGGGACAAGGGCCGGGACTT
CGCCACCGTGCGGAAGGTGCTGAGCATGCCCCAGGTGAACATCGTGAAGAAAACCGAGGTG
CAGACCGGCGGCTTCAGCAAGGAGAGCATCCTGCCCAAGCGGAACAGCGACAAGCTGATCG
CCCGGAAGAAGGACTGGGACCCCAAGAAGTACGGCGGCTTCGACAGCCCCACCGTGGCCTA
CAGCGTGCTGGTGGTGGCCAAGGTGGAGAAGGGCAAGAGCAAGAAGCTGAAATCCGTGAA
GGAGCTGCTGGGCATCACCATCATGGAGCGGAGCAGCTTCGAGAAGAACCCCATCGACTTC
CTGGAGGCCAAGGGCTACAAGGAGGTGAAGAAGGACCTGATCATCAAGCTGCCCAAGTACA
GCCTGTTCGAGCTGGAGAACGGCCGGAAGCGGATGCTGGCCAGCGCCGGCGAGCTGCAGAA
GGGCAACGAGCTGGCCCTGCCCAGCAAGTACGTGAACTTCCTGTACCTGGCCAGCCACTACG
AGAAGCTGAAGGGCAGCCCCGAGGACAACGAGCAGAAGCAGCTGTTCGTGGAGCAGCACA
AGCACTACCTGGACGAGATCATCGAGCAGATCAGCGAGTTCAGCAAGCGGGTGATCCTGGC
CGACGCCAACCTGGACAAGGTGCTGAGCGCCTACAACAAGCACCGGGACAAGCCCATCCGG
GAGCAGGCCGAGAACATCATCCACCTGTTCACCCTGACCAACCTGGGCGCCCCCGCCGCCTT
CAAGTACTTCGACACCACCATCGACCGGAAGCGGTACACCAGCACCAAGGAGGTGCTGGAC
GCCACCCTGATCCACCAGAGCATCACCGGCCTGTACGAGACCCGGATCGACCTGAGCCAGCT
GGGCGGCGAC
(SEQ ID NO: 9)
GCCAAGCGGAACTACATCCTGGGCCTGGCCATCGGCATCACCAGCGTGGGCTACGGCATCAT
CGACTACGAGACCCGGGACGTGATCGACGCCGGCGTGCGGCTGTTCAAGGAGGCCAACGTG
GAGAACAACGAGGGCCGGCGGAGCAAGCGGGGCGCCCGGCGGCTGAAGCGGCGGCGGCGG
CACCGGATCCAGCGGGTGAAGAAGCTGCTGTTCGACTACAACCTGCTGACCGACCACAGCG
AGCTGAGCGGCATCAACCCCTACGAGGCCCGGGTGAAGGGCCTGAGCCAGAAGCTGAGCGA
GGAGGAGTTCAGCGCCGCCCTGCTGCACCTGGCCAAGCGGCGGGGCGTGCACAACGTGAAC
GAGGTGGAGGAGGACACCGGCAACGAGCTGAGCACCAAGGAGCAGATCAGCCGGAACAGC
AAGGCCCTGGAGGAGAAGTACGTGGCCGAGCTGCAGCTGGAGCGGCTGAAGAAGGACGGC
GAGGTGCGGGGCAGCATCAACCGGTTCAAGACCAGCGACTACGTGAAGGAGGCCAAGCAGC
TGCTGAAGGTGCAGAAGGCCTACCACCAGCTGGACCAGAGCTTCATCGACACCTACATCGA
CCTGCTGGAGACCCGGCGGACCTACTACGAGGGCCCCGGCGAGGGCAGCCCCTTCGGCTGG
AAGGACATCAAGGAGTGGTACGAGATGCTGATGGGCCACTGCACCTACTTCCCCGAGGAGC
TGCGGAGCGTGAAGTACGCCTACAACGCCGACCTGTACAACGCCCTGAACGACCTGAACAA
CCTGGTGATCACCCGGGACGAGAACGAGAAGCTGGAGTACTACGAGAAGTTCCAGATCATC
GAGAACGTGTTCAAGCAGAAGAAGAAGCCCACCCTGAAGCAGATCGCCAAGGAGATCCTGG
TGAACGAGGAGGACATCAAGGGCTACCGGGTGACCAGCACCGGCAAGCCCGAGTTCACCAA
CCTGAAGGTGTACCACGACATCAAGGACATCACCGCCCGGAAGGAGATCATCGAGAACGCC
GAGCTGCTGGACCAGATCGCCAAGATCCTGACCATCTACCAGAGCAGCGAGGACATCCAGG
AGGAGCTGACCAACCTGAACAGCGAGCTGACCCAGGAGGAGATCGAGCAGATCAGCAACCT
GAAGGGCTACACCGGCACCCACAACCTGAGCCTGAAGGCCATCAACCTGATCCTGGACGAG
CTGTGGCACACCAACGACAACCAGATCGCCATCTTCAACCGGCTGAAGCTGGTGCCCAAGA
AGGTGGACCTGAGCCAGCAGAAGGAGATCCCCACCACCCTGGTGGACGACTTCATCCTGAG
CCCCGTGGTGAAGCGGAGCTTCATCCAGAGCATCAAGGTGATCAACGCCATCATCAAGAAG
TACGGCCTGCCCAACGACATCATCATCGAGCTGGCCCGGGAGAAGAACAGCAAGGACGCCC
AGAAGATGATCAACGAGATGCAGAAGCGGAACCGGCAGACCAACGAGCGGATCGAGGAGA
TCATCCGGACCACCGGCAAGGAGAACGCCAAGTACCTGATCGAGAAGATCAAGCTGCACGA
CATGCAGGAGGGCAAGTGCCTGTACAGCCTGGAGGCCATCCCCCTGGAGGACCTGCTGAAC
AACCCCTTCAACTACGAGGTGGACGCCATCATCCCCCGGAGCGTGAGCTTCGACAACAGCTT
CAACAACAAGGTGCTGGTGAAGCAGGAGGAGAACAGCAAGAAGGGCAACCGGACCCCCTT
CCAGTACCTGAGCAGCAGCGACAGCAAGATCAGCTACGAGACCTTCAAGAAGCACATCCTG
AACCTGGCCAAGGGCAAGGGCCGGATCAGCAAGACCAAGAAGGAGTACCTGCTGGAGGAG
CGGGACATCAACCGGTTCAGCGTGCAGAAGGACTTCATCAACCGGAACCTGGTGGACACCC
GGTACGCCACCCGGGGCCTGATGAACCTGCTGCGGAGCTACTTCCGGGTGAACAACCTGGA
CGTGAAGGTGaaatccATCAACGGCGGCTTCACCAGCTTCCTGCGGCGGAAGTGGAAGTTCAAG
AAGGAGCGGAACAAGGGCTACAAGCACCACGCCGAGGACGCCCTGATCATCGCCAACGCCG
ACTTCATCTTCAAGGAGTGGAAGAAGCTGGACAAGGCCAAGAAGGTGATGGAGAACCAGAT
GTTCGAGGAGAAGCAGGCCGAGAGCATGCCCGAGATCGAGACCGAGCAGGAGTACAAGGA
GATCTTCATCACCCCCCACCAGATCAAGCACATCAAGGACTTCAAGGACTACAAGTACAGCC
ACCGGGTGGACAAGAAGCCCAACCGGAAGCTGATCAACGACACCCTGTACAGCACCCGGAA
GGACGACAAGGGCAACACCCTGATCGTGAACAACCTGAACGGCCTGTACGACAAGGACAAC
GACAAGCTGAAGAAGCTGATCAACAAGAGCCCCGAGAAGCTGCTGATGTACCACCACGACC
CCCAGACCTACCAGAAGCTGAAGCTGATCATGGAGCAGTACGGCGACGAGAAGAACCCCCT
GTACAAGTACTACGAGGAGACCGGCAACTACCTGACCAAGTACAGCAAGAAGGACAACGGC
CCCGTGATCAAGAAGATCAAGTACTACGGCAACAAGCTGAACGCCCACCTGGACATCACCG
ACGACTACCCCAACAGCCGGAACAAGGTGGTGAAGCTGAGCCTGAAGCCCTACCGGTTCGA
CGTGTACCTGGACAACGGCGTGTACAAGTTCGTGACCGTGAAGAACCTGGACGTGATCAAG
AAGGAGAACTACTACGAGGTGAACAGCAAGTGCTACGAGGAGGCCAAGAAGCTGAAGAAG
ATCAGCAACCAGGCCGAGTTCATCGCCAGCTTCTACAAGAACGACCTGATCAAGATCAACG
GCGAGCTGTACCGGGTGATCGGCGTGAACAACGACCTGCTGAACCGGATCGAGGTGAACAT
GATCGACATCACCTACCGGGAGTACCTGGAGAACATGAACGACAAGCGGCCCCCCCACATC
ATCAAGACCATCGCCAGCAAGACCCAGAGCATCAAGAAGTACAGCACCGACATCCTGGGCA
ACCTGTACGAGGTGaaatccAAGAAGCACCCCCAGATCATCAAGAAGGGC

Guide RNA (gRNA)

In some embodiments, a targeting moiety may comprise a Cas molecule comprising or linked (e.g., covalently) to a gRNA. A gRNA is a short synthetic RNA composed of a “scaffold” sequence necessary for Cas-protein binding and a user-defined ˜20 nucleotide targeting sequence for a genomic target. In practice, guide RNA sequences are generally designed to have a length of between 17-24 nucleotides (e.g., 19, 20, or 21 nucleotides) and be complementary to the targeted nucleic acid sequence. In some embodiments the gRNA comprises 3-6 flanking phosphorothioate (PS) linkages, e.g., 3 flanking PS linkages at each end. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective for use with Cas proteins; see, for example, Hendel et al. (2015) Nature Biotechnol., 985-991.

In some embodiments, a gRNA comprises a nucleic acid sequence that is complementary to a DNA sequence associated with a target gene. In some embodiments, the DNA sequence is, comprises, or overlaps an expression control element that is operably linked to the target gene. In some embodiments, a gRNA comprises a nucleic acid sequence that is at least 90, 95, 99, or 100% complementary to a DNA sequence associated with a target gene. In some embodiments, a gRNA for use with a targeting moiety that comprises a Cas molecule is an sgRNA. In some embodiments, a gRNA binds to a nucleic acid sequence comprising a sequence selected from Table 4, Table 5, Table 6, Table 7 or a sequence having at least 70, 75, 80, 85, 90, 91, 92, 93, 94, 95, 96, 97, 98, or 99% identity thereto, or differing at no more than 1, 2, 3, 4, or 5 positions relative thereto.

In some embodiments, a gRNA for use with a CRISPR/Cas molecule specifically binds a target sequence associated with β-2-microglobulin expression. In some embodiments, a gRNA for use with a CRISPR/Cas molecule specifically binds a target sequence associated with one or more of CXCL1-8 gene expression. Such a gRNA may comprise a target-binding sequence selected from SEQ ID NOs: 20-62.

In some embodiments, a targeting moiety is or comprises a TAL effector molecule. A TAL effector molecule, e.g., a TAL effector molecule that specifically binds a DNA sequence, comprises a plurality of TAL effector domains or fragments thereof, and optionally one or more additional portions of naturally occurring TAL effectors (e.g., N- and/or C-terminal of the plurality of TAL effector domains). Many TAL effectors are known to those of skill in the art and are commercially available, e.g., from Thermo Fisher Scientific.

TALEs are natural effector proteins secreted by numerous species of bacterial pathogens including the plant pathogen Xanthomonas which modulates gene expression in host plants and facilitates bacterial colonization and survival. The specific binding of TAL effectors is based on a central repeat domain of tandemly arranged nearly identical repeats of typically 33 or 34 amino acids (the repeat-variable di-residues, RVD domain).

Members of the TAL effectors family differ mainly in the number and order of their repeats. The number of repeats ranges from 1.5 to 33.5 repeats and the C-terminal repeat is usually shorter in length (e.g., about 20 amino acids) and is generally referred to as a “half-repeat”. Each repeat of the TAL effector feature a one-repeat-to-one-base-pair correlation with different repeat types exhibiting different base-pair specificity (one repeat recognizes one base-pair on the target gene sequence). Generally, the smaller the number of repeats, the weaker the protein-DNA interactions. A number of 6.5 repeats has been shown to be sufficient to activate transcription of a reporter gene (Scholze et al., 2010).

Repeat to repeat variations occur predominantly at amino acid positions 12 and 13, which have therefore been termed “hypervariable” and which are responsible for the specificity of the interaction with the target DNA promoter sequence, as shown in Table 2 listing exemplary repeat variable di-residues (RVD) and their correspondence to nucleic acid base targets.

TABLE 2
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

Accordingly, it is possible to modify the repeats of a TAL effector to target specific DNA sequences. Further studies have shown that the RVD NK can target G. Target sites of TAL effectors also tend to include a T flanking the 5′ base targeted by the first repeat, but the exact mechanism of this recognition is not known. More than 113 TAL effector sequences are known to date. Non-limiting examples of TAL effectors from Xanthomonas include, Hax2, Hax3, Hax4, AvrXa7, AvrXa10 and AvrBs3.

Accordingly, the TAL effector domain of the TAL effector molecule of the present disclosure may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzicola strain BLS256 (Bogdanove et al. 2011). As used herein, the TAL effector domain in accordance with the present disclosure comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL effector molecule of the present disclosure is designed to target a given DNA sequence based on the above code and others known in the art. The number of TAL effector domains (e.g., repeats (monomers or modules)) and their specific sequence are selected based on the desired DNA target sequence. For example, TAL effector domains, e.g., repeats, may be removed or added in order to suit a specific target sequence. In an embodiment, the TAL effector molecule of the present disclosure comprises between 6.5 and 33.5 TAL effector domains, e.g., repeats. In an embodiment, TAL effector molecule of the present disclosure comprises between 8 and 33.5 TAL effector domains, e.g., repeats, e.g., between 10 and 25 TAL effector domains, e.g., repeats, e.g., between 10 and 14 TAL effector domains, e.g., repeats.

In some embodiments, the TAL effector molecule comprises TAL effector domains that correspond to a perfect match to the DNA target sequence. In some embodiments, a mismatch between a repeat and a target base-pair on the DNA target sequence is permitted as along as it allows for the function of the site-specific disrupting agent comprising the TAL effector molecule. In general, TALE binding is inversely correlated with the number of mismatches. In some embodiments, the TAL effector molecule of a site-specific disrupting agent of the present disclosure comprises no more than 7 mismatches, 6 mismatches, 5 mismatches, 4 mismatches, 3 mismatches, 2 mismatches, or 1 mismatch, and optionally no mismatch, with the target DNA sequence. Without wishing to be bound by theory, in general the smaller the number of TAL effector domains in the TAL effector molecule, the smaller the number of mismatches will be tolerated and still allow for the function of the site-specific disrupting agent comprising the TAL effector molecule. The binding affinity is thought to depend on the sum of matching repeat-DNA combinations. For example, TAL effector molecules having 25 TAL effector domains or more may be able to tolerate up to 7 mismatches.

In addition to the TAL effector domains, the TAL effector molecule of the present disclosure may comprise additional sequences derived from a naturally occurring TAL effector. The length of the C-terminal and/or N-terminal sequence(s) included on each side of the TAL effector domain portion of the TAL effector molecule can vary and be selected by one skilled in the art, for example based on the studies of Zhang et al. (2011). Zhang et al., have characterized a number of C-terminal and N-terminal truncation mutants in Hax3 derived TAL-effector based proteins and have identified key elements, which contribute to optimal binding to the target sequence and thus activation of transcription. Generally, it was found that transcriptional activity is inversely correlated with the length of N-terminus. Regarding the C-terminus, an important element for DNA binding residues within the first 68 amino acids of the Hax 3 sequence was identified. Accordingly, in some embodiments, the first 68 amino acids on the C-terminal side of the TAL effector domains of the naturally occurring TAL effector is included in the TAL effector molecule of a site-specific disrupting agent of the present disclosure. Accordingly, in an embodiment, a TAL effector molecule of the present disclosure comprises 1) one or more TAL effector domains derived from a naturally occurring TAL effector; 2) at least 70, 80, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains; and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

In some embodiments, a targeting moiety is or comprises a Zn finger molecule. A Zn finger molecule comprises a Zn finger protein, e.g., a naturally occurring Zn finger protein or engineered Zn finger protein, or fragment thereof. Many Zn finger proteins are known to those of skill in the art and are commercially available, e.g., from Sigma-Aldrich.

In some embodiments, a Zn finger molecule comprises a non-naturally occurring Zn finger protein that is engineered to bind to a target DNA sequence of choice. See, for example, Beerli, et al. (2002) Nature Biotechnol. 20:135-141; Pabo, et al. (2001) Ann. Rev. Biochem. 70:313-340; Isalan, et al. (2001) Nature Biotechnol. 19:656-660; Segal, et al. (2001) Curr. Opin. Biotechnol. 12:632-637; Choo, et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered Zn finger protein may have a novel binding specificity, compared to a naturally-occurring Zn finger protein. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein. In addition, enhancement of binding specificity for zinc finger binding domains has been described, for example, in co-owned International Patent Publication No. WO 02/077227.

Zn finger proteins and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

In addition, as disclosed in these and other references, Zn finger proteins and/or multi-fingered Zn finger proteins may be linked together, e.g., as a fusion protein, using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The Zn finger molecules described herein may include any combination of suitable linkers between the individual zinc finger proteins and/or multi-fingered Zn finger proteins of the Zn finger molecule.

In certain embodiments, the targeting moiety comprises a Zn finger molecule comprising an engineered zinc finger protein that binds (in a sequence-specific manner) to a target DNA sequence. In some embodiments, the Zn finger molecule comprises one Zn finger protein or fragment thereof. In other embodiments, the Zn finger molecule comprises a plurality of Zn finger proteins (or fragments thereof), e.g., 2, 3, 4, 5, 6 or more Zn finger proteins (and optionally no more than 12, 11, 10, 9, 8, 7, 6, 5, 4, 3, or 2 Zn finger proteins). In some embodiments, the Zn finger molecule comprises at least three Zn finger proteins. In some embodiments, the Zn finger molecule comprises four, five or six fingers. In some embodiments, the Zn finger molecule comprises 8, 9, 10, 11 or 12 fingers. In some embodiments, a Zn finger molecule comprising three Zn finger proteins recognizes a target DNA sequence comprising 9 or 10 nucleotides. In some embodiments, a Zn finger molecule comprising four Zn finger proteins recognizes a target DNA sequence comprising 12 to 14 nucleotides. In some embodiments, a Zn finger molecule comprising six Zn finger proteins recognizes a target DNA sequence comprising 18 to 21 nucleotides.

In some embodiments, a Zn finger molecule comprises a two-handed Zn finger protein. Two handed zinc finger proteins are those proteins in which two clusters of zinc finger proteins are separated by intervening amino acids so that the two zinc finger domains bind to two discontinuous target DNA sequences. An example of a two handed type of zinc finger binding protein is SIP1, where a cluster of four zinc finger proteins is located at the amino terminus of the protein and a cluster of three Zn finger proteins is located at the carboxyl terminus (see Remade, et al. (1999) EMBO Journal 18(18):5073-5084). Each cluster of zinc fingers in these proteins is able to bind to a unique target sequence and the spacing between the two target sequences can comprise many nucleotides.

In some embodiments, a targeting moiety is or comprises a DNA-binding domain from a nuclease. For example, the recognition sequences of homing endonucleases and meganucleases such as I-Scel, I-Ceul, PI-PspI, PI-Sce, I-SceIV, I-CsmI, I-PanI, I-Scell, I-PpoI, I-ScellI, I-Crel, I-TevI, I-TevII and I-TevIII are known. See also U.S. Pat. Nos. 5,420,032; 6,833,252; Belfort, et al. (1997) Nucleic Acids Res. 25:3379-3388; Dujon, et al. (1989) Gene 82:115-118; Perler, et al. (1994) Nucleic Acids Res. 22:1125-1127; Jasin (1996) Trends Genet. 12:224-228; Gimble, et al. (1996) J. Mol. Biol. 263:163-180; Argast, et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier, et al. (2002) Molec. Cell 10:895-905; Epinat, et al. (2003) Nucleic Acids Res. 31:2952-2962; Ashworth, et al. (2006) Nature 441:656-659; Paques, et al. (2007) Current Gene Therapy 7:49-66; U.S. Patent Publication No. 2007/0117128.

In some embodiments, a targeting moiety comprises a nucleic acid. In some embodiments, a nucleic acid that may be included in a targeting moiety, may be or comprise DNA, RNA, and/or an artificial or synthetic nucleic acid or nucleic acid analog or mimic. For example, in some embodiments, a nucleic acid may be or include one or more of genomic DNA (gDNA), complementary DNA (cDNA), a peptide nucleic acid (PNA), a peptide-oligonucleotide conjugate, a locked nucleic acid (LNA), a bridged nucleic acid (BNA), a polyamide, a triplex-forming oligonucleotide, an antisense oligonucleotide, tRNA, mRNA, rRNA, miRNA, gRNA, siRNA or other RNAi molecule (e.g., that targets a non-coding RNA as described herein and/or that targets an expression product of a particular gene associated with a targeted genomic complex as described herein), etc. In some embodiments, a nucleic acid may include one or more residues that is not a naturally occurring DNA or RNA residue, may include one or more linkages that is/are not phosphodiester bonds (e.g., that may be, for example, phosphorothioate bonds, etc), and/or may include one or more modifications such as, for example, a 2′O modification such as 2′-OMeP. A variety of nucleic acid structures useful in preparing synthetic nucleic acids is known in the art (see, for example, WO2017/0628621 and WO2014/012081) those skilled in the art will appreciate that these may be utilized in accordance with the present disclosure.

A nucleic acid suitable for use in a site-specific disrupting agent, e.g., in a targeting moiety, may include, but is not limited to, DNA, RNA, modified oligonucleotides (e.g., chemical modifications, such as modifications that alter backbone linkages, sugar molecules, and/or nucleic acid bases), and artificial nucleic acids. In some embodiments, a nucleic acid includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mRNA, rRNA, modified RNA, miRNA, gRNA, and siRNA or other RNA or DNA molecules.

In some embodiments, a targeting moiety comprises a nucleic acid with a length from about 15-200, 20-200, 30-200, 40-200, 50-200, 60-200, 70-200, 80-200, 90-200, 100-200, 110-200, 120-200, 130-200, 140-200, 150-200, 160-200, 170-200, 180-200, 190-200, 215-190, 20-190, 30-190, 40-190, 50-190, 60-190, 70-190, 80-190, 90-190, 100-190, 110-190, 120-190, 130-190, 140-190, 150-190, 160-190, 170-190, 180-190, 15-180, 20-180, 30-180, 40-180, 50-180, 60-180, 70-180, 80-180, 90-180, 100-180, 110-180, 120-180, 130-180, 140-180, 150-180, 160-180, 170-180, 15-170, 20-170, 30-170, 40-170, 50-170, 60-170, 70-170, 80-170, 90-170, 100-170, 110-170, 120-170, 130-170, 140-170, 150-170, 160-170, 15-160, 20-160, 30-160, 40-160, 50-160, 60-160, 70-160, 80-160, 90-160, 100-160, 110-160, 120-160, 130-160, 140-160, 150-160, 215-150, 20-150, 30-150, 40-150, 50-150, 60-150, 70-150, 80-150, 90-150, 100-150, 110-150, 120-150, 130-150, 140-150, 15-140, 20-140, 30-140, 40-140, 50-140, 60-140, 70-140, 80-140, 90-140, 100-140, 110-140, 120-140, 130-140, 15-130, 20-130, 30-130, 40-130, 50-130, 60-130, 70-130, 80-130, 90-130, 100-130, 110-130, 120-130, 215-120, 20-120, 30-120, 40-120, 50-120, 60-120, 70-120, 80-120, 90-120, 100-120, 110-120, 15-110, 20-110, 30-110, 40-110, 50-110, 60-110, 70-110, 80-110, 90-110, 100-110, 15-100, 20-100, 30-100, 40-100, 50-100, 60-100, 70-100, 80-100, 90-100, 15-90, 20-90, 30-90, 40-90, 50-90, 60-90, 70-90, 80-90, 15-80, 20-80, 30-80, 40-80, 50-80, 60-80, 70-80, 15-70, 20-70, 30-70, 40-70, 50-70, 60-70, 15-60, 20-60, 30-60, 40-60, 50-60, 15-50, 20-50, 30-50, 40-50, 15-40, 20-40, 30-40, 15-30, 20-30, or 15-20 nucleotides, or any range therebetween.

Effector Moieties

A site-specific disrupting agent of the present disclosure may comprise one or more effector moieties. An effector moiety has one or more functionalities that, when used as part of a site-specific disrupting agent described herein, modulate, e.g., decrease, expression of a target plurality of genes in a cell. In some embodiments, an effector moiety physically or sterically blocks an anchor sequence, e.g., such that binding of a genomic complex component (e.g., a nucleating polypeptide) to the anchor sequence is inhibited (e.g., prevented). In some embodiments, an effector moiety destabilizes the interaction of a genomic complex component (e.g., nucleating polypeptide) with an anchor sequence, e.g., by altering (e.g., decreasing) the affinity and/or avidity at which the genomic complex component binds the anchor sequence. For example, an effector moiety may recruit a factor that inhibits formation of or destabilizes a genomic complex, e.g., ASMC, or it may inhibit recruitment of a factor (e.g., a genomic complex component or transcription factor) necessary for formation or maintenance of a genomic complex (e.g., ASMC). In some embodiments, an effector moiety has epigenetic modification functionality in that it modulates the epigenetic landscape of the anchor sequence or a sequence proximal to the anchor sequence, e.g., by promoting (e.g., catalyzing) application or removal of one or more epigenetic modifications to the DNA or a histone associated thereto, to decrease expression of a target plurality of genes. In some embodiments, an effector moiety has genetic modification functionality, e.g., it introduces an alteration (e.g., an insertion, deletion, or substitution) to an anchor sequence or a sequence proximal thereto.

In some embodiments, an effector moiety comprises a CRISPR/Cas molecule, a TAL effector molecule, a Zn finger molecule, a tetR domain, or a meganuclease. In some embodiments, an effector moiety has genetic modification functionality, e.g., a CRISPR/Cas molecule, a TAL effector molecule, a Zn finger molecule with endonuclease activity capable of making a genetic alteration in a method described herein.

In some embodiments, an effector moiety comprises a histone modifying functionality, e.g., a histone methyltransferase, histone demethylase, or histone deacetylase activity. In some embodiments, a histone methyltransferase functionality comprises H3K9 targeting methyltransferase activity. In some embodiments, a histone methyltransferase functionality comprises H3K56 targeting methyltransferase activity. In some embodiments, a histone methyltransferase functionality comprises H3K27 targeting methyltransferase activity. In some embodiments, a histone methyltransferase or demethylase functionality transfers one, two, or three methyl groups. In some embodiments, a histone demethylase functionality comprises H3K4 targeting demethylase activity. In some embodiments, an effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof, e.g., a SET domain of any thereof. In some embodiments, an effector moiety comprises a protein chosen from KDM1A (i.e., LSD1), KDM1B (i.e., LSD2), KDM2A, KDM2B, KDM5A, KDM5B, KDM5C, KDM5D, KDM4B, N066, or a functional variant or fragment of any thereof. In some embodiments, an effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof. In some embodiments, an effector moiety comprises a DNA modifying functionality, e.g., a DNA methyltransferase. In some embodiments, an effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, or a functional variant or fragment of any thereof.

In some embodiments, an effector moiety comprises a transcription repressor. In some embodiments the transcription repressor blocks recruitment of a factor that stimulates or promotes transcription, e.g., of the target gene. In some embodiments, the transcription repressor recruits a factor that inhibits transcription, e.g., of the target gene. In some embodiments, an effector moiety, e.g., transcription repressor, is or comprises a protein chosen from KRAB, MeCP2, HP1, RBBP4, REST, FOG1, SUZ12, or a functional variant or fragment of any thereof.

In some embodiments, an effector moiety comprises a protein having a functionality described herein. In some embodiments, an effector moiety comprises a protein selected from:
KRAB (e.g., as according to NP_056209.2 or the protein encoded by NM_015394.5); a SET domain (e.g., the SET domain of:
SETDB1 (e.g., as according to NP_001353347.1 or the protein encoded by NM_001366418.1);
EZH2 (e.g., as according to NP-004447.2 or NP_001190176.1 or the protein encoded by NM_004456.5 or NM_001203247.2);
G9A (e.g., as according to NP_001350618.1 or the protein encoded by NM_001363689.1); or
SUV39H1 (e.g., as according to NP_003164.1 or the protein encoded by NM_003173.4));
histone demethylase LSD1 (e.g., as according to NP_055828.2 or the protein encoded by NM_015013.4);
FOG1 (e.g., the N-terminal residues of FOG1) (e.g., as according to NP_722520.2 or the protein encoded by NM_153813.3); or
KAP1 (e.g., as according to NP_005753.1 or the protein encoded by NM_005762.3); a functional fragment or variant of any thereof, or
a polypeptide with a sequence that has at least 80, 85, 90, 91, 92, 93, 94, 95, 96, 97, 98, or 99% identity to any of the above-referenced sequences. In some embodiments, an effector moiety comprises a protein selected from:
DNMT3A (e.g., human DNMT3A) (e.g., as according to NP_072046.2 or the protein encoded by NM_022552.4);
DNMT3B (e.g., as according to NP_008823.1
or the protein encoded by NM_006892.4);
DNMT3L (e.g., as according to NP_787063.1
or the protein encoded by NM_175867.3);
DNMT3A/3L complex,
bacterial MQ1 (e.g., as according to CAA35058.1 obtained from strain ATCC 33825 or Uniprot ID P15840.3);
a functional fragment of any thereof, or
a polypeptide with a sequence that has at least 80, 85, 90, 91, 92, 93, 94, 95, 96, 97, 98, or 99% identity to any of the above-referenced sequences. In some embodiments, an effector moiety comprises a mature bacterial MQ1 (e.g., as according to CAA35058.1 obtained from strain ATCC 33825 or Uniprot ID P15840.3

An exemplary effector moiety may include, but is not limited to: ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3A, DNMT3B, DNMT3L), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdb1), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDM1A and lysine-specific histone demethylase 1 (LSD1), helicases such as DHX9, deacetylases (e.g., sirtuin 1, 2, 3, 4, 5, 6, or 7), kinases, phosphatases, DNA-intercalating agents such as ethidium bromide, SYBR green, and proflavine, efflux pump inhibitors such as peptidomimetics like phenylalanine arginyl β-naphthylamide or quinoline derivatives, nuclear receptor activators and inhibitors, proteasome inhibitors, competitive inhibitors for enzymes such as those involved in lysosomal storage diseases, protein synthesis inhibitors, nucleases (e.g., Cpf1, Cas9, zinc finger nuclease), fusions of one or more thereof (e.g., dCas9-DNMT, dCas9-APOBEC, dCas9-UG1), and specific domains from proteins, such as a KRAB domain.

In some embodiments, a candidate domain may be determined to be suitable for use as an effector moiety by methods known to those of skill in the art. For example, a candidate effector moiety may be tested by assaying whether, when the candidate effector moiety is present in the nucleus of a cell and appropriately localized (e.g., to a target gene or transcription control element operably linked to said target gene, e.g., via a Targeting moiety), the candidate effector moiety decreases expression of the target gene in the cell, e.g., decreases the level of RNA transcript encoded by the target gene (e.g., as measured by RNASeq or Northern blot) or decreases the level of protein encoded by the target gene (e.g., as measured by ELISA).

In some embodiments, a site-specific disrupting agent comprises an effector moiety that does not bind (e.g., does not detectably bind) to another copy of the effector moiety, e.g., the effector moiety is monomeric and does not associate into multimers. In some embodiments, a site-specific disrupting agent comprises an effector moiety that associates with a further copy of the effector moiety into a multimer, e.g., dimers, trimers, tetramers, or further. In some embodiments, a site-specific disrupting agent comprises a plurality of effector moieties, wherein each effector moiety does not detectably bind, e.g., does not bind, to another effector moiety. In some embodiments, an effector moiety for use in the compositions and methods described herein is functional in a monomeric, e.g., non-dimeric, state.

In some embodiments, an effector moiety comprises an epigenetic modifying moiety, e.g., that modulates the two-dimensional structure of chromatin (i.e., that modulate structure of chromatin in a way that would alter its two-dimensional representation).

Epigenetic modifying moieties useful in methods and compositions of the present disclosure include agents that affect epigenetic markers, e.g., DNA methylation, histone methylation, histone acetylation, histone sumoylation, histone phosphorylation, and RNA-associated silencing. Exemplary epigenetic enzymes that can be targeted to a genomic sequence element as described herein include DNA methylases (e.g., DNMT3a, DNMT3b, DNMTL, MQ1), DNA demethylation (e.g., the TET family), histone methyltransferases, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), and protein-lysine N-methyltransferase (SMYD2). Examples of such epigenetic modifying agents are described, e.g., in de Groote et al. Nuc. Acids Res. (2012):1-18.

In some embodiments, a site-specific disrupting agent, e.g., comprising an epigenetic modifying moiety, useful herein comprises or is a construct described in Koferle et al. Genome Medicine 7.59 (2015):1-3 incorporated herein by reference. For example, in some embodiments, a site-specific disrupting agent comprises or is a construct found in Table 1 of Koferle et al., e.g., histone deacetylase, histone methyltransferase, DNA demethylation, or H3K4 and/or H3K9 histone demethylase described in Table 1 (e.g., dCas9-p300, TALE-TET1, ZF-DNMT3A, or TALE-LSD1).

Additional Moieties

A site-specific disrupting agent may further comprise one or more additional moieties (e.g., in addition to one or more targeting moieties and one or more effector moieties). In some embodiments, an additional moiety is selected from a tagging or monitoring moiety, a cleavable moiety (e.g., a cleavable moiety positioned between a targeting moiety and an effector moiety or at the N- or C-terminal end of a polypeptide), a small molecule, a membrane translocating polypeptide, or a pharmacoagent moiety.

### Exemplary Site-Specific Disrupting Agents

The following exemplary site-specific disrupting agents are presented for illustration purposes only and are not intended to be limiting.

In some embodiments, a site-specific disrupting agent comprises a targeting moiety (e.g., comprising dCas9, e.g., an S. aureus dCas9), and an effector moiety comprising MQ1, e.g., bacterial MQ1. In some embodiments, the site-specific disrupting agent is encoded by the nucleic acid sequence of SEQ ID NOs: 201 (e.g., a plasmid encoding the site-specific disrupting agent), and/or 202 (e.g., a nucleic acid (e.g., mRNA) encoding the site-specific disrupting agent), encoding the site-specific disrupting agent). In some embodiments, a nucleic acid described herein comprises a nucleic acid sequence of SEQ ID NO: 201 or 202 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 9 and/or the effector moiety is encoded by the nucleic acid sequence of SEQ ID NO: 10.

Sa-dCas9-MQ1 (PL-27695) Plasmid DNA sequence:
(SEQ ID NO: 201)
TCGCGCGTTTCGGTGATGACGGTGAAAACCTCTGACACAT
GCAGCTCCCGGAGACGGTCACAGCTTGTCTGTAAGCGGAT
GCCGGGAGCAGACAAGCCCGTCAGGGCGCGTCAGCGGGTG
TTGGCGGGTGTCGGGGCTGGCTTAACTATGCGGCATCAGA
GCAGATTGTACTGAGAGTGCACCATATGCGGTGTGAAATA
CCGCACAGATGCGTAAGGAGAAAATACCGCATCAGGCGCC
ATTCGCCATTCAGGCTGCGCAACTGTTGGGAAGGGCGATC
GGTGCGGGCCTCTTCGCTATTACGCCAGCTGGCGAAAGGG
GGATGTGCTGCAAGGCGATTAAGTTGGGTAACGCCAGGGT
TTTCCCAGTCACGACGTTGTAAAACGACGGCCAGTGAATT
GACGCGTATTGGGATGGTACCTAATACGACTCACTATAAG
GAAATAAGAGAGAAAAGAAGAGTAAGAAGAAATATAAGAG
CCACCATGGCCCCCAAGAAGAAGCGGAAGGTGGGCATCCA
CGGCGTGCCCGCCGCCGCCAAGCGGAACTACATCCTGGGC
CTGGCCATCGGCATCACCAGCGTGGGCTACGGCATCATCG
ACTACGAGACCCGGGACGTGATCGACGCCGGCGTGCGGCT
GTTCAAGGAGGCCAACGTGGAGAACAACGAGGGCCGGCGG
AGCAAGCGGGGCGCCCGGCGGCTGAAGCGGCGGCGGCGGC
ACCGGATCCAGCGGGTGAAGAAGCTGCTGTTCGACTACAA
CCTGCTGACCGACCACAGCGAGCTGAGCGGCATCAACCCC
TACGAGGCCCGGGTGAAGGGCCTGAGCCAGAAGCTGAGCG
AGGAGGAGTTCAGCGCCGCCCTGCTGCACCTGGCCAAGCG
GCGGGGCGTGCACAACGTGAACGAGGTGGAGGAGGACACC
GGCAACGAGCTGAGCACCAAGGAGCAGATCAGCCGGAACA
GCAAGGCCCTGGAGGAGAAGTACGTGGCCGAGCTGCAGCT
GGAGCGGCTGAAGAAGGACGGCGAGGTGCGGGGCAGCATC
AACCGGTTCAAGACCAGCGACTACGTGAAGGAGGCCAAGC
AGCTGCTGAAGGTGCAGAAGGCCTACCACCAGCTGGACCA
GAGCTTCATCGACACCTACATCGACCTGCTGGAGACCCGG
CGGACCTACTACGAGGGCCCCGGCGAGGGCAGCCCCTTCG
GCTGGAAGGACATCAAGGAGTGGTACGAGATGCTGATGGG
CCACTGCACCTACTTCCCCGAGGAGCTGCGGAGCGTGAAG
TACGCCTACAACGCCGACCTGTACAACGCCCTGAACGACC
TGAACAACCTGGTGATCACCCGGGACGAGAACGAGAAGCT
GGAGTACTACGAGAAGTTCCAGATCATCGAGAACGTGTTC
AAGCAGAAGAAGAAGCCCACCCTGAAGCAGATCGCCAAGG
AGATCCTGGTGAACGAGGAGGACATCAAGGGCTACCGGGT
GACCAGCACCGGCAAGCCCGAGTTCACCAACCTGAAGGTG
TACCACGACATCAAGGACATCACCGCCCGGAAGGAGATCA
TCGAGAACGCCGAGCTGCTGGACCAGATCGCCAAGATCCT
GACCATCTACCAGAGCAGCGAGGACATCCAGGAGGAGCTG
ACCAACCTGAACAGCGAGCTGACCCAGGAGGAGATCGAGC
AGATCAGCAACCTGAAGGGCTACACCGGCACCCACAACCT
GAGCCTGAAGGCCATCAACCTGATCCTGGACGAGCTGTGG
CACACCAACGACAACCAGATCGCCATCTTCAACCGGCTGA
AGCTGGTGCCCAAGAAGGTGGACCTGAGCCAGCAGAAGGA
GATCCCCACCACCCTGGTGGACGACTTCATCCTGAGCCCC
GTGGTGAAGCGGAGCTTCATCCAGAGCATCAAGGTGATCA
ACGCCATCATCAAGAAGTACGGCCTGCCCAACGACATCAT
CATCGAGCTGGCCCGGGAGAAGAACAGCAAGGACGCCCAG
AAGATGATCAACGAGATGCAGAAGCGGAACCGGCAGACCA
ACGAGCGGATCGAGGAGATCATCCGGACCACCGGCAAGGA
GAACGCCAAGTACCTGATCGAGAAGATCAAGCTGCACGAC
ATGCAGGAGGGCAAGTGCCTGTACAGCCTGGAGGCCATCC
CCCTGGAGGACCTGCTGAACAACCCCTTCAACTACGAGGT
GGACGCCATCATCCCCCGGAGCGTGAGCTTCGACAACAGC
TTCAACAACAAGGTGCTGGTGAAGCAGGAGGAGAACAGCA
AGAAGGGCAACCGGACCCCCTTCCAGTACCTGAGCAGCAG
CGACAGCAAGATCAGCTACGAGACCTTCAAGAAGCACATC
CTGAACCTGGCCAAGGGCAAGGGCCGGATCAGCAAGACCA
AGAAGGAGTACCTGCTGGAGGAGCGGGACATCAACCGGTT
CAGCGTGCAGAAGGACTTCATCAACCGGAACCTGGTGGAC
ACCCGGTACGCCACCCGGGGCCTGATGAACCTGCTGCGGA
GCTACTTCCGGGTGAACAACCTGGACGTGAAGGTGaaatc
cATCAACGGCGGCTTCACCAGCTTCCTGCGGCGGAAGTGG
AAGTTCAAGAAGGAGCGGAACAAGGGCTACAAGCACCACG
CCGAGGACGCCCTGATCATCGCCAACGCCGACTTCATCTT
CAAGGAGTGGAAGAAGCTGGACAAGGCCAAGAAGGTGATG
GAGAACCAGATGTTCGAGGAGAAGCAGGCCGAGAGCATGC
CCGAGATCGAGACCGAGCAGGAGTACAAGGAGATCTTCAT
CACCCCCCACCAGATCAAGCACATCAAGGACTTCAAGGAC
TACAAGTACAGCCACCGGGTGGACAAGAAGCCCAACCGGA
AGCTGATCAACGACACCCTGTACAGCACCCGGAAGGACGA
CAAGGGCAACACCCTGATCGTGAACAACCTGAACGGCCTG
TACGACAAGGACAACGACAAGCTGAAGAAGCTGATCAACA
AGAGCCCCGAGAAGCTGCTGATGTACCACCACGACCCCCA
GACCTACCAGAAGCTGAAGCTGATCATGGAGCAGTACGGC
GACGAGAAGAACCCCCTGTACAAGTACTACGAGGAGACCG
GCAACTACCTGACCAAGTACAGCAAGAAGGACAACGGCCC
CGTGATCAAGAAGATCAAGTACTACGGCAACAAGCTGAAC
GCCCACCTGGACATCACCGACGACTACCCCAACAGCCGGA
ACAAGGTGGTGAAGCTGAGCCTGAAGCCCTACCGGTTCGA
CGTGTACCTGGACAACGGCGTGTACAAGTTCGTGACCGTG
AAGAACCTGGACGTGATCAAGAAGGAGAACTACTACGAGG
TGAACAGCAAGTGCTACGAGGAGGCCAAGAAGCTGAAGAA
GATCAGCAACCAGGCCGAGTTCATCGCCAGCTTCTACAAG
AACGACCTGATCAAGATCAACGGCGAGCTGTACCGGGTGA
TCGGCGTGAACAACGACCTGCTGAACCGGATCGAGGTGAA
CATGATCGACATCACCTACCGGGAGTACCTGGAGAACATG
AACGACAAGCGGCCCCCCCACATCATCAAGACCATCGCCA
GCAAGACCCAGAGCATCAAGAAGTACAGCACCGACATCCT
GGGCAACCTGTACGAGGTGaaatccAAGAAGCACCCCCAG
ATCATCAAGAAGGGCAAGCGGCCCGCCGCCACCAAGAAGG
CCGGCCAGGCCAAGAAGAAGAAGGCCCGGGACAGCAAGGT
GGAGAACAAGACCAAGAAGCTGCGGGTGTTCGAGGCCTTC
GCCGGCATCGGCGCCCAGCGGAAGGCCCTGGAGAAGGTGC
GGAAGGACGAGTACGAGATCGTGGGCCTGGCCGAGTGGTA
CGTGCCCGCCATCGTGATGTACCAGGCCATCCACAACAAC
TTCCACACCAAGCTGGAGTACAAGAGCGTGAGCCGGGAGG
AGATGATCGACTACCTGGAGAACAAGACCCTGAGCTGGAA
CAGCAAGAACCCCGTGAGCAACGGCTACTGGAAGCGGAAG
AAGGACGACGAGCTGAAGATCATCTACAACGCCATCAAGC
TGAGCGAGAAGGAGGGCAACATCTTCGACATCCGGGACCT
GTACAAGCGGACCCTGAAGAACATCGACCTGCTGACCTAC
AGCTTCCCCTGCCAGGACCTGAGCCAGCAGGGCATCCAGA
AGGGCATGAAGCGGGGCAGCGGCACCCGGAGCGGCCTGCT
GTGGGAGATCGAGCGGGCCCTGGACAGCACCGAGAAGAAC
GACCTGCCCAAGTACCTGCTGATGGAGAACGTGGGCGCCC
TGCTGCACAAGAAGAACGAGGAGGAGCTGAACCAGTGGAA
GCAGAAGCTGGAGAGCCTGGGCTACCAGAACAGCATCGAG
GTGCTGAACGCCGCCGACTTCGGCAGCAGCCAGGCCCGGC
GGCGGGTGTTCATGATCAGCACCCTGAACGAGTTCGTGGA
GCTGCCCAAGGGCGACAAGAAGCCCAAGAGCATCAAGAAG
GTGCTGAACAAGATCGTGAGCGAGAAGGACATCCTGAACA
ACCTGCTGAAGTACAACCTGACCGAGTTCAAGAAaACCAA
GAGCAACATCAACAAGGCCAGCCTGATCGGCTACAGCAAG
TTCAACAGCGAGGGCTACGTGTACGACCCCGAGTTCACCG
GCCCCACCCTGACCGCCAGCGGCGCCAACAGCCGGATCAA
GATCAAGGACGGCAGCAACATCCGGAAGATGAACAGCGAC
GAGACCTTCCTGTACATCGGCTTCGACAGCCAGGACGGCA
AGCGGGTGAACGAGATCGAGTTCCTGACCGAGAACCAGAA
GATCTTCGTGTGCGGCAACAGCATCAGCGTGGAGGTGCTG
GAGGCCATCATCGACAAGATCGGCGGCCCCAGCAGCGGCG
GCAAGCGGCCCGCCGCCACCAAGAAGGCCGGCCAGGCCAA
GAAGAAGAAGGGCAGCTACCCCTACGACGTGCCCGACTAC
GCCTGAGcggccgcttaattaagctgccttctgcggggct
tgccttctggccatgcccttcttctctcccttgcacctgt
acctcttggtctttgaataaagectgagtaggaagtctag
aaaaaaaaaaaaaaaaaaaaaaaaaaaaaaaaaaaaaaaa
aaaaaaaaaaaaaaaaaaaaaaaaaaaaaaaaaaaaaaaa
ttgtcttcttcatcgcctgcagATCCCAATGGCGCGCCGA
GCTTGGCTCGAGCATGGTCATAGCTGTTTCCTGTGTGAAA
TTGTTATCCGCTCACAATTCCACACAACATACGAGCCGGA
AGCATAAAGTGTAAAGCCTGGGGTGCCTAATGAGTGAGCT
AACTCACATTAATTGCGTTGCGCTCACTGCCCGCTTTCCA
GTCGGGAAACCTGTCGTGCCAGCTGCATTAATGAATCGGC
CAACGCGCGGGGAGAGGCGGTTTGCGTATTGGGCGCTCTT
CCGCTTCCTCGCTCACTGACTCGCTGCGCTCGGTCGTTCG
GCTGCGGCGAGCGGTATCAGCTCACTCAAAGGCGGTAATA
CGGTTATCCACAGAATCAGGGGATAACGCAGGAAAGAACA
TGTGAGCAAAAGGCCAGCAAAAGGCCAGGAACCGTAAAAA
GGCCGCGTTGCTGGCGTTTTTCCATAGGCTCCGCCCCCCT
GACGAGCATCACAAAAATCGACGCTCAAGTCAGAGGTGGC
GAAACCCGACAGGACTATAAAGATACCAGGCGTTTCCCCC
TGGAAGCTCCCTCGTGCGCTCTCCTGTTCCGACCCTGCCG
CTTACCGGATACCTGTCCGCCTTTCTCCCTTCGGGAAGCG
TGGCGCTTTCTCATAGCTCACGCTGTAGGTATCTCAGTTC
GGTGTAGGTCGTTCGCTCCAAGCTGGGCTGTGTGCACGAA
CCCCCCGTTCAGCCCGACCGCTGCGCCTTATCCGGTAACT
ATCGTCTTGAGTCCAACCCGGTAAGACACGACTTATCGCC
ACTGGCAGCAGCCACTGGTAACAGGATTAGCAGAGCGAGG
TATGTAGGCGGTGCTACAGAGTTCTTGAAGTGGTGGCCTA
ACTACGGCTACACTAGAAGAACAGTATTTGGTATCTGCGC
TCTGCTGAAGCCAGTTACCTTCGGAAAAAGAGTTGGTAGC
TCTTGATCCGGCAAACAAACCACCGCTGGTAGCGGTGGTT
TTTTTGTTTGCAAGCAGCAGATTACGCGCAGAAAAAAAGG
ATCTCAAGAAGATCCTTTGATCTTTTCTACGGGGTCTGAC
GCTCAGTGGAACGAAAACTCACGTTAAGGGATTTTGGTCA
TGAGATTATCAAAAAGGATCTTCACCTAGATCCTTTTAAA
TTAAAAATGAAGTTTTAAATCAATCTAAAGTATATATGAG
TAAACTTGGTCTGACAGTTAGAAAAACTCATCGAGCATCA
AATGAAACTGCAATTTATTCATATCAGGATTATCAATACC
ATATTTTTGAAAAAGCCGTTTCTGTAATGAAGGAGAAAAC
TCACCGAGGCAGTTCCATAGGATGGCAAGATCCTGGTATC
GGTCTGCGATTCCGACTCGTCCAACATCAATACAACCTAT
TAATTTCCCCTCGTCAAAAATAAGGTTATCAAGTGAGAAA
TCACCATGAGTGACGACTGAATCCGGTGAGAATGGCAAAA
GTTTATGCATTTCTTTCCAGACTTGTTCAACAGGCCAGCC
ATTACGCTCGTCATCAAAATCACTCGCATCAACCAAACCG
TTATTCATTCGTGATTGCGCCTGAGCGAGACGAAATACGC
GATCGCTGTTAAAAGGACAATTACAAACAGGAATCGAATG
CAACCGGCGCAGGAACACTGCCAGCGCATCAACAATATTT
TCACCTGAATCAGGATATTCTTCTAATACCTGGAATGCTG
TTTTCCCAGGGATCGCAGTGGTGAGTAACCATGCATCATC
AGGAGTACGGATAAAATGCTTGATGGTCGGAAGAGGCATA
AATTCCGTCAGCCAGTTTAGTCTGACCATCTCATCTGTAA
CATCATTGGCAACGCTACCTTTGCCATGTTTCAGAAACAA
CTCTGGCGCATCGGGCTTCCCATACAATCGATAGATTGTC
GCACCTGATTGCCCGACATTATCGCGAGCCCATTTATACC
CATATAAATCAGCATCCATGTTGGAATTTAATCGCGGCCT
AGAGCAAGACGTTTCCCGTTGAATATGGCTCATACTCTTC
CTTTTTCAATATTATTGAAGCATTTATCAGGGTTATTGTC
TCATGAGCGGATACATATTTGAATGTATTTAGAAAAATAA
ACAAATAGGGGTTCCGCGCACATTTCCCCGAAAAGTGCCA
CCTGACGTCTAAGAAACCATTATTATCATGACATTAACCT
ATAAAAATAGGCGTATCACGAGGCCCTTTCGTC
Sa-dCas9-MQ1 (MR-28126) Expressed mRNA sequence:
(SEQ ID NO: 202)
AGGAAATAAGAGAGAAAAGAAGAGTAAGAAGAAATATAAGA
GCCACCATGGCCCCCAAGAAGAAGCGGAAGGTGGGCATCC
ACGGCGTGCCCGCCGCCGCCAAGCGGAACTACATCCTGGG
CCTGGCCATCGGCATCACCAGCGTGGGCTACGGCATCATC
GACTACGAGACCCGGGACGTGATCGACGCCGGCGTGCGGC
TGTTCAAGGAGGCCAACGTGGAGAACAACGAGGGCCGGCG
GAGCAAGCGGGGCGCCCGGCGGCTGAAGCGGCGGCGGCGG
CACCGGATCCAGCGGGTGAAGAAGCTGCTGTTCGACTACA
ACCTGCTGACCGACCACAGCGAGCTGAGCGGCATCAACCC
CTACGAGGCCCGGGTGAAGGGCCTGAGCCAGAAGCTGAGC
GAGGAGGAGTTCAGCGCCGCCCTGCTGCACCTGGCCAAGC
GGCGGGGCGTGCACAACGTGAACGAGGTGGAGGAGGACAC
CGGCAACGAGCTGAGCACCAAGGAGCAGATCAGCCGGAAC
AGCAAGGCCCTGGAGGAGAAGTACGTGGCCGAGCTGCAGC
TGGAGCGGCTGAAGAAGGACGGCGAGGTGCGGGGCAGCAT
CAACCGGTTCAAGACCAGCGACTACGTGAAGGAGGCCAAG
CAGCTGCTGAAGGTGCAGAAGGCCTACCACCAGCTGGACC
AGAGCTTCATCGACACCTACATCGACCTGCTGGAGACCCG
GCGGACCTACTACGAGGGCCCCGGCGAGGGCAGCCCCTTC
GGCTGGAAGGACATCAAGGAGTGGTACGAGATGCTGATGG
GCCACTGCACCTACTTCCCCGAGGAGCTGCGGAGCGTGAA
GTACGCCTACAACGCCGACCTGTACAACGCCCTGAACGAC
CTGAACAACCTGGTGATCACCCGGGACGAGAACGAGAAGC
TGGAGTACTACGAGAAGTTCCAGATCATCGAGAACGTGTT
CAAGCAGAAGAAGAAGCCCACCCTGAAGCAGATCGCCAAG
GAGATCCTGGTGAACGAGGAGGACATCAAGGGCTACCGGG
TGACCAGCACCGGCAAGCCCGAGTTCACCAACCTGAAGGT
GTACCACGACATCAAGGACATCACCGCCCGGAAGGAGATC
ATCGAGAACGCCGAGCTGCTGGACCAGATCGCCAAGATCC
TGACCATCTACCAGAGCAGCGAGGACATCCAGGAGGAGCT
GACCAACCTGAACAGCGAGCTGACCCAGGAGGAGATCGAG
CAGATCAGCAACCTGAAGGGCTACACCGGCACCCACAACC
TGAGCCTGAAGGCCATCAACCTGATCCTGGACGAGCTGTG
GCACACCAACGACAACCAGATCGCCATCTTCAACCGGCTG
AAGCTGGTGCCCAAGAAGGTGGACCTGAGCCAGCAGAAGG
AGATCCCCACCACCCTGGTGGACGACTTCATCCTGAGCCC
CGTGGTGAAGCGGAGCTTCATCCAGAGCATCAAGGTGATC
AACGCCATCATCAAGAAGTACGGCCTGCCCAACGACATCA
TCATCGAGCTGGCCCGGGAGAAGAACAGCAAGGACGCCCA
GAAGATGATCAACGAGATGCAGAAGCGGAACCGGCAGACC
AACGAGCGGATCGAGGAGATCATCCGGACCACCGGCAAGG
AGAACGCCAAGTACCTGATCGAGAAGATCAAGCTGCACGA
CATGCAGGAGGGCAAGTGCCTGTACAGCCTGGAGGCCATC
CCCCTGGAGGACCTGCTGAACAACCCCTTCAACTACGAGG
TGGACGCCATCATCCCCCGGAGCGTGAGCTTCGACAACAG
CTTCAACAACAAGGTGCTGGTGAAGCAGGAGGAGAACAGC
AAGAAGGGCAACCGGACCCCCTTCCAGTACCTGAGCAGCA
GCGACAGCAAGATCAGCTACGAGACCTTCAAGAAGCACAT
CCTGAACCTGGCCAAGGGCAAGGGCCGGATCAGCAAGACC
AAGAAGGAGTACCTGCTGGAGGAGCGGGACATCAACCGGT
TCAGCGTGCAGAAGGACTTCATCAACCGGAACCTGGTGGA
CACCCGGTACGCCACCCGGGGCCTGATGAACCTGCTGCGG
AGCTACTTCCGGGTGAACAACCTGGACGTGAAGGTGaaat
ccATCAACGGCGGCTTCACCAGCTTCCTGCGGCGGAAGTG
GAAGTTCAAGAAGGAGCGGAACAAGGGCTACAAGCACCAC
GCCGAGGACGCCCTGATCATCGCCAACGCCGACTTCATCT
TCAAGGAGTGGAAGAAGCTGGACAAGGCCAAGAAGGTGAT
GGAGAACCAGATGTTCGAGGAGAAGCAGGCCGAGAGCATG
CCCGAGATCGAGACCGAGCAGGAGTACAAGGAGATCTTCA
TCACCCCCCACCAGATCAAGCACATCAAGGACTTCAAGGA
CTACAAGTACAGCCACCGGGTGGACAAGAAGCCCAACCGG
AAGCTGATCAACGACACCCTGTACAGCACCCGGAAGGACG
ACAAGGGCAACACCCTGATCGTGAACAACCTGAACGGCCT
GTACGACAAGGACAACGACAAGCTGAAGAAGCTGATCAAC
AAGAGCCCCGAGAAGCTGCTGATGTACCACCACGACCCCC
AGACCTACCAGAAGCTGAAGCTGATCATGGAGCAGTACGG
CGACGAGAAGAACCCCCTGTACAAGTACTACGAGGAGACC
GGCAACTACCTGACCAAGTACAGCAAGAAGGACAACGGCC
CCGTGATCAAGAAGATCAAGTACTACGGCAACAAGCTGAA
CGCCCACCTGGACATCACCGACGACTACCCCAACAGCCGG
AACAAGGTGGTGAAGCTGAGCCTGAAGCCCTACCGGTTCG
ACGTGTACCTGGACAACGGCGTGTACAAGTTCGTGACCGT
GAAGAACCTGGACGTGATCAAGAAGGAGAACTACTACGAG
GTGAACAGCAAGTGCTACGAGGAGGCCAAGAAGCTGAAGA
AGATCAGCAACCAGGCCGAGTTCATCGCCAGCTTCTACAA
GAACGACCTGATCAAGATCAACGGCGAGCTGTACCGGGTG
ATCGGCGTGAACAACGACCTGCTGAACCGGATCGAGGTGA
ACATGATCGACATCACCTACCGGGAGTACCTGGAGAACAT
GAACGACAAGCGGCCCCCCCACATCATCAAGACCATCGCC
AGCAAGACCCAGAGCATCAAGAAGTACAGCACCGACATCC
TGGGCAACCTGTACGAGGTGaaatccAAGAAGCACCCCCA
GATCATCAAGAAGGGCAAGCGGCCCGCCGCCACCAAGAAG
GCCGGCCAGGCCAAGAAGAAGAAGGCCCGGGACAGCAAGG
TGGAGAACAAGACCAAGAAGCTGCGGGTGTTCGAGGCCTT
CGCCGGCATCGGCGCCCAGCGGAAGGCCCTGGAGAAGGTG
CGGAAGGACGAGTACGAGATCGTGGGCCTGGCCGAGTGGT
ACGTGCCCGCCATCGTGATGTACCAGGCCATCCACAACAA
CTTCCACACCAAGCTGGAGTACAAGAGCGTGAGCCGGGAG
GAGATGATCGACTACCTGGAGAACAAGACCCTGAGCTGGA
ACAGCAAGAACCCCGTGAGCAACGGCTACTGGAAGCGGAA
GAAGGACGACGAGCTGAAGATCATCTACAACGCCATCAAG
CTGAGCGAGAAGGAGGGCAACATCTTCGACATCCGGGACC
TGTACAAGCGGACCCTGAAGAACATCGACCTGCTGACCTA
CAGCTTCCCCTGCCAGGACCTGAGCCAGCAGGGCATCCAG
AAGGGCATGAAGCGGGGCAGCGGCACCCGGAGCGGCCTGC
TGTGGGAGATCGAGCGGGCCCTGGACAGCACCGAGAAGAA
CGACCTGCCCAAGTACCTGCTGATGGAGAACGTGGGCGCC
CTGCTGCACAAGAAGAACGAGGAGGAGCTGAACCAGTGGA
AGCAGAAGCTGGAGAGCCTGGGCTACCAGAACAGCATCGA
GGTGCTGAACGCCGCCGACTTCGGCAGCAGCCAGGCCCGG
CGGCGGGTGTTCATGATCAGCACCCTGAACGAGTTCGTGG
AGCTGCCCAAGGGCGACAAGAAGCCCAAGAGCATCAAGAA
GGTGCTGAACAAGATCGTGAGCGAGAAGGACATCCTGAAC
AACCTGCTGAAGTACAACCTGACCGAGTTCAAGAAaACCA
AGAGCAACATCAACAAGGCCAGCCTGATCGGCTACAGCAA
GTTCAACAGCGAGGGCTACGTGTACGACCCCGAGTTCACC
GGCCCCACCCTGACCGCCAGCGGCGCCAACAGCCGGATCA
AGATCAAGGACGGCAGCAACATCCGGAAGATGAACAGCGA
CGAGACCTTCCTGTACATCGGCTTCGACAGCCAGGACGGC
AAGCGGGTGAACGAGATCGAGTTCCTGACCGAGAACCAGA
AGATCTTCGTGTGCGGCAACAGCATCAGCGTGGAGGTGCT
GGAGGCCATCATCGACAAGATCGGCGGCCCCAGCAGCGGC
GGCAAGCGGCCCGCCGCCACCAAGAAGGCCGGCCAGGCCA
AGAAGAAGAAGGGCAGCTACCCCTACGACGTGCCCGACTA
CGCCTGAGcggccgcttaattaagctgccttctgcggggc
ttgccttctggccatgcccttcttctctcccttgcacctg
tacctcttggtctttgaataaagcctgagtaggaagtcta
gaaaaaaaaaaaaaaaaaaaaaaaaaaaaaaaaaaaaaaa
aaaaaaaaaaaaaaaaaaaaaaaaaaaaaaaaaaaaaaaa
a

In some embodiments, a site-specific disrupting agent comprises a targeting moiety (e.g., comprising dCas9, e.g., an S. Pyogenes dCas9 or a functional variant or mutant thereof; e.g., Cas9m4), and an effector moiety comprising MQ1, e.g., bacterial MQ1. In some embodiments, the site-specific disrupting agent is encoded by the nucleic acid sequence of SEQ ID NOs: 207 (e.g., a nucleic acid (e.g., mRNA) encoding the site-specific disrupting agent). In some embodiments, a nucleic acid described herein comprises a nucleic acid sequence of SEQ ID NO: 207 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 8 and/or the effector moiety is encoded by the nucleic acid sequence of SEQ ID NO: 10.

dCas9-MQ1 mRNA sequence (MR28125)
(SEQ ID NO: 207)
AAGGAAAUAAGAGAGAAAAGAAGAGUAAGAAGAAAUAUAAGAGCCACCAUGGCCCCCAA
GAAGAAGCGGAAGGUGGGCAUCCACGGCGUGCCCGCCGCCGACAAGAAGUACAGCAUCGG
CCUGGCCAUCGGCACCAACAGCGUGGGCUGGGCCGUGAUCACCGACGAGUACAAGGUGCC
CAGCAAGAAGUUCAAGGUGCUGGGCAACACCGACCGGCACAGCAUCAAGAAGAACCUGAU
CGGCGCCCUGCUGUUCGACAGCGGCGAGACCGCCGAGGCCACCCGGCUGAAGCGGACCGC
CCGGCGGCGGUACACCCGGCGGAAGAACCGGAUCUGCUACCUGCAGGAGAUCUUCAGCAA
CGAGAUGGCCAAGGUGGACGACAGCUUCUUCCACCGGCUGGAGGAGAGCUUCCUGGUGGA
GGAGGACAAGAAGCACGAGCGGCACCCCAUCUUCGGCAACAUCGUGGACGAGGUGGCCUA
CCACGAGAAGUACCCCACCAUCUACCACCUGCGGAAGAAGCUGGUGGACAGCACCGACAA
GGCCGACCUGCGGCUGAUCUACCUGGCCCUGGCCCACAUGAUCAAGUUCCGGGGCCACUU
CCUGAUCGAGGGCGACCUGAACCCCGACAACAGCGACGUGGACAAGCUGUUCAUCCAGCU
GGUGCAGACCUACAACCAGCUGUUCGAGGAGAACCCCAUCAACGCCAGCGGCGUGGACGC
CAAGGCCAUCCUGAGCGCCCGGCUGAGCAAGAGCCGGCGGCUGGAGAACCUGAUCGCCCA
GCUGCCCGGCGAGAAGAAGAACGGCCUGUUCGGCAACCUGAUCGCCCUGAGCCUGGGCCU
GACCCCCAACUUCAAGAGCAACUUCGACCUGGCCGAGGACGCCAAGCUGCAGCUGAGCAA
GGACACCUACGACGACGACCUGGACAACCUGCUGGCCCAGAUCGGCGACCAGUACGCCGA
CCUGUUCCUGGCCGCCAAGAACCUGAGCGACGCCAUCCUGCUGAGCGACAUCCUGCGGGU
GAACACCGAGAUCACCAAGGCCCCCCUGAGCGCCAGCAUGAUCAAGCGGUACGACGAGCA
CCACCAGGACCUGACCCUGCUGAAGGCCCUGGUGCGGCAGCAGCUGCCCGAGAAGUACAA
GGAGAUCUUCUUCGACCAGAGCAAGAACGGCUACGCCGGCUACAUCGACGGCGGCGCCAG
CCAGGAGGAGUUCUACAAGUUCAUCAAGCCCAUCCUGGAGAAGAUGGACGGCACCGAGGA
GCUGCUGGUGAAGCUGAACCGGGAGGACCUGCUGCGGAAGCAGCGGACCUUCGACAACGG
CAGCAUCCCCCACCAGAUCCACCUGGGCGAGCUGCACGCCAUCCUGCGGCGGCAGGAGGA
CUUCUACCCCUUCCUGAAGGACAACCGGGAGAAGAUCGAGAAGAUCCUGACCUUCCGGAU
CCCCUACUACGUGGGCCCCCUGGCCCGGGGCAACAGCCGGUUCGCCUGGAUGACCCGGAA
AUCCGAGGAGACCAUCACCCCCUGGAACUUCGAGGAGGUGGUGGACAAGGGCGCCAGCGC
CCAGAGCUUCAUCGAGCGGAUGACCAACUUCGACAAGAACCUGCCCAACGAGAAGGUGCU
GCCCAAGCACAGCCUGCUGUACGAGUACUUCACCGUGUACAACGAGCUGACCAAGGUGAA
GUACGUGACCGAGGGCAUGCGGAAGCCCGCCUUCCUGAGCGGCGAGCAGAAGAAGGCCAU
CGUGGACCUGCUGUUCAAGACCAACCGGAAGGUGACCGUGAAGCAGCUGAAGGAGGACU
ACUUCAAGAAGAUCGAGUGCUUCGACAGCGUGGAGAUCAGCGGCGUGGAGGACCGGUUC
AACGCCAGCCUGGGCACCUACCACGACCUGCUGAAGAUCAUCAAGGACAAGGACUUCCUG
GACAACGAGGAGAACGAGGACAUCCUGGAGGACAUCGUGCUGACCCUGACCCUGUUCGAG
GACCGGGAGAUGAUCGAGGAGCGGCUGAAAACCUACGCCCACCUGUUCGACGACAAGGUG
AUGAAGCAGCUGAAGCGGCGGCGGUACACCGGCUGGGGCCGGCUGAGCCGGAAGCUGAUC
AACGGCAUCCGGGACAAGCAGAGCGGCAAGACCAUCCUGGACUUCCUGAAAUCCGACGGC
UUCGCCAACCGGAACUUCAUGCAGCUGAUCCACGACGACAGCCUGACCUUCAAGGAGGAC
AUCCAGAAGGCCCAGGUGAGCGGCCAGGGCGACAGCCUGCACGAGCACAUCGCCAACCUG
GCCGGCAGCCCCGCCAUCAAGAAGGGCAUCCUGCAGACCGUGAAGGUGGUGGACGAGCUG
GUGAAGGUGAUGGGCCGGCACAAGCCCGAGAACAUCGUGAUCGAGAUGGCCCGGGAGAA
CCAGACCACCCAGAAGGGCCAGAAGAACAGCCGGGAGCGGAUGAAGCGGAUCGAGGAGGG
CAUCAAGGAGCUGGGCAGCCAGAUCCUGAAGGAGCACCCCGUGGAGAACACCCAGCUGCA
GAACGAGAAGCUGUACCUGUACUACCUGCAGAACGGCCGGGACAUGUACGUGGACCAGGA
GCUGGACAUCAACCGGCUGAGCGACUACGACGUGGCCGCCAUCGUGCCCCAGAGCUUCCU
GAAGGACGACAGCAUCGACAACAAGGUGCUGACCCGGAGCGACAAGGCCCGGGGCAAGAG
CGACAACGUGCCCAGCGAGGAGGUGGUGAAGAAGAUGAAGAACUACUGGCGGCAGCUGC
UGAACGCCAAGCUGAUCACCCAGCGGAAGUUCGACAACCUGACCAAGGCCGAGCGGGGCG
GCCUGAGCGAGCUGGACAAGGCCGGCUUCAUCAAGCGGCAGCUGGUGGAGACCCGGCAGA
UCACCAAGCACGUGGCCCAGAUCCUGGACAGCCGGAUGAACACCAAGUACGACGAGAACG
ACAAGCUGAUCCGGGAGGUGAAGGUGAUCACCCUGAAAUCCAAGCUGGUGAGCGACUUCC
GGAAGGACUUCCAGUUCUACAAGGUGCGGGAGAUCAACAACUACCACCACGCCCACGACG
CCUACCUGAACGCCGUGGUGGGCACCGCCCUGAUCAAGAAGUACCCCAAGCUGGAGAGCG
AGUUCGUGUACGGCGACUACAAGGUGUACGACGUGCGGAAGAUGAUCGCCAAGAGCGAG
CAGGAGAUCGGCAAGGCCACCGCCAAGUACUUCUUCUACAGCAACAUCAUGAACUUCUUC
AAGACCGAGAUCACCCUGGCCAACGGCGAGAUCCGGAAGCGGCCCCUGAUCGAGACCAAC
GGCGAGACCGGCGAGAUCGUGUGGGACAAGGGCCGGGACUUCGCCACCGUGCGGAAGGUG
CUGAGCAUGCCCCAGGUGAACAUCGUGAAGAAAACCGAGGUGCAGACCGGCGGCUUCAGC
AAGGAGAGCAUCCUGCCCAAGCGGAACAGCGACAAGCUGAUCGCCCGGAAGAAGGACUGG
GACCCCAAGAAGUACGGCGGCUUCGACAGCCCCACCGUGGCCUACAGCGUGCUGGUGGUG
GCCAAGGUGGAGAAGGGCAAGAGCAAGAAGCUGAAAUCCGUGAAGGAGCUGCUGGGCAU
CACCAUCAUGGAGCGGAGCAGCUUCGAGAAGAACCCCAUCGACUUCCUGGAGGCCAAGGG
CUACAAGGAGGUGAAGAAGGACCUGAUCAUCAAGCUGCCCAAGUACAGCCUGUUCGAGCU
GGAGAACGGCCGGAAGCGGAUGCUGGCCAGCGCCGGCGAGCUGCAGAAGGGCAACGAGCU
GGCCCUGCCCAGCAAGUACGUGAACUUCCUGUACCUGGCCAGCCACUACGAGAAGCUGAA
GGGCAGCCCCGAGGACAACGAGCAGAAGCAGCUGUUCGUGGAGCAGCACAAGCACUACCU
GGACGAGAUCAUCGAGCAGAUCAGCGAGUUCAGCAAGCGGGUGAUCCUGGCCGACGCCAA
CCUGGACAAGGUGCUGAGCGCCUACAACAAGCACCGGGACAAGCCCAUCCGGGAGCAGGC
CGAGAACAUCAUCCACCUGUUCACCCUGACCAACCUGGGCGCCCCCGCCGCCUUCAAGUA
CUUCGACACCACCAUCGACCGGAAGCGGUACACCAGCACCAAGGAGGUGCUGGACGCCAC
CCUGAUCCACCAGAGCAUCACCGGCCUGUACGAGACCCGGAUCGACCUGAGCCAGCUGGG
CGGCGACAAGCGGCCCGCCGCCACCAAGAAGGCCGGCCAGGCCAAGAAGAAGAAGGCCCG
GGACAGCAAGGUGGAGAACAAGACCAAGAAGCUGCGGGUGUUCGAGGCCUUCGCCGGCA
UCGGCGCCCAGCGGAAGGCCCUGGAGAAGGUGCGGAAGGACGAGUACGAGAUCGUGGGCC
UGGCCGAGUGGUACGUGCCCGCCAUCGUGAUGUACCAGGCCAUCCACAACAACUUCCACA
CCAAGCUGGAGUACAAGAGCGUGAGCCGGGAGGAGAUGAUCGACUACCUGGAGAACAAG
ACCCUGAGCUGGAACAGCAAGAACCCCGUGAGCAACGGCUACUGGAAGCGGAAGAAGGAC
GACGAGCUGAAGAUCAUCUACAACGCCAUCAAGCUGAGCGAGAAGGAGGGCAACAUCUUC
GACAUCCGGGACCUGUACAAGCGGACCCUGAAGAACAUCGACCUGCUGACCUACAGCUUC
CCCUGCCAGGACCUGAGCCAGCAGGGCAUCCAGAAGGGCAUGAAGCGGGGCAGCGGCACC
CGGAGCGGCCUGCUGUGGGAGAUCGAGCGGGCCCUGGACAGCACCGAGAAGAACGACCUG
CCCAAGUACCUGCUGAUGGAGAACGUGGGCGCCCUGCUGCACAAGAAGAACGAGGAGGAG
CUGAACCAGUGGAAGCAGAAGCUGGAGAGCCUGGGCUACCAGAACAGCAUCGAGGUGCU
GAACGCCGCCGACUUCGGCAGCAGCCAGGCCCGGCGGCGGGUGUUCAUGAUCAGCACCCU
GAACGAGUUCGUGGAGCUGCCCAAGGGCGACAAGAAGCCCAAGAGCAUCAAGAAGGUGC
UGAACAAGAUCGUGAGCGAGAAGGACAUCCUGAACAACCUGCUGAAGUACAACCUGACCG
AGUUCAAGAAAACCAAGAGCAACAUCAACAAGGCCAGCCUGAUCGGCUACAGCAAGUUCA
ACAGCGAGGGCUACGUGUACGACCCCGAGUUCACCGGCCCCACCCUGACCGCCAGCGGCG
CCAACAGCCGGAUCAAGAUCAAGGACGGCAGCAACAUCCGGAAGAUGAACAGCGACGAGA
CCUUCCUGUACAUCGGCUUCGACAGCCAGGACGGCAAGCGGGUGAACGAGAUCGAGUUCC
UGACCGAGAACCAGAAGAUCUUCGUGUGCGGCAACAGCAUCAGCGUGGAGGUGCUGGAG
GCCAUCAUCGACAAGAUCGGCGGCCCCAGCAGCGGCGGCAAGCGGCCCGCCGCCACCAAG
AAGGCCGGCCAGGCCAAGAAGAAGAAGGGCAGCUACCCCUACGACGUGCCCGACUACGCC
UGAGCGGCCGCUUAAUUAAGCUGCCUUCUGCGGGGCUUGCCUUCUGGCCAUGCCCUUCUU
CUCUCCCUUGCACCUGUACCUCUUGGUCUUUGAAUAAAGCCUGAGUAGGAAGUCUAGAAA
AAAAAAAAAAAAAAAAAAAAAAAAAAAAAAAAAAAAAAAAAAAAAAAAAAAAAAAAAA
AAAAAAAAAAAAAAAAAAA

In some embodiments, a site-specific disrupting agent comprises the amino acid sequence of SEQ ID NOs: 203, 208, 73 or 74. In some embodiments, a site-specific disrupting agent described herein comprises an amino acid sequence of SEQ ID NO: 203, 208, 73 or 74, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Sa-dCas9-MQ1 Protein sequence:
(SEQ ID NO: 203)
MAPKKKRKVGIHGVPAAAKRNYILGLAIGITSVGYGIIDYETRDVIDAGVRLFKEANVENNEGRR
SKRGARRLKRRRRHRIQRVKKLLFDYNLLTDHSELSGINPYEARVKGLSQKLSEEEFSAALLHLA
KRRGVHNVNEVEEDTGNELSTKEQISRNSKALEEKYVAELQLERLKKDGEVRGSINRFKTSDYV
KEAKQLLKVQKAYHQLDQSFIDTYIDLLETRRTYYEGPGEGSPFGWKDIKEWYEMLMGHCTYF
PEELRSVKYAYNADLYNALNDLNNLVITRDENEKLEYYEKFQIIENVFKQKKKPTLKQIAKEILV
NEEDIKGYRVTSTGKPEFTNLKVYHDIKDITARKEIIENAELLDQIAKILTIYQSSEDIQEELTNLNS
ELTQEEIEQISNLKGYTGTHNLSLKAINLILDELWHTNDNQIAIFNRLKLVPKKVDLSQQKEIPTTL
VDDFILSPVVKRSFIQSIKVINAIIKKYGLPNDIIIELAREKNSKDAQKMINEMQKRNRQTNERIEEII
RTTGKENAKYLIEKIKLHDMQEGKCLYSLEAIPLEDLLNNPFNYEVDAIIPRSVSFDNSFNNKVLV
KQEENSKKGNRTPFQYLSSSDSKISYETFKKHILNLAKGKGRISKTKKEYLLEERDINRFSVQKDFI
NRNLVDTRYATRGLMNLLRSYFRVNNLDVKVKSINGGFTSFLRRKWKFKKERNKGYKHHAED
ALIIANADFIFKEWKKLDKAKKVMENQMFEEKQAESMPEIETEQEYKEIFITPHQIKHIKDFKDYK
YSHRVDKKPNRKLINDTLYSTRKDDKGNTLIVNNLNGLYDKDNDKLKKLINKSPEKLLMYHHD
PQTYQKLKLIMEQYGDEKNPLYKYYEETGNYLTKYSKKDNGPVIKKIKYYGNKLNAHLDITDD
YPNSRNKVVKLSLKPYRFDVYLDNGVYKFVTVKNLDVIKKENYYEVNSKCYEEAKKLKKISNQ
AEFIASFYKNDLIKINGELYRVIGVNNDLLNRIEVNMIDITYREYLENMNDKRPPHIIKTIASKTQSI
KKYSTDILGNLYEVKSKKHPQIIKKGKRPAATKKAGQAKKKKARDSKVENKTKKLRVFEAFAGI
GAQRKALEKVRKDEYEIVGLAEWYVPAIVMYQAIHNNFHTKLEYKSVSREEMIDYLENKTLSW
NSKNPVSNGYWKRKKDDELKIIYNAIKLSEKEGNIFDIRDLYKRTLKNIDLLTYSFPCQDLSQQGI
QKGMKRGSGTRSGLLWEIERALDSTEKNDLPKYLLMENVGALLHKKNEEELNQWKQKLESLG
YQNSIEVLNAADFGSSQARRRVFMISTLNEFVELPKGDKKPKSIKKVLNKIVSEKDILNNLLKYNL
TEFKKTKSNINKASLIGYSKFNSEGYVYDPEFTGPTLTASGANSRIKIKDGSNIRKMNSDETFLYIG
FDSQDGKRVNEIEFLTENQKIFVCGNSISVEVLEAIIDKIGGPSSGGKRPAATKKAGQAKKKKGSY
PYDVPDYA
dCas9-MQ1 Protein sequence (corresponding to MR-28125):
(SEQ ID NO: 208)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSN
GYWKRKKDDELKIIYNAIKLSEKEGNIFDIRDLYKRTLKNIDLLTYSFPCQDLSQQGIQKGMKRG
SGTRSGLLWEIERALDSTEKNDLPKYLLMENVGALLHKKNEEELNQWKQKLESLGYQNSIEVLN
AADFGSSQARRRVFMISTLNEFVELPKGDKKPKSIKKVLNKIVSEKDILNNLLKYNLTEFKKTKS
NINKASLIGYSKFNSEGYVYDPEFTGPTLTASGANSRIKIKDGSNIRKMNSDETFLYIGFDSQDGK
RVNEIEFLTENQKIFVCGNSISVEVLEAIIDKIGGPSSGGKRPAATKKAGQAKKKKGSYPYDVPDY
A
Sa-dCas9-MQ1 without HA tag
(SEQ ID NO: 73)
MAPKKKRKVGIHGVPAAAKRNYILGLAIGITSVGYGIIDYETRDVIDAGVRLFKEANVENNEGRR
SKRGARRLKRRRRHRIQRVKKLLFDYNLLTDHSELSGINPYEARVKGLSQKLSEEEFSAALLHLA
KRRGVHNVNEVEEDTGNELSTKEQISRNSKALEEKYVAELQLERLKKDGEVRGSINRFKTSDYV
KEAKQLLKVQKAYHQLDQSFIDTYIDLLETRRTYYEGPGEGSPFGWKDIKEWYEMLMGHCTYF
PEELRSVKYAYNADLYNALNDLNNLVITRDENEKLEYYEKFQIIENVFKQKKKPTLKQIAKEILV
NEEDIKGYRVTSTGKPEFTNLKVYHDIKDITARKEIIENAELLDQIAKILTIYQSSEDIQEELTNLNS
ELTQEEIEQISNLKGYTGTHNLSLKAINLILDELWHTNDNQIAIFNRLKLVPKKVDLSQQKEIPTTL
VDDFILSPVVKRSFIQSIKVINAIIKKYGLPNDIIIELAREKNSKDAQKMINEMQKRNRQTNERIEEII
RTTGKENAKYLIEKIKLHDMQEGKCLYSLEAIPLEDLLNNPFNYEVDAIIPRSVSFDNSFNNKVLV
KQEENSKKGNRTPFQYLSSSDSKISYETFKKHILNLAKGKGRISKTKKEYLLEERDINRFSVQKDFI
NRNLVDTRYATRGLMNLLRSYFRVNNLDVKVKSINGGFTSFLRRKWKFKKERNKGYKHHAED
ALIIANADFIFKEWKKLDKAKKVMENQMFEEKQAESMPEIETEQEYKEIFITPHQIKHIKDFKDYK
YSHRVDKKPNRKLINDTLYSTRKDDKGNTLIVNNLNGLYDKDNDKLKKLINKSPEKLLMYHHD
PQTYQKLKLIMEQYGDEKNPLYKYYEETGNYLTKYSKKDNGPVIKKIKYYGNKLNAHLDITDD
YPNSRNKVVKLSLKPYRFDVYLDNGVYKFVTVKNLDVIKKENYYEVNSKCYEEAKKLKKISNQ
AEFIASFYKNDLIKINGELYRVIGVNNDLLNRIEVNMIDITYREYLENMNDKRPPHIIKTIASKTQSI
KKYSTDILGNLYEVKSKKHPQIIKKGKRPAATKKAGQAKKKKARDSKVENKTKKLRVFEAFAGI
GAQRKALEKVRKDEYEIVGLAEWYVPAIVMYQAIHNNFHTKLEYKSVSREEMIDYLENKTLSW
NSKNPVSNGYWKRKKDDELKIIYNAIKLSEKEGNIFDIRDLYKRTLKNIDLLTYSFPCQDLSQQGI
QKGMKRGSGTRSGLLWEIERALDSTEKNDLPKYLLMENVGALLHKKNEEELNQWKQKLESLG
YQNSIEVLNAADFGSSQARRRVFMISTLNEFVELPKGDKKPKSIKKVLNKIVSEKDILNNLLKYNL
TEFKKTKSNINKASLIGYSKFNSEGYVYDPEFTGPTLTASGANSRIKIKDGSNIRKMNSDETFLYIG
FDSQDGKRVNEIEFLTENQKIFVCGNSISVEVLEAIIDKIGGPSSGGKRPAATKKAGQAKKKKGS
dCas9-MQ1 without HA tag
(SEQ ID NO: 74)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKARDSKVENKTKKLRVFEAFAGIGAQRKALE
KVRKDEYEIVGLAEWYVPAIVMYQAIHNNFHTKLEYKSVSREEMIDYLENKTLSWNSKNPVSN
GYWKRKKDDELKIIYNAIKLSEKEGNIFDIRDLYKRTLKNIDLLTYSFPCQDLSQQGIQKGMKRG
SGTRSGLLWEIERALDSTEKNDLPKYLLMENVGALLHKKNEEELNQWKQKLESLGYQNSIEVLN
AADFGSSQARRRVFMISTLNEFVELPKGDKKPKSIKKVLNKIVSEKDILNNLLKYNLTEFKKTKS
NINKASLIGYSKFNSEGYVYDPEFTGPTLTASGANSRIKIKDGSNIRKMNSDETFLYIGFDSQDGK
RVNEIEFLTENQKIFVCGNSISVEVLEAIIDKIGGPSSGGKRPAATKKAGQAKKKKGS

In some embodiments, a site-specific disrupting agent comprises a targeting moiety (e.g., comprising dCas9, e.g., an S. pyogenes dCas9), and an effector moiety comprising KRAB, e.g., a KRAB domain. In some embodiments, the site-specific disrupting agent is encoded by the nucleic acid sequence of SEQ ID NOs: 204 (e.g., a plasmid encoding the site-specific disrupting agent) and/or 205 (e.g., a nucleic acid (e.g., mRNA) encoding the site-specific disrupting agent). In some embodiments, a nucleic acid described herein comprises a nucleic acid sequence of SEQ ID NO: 204 or 205 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO. 8 and the effector moiety is encoded by the nucleic acid sequence of SEQ ID NO. 14.

Sp-dCas9-KRAB (PL-27687) Plasmid DNA sequence:
(SEQ ID NO: 204)
TCGCGCGTTTCGGTGATGACGGTGAAAACCTCTGACACATGCAGCTCCCGGAGACGGTCACA
GCTTGTCTGTAAGCGGATGCCGGGAGCAGACAAGCCCGTCAGGGCGCGTCAGCGGGTGTTG
GCGGGTGTCGGGGCTGGCTTAACTATGCGGCATCAGAGCAGATTGTACTGAGAGTGCACCAT
ATGCGGTGTGAAATACCGCACAGATGCGTAAGGAGAAAATACCGCATCAGGCGCCATTCGC
CATTCAGGCTGCGCAACTGTTGGGAAGGGCGATCGGTGCGGGCCTCTTCGCTATTACGCCAG
CTGGCGAAAGGGGGATGTGCTGCAAGGCGATTAAGTTGGGTAACGCCAGGGTTTTCCCAGT
CACGACGTTGTAAAACGACGGCCAGTGAATTGACGCGTATTGGGATGGTACCTAATACGACT
CACTATAAGGAAATAAGAGAGAAAAGAAGAGTAAGAAGAAATATAAGAGCCACCATGGCC
CCCAAGAAGAAGCGGAAGGTGGGCATCCACGGCGTGCCCGCCGCCGACAAGAAGTACAGC
ATCGGCCTGGCCATCGGCACCAACAGCGTGGGCTGGGCCGTGATCACCGACGAGTACAAGG
TGCCCAGCAAGAAGTTCAAGGTGCTGGGCAACACCGACCGGCACAGCATCAAGAAGAACCT
GATCGGCGCCCTGCTGTTCGACAGCGGCGAGACCGCCGAGGCCACCCGGCTGAAGCGGACC
GCCCGGCGGCGGTACACCCGGCGGAAGAACCGGATCTGCTACCTGCAGGAGATCTTCAGCA
ACGAGATGGCCAAGGTGGACGACAGCTTCTTCCACCGGCTGGAGGAGAGCTTCCTGGTGGA
GGAGGACAAGAAGCACGAGCGGCACCCCATCTTCGGCAACATCGTGGACGAGGTGGCCTAC
CACGAGAAGTACCCCACCATCTACCACCTGCGGAAGAAGCTGGTGGACAGCACCGACAAGG
CCGACCTGCGGCTGATCTACCTGGCCCTGGCCCACATGATCAAGTTCCGGGGCCACTTCCTG
ATCGAGGGCGACCTGAACCCCGACAACAGCGACGTGGACAAGCTGTTCATCCAGCTGGTGC
AGACCTACAACCAGCTGTTCGAGGAGAACCCCATCAACGCCAGCGGCGTGGACGCCAAGGC
CATCCTGAGCGCCCGGCTGAGCAAGAGCCGGCGGCTGGAGAACCTGATCGCCCAGCTGCCC
GGCGAGAAGAAGAACGGCCTGTTCGGCAACCTGATCGCCCTGAGCCTGGGCCTGACCCCCA
ACTTCAAGAGCAACTTCGACCTGGCCGAGGACGCCAAGCTGCAGCTGAGCAAGGACACCTA
CGACGACGACCTGGACAACCTGCTGGCCCAGATCGGCGACCAGTACGCCGACCTGTTCCTGG
CCGCCAAGAACCTGAGCGACGCCATCCTGCTGAGCGACATCCTGCGGGTGAACACCGAGAT
CACCAAGGCCCCCCTGAGCGCCAGCATGATCAAGCGGTACGACGAGCACCACCAGGACCTG
ACCCTGCTGAAGGCCCTGGTGCGGCAGCAGCTGCCCGAGAAGTACAAGGAGATCTTCTTCG
ACCAGAGCAAGAACGGCTACGCCGGCTACATCGACGGCGGCGCCAGCCAGGAGGAGTTCTA
CAAGTTCATCAAGCCCATCCTGGAGAAGATGGACGGCACCGAGGAGCTGCTGGTGAAGCTG
AACCGGGAGGACCTGCTGCGGAAGCAGCGGACCTTCGACAACGGCAGCATCCCCCACCAGA
TCCACCTGGGCGAGCTGCACGCCATCCTGCGGCGGCAGGAGGACTTCTACCCCTTCCTGAAG
GACAACCGGGAGAAGATCGAGAAGATCCTGACCTTCCGGATCCCCTACTACGTGGGCCCCCT
GGCCCGGGGCAACAGCCGGTTCGCCTGGATGACCCGGaaatccGAGGAGACCATCACCCCCTG
GAACTTCGAGGAGGTGGTGGACAAGGGCGCCAGCGCCCAGAGCTTCATCGAGCGGATGACC
AACTTCGACAAGAACCTGCCCAACGAGAAGGTGCTGCCCAAGCACAGCCTGCTGTACGAGT
ACTTCACCGTGTACAACGAGCTGACCAAGGTGAAGTACGTGACCGAGGGCATGCGGAAGCC
CGCCTTCCTGAGCGGCGAGCAGAAGAAGGCCATCGTGGACCTGCTGTTCAAGACCAACCGG
AAGGTGACCGTGAAGCAGCTGAAGGAGGACTACTTCAAGAAGATCGAGTGCTTCGACAGCG
TGGAGATCAGCGGCGTGGAGGACCGGTTCAACGCCAGCCTGGGCACCTACCACGACCTGCT
GAAGATCATCAAGGACAAGGACTTCCTGGACAACGAGGAGAACGAGGACATCCTGGAGGA
CATCGTGCTGACCCTGACCCTGTTCGAGGACCGGGAGATGATCGAGGAGCGGCTGAAaACCT
ACGCCCACCTGTTCGACGACAAGGTGATGAAGCAGCTGAAGCGGCGGCGGTACACCGGCTG
GGGCCGGCTGAGCCGGAAGCTGATCAACGGCATCCGGGACAAGCAGAGCGGCAAGACCATC
CTGGACTTCCTGaaatccGACGGCTTCGCCAACCGGAACTTCATGCAGCTGATCCACGACGACA
GCCTGACCTTCAAGGAGGACATCCAGAAGGCCCAGGTGAGCGGCCAGGGCGACAGCCTGCA
CGAGCACATCGCCAACCTGGCCGGCAGCCCCGCCATCAAGAAGGGCATCCTGCAGACCGTG
AAGGTGGTGGACGAGCTGGTGAAGGTGATGGGCCGGCACAAGCCCGAGAACATCGTGATCG
AGATGGCCCGGGAGAACCAGACCACCCAGAAGGGCCAGAAGAACAGCCGGGAGCGGATGA
AGCGGATCGAGGAGGGCATCAAGGAGCTGGGCAGCCAGATCCTGAAGGAGCACCCCGTGG
AGAACACCCAGCTGCAGAACGAGAAGCTGTACCTGTACTACCTGCAGAACGGCCGGGACAT
GTACGTGGACCAGGAGCTGGACATCAACCGGCTGAGCGACTACGACGTGGCCGCCATCGTG
CCCCAGAGCTTCCTGAAGGACGACAGCATCGACAACAAGGTGCTGACCCGGAGCGACAAGG
CCCGGGGCAAGAGCGACAACGTGCCCAGCGAGGAGGTGGTGAAGAAGATGAAGAACTACT
GGCGGCAGCTGCTGAACGCCAAGCTGATCACCCAGCGGAAGTTCGACAACCTGACCAAGGC
CGAGCGGGGCGGCCTGAGCGAGCTGGACAAGGCCGGCTTCATCAAGCGGCAGCTGGTGGAG
ACCCGGCAGATCACCAAGCACGTGGCCCAGATCCTGGACAGCCGGATGAACACCAAGTACG
ACGAGAACGACAAGCTGATCCGGGAGGTGAAGGTGATCACCCTGaaatccAAGCTGGTGAGCG
ACTTCCGGAAGGACTTCCAGTTCTACAAGGTGCGGGAGATCAACAACTACCACCACGCCCAC
GACGCCTACCTGAACGCCGTGGTGGGCACCGCCCTGATCAAGAAGTACCCCAAGCTGGAGA
GCGAGTTCGTGTACGGCGACTACAAGGTGTACGACGTGCGGAAGATGATCGCCAAGAGCGA
GCAGGAGATCGGCAAGGCCACCGCCAAGTACTTCTTCTACAGCAACATCATGAACTTCTTCA
AGACCGAGATCACCCTGGCCAACGGCGAGATCCGGAAGCGGCCCCTGATCGAGACCAACGG
CGAGACCGGCGAGATCGTGTGGGACAAGGGCCGGGACTTCGCCACCGTGCGGAAGGTGCTG
AGCATGCCCCAGGTGAACATCGTGAAGAAaACCGAGGTGCAGACCGGCGGCTTCAGCAAGG
AGAGCATCCTGCCCAAGCGGAACAGCGACAAGCTGATCGCCCGGAAGAAGGACTGGGACCC
CAAGAAGTACGGCGGCTTCGACAGCCCCACCGTGGCCTACAGCGTGCTGGTGGTGGCCAAG
GTGGAGAAGGGCAAGAGCAAGAAGCTGaaatccGTGAAGGAGCTGCTGGGCATCACCATCATG
GAGCGGAGCAGCTTCGAGAAGAACCCCATCGACTTCCTGGAGGCCAAGGGCTACAAGGAGG
TGAAGAAGGACCTGATCATCAAGCTGCCCAAGTACAGCCTGTTCGAGCTGGAGAACGGCCG
GAAGCGGATGCTGGCCAGCGCCGGCGAGCTGCAGAAGGGCAACGAGCTGGCCCTGCCCAGC
AAGTACGTGAACTTCCTGTACCTGGCCAGCCACTACGAGAAGCTGAAGGGCAGCCCCGAGG
ACAACGAGCAGAAGCAGCTGTTCGTGGAGCAGCACAAGCACTACCTGGACGAGATCATCGA
GCAGATCAGCGAGTTCAGCAAGCGGGTGATCCTGGCCGACGCCAACCTGGACAAGGTGCTG
AGCGCCTACAACAAGCACCGGGACAAGCCCATCCGGGAGCAGGCCGAGAACATCATCCACC
TGTTCACCCTGACCAACCTGGGCGCCCCCGCCGCCTTCAAGTACTTCGACACCACCATCGAC
CGGAAGCGGTACACCAGCACCAAGGAGGTGCTGGACGCCACCCTGATCCACCAGAGCATCA
CCGGCCTGTACGAGACCCGGATCGACCTGAGCCAGCTGGGCGGCGACAAGCGGCCCGCCGC
CACCAAGAAGGCCGGCCAGGCCAAGAAGAAGAAGGCCAGCGACGCCAAGAGCCTGACCGC
CTGGAGCCGGACCCTGGTGACCTTCAAGGACGTGTTCGTGGACTTCACCCGGGAGGAGTGG
AAGCTGCTGGACACCGCCCAGCAGATCCTGTACCGGAACGTGATGCTGGAGAACTACAAGA
ACCTGGTGAGCCTGGGCTACCAGCTGACCAAGCCCGACGTGATCCTGCGGCTGGAGAAGGG
CGAGGAGCCCTGGCTGGTGGAGCGGGAGATCCACCAGGAGACCCACCCCGACAGCGAGACC
GCCTTCGAGATCAAGAGCAGCGTGAGCGGCGGCAAGCGGCCCGCCGCCACCAAGAAGGCCG
GCCAGGCCAAGAAGAAGAAGGGCAGCTACCCCTACGACGTGCCCGACTACGCCTGAGcggccgctta
attaagetgccttctgcggggcttgccttctggccatgcccttcttctctcccttgcacctgtacctc
ttggtctttgaataaagcctgagtaggaagtctagaaaaaaaaaaaaaaaaaaaaaaaaaaaaaaaaa
aaaaaaaaaaaaaaaaaaaaaaaaaaaaaaaaaaaaaaaaaaaaaaattgtcttcttcatcgcc
tgcagATCCCAATGGCGCGCCGAGCTTGGCTCGAGCATGGTCATAGCTGTTTCCTGTGTGAAAT
TGTTATCCGCTCACAATTCCACACAACATACGAGCCGGAAGCATAAAGTGTAAAGCCTGGG
GTGCCTAATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGTTTGCG
TATTGGGCGCTCTTCCGCTTCCTCGCTCACTGACTCGCTGCGCTCGGTCGTTCGGCTGCGGCG
AGCGGTATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCGCGTTG
CTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTC
GTGCGCTCTCCTGTTCCGACCCTGCCGCTTACCGGATACCTGTCCGCCTTTCTCCCTTCGGGA
AGCGTGGCGCTTTCTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTAAAGTATA
TATGAGTAAACTTGGTCTGACAGTTAGAAAAACTCATCGAGCATCAAATGAAACTGCAATTT
ATTCATATCAGGATTATCAATACCATATTTTTGAAAAAGCCGTTTCTGTAATGAAGGAGAAA
ACTCACCGAGGCAGTTCCATAGGATGGCAAGATCCTGGTATCGGTCTGCGATTCCGACTCGT
CCAACATCAATACAACCTATTAATTTCCCCTCGTCAAAAATAAGGTTATCAAGTGAGAAATC
ACCATGAGTGACGACTGAATCCGGTGAGAATGGCAAAAGTTTATGCATTTCTTTCCAGACTT
GTTCAACAGGCCAGCCATTACGCTCGTCATCAAAATCACTCGCATCAACCAAACCGTTATTC
ATTCGTGATTGCGCCTGAGCGAGACGAAATACGCGATCGCTGTTAAAAGGACAATTACAAA
CAGGAATCGAATGCAACCGGCGCAGGAACACTGCCAGCGCATCAACAATATTTTCACCTGA
ATCAGGATATTCTTCTAATACCTGGAATGCTGTTTTCCCAGGGATCGCAGTGGTGAGTAACC
ATGCATCATCAGGAGTACGGATAAAATGCTTGATGGTCGGAAGAGGCATAAATTCCGTCAG
CCAGTTTAGTCTGACCATCTCATCTGTAACATCATTGGCAACGCTACCTTTGCCATGTTTCAG
AAACAACTCTGGCGCATCGGGCTTCCCATACAATCGATAGATTGTCGCACCTGATTGCCCGA
CATTATCGCGAGCCCATTTATACCCATATAAATCAGCATCCATGTTGGAATTTAATCGCGGC
CTAGAGCAAGACGTTTCCCGTTGAATATGGCTCATACTCTTCCTTTTTCAATATTATTGAAGC
ATTTATCAGGGTTATTGTCTCATGAGCGGATACATATTTGAATGTATTTAGAAAAATAAACA
AATAGGGGTTCCGCGCACATTTCCCCGAAAAGTGCCACCTGACGTCTAAGAAACCATTATTA
TCATGACATTAACCTATAAAAATAGGCGTATCACGAGGCCCTTTCGTC
Sp-dCas9-KRAB (MR-28122) Expressed mRNA sequence:
(SEQ ID NO: 205)
AGGAAATAAGAGAGAAAAGAAGAGTAAGAAGAAATATAAGAGCCACCATGGCCCCCAAGA
AGAAGCGGAAGGTGGGCATCCACGGCGTGCCCGCCGCCGACAAGAAGTACAGCATCGGCCT
GGCCATCGGCACCAACAGCGTGGGCTGGGCCGTGATCACCGACGAGTACAAGGTGCCCAGC
AAGAAGTTCAAGGTGCTGGGCAACACCGACCGGCACAGCATCAAGAAGAACCTGATCGGCG
CCCTGCTGTTCGACAGCGGCGAGACCGCCGAGGCCACCCGGCTGAAGCGGACCGCCCGGCG
GCGGTACACCCGGCGGAAGAACCGGATCTGCTACCTGCAGGAGATCTTCAGCAACGAGATG
GCCAAGGTGGACGACAGCTTCTTCCACCGGCTGGAGGAGAGCTTCCTGGTGGAGGAGGACA
AGAAGCACGAGCGGCACCCCATCTTCGGCAACATCGTGGACGAGGTGGCCTACCACGAGAA
GTACCCCACCATCTACCACCTGCGGAAGAAGCTGGTGGACAGCACCGACAAGGCCGACCTG
CGGCTGATCTACCTGGCCCTGGCCCACATGATCAAGTTCCGGGGCCACTTCCTGATCGAGGG
CGACCTGAACCCCGACAACAGCGACGTGGACAAGCTGTTCATCCAGCTGGTGCAGACCTAC
AACCAGCTGTTCGAGGAGAACCCCATCAACGCCAGCGGCGTGGACGCCAAGGCCATCCTGA
GCGCCCGGCTGAGCAAGAGCCGGCGGCTGGAGAACCTGATCGCCCAGCTGCCCGGCGAGAA
GAAGAACGGCCTGTTCGGCAACCTGATCGCCCTGAGCCTGGGCCTGACCCCCAACTTCAAGA
GCAACTTCGACCTGGCCGAGGACGCCAAGCTGCAGCTGAGCAAGGACACCTACGACGACGA
CCTGGACAACCTGCTGGCCCAGATCGGCGACCAGTACGCCGACCTGTTCCTGGCCGCCAAGA
ACCTGAGCGACGCCATCCTGCTGAGCGACATCCTGCGGGTGAACACCGAGATCACCAAGGC
CCCCCTGAGCGCCAGCATGATCAAGCGGTACGACGAGCACCACCAGGACCTGACCCTGCTG
AAGGCCCTGGTGCGGCAGCAGCTGCCCGAGAAGTACAAGGAGATCTTCTTCGACCAGAGCA
AGAACGGCTACGCCGGCTACATCGACGGCGGCGCCAGCCAGGAGGAGTTCTACAAGTTCAT
CAAGCCCATCCTGGAGAAGATGGACGGCACCGAGGAGCTGCTGGTGAAGCTGAACCGGGAG
GACCTGCTGCGGAAGCAGCGGACCTTCGACAACGGCAGCATCCCCCACCAGATCCACCTGG
GCGAGCTGCACGCCATCCTGCGGCGGCAGGAGGACTTCTACCCCTTCCTGAAGGACAACCG
GGAGAAGATCGAGAAGATCCTGACCTTCCGGATCCCCTACTACGTGGGCCCCCTGGCCCGGG
GCAACAGCCGGTTCGCCTGGATGACCCGGaaatccGAGGAGACCATCACCCCCTGGAACTTCGA
GGAGGTGGTGGACAAGGGCGCCAGCGCCCAGAGCTTCATCGAGCGGATGACCAACTTCGAC
AAGAACCTGCCCAACGAGAAGGTGCTGCCCAAGCACAGCCTGCTGTACGAGTACTTCACCG
TGTACAACGAGCTGACCAAGGTGAAGTACGTGACCGAGGGCATGCGGAAGCCCGCCTTCCT
GAGCGGCGAGCAGAAGAAGGCCATCGTGGACCTGCTGTTCAAGACCAACCGGAAGGTGACC
GTGAAGCAGCTGAAGGAGGACTACTTCAAGAAGATCGAGTGCTTCGACAGCGTGGAGATCA
GCGGCGTGGAGGACCGGTTCAACGCCAGCCTGGGCACCTACCACGACCTGCTGAAGATCAT
CAAGGACAAGGACTTCCTGGACAACGAGGAGAACGAGGACATCCTGGAGGACATCGTGCTG
ACCCTGACCCTGTTCGAGGACCGGGAGATGATCGAGGAGCGGCTGAAaACCTACGCCCACCT
GTTCGACGACAAGGTGATGAAGCAGCTGAAGCGGCGGCGGTACACCGGCTGGGGCCGGCTG
AGCCGGAAGCTGATCAACGGCATCCGGGACAAGCAGAGCGGCAAGACCATCCTGGACTTCC
TGaaatccGACGGCTTCGCCAACCGGAACTTCATGCAGCTGATCCACGACGACAGCCTGACCTT
CAAGGAGGACATCCAGAAGGCCCAGGTGAGCGGCCAGGGCGACAGCCTGCACGAGCACAT
CGCCAACCTGGCCGGCAGCCCCGCCATCAAGAAGGGCATCCTGCAGACCGTGAAGGTGGTG
GACGAGCTGGTGAAGGTGATGGGCCGGCACAAGCCCGAGAACATCGTGATCGAGATGGCCC
GGGAGAACCAGACCACCCAGAAGGGCCAGAAGAACAGCCGGGAGCGGATGAAGCGGATCG
AGGAGGGCATCAAGGAGCTGGGCAGCCAGATCCTGAAGGAGCACCCCGTGGAGAACACCC
AGCTGCAGAACGAGAAGCTGTACCTGTACTACCTGCAGAACGGCCGGGACATGTACGTGGA
CCAGGAGCTGGACATCAACCGGCTGAGCGACTACGACGTGGCCGCCATCGTGCCCCAGAGC
TTCCTGAAGGACGACAGCATCGACAACAAGGTGCTGACCCGGAGCGACAAGGCCCGGGGCA
AGAGCGACAACGTGCCCAGCGAGGAGGTGGTGAAGAAGATGAAGAACTACTGGCGGCAGC
TGCTGAACGCCAAGCTGATCACCCAGCGGAAGTTCGACAACCTGACCAAGGCCGAGCGGGG
CGGCCTGAGCGAGCTGGACAAGGCCGGCTTCATCAAGCGGCAGCTGGTGGAGACCCGGCAG
ATCACCAAGCACGTGGCCCAGATCCTGGACAGCCGGATGAACACCAAGTACGACGAGAACG
ACAAGCTGATCCGGGAGGTGAAGGTGATCACCCTGaaatccAAGCTGGTGAGCGACTTCCGGA
AGGACTTCCAGTTCTACAAGGTGCGGGAGATCAACAACTACCACCACGCCCACGACGCCTA
CCTGAACGCCGTGGTGGGCACCGCCCTGATCAAGAAGTACCCCAAGCTGGAGAGCGAGTTC
GTGTACGGCGACTACAAGGTGTACGACGTGCGGAAGATGATCGCCAAGAGCGAGCAGGAGA
TCGGCAAGGCCACCGCCAAGTACTTCTTCTACAGCAACATCATGAACTTCTTCAAGACCGAG
ATCACCCTGGCCAACGGCGAGATCCGGAAGCGGCCCCTGATCGAGACCAACGGCGAGACCG
GCGAGATCGTGTGGGACAAGGGCCGGGACTTCGCCACCGTGCGGAAGGTGCTGAGCATGCC
CCAGGTGAACATCGTGAAGAAaACCGAGGTGCAGACCGGCGGCTTCAGCAAGGAGAGCATC
CTGCCCAAGCGGAACAGCGACAAGCTGATCGCCCGGAAGAAGGACTGGGACCCCAAGAAGT
ACGGCGGCTTCGACAGCCCCACCGTGGCCTACAGCGTGCTGGTGGTGGCCAAGGTGGAGAA
GGGCAAGAGCAAGAAGCTGaaatccGTGAAGGAGCTGCTGGGCATCACCATCATGGAGCGGAG
CAGCTTCGAGAAGAACCCCATCGACTTCCTGGAGGCCAAGGGCTACAAGGAGGTGAAGAAG
GACCTGATCATCAAGCTGCCCAAGTACAGCCTGTTCGAGCTGGAGAACGGCCGGAAGCGGA
TGCTGGCCAGCGCCGGCGAGCTGCAGAAGGGCAACGAGCTGGCCCTGCCCAGCAAGTACGT
GAACTTCCTGTACCTGGCCAGCCACTACGAGAAGCTGAAGGGCAGCCCCGAGGACAACGAG
CAGAAGCAGCTGTTCGTGGAGCAGCACAAGCACTACCTGGACGAGATCATCGAGCAGATCA
GCGAGTTCAGCAAGCGGGTGATCCTGGCCGACGCCAACCTGGACAAGGTGCTGAGCGCCTA
CAACAAGCACCGGGACAAGCCCATCCGGGAGCAGGCCGAGAACATCATCCACCTGTTCACC
CTGACCAACCTGGGCGCCCCCGCCGCCTTCAAGTACTTCGACACCACCATCGACCGGAAGCG
GTACACCAGCACCAAGGAGGTGCTGGACGCCACCCTGATCCACCAGAGCATCACCGGCCTG
TACGAGACCCGGATCGACCTGAGCCAGCTGGGCGGCGACAAGCGGCCCGCCGCCACCAAGA
AGGCCGGCCAGGCCAAGAAGAAGAAGGCCAGCGACGCCAAGAGCCTGACCGCCTGGAGCC
GGACCCTGGTGACCTTCAAGGACGTGTTCGTGGACTTCACCCGGGAGGAGTGGAAGCTGCTG
GACACCGCCCAGCAGATCCTGTACCGGAACGTGATGCTGGAGAACTACAAGAACCTGGTGA
GCCTGGGCTACCAGCTGACCAAGCCCGACGTGATCCTGCGGCTGGAGAAGGGCGAGGAGCC
CTGGCTGGTGGAGCGGGAGATCCACCAGGAGACCCACCCCGACAGCGAGACCGCCTTCGAG
ATCAAGAGCAGCGTGAGCGGCGGCAAGCGGCCCGCCGCCACCAAGAAGGCCGGCCAGGCC
AAGAAGAAGAAGGGCAGCTACCCCTACGACGTGCCCGACTACGCCTGAGcggccgcttaatta
agctgccttctgcggggcttgccttctggccatgcccttcttctctcccttgcacctgtacct
cttggtctttgaataaagcctgagtaggaagtctagaaaaaaaaaaaaaaaaaaaaaaaaaaa
aaaaaaaaaaaaaaaaaaaaaaaaaaaaaaaaaaaaaaaaaaaaaaaaaaaaa

In some embodiments, a site-specific disrupting agent comprises the amino acid sequence of SEQ ID NOs: 206 or 75. In some embodiments, a nucleic acid described herein comprises a nucleic acid sequence of SEQ ID NO: 206 or 75 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Sp-dCas9-KRAB Protein sequence:
(SEQ ID NO: 206)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKE
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVN]
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEF
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLK:
YAHLFDDKVMKQLKRRRYTGWGRLSRKLINGIRDKQSGKTILDFLKSDGFANRNFMQLIHDDSI
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKASDAKSLTAWSRTLVTFKDVFVDFTREEWK
LLDTAQQILYRNVMLENYKNLVSLGYQLTKPDVILRLEKGEEPWLVEREIHQETHPDSETAFEIK
SSVSGGKRPAATKKAGQAKKKKGSYPYDVPDYA
Sp-dCas9-KRAB Protein sequence without HA tag:
(SEQ ID NO: 75)
MAPKKKRKVGIHGVPAADKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEEDKKH
ERHPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIALS
LGLTPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEFYKF
IKPILEKMDGTEELLVKLNREDLLRKQRTFDNGSIPHQIHLGELHAILRRQEDFYPFLKDNREKIEK
ILTFRIPYYVGPLARGNSRFAWMTRKSEETITPWNFEEVVDKGASAQSFIERMTNFDKNLPNEKV
LPKHSLLYEYFTVYNELTKVKYVTEGMRKPAFLSGEQKKAIVDLLFKTNRKVTVKQLKEDYFK
KIECFDSVEISGVEDRFNASLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMGRHKPENIVIEMAREN
QTTQKGQKNSRERMKRIEEGIKELGSQILKEHPVENTQLQNEKLYLYYLQNGRDMYVDQELDIN
RLSDYDVAAIVPQSFLKDDSIDNKVLTRSDKARGKSDNVPSEEVVKKMKNYWRQLLNAKLITQ
RKFDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRKMI
AKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLASAG
ELQKGNELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKRYTSTKEVLDATLIHQ
SITGLYETRIDLSQLGGDKRPAATKKAGQAKKKKASDAKSLTAWSRTLVTFKDVFVDFTREEWK
LLDTAQQILYRNVMLENYKNLVSLGYQLTKPDVILRLEKGEEPWLVEREIHQETHPDSETAFEIK
SSVSGGKRPAATKKAGQAKKKKGS

In some embodiments, a site-specific disrupting agent comprises a targeting moiety (e.g., comprising dCas9, e.g., an S. aureus dCas9) and an effector moiety comprising EZH2. In some embodiments, a site-specific disrupting agent comprises a targeting moiety comprising dCas9, e.g., an S. pyogenes dCas9, e.g., mutant S. pyogenes Cas9, e.g., Cas9m4 and an effector moiety comprising EZH2. In some embodiments, the site-specific disrupting agent is encoded by the nucleic acid sequence of SEQ ID NO: 209 (e.g., mRNA encoding the site-specific disrupting agent). In some embodiments, a nucleic acid described herein comprises a nucleic acid sequence of SEQ ID NO: 209 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto. In some embodiments, the targeting moiety is encoded by the nucleic acid sequence of SEQ ID NO: 8 and/or the effector moiety is encoded by the nucleic acid sequence of SEQ ID NO: 18.

EZH2-dCas9 mRNA (MR28938)
(SEQ ID NO: 209)
AGGAAAUAAGAGAGAAAAGAAGAGUAAGAAGAAAUAUAAGAGCCACCAUGGCCCCCAAG
AAGAAGCGGAAGGUGGGCGGCAGCGGCGGCAGCGGCCAGACCGGCAAGAAGAGCGAGAA
GGGCCCCGUGUGCUGGCGGAAGCGGGUGAAGAGCGAGUACAUGCGGCUGCGGCAGCUGA
AGCGGUUCCGGCGGGCCGACGAGGUGAAGAGCAUGUUCAGCAGCAACCGGCAGAAGAUCC
UGGAGCGGACCGAGAUCCUGAACCAGGAGUGGAAGCAGCGGCGAAUCCAGCCCGUGCACA
UCCUGACCAGCGUGAGCAGCCUGCGGGGCACCCGGGAGUGCAGCGUGACCAGCGACCUGG
ACUUCCCCACCCAGGUGAUCCCCCUAAAGACCCUGAACGCCGUGGCCAGCGUGCCCAUCA
UGUACAGCUGGAGCCCCCUGCAGCAGAACUUCAUGGUGGAGGACGAGACCGUGCUGCACA
ACAUCCCCUACAUGGGCGACGAGGUGCUGGACCAGGACGGCACCUUCAUCGAGGAGCUGA
UCAAGAACUACGACGGCAAGGUGCACGGCGACCGGGAGUGCGGCUUCAUCAACGACGAGA
UCUUCGUGGAGCUGGUGAACGCCCUGGGCCAGUACAACGACGACGACGACGACGACGACG
GCGACGACCCCGAGGAGCGGGAGGAGAAGCAGAAGGACCUGGAGGACCACCGGGACGACA
AGGAGAGCCGGCCCCCCCGGAAGUUCCCCAGCGACAAGAUCUUCGAGGCCAUCAGCAGCA
UGUUCCCCGACAAGGGCACCGCCGAGGAGCUGAAGGAGAAGUACAAGGAGCUGACCGAGC
AGCAGCUGCCCGGCGCCCUGCCCCCCGAGUGCACCCCCAACAUCGACGGCCCCAACGCCAA
GAGCGUGCAGCGGGAGCAGAGCCUGCACAGCUUCCACACCCUGUUCUGCCGGCGGUGCUU
CAAGUACGACUGCUUCCUGCACCCCUUCCACGCCACCCCCAACACCUACAAGCGGAAGAA
CACCGAGACCGCCCUGGACAACAAGCCCUGCGGCCCCCAGUGCUACCAGCACCUGGAGGG
CGCCAAGGAGUUCGCCGCCGCCCUGACCGCCGAGCGGAUCAAGACCCCCCCCAAGCGGCCC
GGCGGCCGGCGGCGGGGCCGGCUGCCCAACAACAGCAGCCGGCCCAGCACCCCCACCAUCA
ACGUGCUGGAGAGCAAGGACACCGACAGCGACCGGGAGGCCGGCACCGAGACCGGCGGCG
AGAACAACGACAAGGAGGAGGAGGAGAAGAAGGACGAGACCAGCAGCAGCAGCGAGGCC
AACAGCCGGUGCCAGACCCCCAUCAAGAUGAAGCCCAACAUCGAGCCCCCCGAGAACGUG
GAGUGGAGCGGCGCCGAGGCCAGCAUGUUCCGGGUGCUGAUCGGCACCUACUACGACAAC
UUCUGCGCCAUCGCCCGGCUGAUCGGCACCAAGACCUGCCGGCAGGUGUACGAGUUCCGG
GUGAAGGAGAGCAGCAUCAUCGCCCCCGCCCCCGCCGAGGACGUGGACACCCCCCCCCGG
AAGAAGAAGCGGAAGCACCGGCUGUGGGCCGCCCACUGCCGGAAGAUCCAGCUGAAGAAG
GACGGCAGCAGCAACCACGUGUACAACUACCAGCCCUGCGACCACCCCCGGCAGCCCUGC
GACAGCAGCUGCCCCUGCGUGAUCGCCCAGAACUUCUGCGAGAAGUUCUGCCAGUGCAGC
AGCGAGUGCCAGAACCGGUUCCCCGGCUGCCGGUGCAAGGCCCAGUGCAACACCAAGCAG
UGCCCCUGCUACCUGGCCGUGCGGGAGUGCGACCCCGACCUGUGCCUGACCUGCGGCGCC
GCCGACCACUGGGACAGCAAGAACGUGAGCUGCAAGAACUGCAGCAUCCAGCGGGGCAGC
AAGAAGCACCUGCUGCUGGCCCCCAGCGACGUGGCCGGCUGGGGCAUCUUCAUCAAGGAC
CCCGUGCAGAAGAACGAGUUCAUCAGCGAGUACUGCGGCGAGAUCAUCAGCCAGGACGAG
GCCGACCGGCGGGGCAAGGUGUACGACAAGUACAUGUGCAGCUUCCUGUUCAACCUGAAC
AACGACUUCGUGGUGGACGCCACCCGGAAGGGCAACAAGAUCCGGUUCGCCAACCACAGC
GUGAACCCCAACUGCUACGCCAAGGUGAUGAUGGUGAACGGCGACCACCGGAUCGGCAUC
UUCGCCAAGCGGGCCAUCCAGACCGGCGAGGAGCUGUUCUUCGACUACCGGUACAGCCAG
GCCGACGCCCUGAAGUACGUGGGCAUCGAGCGGGAGAUGGAGAUCCCCAGCACCGGCGGC
AGCGGCGGCAGCGGCGGCAGCGGCGGCAGCGGCGGCAGCGGCCGACCCGACAAGAAGUAC
AGCAUCGGCCUGGCCAUCGGCACCAACAGCGUGGGCUGGGCCGUGAUCACCGACGAGUAC
AAGGUGCCCAGCAAGAAGUUCAAGGUGCUGGGCAACACCGACCGGCACAGCAUCAAGAAG
AACCUGAUCGGCGCCCUGCUGUUCGACAGCGGCGAGACCGCCGAGGCCACCCGGCUGAAG
CGGACCGCCCGGCGGCGGUACACCCGGCGGAAGAACCGGAUCUGCUACCUGCAGGAGAUC
UUCAGCAACGAGAUGGCCAAGGUGGACGACAGCUUCUUCCACCGGCUGGAGGAGAGCUUC
CUGGUGGAGGAGGACAAGAAGCACGAGCGGCACCCCAUCUUCGGCAACAUCGUGGACGAG
GUGGCCUACCACGAGAAGUACCCCACCAUCUACCACCUGCGGAAGAAGCUGGUGGACAGC
ACCGACAAGGCCGACCUGCGGCUGAUCUACCUGGCCCUGGCCCACAUGAUCAAGUUCCGG
GGCCACUUCCUGAUCGAGGGCGACCUGAACCCCGACAACAGCGACGUGGACAAGCUGUUC
AUCCAGCUGGUGCAGACCUACAACCAGCUGUUCGAGGAGAACCCCAUCAACGCCAGCGGC
GUGGACGCCAAGGCCAUCCUGAGCGCCCGGCUGAGCAAGAGCCGGCGGCUGGAGAACCUG
AUCGCCCAGCUGCCCGGCGAGAAGAAGAACGGCCUGUUCGGCAACCUGAUCGCCCUGAGC
CUGGGCCUGACCCCCAACUUCAAGAGCAACUUCGACCUGGCCGAGGACGCCAAGCUGCAG
CUGAGCAAGGACACCUACGACGACGACCUGGACAACCUGCUGGCCCAGAUCGGCGACCAG
UACGCCGACCUGUUCCUGGCCGCCAAGAACCUGAGCGACGCCAUCCUGCUGAGCGACAUC
CUGCGGGUGAACACCGAGAUCACCAAGGCCCCCCUGAGCGCCAGCAUGAUCAAGCGGUAC
GACGAGCACCACCAGGACCUGACCCUGCUGAAGGCCCUGGUGCGGCAGCAGCUGCCCGAG
AAGUACAAGGAGAUCUUCUUCGACCAGAGCAAGAACGGCUACGCCGGCUACAUCGACGGC
GGCGCCAGCCAGGAGGAGUUCUACAAGUUCAUCAAGCCCAUCCUGGAGAAGAUGGACGGC
ACCGAGGAGCUGCUGGUGAAGCUGAACCGGGAGGACCUGCUGCGGAAGCAGCGGACCUUC
GACAACGGCAGCAUCCCCCACCAGAUCCACCUGGGCGAGCUGCACGCCAUCCUGCGGCGG
CAGGAGGACUUCUACCCCUUCCUGAAGGACAACCGGGAGAAGAUCGAGAAGAUCCUGACC
UUCCGGAUCCCCUACUACGUGGGCCCCCUGGCCCGGGGCAACAGCCGGUUCGCCUGGAUG
ACCCGGAAAUCCGAGGAGACCAUCACCCCCUGGAACUUCGAGGAGGUGGUGGACAAGGGC
GCCAGCGCCCAGAGCUUCAUCGAGCGGAUGACCAACUUCGACAAGAACCUGCCCAACGAG
AAGGUGCUGCCCAAGCACAGCCUGCUGUACGAGUACUUCACCGUGUACAACGAGCUGACC
AAGGUGAAGUACGUGACCGAGGGCAUGCGGAAGCCCGCCUUCCUGAGCGGCGAGCAGAAG
AAGGCCAUCGUGGACCUGCUGUUCAAGACCAACCGGAAGGUGACCGUGAAGCAGCUGAAG
GAGGACUACUUCAAGAAGAUCGAGUGCUUCGACAGCGUGGAGAUCAGCGGCGUGGAGGA
CCGGUUCAACGCCAGCCUGGGCACCUACCACGACCUGCUGAAGAUCAUCAAGGACAAGGA
CUUCCUGGACAACGAGGAGAACGAGGACAUCCUGGAGGACAUCGUGCUGACCCUGACCCU
GUUCGAGGACCGGGAGAUGAUCGAGGAGCGGCUGAAAACCUACGCCCACCUGUUCGACGA
CAAGGUGAUGAAGCAGCUGAAGCGGCGGCGGUACACCGGCUGGGGCCGGCUGAGCCGGAA
GCUGAUCAACGGCAUCCGGGACAAGCAGAGCGGCAAGACCAUCCUGGACUUCCUGAAAUC
CGACGGCUUCGCCAACCGGAACUUCAUGCAGCUGAUCCACGACGACAGCCUGACCUUCAA
GGAGGACAUCCAGAAGGCCCAGGUGAGCGGCCAGGGCGACAGCCUGCACGAGCACAUCGC
CAACCUGGCCGGCAGCCCCGCCAUCAAGAAGGGCAUCCUGCAGACCGUGAAGGUGGUGGA
CGAGCUGGUGAAGGUGAUGGGCCGGCACAAGCCCGAGAACAUCGUGAUCGAGAUGGCCCG
GGAGAACCAGACCACCCAGAAGGGCCAGAAGAACAGCCGGGAGCGGAUGAAGCGGAUCGA
GGAGGGCAUCAAGGAGCUGGGCAGCCAGAUCCUGAAGGAGCACCCCGUGGAGAACACCCA
GCUGCAGAACGAGAAGCUGUACCUGUACUACCUGCAGAACGGCCGGGACAUGUACGUGGA
CCAGGAGCUGGACAUCAACCGGCUGAGCGACUACGACGUGGCCGCCAUCGUGCCCCAGAG
CUUCCUGAAGGACGACAGCAUCGACAACAAGGUGCUGACCCGGAGCGACAAGGCCCGGGG
CAAGAGCGACAACGUGCCCAGCGAGGAGGUGGUGAAGAAGAUGAAGAACUACUGGCGGC
AGCUGCUGAACGCCAAGCUGAUCACCCAGCGGAAGUUCGACAACCUGACCAAGGCCGAGC
GGGGCGGCCUGAGCGAGCUGGACAAGGCCGGCUUCAUCAAGCGGCAGCUGGUGGAGACCC
GGCAGAUCACCAAGCACGUGGCCCAGAUCCUGGACAGCCGGAUGAACACCAAGUACGACG
AGAACGACAAGCUGAUCCGGGAGGUGAAGGUGAUCACCCUGAAAUCCAAGCUGGUGAGC
GACUUCCGGAAGGACUUCCAGUUCUACAAGGUGCGGGAGAUCAACAACUACCACCACGCC
CACGACGCCUACCUGAACGCCGUGGUGGGCACCGCCCUGAUCAAGAAGUACCCCAAGCUG
GAGAGCGAGUUCGUGUACGGCGACUACAAGGUGUACGACGUGCGGAAGAUGAUCGCCAA
GAGCGAGCAGGAGAUCGGCAAGGCCACCGCCAAGUACUUCUUCUACAGCAACAUCAUGAA
CUUCUUCAAGACCGAGAUCACCCUGGCCAACGGCGAGAUCCGGAAGCGGCCCCUGAUCGA
GACCAACGGCGAGACCGGCGAGAUCGUGUGGGACAAGGGCCGGGACUUCGCCACCGUGCG
GAAGGUGCUGAGCAUGCCCCAGGUGAACAUCGUGAAGAAAACCGAGGUGCAGACCGGCG
GCUUCAGCAAGGAGAGCAUCCUGCCCAAGCGGAACAGCGACAAGCUGAUCGCCCGGAAGA
AGGACUGGGACCCCAAGAAGUACGGCGGCUUCGACAGCCCCACCGUGGCCUACAGCGUGC
UGGUGGUGGCCAAGGUGGAGAAGGGCAAGAGCAAGAAGCUGAAAUCCGUGAAGGAGCUG
CUGGGCAUCACCAUCAUGGAGCGGAGCAGCUUCGAGAAGAACCCCAUCGACUUCCUGGAG
GCCAAGGGCUACAAGGAGGUGAAGAAGGACCUGAUCAUCAAGCUGCCCAAGUACAGCCUG
UUCGAGCUGGAGAACGGCCGGAAGCGGAUGCUGGCCAGCGCCGGCGAGCUGCAGAAGGGC
AACGAGCUGGCCCUGCCCAGCAAGUACGUGAACUUCCUGUACCUGGCCAGCCACUACGAG
AAGCUGAAGGGCAGCCCCGAGGACAACGAGCAGAAGCAGCUGUUCGUGGAGCAGCACAAG
CACUACCUGGACGAGAUCAUCGAGCAGAUCAGCGAGUUCAGCAAGCGGGUGAUCCUGGCC
GACGCCAACCUGGACAAGGUGCUGAGCGCCUACAACAAGCACCGGGACAAGCCCAUCCGG
GAGCAGGCCGAGAACAUCAUCCACCUGUUCACCCUGACCAACCUGGGCGCCCCCGCCGCC
UUCAAGUACUUCGACACCACCAUCGACCGGAAGCGGUACACCAGCACCAAGGAGGUGCUG
GACGCCACCCUGAUCCACCAGAGCAUCACCGGCCUGUACGAGACCCGGAUCGACCUGAGC
CAGCUGGGCGGCGACAGCGGCGGCAAGCGGCCCGCCGCCACCAAGAAGGCCGGCCAGGCC
AAGAAGAAGAAGGGCAGCUACCCCUACGACGUGCCCGACUACGCCUGAGCGGCCGCUUAA
UUAAGCUGCCUUCUGCGGGGCUUGCCUUCUGGCCAUGCCCUUCUUCUCUCCCUUGCACCU
GUACCUCUUGGUCUUUGAAUAAAGCCUGAGUAGGAAGUCUAGAAAAAAAAAAAAAAAAA
AAAAAAAAAAAAAAAAAAAAAAAAAAAAAAAAAAAAAAAAAAAAAAAAAAAAAAAAAA
AAAAA

In some embodiments, a site-specific disrupting agent comprises the amino acid sequence of SEQ ID NOs: 210 or 76. In some embodiments, a nucleic acid described herein comprises a nucleic acid sequence of SEQ ID NO: 210 or 76 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9 Protein Sequence
(corresponding to MR-28938)
(SEQ ID NO: 210)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALYNKHRDKPIREQAENIIH
LFTLTNLGAPAAFKYFDTTIDRKRYTSTKEVLDATLIHQSITGLY
ETRIDLSQLGGDSGGKRPAATKKAGQAKKKKGSYPYDVPDYA
EZH2-dCas9 without HA tag
(SEQ ID NO: 76)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKG
RDFATVRKVLSMPQVNIVKKTEVQTGGFSKESILPKRNSDKLIAR
KKDWDPKKYGGFDSPTVAYSVLVVAKVEKGKSKKLKSVKELLGIT
IMERSSFEKNPIDFLEAKGYKEVKKDLIIKLPKYSLFELENGRKR
MLASAGELQKGNELALYNKHRDKPIREQAENIIHLFTLTNLGAPA
AFKYFDTTIDRKRYTSTKEVLDATLIHQSITGLYETRIDLSQLGG
DSGGKRPAATKKAGQAKKKKGS

In some embodiments, a site-specific disrupting agent comprises a targeting moiety (e.g., comprising dCas9, e.g., an S. aureus dCas9), and an effector moiety comprising DNMT3 (e.g., DNMT3a/3L). In some embodiments, a site-specific disrupting agent comprises a targeting moiety comprising dCas9, e.g., an S. pyogenes dCas9, e.g., mutant S. pyogenes Cas9, e.g., Cas9m4 and an effector moiety comprising DNMT3 (e.g., DNMT3a/3L). In some embodiments, the site-specific disrupting agent is encoded by the nucleic acid sequence of SEQ ID NO: 211 (e.g., mRNA encoding the site-specific disrupting agent). In some embodiments, a nucleic acid described herein comprises a nucleic acid sequence of SEQ ID NO: 211 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3a/3L mRNA (MR-29414)
(SEQ ID NO: 211)
AGGAAAUAAGAGAGAAAAGAAGAGUAAGAAGAAAUAUAAGAGCCA
CCAUGGCCCCCAAGAAGAAGCGGAAGGUGGGCAUCCACGGCGUGC
CCGCCGCCGACAAGAAGUACAGCAUCGGCCUGGCCAUCGGCACCA
ACAGCGUGGGCUGGGCCGUGAUCACCGACGAGUACAAGGUGCCCA
GCAAGAAGUUCAAGGUGCUGGGCAACACCGACCGGCACAGCAUCA
AGAAGAACCUGAUCGGCGCCCUGCUGUUCGACAGCGGCGAGACCG
CCGAGGCCACCCGGCUGAAGCGGACCGCCCGGCGGCGGUACACCC
GGCGGAAGAACCGGAUCUGCUACCUGCAGGAGAUCUUCAGCAACG
AGAUGGCCAAGGUGGACGACAGCUUCUUCCACCGGCUGGAGGAGA
GCUUCCUGGUGGAGGAGGACAAGAAGCACGAGCGGCACCCCAUCU
UCGGCAACAUCGUGGACGAGGUGGCCUACCACGAGAAGUACCCCA
CCAUCUACCACCUGCGGAAGAAGCUGGUGGACAGCACCGACAAGG
CCGACCUGCGGCUGAUCUACCUGGCCCUGGCCCACAUGAUCAAGU
UCCGGGGCCACUUCCUGAUCGAGGGCGACCUGAACCCCGACAACA
GCGACGUGGACAAGCUGUUCAUCCAGCUGGUGCAGACCUACAACC
AGCUGUUCGAGGAGAACCCCAUCAACGCCAGCGGCGUGGACGCCA
AGGCCAUCCUGAGCGCCCGGCUGAGCAAGAGCCGGCGGCUGGAGA
ACCUGAUCGCCCAGCUGCCCGGCGAGAAGAAGAACGGCCUGUUCG
GCAACCUGAUCGCCCUGAGCCUGGGCCUGACCCCCAACUUCAAGA
GCAACUUCGACCUGGCCGAGGACGCCAAGCUGCAGCUGAGCAAGG
ACACCUACGACGACGACCUGGACAACCUGCUGGCCCAGAUCGGCG
ACCAGUACGCCGACCUGUUCCUGGCCGCCAAGAACCUGAGCGACG
CCAUCCUGCUGAGCGACAUCCUGCGGGUGAACACCGAGAUCACCA
AGGCCCCCCUGAGCGCCAGCAUGAUCAAGCGGUACGACGAGCACC
ACCAGGACCUGACCCUGCUGAAGGCCCUGGUGCGGCAGCAGCUGC
CCGAGAAGUACAAGGAGAUCUUCUUCGACCAGAGCAAGAACGGCU
ACGCCGGCUACAUCGACGGCGGCGCCAGCCAGGAGGAGUUCUACA
AGUUCAUCAAGCCCAUCCUGGAGAAGAUGGACGGCACCGAGGAGC
UGCUGGUGAAGCUGAACCGGGAGGACCUGCUGCGGAAGCAGCGGA
CCUUCGACAACGGCAGCAUCCCCCACCAGAUCCACCUGGGCGAGC
UGCACGCCAUCCUGCGGCGGCAGGAGGACUUCUACCCCUUCCUGA
AGGACAACCGGGAGAAGAUCGAGAAGAUCCUGACCUUCCGGAUCC
CCUACUACGUGGGCCCCCUGGCCCGGGGCAACAGCCGGUUCGCCU
GGAUGACCCGGAAAUCCGAGGAGACCAUCACCCCCUGGAACUUCG
AGGAGGUGGUGGACAAGGGCGCCAGCGCCCAGAGCUUCAUCGAGC
GGAUGACCAACUUCGACAAGAACCUGCCCAACGAGAAGGUGCUGC
CCAAGCACAGCCUGCUGUACGAGUACUUCACCGUGUACAACGAGC
UGACCAAGGUGAAGUACGUGACCGAGGGCAUGCGGAAGCCCGCCU
UCCUGAGCGGCGAGCAGAAGAAGGCCAUCGUGGACCUGCUGUUCA
AGACCAACCGGAAGGUGACCGUGAAGCAGCUGAAGGAGGACUACU
UCAAGAAGAUCGAGUGCUUCGACAGCGUGGAGAUCAGCGGCGUGG
AGGACCGGUUCAACGCCAGCCUGGGCACCUACCACGACCUGCUGA
AGAUCAUCAAGGACAAGGACUUCCUGGACAACGAGGAGAACGAGG
ACAUCCUGGAGGACAUCGUGCUGACCCUGACCCUGUUCGAGGACC
GGGAGAUGAUCGAGGAGCGGCUGAAAACCUACGCCCACCUGUUCG
ACGACAAGGUGAUGAAGCAGCUGAAGCGGCGGCGGUACACCGGCU
GGGGCCGGCUGAGCCGGAAGCUGAUCAACGGCAUCCGGGACAAGC
AGAGCGGCAAGACCAUCCUGGACUUCCUGAAAUCCGACGGCUUCG
CCAACCGGAACUUCAUGCAGCUGAUCCACGACGACAGCCUGACCU
UCAAGGAGGACAUCCAGAAGGCCCAGGUGAGCGGCCAGGGCGACA
GCCUGCACGAGCACAUCGCCAACCUGGCCGGCAGCCCCGCCAUCA
AGAAGGGCAUCCUGCAGACCGUGAAGGUGGUGGACGAGCUGGUGA
AGGUGAUGGGCCGGCACAAGCCCGAGAACAUCGUGAUCGAGAUGG
CCCGGGAGAACCAGACCACCCAGAAGGGCCAGAAGAACAGCCGGG
AGCGGAUGAAGCGGAUCGAGGAGGGCAUCAAGGAGCUGGGCAGCC
AGAUCCUGAAGGAGCACCCCGUGGAGAACACCCAGCUGCAGAACG
AGAAGCUGUACCUGUACUACCUGCAGAACGGCCGGGACAUGUACG
UGGACCAGGAGCUGGACAUCAACCGGCUGAGCGACUACGACGUGG
CCGCCAUCGUGCCCCAGAGCUUCCUGAAGGACGACAGCAUCGACA
ACAAGGUGCUGACCCGGAGCGACAAGGCCCGGGGCAAGAGCGACA
ACGUGCCCAGCGAGGAGGUGGUGAAGAAGAUGAAGAACUACUGGC
GGCAGCUGCUGAACGCCAAGCUGAUCACCCAGCGGAAGUUCGACA
ACCUGACCAAGGCCGAGCGGGGCGGCCUGAGCGAGCUGGACAAGG
CCGGCUUCAUCAAGCGGCAGCUGGUGGAGACCCGGCAGAUCACCA
AGCACGUGGCCCAGAUCCUGGACAGCCGGAUGAACACCAAGUACG
ACGAGAACGACAAGCUGAUCCGGGAGGUGAAGGUGAUCACCCUGA
AAUCCAAGCUGGUGAGCGACUUCCGGAAGGACUUCCAGUUCUACA
AGGUGCGGGAGAUCAACAACUACCACCACGCCCACGACGCCUACC
UGAACGCCGUGGUGGGCACCGCCCUGAUCAAGAAGUACCCCAAGC
UGGAGAGCGAGUUCGUGUACGGCGACUACAAGGUGUACGACGUGC
GGAAGAUGAUCGCCAAGAGCGAGCAGGAGAUCGGCAAGGCCACCG
CCAAGUACUUCUUCUACAGCAACAUCAUGAACUUCUUCAAGACCG
AGAUCACCCUGGCCAACGGCGAGAUCCGGAAGCGGCCCCUGAUCG
AGACCAACGGCGAGACCGGCGAGAUCGUGUGGGACAAGGGCCGGG
ACUUCGCCACCGUGCGGAAGGUGCUGAGCAUGCCCCAGGUGAACA
UCGUGAAGAAAACCGAGGUGCAGACCGGCGGCUUCAGCAAGGAGA
GCAUCCUGCCCAAGCGGAACAGCGACAAGCUGAUCGCCCGGAAGA
AGGACUGGGACCCCAAGAAGUACGGCGGCUUCGACAGCCCCACCG
UGGCCUACAGCGUGCUGGUGGUGGCCAAGGUGGAGAAGGGCAAGA
GCAAGAAGCUGAAAUCCGUGAAGGAGCUGCUGGGCAUCACCAUCA
UGGAGCGGAGCAGCUUCGAGAAGAACCCCAUCGACUUCCUGGAGG
CCAAGGGCUACAAGGAGGUGAAGAAGGACCUGAUCAUCAAGCUGC
CCAAGUACAGCCUGUUCGAGCUGGAGAACGGCCGGAAGCGGAUGC
UGGCCAGCGCCGGCGAGCUGCAGAAGGGCAACGAGCUGGCCCUGC
CCAGCAAGUACGUGAACUUCCUGUACCUGGCCAGCCACUACGAGA
AGCUGAAGGGCAGCCCCGAGGACAACGAGCAGAAGCAGCUGUUCG
UGGAGCAGCACAAGCACUACCUGGACGAGAUCAUCGAGCAGAUCA
GCGAGUUCAGCAAGCGGGUGAUCCUGGCCGACGCCAACCUGGACA
AGGUGCUGAGCGCCUACAACAAGCACCGGGACAAGCCCAUCCGGG
AGCAGGCCGAGAACAUCAUCCACCUGUUCACCCUGACCAACCUGG
GCGCCCCCGCCGCCUUCAAGUACUUCGACACCACCAUCGACCGGA
AGCGGUACACCAGCACCAAGGAGGUGCUGGACGCCACCCUGAUCC
ACCAGAGCAUCACCGGCCUGUACGAGACCCGGAUCGACCUGAGCC
AGCUGGGCGGCGACAGCGCCGGCGGCGGCGGCAGCGGCGGCGGCG
GCAGCGGCGGCGGCGGCAGCGGCCCCAAGAAGAAGCGGAAGGUGG
CCGCCGCCGGCAGCAACCACGACCAGGAGUUCGACCCCCCCAAGG
UGUACCCCCCCGUGCCCGCCGAGAAGCGGAAGCCCAUCCGGGUGC
UGAGCCUGUUCGACGGCAUCGCCACCGGCCUGCUGGUGCUGAAGG
ACCUGGGCAUCCAGGUGGACCGGUACAUCGCCAGCGAGGUGUGCG
AGGACAGCAUCACCGUGGGCAUGGUGCGGCACCAGGGCAAGAUCA
UGUACGUGGGCGACGUGCGGAGCGUGACCCAGAAGCACAUCCAGG
AGUGGGGCCCCUUCGACCUGGUGAUCGGCGGCAGCCCCUGCAACG
ACCUGAGCAUCGUGAACCCCGCCCGGAAGGGCCUGUACGAGGGCA
CCGGCCGGCUGUUCUUCGAGUUCUACCGGCUGCUGCACGACGCCC
GGCCCAAGGAGGGCGACGACCGGCCCUUCUUCUGGCUGUUCGAGA
ACGUGGUGGCCAUGGGCGUGAGCGACAAGCGGGACAUCAGCCGGU
UCCUGGAGAGCAACCCCGUGAUGAUCGACGCCAAGGAGGUGAGCG
CCGCCCACCGGGCCCGGUACUUCUGGGGCAACCUGCCCGGCAUGA
ACCGGCCCCUGGCCAGCACCGUGAACGACAAGCUGGAGCUGCAGG
AGUGCCUGGAGCACGGCCGGAUCGCCAAGUUCAGCAAGGUGCGGA
CCAUCACCACCCGGAGCAACAGCAUCAAGCAGGGCAAGGACCAGC
ACUUCCCCGUGUUCAUGAACGAGAAGGAGGACAUCCUGUGGUGCA
CCGAGAUGGAGCGGGUGUUCGGCUUCCCCGUGCACUACACCGACG
UGAGCAACAUGAGCCGGCUGGCCCGGCAGCGGCUGCUGGGCCGGA
GCUGGAGCGUGCCCGUGAUCCGGCACCUGUUCGCCCCCCUGAAGG
AGUACUUCGCCUGCGUGAGCAGCGGCAACAGCAACGCCAACAGCC
GGGGCCCCAGCUUCAGCAGCGGCCUGGUGCCCCUGAGCCUGCGGG
GCAGCCACAUGAAUCCUCUGGAGAUGUUCGAGACAGUGCCCGUGU
GGAGAAGGCAACCCGUGAGGGUGCUGAGCCUCUUCGAGGACAUUA
AGAAGGAGCUGACCUCUCUGGGCUUUCUGGAAUCCGGCAGCGACC
CCGGCCAGCUGAAACACGUGGUGGACGUGACCGACACAGUGAGGA
AGGACGUGGAAGAGUGGGGCCCCUUUGACCUCGUGUAUGGAGCCA
CACCUCCUCUCGGCCACACAUGCGAUAGGCCUCCCAGCUGGUAUC
UCUUCCAGUUCCACAGACUGCUCCAGUACGCCAGACCUAAGCCCG
GCAGCCCCAGACCCUUCUUCUGGAUGUUCGUGGACAAUCUGGUGC
UGAACAAGGAGGAUCUGGAUGUGGCCAGCAGAUUUCUGGAGAUGG
AACCCGUGACAAUCCCCGACGUGCAUGGCGGCUCUCUGCAGAACG
CCGUGAGAGUGUGGUCCAACAUCCCCGCCAUUAGAAGCAGACACU
GGGCUCUGGUGAGCGAGGAGGAACUGUCUCUGCUGGCCCAGAAUA
AGCAGUCCUCCAAGCUGGCCGCCAAGUGGCCCACCAAGCUGGUGA
AGAACUGCUUUCUGCCUCUGAGGGAGUAUUUCAAGUAUUUCAGCA
CCGAACUGACCAGCAGCCUGAGCGGCGGCAAGCGGCCCGCCGCCA
CCAAGAAGGCCGGCCAGGCCAAGAAGAAGAAGGGCAGCUACCCCU
ACGACGUGCCCGACUACGCCUGAGCGGCCGCUUAAUUAAGCUGCC
UUCUGCGGGGCUUGCCUUCUGGCCAUGCCCUUCUUCUCUCCCUUG
CACCUGUACCUCUUGGUCUUUGAAUAAAGCCUGAGUAGGAAGUCU
AGAAAAAAAAAAAAAAAAAAAAAAAAAAAAAAAAAAAAAAAAAAA
AAAAAAAAAAAAAAAAAAAAAAAAAAAAAAAAAAAAA

In some embodiments, a site-specific disrupting agent comprises the amino acid sequence of SEQ ID NOs: 211 or 77. In some embodiments, a construct described herein comprises an amino acid sequence of SEQ ID NO: 211 or 77 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-DNMT3a/3L Protein Sequence
(corresponding to MR-29414)
(SEQ ID NO: 212)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSAGGGGSGGGG
SGGGGSGPKKKRKVAAAGSNHDQEFDPPKVYPPVPAEKRKPIRVL
SLFDGIATGLLVLKDLGIQVDRYIASEVCEDSITVGMVRHQGKIM
YVGDVRSVTQKHIQEWGPFDLVIGGSPCNDLSIVNPARKGLYEGT
GRLFFEFYRLLHDARPKEGDDRPFFWLFENVVAMGVSDKRDISRF
LESNPVMIDAKEVSAAHRARYFWGNLPGMNRPLASTVNDKLELQE
CLEHGRIAKFSKVRTITTRSNSIKQGKDQHFPVFMNEKEDILWCT
EMERVFGFPVHYTDVSNMSRLARQRLLGRSWSVPVIRHLFAPLKE
YFACVSSGNSNANSRGPSFSSGLVPLSLRGSHMNPLEMFETVPVW
RRQPVRVLSLFEDIKKELTSLGFLESGSDPGQLKHVVDVTDTVRK
DVEEWGPFDLVYGATPPLGHTCDRPPSWYLFQFHRLLQYARPKPG
SPRPFFWMFVDNLVLNKEDLDVASRFLEMEPVTIPDVHGGSLQNA
VRVWSNIPAIRSRHWALVSEEELSLLAQNKQSSKLAAKWPTKLVK
NCFLPLREYFKYFSTELTSSLSGGKRPAATKKAGQAKKKKGSYPY
DVPDYA
dCas-DNMT3a/3L (h) without HA tag
(SEQ ID NO: 77)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSAGGGGSGGGG
SGGGGSGPKKKRKVAAAGSNHDQEFDPPKVYPPVPAEKRKPIRVL
SLFDGIATGLLVLKDLGIQVDRYIASEVCEDSITVGMVRHQGKIM
YVGDVRSVTQKHIQEWGPFDLVIGGSPCNDLSIVNPARKGLYEGT
GRLFFEFYRLLHDARPKEGDDRPFFWLFENVVAMGVSDKRDISRF
LESNPVMIDAKEVSAAHRARYFWGNLPGMNRPLASTVNDKLELQE
CLEHGRIAKFSKVRTITTRSNSIKQGKDQHFPVFMNEKEDILWCT
EMERVFGFPVHYTDVSNMSRLARQRLLGRSWSVPVIRHLFAPLKE
YFACVSSGNSNANSRGPSFSSGLVPLSLRGSHMNPLEMFETVPVW
RRQPVRVLSLFEDIKKELTSLGFLESGSDPGQLKHVVDVTDTVRK
DVEEWGPFDLVYGATPPLGHTCDRPPSWYLFQFHRLLQYARPKPG
SPRPFFWMFVDNLVLNKEDLDVASRFLEMEPVTIPDVHGGSLQNA
VRVWSNIPAIRSRHWALVSEEELSLLAQNKQSSKLAAKWPTKLVK
NCFLPLREYFKYFSTELTSSLSGGKRPAATKKAGQAKKKKGS

In some embodiments, a site-specific disrupting agent comprises a targeting moiety (e.g., comprising dCas9, e.g., an S. aureus dCas9), and an effector moiety comprising HDAC8, e.g., a HDAC8 domain. In some embodiments, a site-specific disrupting agent comprises a targeting moiety comprising dCas9, e.g., an S. pyogenes dCas9, e.g., mutant S. pyogenes Cas9, e.g., Cas9m4, and an effector moiety comprising HDAC8, e.g., a HDAC8 domain. In some embodiments, the site-specific disrupting agent is encoded by the nucleic acid sequence of SEQ ID NO: 213 (e.g., mRNA encoding the site-specific disrupting agent).

In some embodiments, a nucleic acid described herein comprises a nucleic acid sequence of SEQ ID NO: 213 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-HDAC8 mRNA (MR-29439)
(SEQ ID NO: 213)
AGGAAAUAAGAGAGAAAAGAAGAGUAAGAAGAAAUAUAAGAGCCA
CCAUGGCCCCCAAGAAGAAGCGGAAGGUGGGCAUCCACGGCGUGC
CCGCCGCCGACAAGAAGUACAGCAUCGGCCUGGCCAUCGGCACCA
ACAGCGUGGGCUGGGCCGUGAUCACCGACGAGUACAAGGUGCCCA
GCAAGAAGUUCAAGGUGCUGGGCAACACCGACCGGCACAGCAUCA
AGAAGAACCUGAUCGGCGCCCUGCUGUUCGACAGCGGCGAGACCG
CCGAGGCCACCCGGCUGAAGCGGACCGCCCGGCGGCGGUACACCC
GGCGGAAGAACCGGAUCUGCUACCUGCAGGAGAUCUUCAGCAACG
AGAUGGCCAAGGUGGACGACAGCUUCUUCCACCGGCUGGAGGAGA
GCUUCCUGGUGGAGGAGGACAAGAAGCACGAGCGGCACCCCAUCU
UCGGCAACAUCGUGGACGAGGUGGCCUACCACGAGAAGUACCCCA
CCAUCUACCACCUGCGGAAGAAGCUGGUGGACAGCACCGACAAGG
CCGACCUGCGGCUGAUCUACCUGGCCCUGGCCCACAUGAUCAAGU
UCCGGGGCCACUUCCUGAUCGAGGGCGACCUGAACCCCGACAACA
GCGACGUGGACAAGCUGUUCAUCCAGCUGGUGCAGACCUACAACC
AGCUGUUCGAGGAGAACCCCAUCAACGCCAGCGGCGUGGACGCCA
AGGCCAUCCUGAGCGCCCGGCUGAGCAAGAGCCGGCGGCUGGAGA
ACCUGAUCGCCCAGCUGCCCGGCGAGAAGAAGAACGGCCUGUUCG
GCAACCUGAUCGCCCUGAGCCUGGGCCUGACCCCCAACUUCAAGA
GCAACUUCGACCUGGCCGAGGACGCCAAGCUGCAGCUGAGCAAGG
ACACCUACGACGACGACCUGGACAACCUGCUGGCCCAGAUCGGCG
ACCAGUACGCCGACCUGUUCCUGGCCGCCAAGAACCUGAGCGACG
CCAUCCUGCUGAGCGACAUCCUGCGGGUGAACACCGAGAUCACCA
AGGCCCCCCUGAGCGCCAGCAUGAUCAAGCGGUACGACGAGCACC
ACCAGGACCUGACCCUGCUGAAGGCCCUGGUGCGGCAGCAGCUGC
CCGAGAAGUACAAGGAGAUCUUCUUCGACCAGAGCAAGAACGGCU
ACGCCGGCUACAUCGACGGCGGCGCCAGCCAGGAGGAGUUCUACA
AGUUCAUCAAGCCCAUCCUGGAGAAGAUGGACGGCACCGAGGAGC
UGCUGGUGAAGCUGAACCGGGAGGACCUGCUGCGGAAGCAGCGGA
CCUUCGACAACGGCAGCAUCCCCCACCAGAUCCACCUGGGCGAGC
UGCACGCCAUCCUGCGGCGGCAGGAGGACUUCUACCCCUUCCUGA
AGGACAACCGGGAGAAGAUCGAGAAGAUCCUGACCUUCCGGAUCC
CCUACUACGUGGGCCCCCUGGCCCGGGGCAACAGCCGGUUCGCCU
GGAUGACCCGGAAAUCCGAGGAGACCAUCACCCCCUGGAACUUCG
AGGAGGUGGUGGACAAGGGCGCCAGCGCCCAGAGCUUCAUCGAGC
GGAUGACCAACUUCGACAAGAACCUGCCCAACGAGAAGGUGCUGC
CCAAGCACAGCCUGCUGUACGAGUACUUCACCGUGUACAACGAGC
UGACCAAGGUGAAGUACGUGACCGAGGGCAUGCGGAAGCCCGCCU
UCCUGAGCGGCGAGCAGAAGAAGGCCAUCGUGGACCUGCUGUUCA
AGACCAACCGGAAGGUGACCGUGAAGCAGCUGAAGGAGGACUACU
UCAAGAAGAUCGAGUGCUUCGACAGCGUGGAGAUCAGCGGCGUGG
AGGACCGGUUCAACGCCAGCCUGGGCACCUACCACGACCUGCUGA
AGAUCAUCAAGGACAAGGACUUCCUGGACAACGAGGAGAACGAGG
ACAUCCUGGAGGACAUCGUGCUGACCCUGACCCUGUUCGAGGACC
GGGAGAUGAUCGAGGAGCGGCUGAAAACCUACGCCCACCUGUUCG
ACGACAAGGUGAUGAAGCAGCUGAAGCGGCGGCGGUACACCGGCU
GGGGCCGGCUGAGCCGGAAGCUGAUCAACGGCAUCCGGGACAAGC
AGAGCGGCAAGACCAUCCUGGACUUCCUGAAAUCCGACGGCUUCG
CCAACCGGAACUUCAUGCAGCUGAUCCACGACGACAGCCUGACCU
UCAAGGAGGACAUCCAGAAGGCCCAGGUGAGCGGCCAGGGCGACA
GCCUGCACGAGCACAUCGCCAACCUGGCCGGCAGCCCCGCCAUCA
AGAAGGGCAUCCUGCAGACCGUGAAGGUGGUGGACGAGCUGGUGA
AGGUGAUGGGCCGGCACAAGCCCGAGAACAUCGUGAUCGAGAUGG
CCCGGGAGAACCAGACCACCCAGAAGGGCCAGAAGAACAGCCGGG
AGCGGAUGAAGCGGAUCGAGGAGGGCAUCAAGGAGCUGGGCAGCC
AGAUCCUGAAGGAGCACCCCGUGGAGAACACCCAGCUGCAGAACG
AGAAGCUGUACCUGUACUACCUGCAGAACGGCCGGGACAUGUACG
UGGACCAGGAGCUGGACAUCAACCGGCUGAGCGACUACGACGUGG
CCGCCAUCGUGCCCCAGAGCUUCCUGAAGGACGACAGCAUCGACA
ACAAGGUGCUGACCCGGAGCGACAAGGCCCGGGGCAAGAGCGACA
ACGUGCCCAGCGAGGAGGUGGUGAAGAAGAUGAAGAACUACUGGC
GGCAGCUGCUGAACGCCAAGCUGAUCACCCAGCGGAAGUUCGACA
ACCUGACCAAGGCCGAGCGGGGCGGCCUGAGCGAGCUGGACAAGG
CCGGCUUCAUCAAGCGGCAGCUGGUGGAGACCCGGCAGAUCACCA
AGCACGUGGCCCAGAUCCUGGACAGCCGGAUGAACACCAAGUACG
ACGAGAACGACAAGCUGAUCCGGGAGGUGAAGGUGAUCACCCUGA
AAUCCAAGCUGGUGAGCGACUUCCGGAAGGACUUCCAGUUCUACA
AGGUGCGGGAGAUCAACAACUACCACCACGCCCACGACGCCUACC
UGAACGCCGUGGUGGGCACCGCCCUGAUCAAGAAGUACCCCAAGC
UGGAGAGCGAGUUCGUGUACGGCGACUACAAGGUGUACGACGUGC
GGAAGAUGAUCGCCAAGAGCGAGCAGGAGAUCGGCAAGGCCACCG
CCAAGUACUUCUUCUACAGCAACAUCAUGAACUUCUUCAAGACCG
AGAUCACCCUGGCCAACGGCGAGAUCCGGAAGCGGCCCCUGAUCG
AGACCAACGGCGAGACCGGCGAGAUCGUGUGGGACAAGGGCCGGG
ACUUCGCCACCGUGCGGAAGGUGCUGAGCAUGCCCCAGGUGAACA
UCGUGAAGAAAACCGAGGUGCAGACCGGCGGCUUCAGCAAGGAGA
GCAUCCUGCCCAAGCGGAACAGCGACAAGCUGAUCGCCCGGAAGA
AGGACUGGGACCCCAAGAAGUACGGCGGCUUCGACAGCCCCACCG
UGGCCUACAGCGUGCUGGUGGUGGCCAAGGUGGAGAAGGGCAAGA
GCAAGAAGCUGAAAUCCGUGAAGGAGCUGCUGGGCAUCACCAUCA
UGGAGCGGAGCAGCUUCGAGAAGAACCCCAUCGACUUCCUGGAGG
CCAAGGGCUACAAGGAGGUGAAGAAGGACCUGAUCAUCAAGCUGC
CCAAGUACAGCCUGUUCGAGCUGGAGAACGGCCGGAAGCGGAUGC
UGGCCAGCGCCGGCGAGCUGCAGAAGGGCAACGAGCUGGCCCUGC
CCAGCAAGUACGUGAACUUCCUGUACCUGGCCAGCCACUACGAGA
AGCUGAAGGGCAGCCCCGAGGACAACGAGCAGAAGCAGCUGUUCG
UGGAGCAGCACAAGCACUACCUGGACGAGAUCAUCGAGCAGAUCA
GCGAGUUCAGCAAGCGGGUGAUCCUGGCCGACGCCAACCUGGACA
AGGUGCUGAGCGCCUACAACAAGCACCGGGACAAGCCCAUCCGGG
AGCAGGCCGAGAACAUCAUCCACCUGUUCACCCUGACCAACCUGG
GCGCCCCCGCCGCCUUCAAGUACUUCGACACCACCAUCGACCGGA
AGCGGUACACCAGCACCAAGGAGGUGCUGGACGCCACCCUGAUCC
ACCAGAGCAUCACCGGCCUGUACGAGACCCGGAUCGACCUGAGCC
AGCUGGGCGGCGACAGCGGCGGCAAGCGGCCCGCCGCCACCAAGA
AGGCCGGCCAGGCCAAGAAGAAGAAGUCGGGCGGGGGUGGCUCAG
AGGAGCCCGAGGAGCCCGCCGAUAGCGGACAAUCUCUGGUGCCCG
UCUACAUCUACAGCCCCGAAUAUGUGAGCAUGUGUGAUUCCCUCG
CCAAGAUCCCUAAGAGAGCCAGCAUGGUGCAUUCUCUGAUCGAGG
CCUACGCUCUGCAUAAGCAAAUGAGGAUCGUGAAGCCCAAGGUCG
CCAGCAUGGAAGAGAUGGCCACCUUUCACACCGAUGCCUACCUCC
AACAUCUCCAGAAGGUGUCCCAAGAGGGCGACGACGACCACCCCG
ACUCCAUUGAGUACGGACUGGGCUAUGAUUGCCCCGCCACCGAGG
GCAUCUUUGACUAUGCCGCCGCUAUCGGCGGAGCUACCAUCACAG
CCGCCCAGUGUCUGAUUGAUGGCAUGUGCAAGGUCGCCAUCAACU
GGUCCGGAGGCUGGCAUCAUGCCAAGAAGGAUGAGGCCUCCGGCU
UCUGUUAUCUGAAUGACGCCGUGCUGGGCAUUCUGAGACUGAGGA
GGAAAUUCGAGAGGAUUCUGUACGUGGAUCUGGAUCUGCAUCACG
GAGAUGGAGUCGAAGAUGCCUUCAGCUUCACCAGCAAGGUGAUGA
CAGUCUCUCUGCACAAGUUCUCCCCCGGCUUCUUUCCCGGAACCG
GCGACGUGUCCGACGUGGGACUGGGCAAGGGAAGGUACUACAGCG
UGAACGUGCCCAUUCAAGACGGCAUCCAAGACGAGAAGUACUACC
AGAUCUGCGAGUCCGUGCUCAAGGAGGUCUACCAAGCCUUCAAUC
CUAAGGCUGUCGUGCUCCAACUGGGAGCUGAUACCAUUGCUGGCG
AUCCCAUGUGCAGCUUCAAUAUGACACCCGUCGGAAUCGGCAAGU
GCCUCAAGUACAUCCUCCAGUGGCAGCUCGCCACCCUCAUUCUCG
GAGGAGGCGGAUACAAUCUGGCUAAUACCGCCAGAUGCUGGACCU
AUCUGACCGGCGUGAUUCUGGGCAAAACACUGAGCAGCGAAAUCC
CCGACCACGAGUUUUUCACCGCUUACGGCCCCGACUACGUGCUGG
AGAUCACCCCCAGCUGCAGACCCGAUAGAAACGAACCCCAUAGAA
UCCAGCAAAUUCUGAACUAUAUCAAGGGCAACCUCAAGCACGUCG
UGGGAGGUGGCGGAUCGGGAAAGCGGCCCGCCGCCACCAAGAAGG
CCGGUCAGGCCAAGAAGAAGAAGGGCAGCUACCCCUACGACGUGC
CCGACUACGCCUGAGCGGCCGCUUAAUUAAGCUGCCUUCUGCGGG
GCUUGCCUUCUGGCCAUGCCCUUCUUCUCUCCCUUGCACCUGUAC
CUCUUGGUCUUUGAAUAAAGCCUGAGUAGGAAGUCUAGAAAAAAA
AAAAAAAAAAAAAAAAAAAAAAAAAAAAAAAAAAAAAAAAAAAAA
AAAAAAAAAAAAAAAAAAAAAAAAAAAA

In some embodiments, a site-specific disrupting agent comprises the amino acid sequence of SEQ ID NOs: 214 or 78. In some embodiments, a site-specific disrupting agent described herein comprises an amino acid sequence of SEQ ID NO: 214 or 78 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

dCas9-HDAC8 Protein Sequence
(corresponding to MR 29439)
(SEQ ID NO: 214)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSGGKRPAATKK
AGQAKKKKSGGGGSEEPEEPADSGQSLVPVYIYSPEYVSMCDSLA
KIPKRASMVHSLIEAYALHKQMRIVKPKVASMEEMATFHTDAYLQ
HLQKVSQEGDDDHPDSIEYGLGYDCPATEGIFDYAAAIGGATITA
AQCLIDGMCKVAINWSGGWHHAKKDEASGFCYLNDAVLGILRLRR
KFERILYVDLDLHHGDGVEDAFSFTSKVMTVSLHKFSPGFFPGTG
DVSDVGLGKGRYYSVNVPIQDGIQDEKYYQICESVLKEVYQAFNP
KAVVLQLGADTIAGDPMCSFNMTPVGIGKCLKYILQWQLATLILG
GGGYNLANTARCWTYLTGVILGKTLSSEIPDHEFFTAYGPDYVLE
ITPSCRPDRNEPHRIQQILNYIKGNLKHVVGGGGSGKRPAATKKA
GQAKKKKGSYPYDVPDYA
dCas9-HDAC8 without HA tag
(SEQ ID NO: 78)
MAPKKKRKVGIHGVPAADKKYSIGLAIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVA
AIVPQSFLKDDSIDNKVLTRSDKARGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSGGKRPAATKK
AGQAKKKKSGGGGSEEPEEPADSGQSLVPVYIYSPEYVSMCDSLA
KIPKRASMVHSLIEAYALHKQMRIVKPKVASMEEMATFHTDAYLQ
HLQKVSQEGDDDHPDSIEYGLGYDCPATEGIFDYAAAIGGATITA
AQCLIDGMCKVAINWSGGWHHAKKDEASGFCYLNDAVLGILRLRR
KFERILYVDLDLHHGDGVEDAFSFTSKVMTVSLHKFSPGFFPGTG
DVSDVGLGKGRYYSVNVPIQDGIQDEKYYQICESVLKEVYQAFNP
KAVVLQLGADTIAGDPMCSFNMTPVGIGKCLKYILQWQLATLILG
GGGYNLANTARCWTYLTGVILGKTLSSEIPDHEFFTAYGPDYVLE
ITPSCRPDRNEPHRIQQILNYIKGNLKHVVGGGGSGKRPAATKKA
GQAKKKKGS

In some embodiments, a site-specific disrupting agent comprises a targeting moiety (e.g., comprising dCas9, e.g., an S. aureus dCas9), a first effector moiety comprising EZH2; e.g., an EZH2 domain, and a second effector moiety comprising HDAC8, e.g., an HDAC8 domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising EZH2, e.g., an EZH2 domain; and a second effector moiety comprising HDAC8, e.g., an HDAC8 domain. In some embodiments, the site-specific disrupting agent is encoded by the nucleic acid sequence of SEQ ID NO: 215 (e.g., mRNA encoding the site-specific disrupting agent). In some embodiments, a nucleic acid described herein comprises a nucleic acid sequence of SEQ ID NO: 215 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-HDAC8 mRNA (MR-29447)
(SEQ ID NO: 215)
AGGAAAUAAGAGAGAAAAGAAGAGUAAGAAGAAAUAUAAGAGCCA
CCAUGGCCCCCAAGAAGAAGCGGAAGGUGGGCGGCAGCGGCGGCA
GCGGCCAGACCGGCAAGAAGAGCGAGAAGGGCCCCGUGUGCUGGC
GGAAGCGGGUGAAGAGCGAGUACAUGCGGCUGCGGCAGCUGAAGC
GGUUCCGGCGGGCCGACGAGGUGAAGAGCAUGUUCAGCAGCAACC
GGCAGAAGAUCCUGGAGCGGACCGAGAUCCUGAACCAGGAGUGGA
AGCAGCGGCGAAUCCAGCCCGUGCACAUCCUGACCAGCGUGAGCA
GCCUGCGGGGCACCCGGGAGUGCAGCGUGACCAGCGACCUGGACU
UCCCCACCCAGGUGAUCCCCCUAAAGACCCUGAACGCCGUGGCCA
GCGUGCCCAUCAUGUACAGCUGGAGCCCCCUGCAGCAGAACUUCA
UGGUGGAGGACGAGACCGUGCUGCACAACAUCCCCUACAUGGGCG
ACGAGGUGCUGGACCAGGACGGCACCUUCAUCGAGGAGCUGAUCA
AGAACUACGACGGCAAGGUGCACGGCGACCGGGAGUGCGGCUUCA
UCAACGACGAGAUCUUCGUGGAGCUGGUGAACGCCCUGGGCCAGU
ACAACGACGACGACGACGACGACGACGGCGACGACCCCGAGGAGC
GGGAGGAGAAGCAGAAGGACCUGGAGGACCACCGGGACGACAAGG
AGAGCCGGCCCCCCCGGAAGUUCCCCAGCGACAAGAUCUUCGAGG
CCAUCAGCAGCAUGUUCCCCGACAAGGGCACCGCCGAGGAGCUGA
AGGAGAAGUACAAGGAGCUGACCGAGCAGCAGCUGCCCGGCGCCC
UGCCCCCCGAGUGCACCCCCAACAUCGACGGCCCCAACGCCAAGA
GCGUGCAGCGGGAGCAGAGCCUGCACAGCUUCCACACCCUGUUCU
GCCGGCGGUGCUUCAAGUACGACUGCUUCCUGCACCCCUUCCACG
CCACCCCCAACACCUACAAGCGGAAGAACACCGAGACCGCCCUGG
ACAACAAGCCCUGCGGCCCCCAGUGCUACCAGCACCUGGAGGGCG
CCAAGGAGUUCGCCGCCGCCCUGACCGCCGAGCGGAUCAAGACCC
CCCCCAAGCGGCCCGGCGGCCGGCGGCGGGGCCGGCUGCCCAACA
ACAGCAGCCGGCCCAGCACCCCCACCAUCAACGUGCUGGAGAGCA
AGGACACCGACAGCGACCGGGAGGCCGGCACCGAGACCGGCGGCG
AGAACAACGACAAGGAGGAGGAGGAGAAGAAGGACGAGACCAGCA
GCAGCAGCGAGGCCAACAGCCGGUGCCAGACCCCCAUCAAGAUGA
AGCCCAACAUCGAGCCCCCCGAGAACGUGGAGUGGAGCGGCGCCG
AGGCCAGCAUGUUCCGGGUGCUGAUCGGCACCUACUACGACAACU
UCUGCGCCAUCGCCCGGCUGAUCGGCACCAAGACCUGCCGGCAGG
UGUACGAGUUCCGGGUGAAGGAGAGCAGCAUCAUCGCCCCCGCCC
CCGCCGAGGACGUGGACACCCCCCCCCGGAAGAAGAAGCGGAAGC
ACCGGCUGUGGGCCGCCCACUGCCGGAAGAUCCAGCUGAAGAAGG
ACGGCAGCAGCAACCACGUGUACAACUACCAGCCCUGCGACCACC
CCCGGCAGCCCUGCGACAGCAGCUGCCCCUGCGUGAUCGCCCAGA
ACUUCUGCGAGAAGUUCUGCCAGUGCAGCAGCGAGUGCCAGAACC
GGUUCCCCGGCUGCCGGUGCAAGGCCCAGUGCAACACCAAGCAGU
GCCCCUGCUACCUGGCCGUGCGGGAGUGCGACCCCGACCUGUGCC
UGACCUGCGGCGCCGCCGACCACUGGGACAGCAAGAACGUGAGCU
GCAAGAACUGCAGCAUCCAGCGGGGCAGCAAGAAGCACCUGCUGC
UGGCCCCCAGCGACGUGGCCGGCUGGGGCAUCUUCAUCAAGGACC
CCGUGCAGAAGAACGAGUUCAUCAGCGAGUACUGCGGCGAGAUCA
UCAGCCAGGACGAGGCCGACCGGCGGGGCAAGGUGUACGACAAGU
ACAUGUGCAGCUUCCUGUUCAACCUGAACAACGACUUCGUGGUGG
ACGCCACCCGGAAGGGCAACAAGAUCCGGUUCGCCAACCACAGCG
UGAACCCCAACUGCUACGCCAAGGUGAUGAUGGUGAACGGCGACC
ACCGGAUCGGCAUCUUCGCCAAGCGGGCCAUCCAGACCGGCGAGG
AGCUGUUCUUCGACUACCGGUACAGCCAGGCCGACGCCCUGAAGU
ACGUGGGCAUCGAGCGGGAGAUGGAGAUCCCCAGCACCGGCGGCA
GCGGCGGCAGCGGCGGCAGCGGCGGCAGCGGCGGCAGCGGCCGAC
CCGACAAGAAGUACAGCAUCGGCCUGGCCAUCGGCACCAACAGCG
UGGGCUGGGCCGUGAUCACCGACGAGUACAAGGUGCCCAGCAAGA
AGUUCAAGGUGCUGGGCAACACCGACCGGCACAGCAUCAAGAAGA
ACCUGAUCGGCGCCCUGCUGUUCGACAGCGGCGAGACCGCCGAGG
CCACCCGGCUGAAGCGGACCGCCCGGCGGCGGUACACCCGGCGGA
AGAACCGGAUCUGCUACCUGCAGGAGAUCUUCAGCAACGAGAUGG
CCAAGGUGGACGACAGCUUCUUCCACCGGCUGGAGGAGAGCUUCC
UGGUGGAGGAGGACAAGAAGCACGAGCGGCACCCCAUCUUCGGCA
ACAUCGUGGACGAGGUGGCCUACCACGAGAAGUACCCCACCAUCU
ACCACCUGCGGAAGAAGCUGGUGGACAGCACCGACAAGGCCGACC
UGCGGCUGAUCUACCUGGCCCUGGCCCACAUGAUCAAGUUCCGGG
GCCACUUCCUGAUCGAGGGCGACCUGAACCCCGACAACAGCGACG
UGGACAAGCUGUUCAUCCAGCUGGUGCAGACCUACAACCAGCUGU
UCGAGGAGAACCCCAUCAACGCCAGCGGCGUGGACGCCAAGGCCA
UCCUGAGCGCCCGGCUGAGCAAGAGCCGGCGGCUGGAGAACCUGA
UCGCCCAGCUGCCCGGCGAGAAGAAGAACGGCCUGUUCGGCAACC
UGAUCGCCCUGAGCCUGGGCCUGACCCCCAACUUCAAGAGCAACU
UCGACCUGGCCGAGGACGCCAAGCUGCAGCUGAGCAAGGACACCU
ACGACGACGACCUGGACAACCUGCUGGCCCAGAUCGGCGACCAGU
ACGCCGACCUGUUCCUGGCCGCCAAGAACCUGAGCGACGCCAUCC
UGCUGAGCGACAUCCUGCGGGUGAACACCGAGAUCACCAAGGCCC
CCCUGAGCGCCAGCAUGAUCAAGCGGUACGACGAGCACCACCAGG
ACCUGACCCUGCUGAAGGCCCUGGUGCGGCAGCAGCUGCCCGAGA
AGUACAAGGAGAUCUUCUUCGACCAGAGCAAGAACGGCUACGCCG
GCUACAUCGACGGCGGCGCCAGCCAGGAGGAGUUCUACAAGUUCA
UCAAGCCCAUCCUGGAGAAGAUGGACGGCACCGAGGAGCUGCUGG
UGAAGCUGAACCGGGAGGACCUGCUGCGGAAGCAGCGGACCUUCG
ACAACGGCAGCAUCCCCCACCAGAUCCACCUGGGCGAGCUGCACG
CCAUCCUGCGGCGGCAGGAGGACUUCUACCCCUUCCUGAAGGACA
ACCGGGAGAAGAUCGAGAAGAUCCUGACCUUCCGGAUCCCCUACU
ACGUGGGCCCCCUGGCCCGGGGCAACAGCCGGUUCGCCUGGAUGA
CCCGGAAAUCCGAGGAGACCAUCACCCCCUGGAACUUCGAGGAGG
UGGUGGACAAGGGCGCCAGCGCCCAGAGCUUCAUCGAGCGGAUGA
CCAACUUCGACAAGAACCUGCCCAACGAGAAGGUGCUGCCCAAGC
ACAGCCUGCUGUACGAGUACUUCACCGUGUACAACGAGCUGACCA
AGGUGAAGUACGUGACCGAGGGCAUGCGGAAGCCCGCCUUCCUGA
GCGGCGAGCAGAAGAAGGCCAUCGUGGACCUGCUGUUCAAGACCA
ACCGGAAGGUGACCGUGAAGCAGCUGAAGGAGGACUACUUCAAGA
AGAUCGAGUGCUUCGACAGCGUGGAGAUCAGCGGCGUGGAGGACC
GGUUCAACGCCAGCCUGGGCACCUACCACGACCUGCUGAAGAUCA
UCAAGGACAAGGACUUCCUGGACAACGAGGAGAACGAGGACAUCC
UGGAGGACAUCGUGCUGACCCUGACCCUGUUCGAGGACCGGGAGA
UGAUCGAGGAGCGGCUGAAAACCUACGCCCACCUGUUCGACGACA
AGGUGAUGAAGCAGCUGAAGCGGCGGCGGUACACCGGCUGGGGCC
GGCUGAGCCGGAAGCUGAUCAACGGCAUCCGGGACAAGCAGAGCG
GCAAGACCAUCCUGGACUUCCUGAAAUCCGACGGCUUCGCCAACC
GGAACUUCAUGCAGCUGAUCCACGACGACAGCCUGACCUUCAAGG
AGGACAUCCAGAAGGCCCAGGUGAGCGGCCAGGGCGACAGCCUGC
ACGAGCACAUCGCCAACCUGGCCGGCAGCCCCGCCAUCAAGAAGG
GCAUCCUGCAGACCGUGAAGGUGGUGGACGAGCUGGUGAAGGUGA
UGGGCCGGCACAAGCCCGAGAACAUCGUGAUCGAGAUGGCCCGGG
AGAACCAGACCACCCAGAAGGGCCAGAAGAACAGCCGGGAGCGGA
UGAAGCGGAUCGAGGAGGGCAUCAAGGAGCUGGGCAGCCAGAUCC
UGAAGGAGCACCCCGUGGAGAACACCCAGCUGCAGAACGAGAAGC
UGUACCUGUACUACCUGCAGAACGGCCGGGACAUGUACGUGGACC
AGGAGCUGGACAUCAACCGGCUGAGCGACUACGACGUGGCCGCCA
UCGUGCCCCAGAGCUUCCUGAAGGACGACAGCAUCGACAACAAGG
UGCUGACCCGGAGCGACAAGGCCCGGGGCAAGAGCGACAACGUGC
CCAGCGAGGAGGUGGUGAAGAAGAUGAAGAACUACUGGCGGCAGC
UGCUGAACGCCAAGCUGAUCACCCAGCGGAAGUUCGACAACCUGA
CCAAGGCCGAGCGGGGCGGCCUGAGCGAGCUGGACAAGGCCGGCU
UCAUCAAGCGGCAGCUGGUGGAGACCCGGCAGAUCACCAAGCACG
UGGCCCAGAUCCUGGACAGCCGGAUGAACACCAAGUACGACGAGA
ACGACAAGCUGAUCCGGGAGGUGAAGGUGAUCACCCUGAAAUCCA
AGCUGGUGAGCGACUUCCGGAAGGACUUCCAGUUCUACAAGGUGC
GGGAGAUCAACAACUACCACCACGCCCACGACGCCUACCUGAACG
CCGUGGUGGGCACCGCCCUGAUCAAGAAGUACCCCAAGCUGGAGA
GCGAGUUCGUGUACGGCGACUACAAGGUGUACGACGUGCGGAAGA
UGAUCGCCAAGAGCGAGCAGGAGAUCGGCAAGGCCACCGCCAAGU
ACUUCUUCUACAGCAACAUCAUGAACUUCUUCAAGACCGAGAUCA
CCCUGGCCAACGGCGAGAUCCGGAAGCGGCCCCUGAUCGAGACCA
ACGGCGAGACCGGCGAGAUCGUGUGGGACAAGGGCCGGGACUUCG
CCACCGUGCGGAAGGUGCUGAGCAUGCCCCAGGUGAACAUCGUGA
AGAAAACCGAGGUGCAGACCGGCGGCUUCAGCAAGGAGAGCAUCC
UGCCCAAGCGGAACAGCGACAAGCUGAUCGCCCGGAAGAAGGACU
GGGACCCCAAGAAGUACGGCGGCUUCGACAGCCCCACCGUGGCCU
ACAGCGUGCUGGUGGUGGCCAAGGUGGAGAAGGGCAAGAGCAAGA
AGCUGAAAUCCGUGAAGGAGCUGCUGGGCAUCACCAUCAUGGAGC
GGAGCAGCUUCGAGAAGAACCCCAUCGACUUCCUGGAGGCCAAGG
GCUACAAGGAGGUGAAGAAGGACCUGAUCAUCAAGCUGCCCAAGU
ACAGCCUGUUCGAGCUGGAGAACGGCCGGAAGCGGAUGCUGGCCA
GCGCCGGCGAGCUGCAGAAGGGCAACGAGCUGGCCCUGCCCAGCA
AGUACGUGAACUUCCUGUACCUGGCCAGCCACUACGAGAAGCUGA
AGGGCAGCCCCGAGGACAACGAGCAGAAGCAGCUGUUCGUGGAGC
AGCACAAGCACUACCUGGACGAGAUCAUCGAGCAGAUCAGCGAGU
UCAGCAAGCGGGUGAUCCUGGCCGACGCCAACCUGGACAAGGUGC
UGAGCGCCUACAACAAGCACCGGGACAAGCCCAUCCGGGAGCAGG
CCGAGAACAUCAUCCACCUGUUCACCCUGACCAACCUGGGCGCCC
CCGCCGCCUUCAAGUACUUCGACACCACCAUCGACCGGAAGCGGU
ACACCAGCACCAAGGAGGUGCUGGACGCCACCCUGAUCCACCAGA
GCAUCACCGGCCUGUACGAGACCCGGAUCGACCUGAGCCAGCUGG
GCGGCGACAGCGGCGGCAAGCGGCCCGCCGCCACCAAGAAGGCCG
GCCAGGCCAAGAAGAAGAAGUCGGGCGGGGGUGGCUCAGAGGAGC
CCGAGGAGCCCGCCGAUAGCGGACAAUCUCUGGUGCCCGUCUACA
UCUACAGCCCCGAAUAUGUGAGCAUGUGUGAUUCCCUCGCCAAGA
UCCCUAAGAGAGCCAGCAUGGUGCAUUCUCUGAUCGAGGCCUACG
CUCUGCAUAAGCAAAUGAGGAUCGUGAAGCCCAAGGUCGCCAGCA
UGGAAGAGAUGGCCACCUUUCACACCGAUGCCUACCUCCAACAUC
UCCAGAAGGUGUCCCAAGAGGGCGACGACGACCACCCCGACUCCA
UUGAGUACGGACUGGGCUAUGAUUGCCCCGCCACCGAGGGCAUCU
UUGACUAUGCCGCCGCUAUCGGCGGAGCUACCAUCACAGCCGCCC
AGUGUCUGAUUGAUGGCAUGUGCAAGGUCGCCAUCAACUGGUCCG
GAGGCUGGCAUCAUGCCAAGAAGGAUGAGGCCUCCGGCUUCUGUU
AUCUGAAUGACGCCGUGCUGGGCAUUCUGAGACUGAGGAGGAAAU
UCGAGAGGAUUCUGUACGUGGAUCUGGAUCUGCAUCACGGAGAUG
GAGUCGAAGAUGCCUUCAGCUUCACCAGCAAGGUGAUGACAGUCU
CUCUGCACAAGUUCUCCCCCGGCUUCUUUCCCGGAACCGGCGACG
UGUCCGACGUGGGACUGGGCAAGGGAAGGUACUACAGCGUGAACG
UGCCCAUUCAAGACGGCAUCCAAGACGAGAAGUACUACCAGAUCU
GCGAGUCCGUGCUCAAGGAGGUCUACCAAGCCUUCAAUCCUAAGG
CUGUCGUGCUCCAACUGGGAGCUGAUACCAUUGCUGGCGAUCCCA
UGUGCAGCUUCAAUAUGACACCCGUCGGAAUCGGCAAGUGCCUCA
AGUACAUCCUCCAGUGGCAGCUCGCCACCCUCAUUCUCGGAGGAG
GCGGAUACAAUCUGGCUAAUACCGCCAGAUGCUGGACCUAUCUGA
CCGGCGUGAUUCUGGGCAAAACACUGAGCAGCGAAAUCCCCGACC
ACGAGUUUUUCACCGCUUACGGCCCCGACUACGUGCUGGAGAUCA
CCCCCAGCUGCAGACCCGAUAGAAACGAACCCCAUAGAAUCCAGC
AAAUUCUGAACUAUAUCAAGGGCAACCUCAAGCACGUCGUGGGAG
GUGGCGGAUCGGGAAAGCGGCCCGCCGCCACCAAGAAGGCCGGUC
AGGCCAAGAAGAAGAAGGGCAGCUACCCCUACGACGUGCCCGACU
ACGCCUGAGCGGCCGCUUAAUUAAGCUGCCUUCUGCGGGGCUUGC
CUUCUGGCCAUGCCCUUCUUCUCUCCCUUGCACCUGUACCUCUUG
GUCUUUGAAUAAAGCCUGAGUAGGAAGUCUAGAAAAAAAAAAAAA
AAAAAAAAAAAAAAAAAAAAAAAAAAAAAAAAAAAAAAAAAAAAA
AAAAAAAAAAAAAAAAAAAAAA

In some embodiments, a site-specific disrupting agent comprises the amino acid sequence of SEQ ID NOs: 216 or 79. In some embodiments, a site-specific disrupting agent described herein comprises an amino acid sequence of SEQ ID NO: 216 or 79 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-HDAC8 Protein Sequence
(corresponding to MR-29447)
(SEQ ID NO: 216)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALYNKHRDKPIREQAENIIH
LFTLTNLGAPAAFKYFDTTIDRKRYTSTKEVLDATLIHQSITGLY
ETRIDLSQLGGDSGGKRPAATKKAGQAKKKKSGGGGSEEPEEPAD
SGQSLVPVYIYSPEYVSMCDSLAKIPKRASMVHSLIEAYALHKQM
RIVKPKVASMEEMATFHTDAYLQHLQKVSQEGDDDHPDSIEYGLG
YDCPATEGIFDYAAAIGGATITAAQCLIDGMCKVAINWSGGWHHA
KKDEASGFCYLNDAVLGILRLRRKFERILYVDLDLHHGDGVEDAF
SFTSKVMTVSLHKFSPGFFPGTGDVSDVGLGKGRYYSVNVPIQDG
IQDEKYYQICESVLKEVYQAFNPKAVVLQLGADTIAGDPMCSFNM
TPVGIGKCLKYILQWQLATLILGGGGYNLANTARCWTYLTGVILG
KTLSSEIPDHEFFTAYGPDYVLEITPSCRPDRNEPHRIQQILNYI
KGNLKHVVGGGGSGKRPAATKKAGQAKKKKGSYPYDVPDYA
EZH2-dCas9-HDAC8 Protein Sequence
(corresponding to MR-29447)
(SEQ ID NO: 79)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALPSKYVNFLYLASHYEKLK
GSPEDNEQKQLFVEQHKHYLDEIIEQISEFSKRVILADANLDKVL
SAYNKHRDKPIREQAENIIHLFTLTNLGAPAAFKYFDTTIDRKRY
TSTKEVLDATLIHQSITGLYETRIDLSQLGGDSGGKRPAATKKAG
QAKKKKSGGGGSEEPEEPADSGQSLVPVYIYSPEYVSMCDSLAKI
PKRASMVHSLIEAYALHKQMRIVKPKVASMEEMATFHTDAYLQHL
QKVSQEGDDDHPDSIEYGLGYDCPATEGIFDYAAAIGGATITAAQ
CLIDGMCKVAINWSGGWHHAKKDEASGFCYLNDAVLGILRLRRKF
ERILYVDLDLHHGDGVEDAFSFTSKVMTVSLHKFSPGFFPGTGDV
SDVGLGKGRYYSVNVPIQDGIQDEKYYQICESVLKEVYQAFNPKA
VVLQLGADTIAGDPMCSFNMTPVGIGKCLKYILQWQLATLILGGG
GYNLANTARCWTYLTGVILGKTLSSEIPDHEFFTAYGPDYVLEIT
PSCRPDRNEPHRIQQILNYIKGNLKHVVGGGGSGKRPAATKKAGQ
AKKKKGS

In some embodiments, a site-specific disrupting agent comprises a targeting moiety (e.g., comprising dCas9, e.g., an S. aureus dCas9), a first effector moiety comprising G9A; e.g., a G9A domain, and a second effector moiety comprising EZH2, e.g., an EZH2 domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising G9A, e.g., a G9A domain; and a second effector moiety comprising EZH2, e.g., a EZH2 domain. In some embodiments, the site-specific disrupting agent is encoded by the nucleic acid sequence of SEQ ID NO: 69 (e.g., mRNA encoding the site-specific disrupting agent). In some embodiments, a nucleic acid described herein comprises a nucleic acid sequence of SEQ ID NO: 69 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-EZH2 (MR-29441) mRNA sequence
(SEQ ID NO. 69)
AGGAAATAAGAGAGAAAAGAAGAGTAAGAAGAAATATAAGAGCCA
CCATGGCCCCCAAGAAGAAGCGGAAGGTGGGCATCCACGGCGTGC
CCGCCGCCTCGGGCGGGGGTGGCTCAGGAAGCGCCGCTATCGCCG
AAGTGCTGCTGAATGCCAGATGCGATCTGCATGCCGTGAACTACC
ACGGCGACACCCCTCTGCATATCGCCGCTAGAGAGAGCTACCATG
ACTGTGTGCTGCTGTTTCTGAGCAGAGGCGCCAATCCCGAGCTCA
GAAACAAAGAGGGCGACACCGCTTGGGATCTGACACCCGAGAGAT
CCGACGTGTGGTTCGCTCTGCAACTGAATAGAAAACTGAGACTGG
GCGTCGGCAATAGGGCCATTAGAACCGAGAAGATCATCTGTAGGG
ACGTGGCTAGGGGCTACGAGAACGTGCCCATCCCTTGTGTGAACG
GAGTGGATGGAGAGCCTTGCCCCGAGGATTACAAATACATCAGCG
AGAACTGCGAAACCTCCACCATGAATATCGATAGAAACATTACAC
ACCTCCAGCACTGTACATGCGTGGACGATTGCAGCAGCAGCAACT
GTCTGTGCGGCCAACTGAGCATCAGATGCTGGTACGACAAGGATG
GCAGACTGCTGCAAGAGTTCAACAAGATCGAACCCCCTCTGATCT
TCGAGTGTAACCAAGCTTGCAGCTGTTGGAGGAACTGCAAGAATA
GGGTCGTGCAGTCCGGAATCAAGGTGAGACTGCAGCTGTATAGAA
CAGCTAAGATGGGATGGGGAGTCAGAGCTCTGCAGACCATCCCCC
AAGGCACATTCATCTGTGAGTACGTCGGCGAACTCATCAGCGACG
CTGAGGCCGATGTGAGGGAGGACGACAGCTATCTCTTCGACCTCG
ACAACAAGGACGGCGAGGTGTACTGCATCGACGCTAGATATTACG
GCAACATCAGCAGATTCATCAACCACCTCTGCGACCCCAATATCA
TCCCCGTGAGAGTGTTCATGCTCCATCAAGATCTGAGATTCCCTA
GGATCGCCTTCTTCAGCTCTAGAGACATTAGAACCGGCGAGGAGC
TGGGATTCGACTACGGCGACAGGTTCTGGGACATCAAGAGCAAGT
ACTTCACATGCCAATGCGGCAGCGAGAAATGCAAGCATAGCGCCG
AGGCCATTGCTCTGGAGCAGTCTAGACTGGCTAGGCTGGACCCTC
ACCCCGAGCTGCTGCCCGAACTGGGATCTCTGCCTCCCGTGAATA
CCGGAGGTGGCGGATCGGGAGACAAGAAGTACAGCATCGGCCTGG
CCATCGGCACCAACAGCGTGGGCTGGGCCGTGATCACCGACGAGT
ACAAGGTGCCCAGCAAGAAGTTCAAGGTGCTGGGCAACACCGACC
GGCACAGCATCAAGAAGAACCTGATCGGCGCCCTGCTGTTCGACA
GCGGCGAGACCGCCGAGGCCACCCGGCTGAAGCGGACCGCCCGGC
GGCGGTACACCCGGCGGAAGAACCGGATCTGCTACCTGCAGGAGA
TCTTCAGCAACGAGATGGCCAAGGTGGACGACAGCTTCTTCCACC
GGCTGGAGGAGAGCTTCCTGGTGGAGGAGGACAAGAAGCACGAGC
GGCACCCCATCTTCGGCAACATCGTGGACGAGGTGGCCTACCACG
AGAAGTACCCCACCATCTACCACCTGCGGAAGAAGCTGGTGGACA
GCACCGACAAGGCCGACCTGCGGCTGATCTACCTGGCCCTGGCCC
ACATGATCAAGTTCCGGGGCCACTTCCTGATCGAGGGCGACCTGA
ACCCCGACAACAGCGACGTGGACAAGCTGTTCATCCAGCTGGTGC
AGACCTACAACCAGCTGTTCGAGGAGAACCCCATCAACGCCAGCG
GCGTGGACGCCAAGGCCATCCTGAGCGCCCGGCTGAGCAAGAGCC
GGCGGCTGGAGAACCTGATCGCCCAGCTGCCCGGCGAGAAGAAGA
ACGGCCTGTTCGGCAACCTGATCGCCCTGAGCCTGGGCCTGACCC
CCAACTTCAAGAGCAACTTCGACCTGGCCGAGGACGCCAAGCTGC
AGCTGAGCAAGGACACCTACGACGACGACCTGGACAACCTGCTGG
CCCAGATCGGCGACCAGTACGCCGACCTGTTCCTGGCCGCCAAGA
ACCTGAGCGACGCCATCCTGCTGAGCGACATCCTGCGGGTGAACA
CCGAGATCACCAAGGCCCCCCTGAGCGCCAGCATGATCAAGCGGT
ACGACGAGCACCACCAGGACCTGACCCTGCTGAAGGCCCTGGTGC
GGCAGCAGCTGCCCGAGAAGTACAAGGAGATCTTCTTCGACCAGA
GCAAGAACGGCTACGCCGGCTACATCGACGGCGGCGCCAGCCAGG
AGGAGTTCTACAAGTTCATCAAGCCCATCCTGGAGAAGATGGACG
GCACCGAGGAGCTGCTGGTGAAGCTGAACCGGGAGGACCTGCTGC
GGAAGCAGCGGACCTTCGACAACGGCAGCATCCCCCACCAGATCC
ACCTGGGCGAGCTGCACGCCATCCTGCGGCGGCAGGAGGACTTCT
ACCCCTTCCTGAAGGACAACCGGGAGAAGATCGAGAAGATCCTGA
CCTTCCGGATCCCCTACTACGTGGGCCCCCTGGCCCGGGGCAACA
GCCGGTTCGCCTGGATGACCCGGAAATCCGAGGAGACCATCACCC
CCTGGAACTTCGAGGAGGTGGTGGACAAGGGCGCCAGCGCCCAGA
GCTTCATCGAGCGGATGACCAACTTCGACAAGAACCTGCCCAACG
AGAAGGTGCTGCCCAAGCACAGCCTGCTGTACGAGTACTTCACCG
TGTACAACGAGCTGACCAAGGTGAAGTACGTGACCGAGGGCATGC
GGAAGCCCGCCTTCCTGAGCGGCGAGCAGAAGAAGGCCATCGTGG
ACCTGCTGTTCAAGACCAACCGGAAGGTGACCGTGAAGCAGCTGA
AGGAGGACTACTTCAAGAAGATCGAGTGCTTCGACAGCGTGGAGA
TCAGCGGCGTGGAGGACCGGTTCAACGCCAGCCTGGGCACCTACC
ACGACCTGCTGAAGATCATCAAGGACAAGGACTTCCTGGACAACG
AGGAGAACGAGGACATCCTGGAGGACATCGTGCTGACCCTGACCC
TGTTCGAGGACCGGGAGATGATCGAGGAGCGGCTGAAAACCTACG
CCCACCTGTTCGACGACAAGGTGATGAAGCAGCTGAAGCGGCGGC
GGTACACCGGCTGGGGCCGGCTGAGCCGGAAGCTGATCAACGGCA
TCCGGGACAAGCAGAGCGGCAAGACCATCCTGGACTTCCTGAAAT
CCGACGGCTTCGCCAACCGGAACTTCATGCAGCTGATCCACGACG
ACAGCCTGACCTTCAAGGAGGACATCCAGAAGGCCCAGGTGAGCG
GCCAGGGCGACAGCCTGCACGAGCACATCGCCAACCTGGCCGGCA
GCCCCGCCATCAAGAAGGGCATCCTGCAGACCGTGAAGGTGGTGG
ACGAGCTGGTGAAGGTGATGGGCCGGCACAAGCCCGAGAACATCG
TGATCGAGATGGCCCGGGAGAACCAGACCACCCAGAAGGGCCAGA
AGAACAGCCGGGAGCGGATGAAGCGGATCGAGGAGGGCATCAAGG
AGCTGGGCAGCCAGATCCTGAAGGAGCACCCCGTGGAGAACACCC
AGCTGCAGAACGAGAAGCTGTACCTGTACTACCTGCAGAACGGCC
GGGACATGTACGTGGACCAGGAGCTGGACATCAACCGGCTGAGCG
ACTACGACGTGGCCGCCATCGTGCCCCAGAGCTTCCTGAAGGACG
ACAGCATCGACAACAAGGTGCTGACCCGGAGCGACAAGGCCCGGG
GCAAGAGCGACAACGTGCCCAGCGAGGAGGTGGTGAAGAAGATGA
AGAACTACTGGCGGCAGCTGCTGAACGCCAAGCTGATCACCCAGC
GGAAGTTCGACAACCTGACCAAGGCCGAGCGGGGCGGCCTGAGCG
AGCTGGACAAGGCCGGCTTCATCAAGCGGCAGCTGGTGGAGACCC
GGCAGATCACCAAGCACGTGGCCCAGATCCTGGACAGCCGGATGA
ACACCAAGTACGACGAGAACGACAAGCTGATCCGGGAGGTGAAGG
TGATCACCCTGAAATCCAAGCTGGTGAGCGACTTCCGGAAGGACT
TCCAGTTCTACAAGGTGCGGGAGATCAACAACTACCACCACGCCC
ACGACGCCTACCTGAACGCCGTGGTGGGCACCGCCCTGATCAAGA
AGTACCCCAAGCTGGAGAGCGAGTTCGTGTACGGCGACTACAAGG
TGTACGACGTGCGGAAGATGATCGCCAAGAGCGAGCAGGAGATCG
GCAAGGCCACCGCCAAGTACTTCTTCTACAGCAACATCATGAACT
TCTTCAAGACCGAGATCACCCTGGCCAACGGCGAGATCCGGAAGC
GGCCCCTGATCGAGACCAACGGCGAGACCGGCGAGATCGTGTGGG
ACAAGGGCCGGGACTTCGCCACCGTGCGGAAGGTGCTGAGCATGC
CCCAGGTGAACATCGTGAAGAAAACCGAGGTGCAGACCGGCGGCT
TCAGCAAGGAGAGCATCCTGCCCAAGCGGAACAGCGACAAGCTGA
TCGCCCGGAAGAAGGACTGGGACCCCAAGAAGTACGGCGGCTTCG
ACAGCCCCACCGTGGCCTACAGCGTGCTGGTGGTGGCCAAGGTGG
AGAAGGGCAAGAGCAAGAAGCTGAAATCCGTGAAGGAGCTGCTGG
GCATCACCATCATGGAGCGGAGCAGCTTCGAGAAGAACCCCATCG
ACTTCCTGGAGGCCAAGGGCTACAAGGAGGTGAAGAAGGACCTGA
TCATCAAGCTGCCCAAGTACAGCCTGTTCGAGCTGGAGAACGGCC
GGAAGCGGATGCTGGCCAGCGCCGGCGAGCTGCAGAAGGGCAACG
AGCTGGCCCTGCCCAGCAAGTACGTGAACTTCCTGTACCTGGCCA
GCCACTACGAGAAGCTGAAGGGCAGCCCCGAGGACAACGAGCAGA
AGCAGCTGTTCGTGGAGCAGCACAAGCACTACCTGGACGAGATCA
TCGAGCAGATCAGCGAGTTCAGCAAGCGGGTGATCCTGGCCGACG
CCAACCTGGACAAGGTGCTGAGCGCCTACAACAAGCACCGGGACA
AGCCCATCCGGGAGCAGGCCGAGAACATCATCCACCTGTTCACCC
TGACCAACCTGGGCGCCCCCGCCGCCTTCAAGTACTTCGACACCA
CCATCGACCGGAAGCGGTACACCAGCACCAAGGAGGTGCTGGACG
CCACCCTGATCCACCAGAGCATCACCGGCCTGTACGAGACCCGGA
TCGACCTGAGCCAGCTGGGCGGCGACAGCGGCGGCAAGCGGCCCG
CCGCCACCAAGAAGGCCGGCCAGGCCAAGAAGAAGAAGTCGGGCG
GGGGTGGCTCAGGACAGACCGGCAAAAAGTCCGAAAAGGGCCCCG
TGTGCTGGAGGAAGAGGGTCAAGAGCGAGTACATGAGGCTGAGAC
AGCTCAAGAGATTTAGGAGAGCCGATGAGGTGAAGTCCATGTTCT
CCAGCAACAGACAAAAGATTCTGGAGAGGACCGAGATCCTCAACC
AAGAGTGGAAGCAGAGAAGAATCCAGCCCGTGCACATTCTGACCT
CCGTGAGCTCTCTGAGGGGCACAAGAGAATGCTCCGTCACCAGCG
ATCTGGACTTCCCCACACAAGTGATCCCCCTCAAGACACTGAACG
CTGTGGCCAGCGTGCCCATCATGTATAGCTGGTCCCCTCTGCAAC
AGAACTTCATGGTGGAGGACGAGACAGTGCTGCACAATATCCCCT
ACATGGGAGATGAGGTGCTGGACCAAGACGGCACCTTTATTGAGG
AGCTGATTAAAAACTACGATGGCAAGGTGCACGGCGATAGGGAGT
GTGGCTTCATCAACGACGAGATCTTCGTCGAGCTGGTGAATGCTC
TGGGCCAGTATAATGACGATGATGACGACGATGACGGCGACGACC
CCGAAGAGAGAGAGGAGAAGCAAAAGGATCTGGAGGACCATAGGG
ACGACAAAGAGTCTAGACCTCCTAGAAAGTTCCCCTCCGACAAGA
TCTTCGAAGCCATCTCCTCCATGTTCCCCGACAAGGGCACAGCCG
AGGAACTGAAGGAGAAGTATAAGGAACTCACAGAGCAACAGCTGC
CCGGAGCTCTGCCTCCCGAGTGCACCCCTAACATCGACGGCCCCA
ACGCCAAGAGCGTGCAGAGGGAGCAATCCCTCCACAGCTTCCATA
CCCTCTTCTGCAGAAGATGCTTTAAATACGATTGCTTTCTCCATC
CTTTCCACGCCACACCCAACACCTACAAGAGGAAGAACACCGAAA
CCGCTCTGGACAATAAACCTTGCGGACCCCAGTGCTACCAGCATC
TGGAAGGAGCCAAGGAATTTGCCGCTGCTCTGACAGCCGAGAGAA
TTAAAACCCCTCCCAAAAGACCCGGCGGCAGAAGGAGGGGCAGAC
TGCCTAATAACAGCAGCAGACCCAGCACCCCTACCATTAACGTGC
TGGAATCCAAGGACACCGACAGCGATAGAGAGGCCGGCACAGAAA
CCGGCGGAGAGAACAACGACAAGGAGGAGGAGGAGAAGAAAGACG
AGACATCCTCCAGCAGCGAGGCTAATAGCAGATGCCAGACCCCTA
TCAAGATGAAACCTAATATCGAGCCCCCCGAGAATGTGGAGTGGA
GCGGCGCTGAGGCCTCCATGTTTAGAGTGCTGATCGGAACCTACT
ACGACAACTTCTGCGCTATCGCTAGACTGATTGGCACCAAGACAT
GCAGACAAGTGTACGAGTTCAGAGTCAAGGAGAGCTCCATTATCG
CCCCCGCCCCCGCCGAAGATGTGGACACCCCCCCCAGAAAGAAGA
AAAGGAAGCATAGACTGTGGGCCGCCCACTGTAGAAAGATCCAGC
TCAAAAAGGACGGCAGCAGCAACCACGTGTACAACTATCAGCCTT
GTGACCACCCCAGACAACCTTGTGATTCCAGCTGCCCTTGCGTGA
TCGCCCAGAACTTCTGCGAGAAGTTCTGTCAGTGCAGCAGCGAGT
GCCAAAATAGATTTCCCGGATGTAGGTGCAAAGCCCAGTGCAATA
CCAAGCAGTGCCCTTGCTATCTGGCCGTGAGAGAGTGCGATCCCG
ATCTGTGTCTGACATGTGGAGCTGCCGACCATTGGGACAGCAAGA
ATGTGAGCTGCAAGAACTGCAGCATCCAAAGGGGAAGCAAAAAAC
ATCTGCTGCTCGCCCCTTCCGATGTGGCCGGATGGGGAATCTTTA
TCAAGGACCCCGTCCAGAAAAACGAGTTCATTTCCGAGTATTGCG
GCGAGATCATCAGCCAAGACGAAGCTGATAGAAGAGGCAAAGTGT
ATGACAAATACATGTGCTCCTTCCTCTTCAACCTCAATAATGATT
TCGTGGTGGACGCCACAAGGAAGGGCAACAAGATTAGATTCGCCA
ACCACAGCGTCAATCCTAACTGCTATGCCAAGGTCATGATGGTCA
ACGGCGACCACAGAATTGGCATCTTCGCTAAGAGGGCCATCCAGA
CCGGCGAGGAACTGTTCTTCGACTATAGATACTCCCAAGCCGACG
CTCTGAAGTACGTGGGCATCGAGAGAGAGATGGAAATCCCCGGAG
GTGGCGGATCGGGAAAGCGGCCCGCCGCCACCAAGAAGGCCGGTC
AGGCCAAGAAGAAGAAGGGCAGCTACCCCTACGACGTGCCCGACT
ACGCCTGAGCGGCCGCTTAATTAAGCTGCCTTCTGCGGGGCTTGC
CTTCTGGCCATGCCCTTCTTCTCTCCCTTGCACCTGTACCTCTTG
GTCTTTGAATAAAGCCTGAGTAGGAAGTCTAGAAAAAAAAAAAAA
AAAAAAAAAAAAAAAAAAAAAAAAAAAAAAAAAAAAAAAAAAAAA
AAAAAAAAAAAAAAAAAAAAAA

In some embodiments, a site-specific disrupting agent comprises the amino acid sequence of SEQ ID NOs: 70 or 80. In some embodiments, a site-specific disrupting agent described herein comprises an amino acid sequence of SEQ ID NO: 70 or 80 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-EZH2 protein
(SEQ ID NO. 70)
MAPKKKRKVGIHGVPAASGGGGSGSAAIAEVLLNARCDLHAVNYH
GDTPLHIAARESYHDCVLLFLSRGANPELRNKEGDTAWDLTPERS
DVWFALQLNRKLRLGVGNRAIRTEKIICRDVARGYENVPIPCVNG
VDGEPCPEDYKYISENCETSTMNIDRNITHLQHCTCVDDCSSSNC
LCGQLSIRCWYDKDGRLLQEFNKIEPPLIFECNQACSCWRNCKNR
VVQSGIKVRLQLYRTAKMGWGVRALQTIPQGTFICEYVGELISDA
EADVREDDSYLFDLDNKDGEVYCIDARYYGNISRFINHLCDPNII
PVRVFMLHQDLRFPRIAFFSSRDIRTGEELGFDYGDRFWDIKSKY
FTCQCGSEKCKHSAEAIALEQSRLARLDPHPELLPELGSLPPVNT
GGGGSGDKKYSIGLAIGTNSVGWAVITDEYKVPSKKFKVLGNTDR
HSIKKNLIGALLFDSGETAEATRLKRTARRRYTRRKNRICYLQEI
FSNEMAKVDDSFFHRLEESFLVEEDKKHERHPIFGNIVDEVAYHE
KYPTIYHLRKKLVDSTDKADLRLIYLALAHMIKFRGHFLIEGDLN
PDNSDVDKLFIQLVQTYNQLFEENPINASGVDAKAILSARLSKSR
RLENLIAQLPGEKKNGLFGNLIALSLGLTPNFKSNFDLAEDAKLQ
LSKDTYDDDLDNLLAQIGDQYADLFLAAKNLSDAILLSDILRVNT
EITKAPLSASMIKRYDEHHQDLTLLKALVRQQLPEKYKEIFFDQS
KNGYAGYIDGGASQEEFYKFIKPILEKMDGTEELLVKLNREDLLR
KQRTFDNGSIPHQIHLGELHAILRRQEDFYPFLKDNREKIEKILT
FRIPYYVGPLARGNSRFAWMTRKSEETITPWNFEEVVDKGASAQS
FIERMTNFDKNLPNEKVLPKHSLLYEYFTVYNELTKVKYVTEGMR
KPAFLSGEQKKAIVDLLFKTNRKVTVKQLKEDYFKKIECFDSVEI
SGVEDRFNASLGTYHDLLKIIKDKDFLDNEENEDILEDIVLTLTL
FEDREMIEERLKTYAHLFDDKVMKQLKRRRYTGWGRLSRKLINGI
RDKQSGKTILDFLKSDGFANRNFMQLIHDDSLTFKEDIQKAQVSG
QGDSLHEHIANLAGSPAIKKGILQTVKVVDELVKVMGRHKPENIV
IEMARENQTTQKGQKNSRERMKRIEEGIKELGSQILKEHPVENTQ
LQNEKLYLYYLQNGRDMYVDQELDINRLSDYDVAAIVPQSFLKDD
SIDNKVLTRSDKARGKSDNVPSEEVVKKMKNYWRQLLNAKLITQR
KFDNLTKAERGGLSELDKAGFIKRQLVETRQITKHVAQILDSRMN
TKYDENDKLIREVKVITLKSKLVSDFRKDFQFYKVREINNYHHAH
DAYLNAVVGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKRNSDKLI
ARKKDWDPKKYGGFDSPTVAYSVLVVAKVEKGKSKKLKSVKELLG
ITIMERSSFEKNPIDFLEAKGYKEVKKDLIIKLPKYSLFELENGR
KRMLASAGELQKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGAPAAFKYFDTTIDRKRYTSTKEVLDA
TLIHQSITGLYETRIDLSQLGGDSGGKRPAATKKAGQAKKKKSGG
GGSGQTGKKSEKGPVCWRKRVKSEYMRLRQLKRFRRADEVKSMFS
SNRQKILERTEILNQEWKQRRIQPVHILTSVSSLRGTRECSVTSD
LDFPTQVIPLKTLNAVASVPIMYSWSPLQQNFMVEDETVLHNIPY
MGDEVLDQDGTFIEELIKNYDGKVHGDRECGFINDEIFVELVNAL
GQYNDDDDDDDGDDPEEREEKQKDLEDHRDDKESRPPRKFPSDKI
FEAISSMFPDKGTAEELKEKYKELTEQQLPGALPPECTPNIDGPN
AKSVQREQSLHSFHTLFCRRCFKYDCFLHPFHATPNTYKRKNTET
ALDNKPCGPQCYQHLEGAKEFAAALTAERIKTPPKRPGGRRRGRL
PNNSSRPSTPTINVLESKDTDSDREAGTETGGENNDKEEEEKKDE
TSSSSEANSRCQTPIKMKPNIEPPENVEWSGAEASMFRVLIGTYY
DNFCAIARLIGTKTCRQVYEFRVKESSIIAPAPAEDVDTPPRKKK
RKHRLWAAHCRKIQLKKDGSSNHVYNYQPCDHPRQPCDSSCPCVI
AQNFCEKFCQCSSECQNRFPGCRCKAQCNTKQCPCYLAVRECDPD
LCLTCGAADHWDSKNVSCKNCSIQRGSKKHLLLAPSDVAGWGIFI
KDPVQKNEFISEYCGEIISQDEADRRGKVYDKYMCSFLFNLNNDF
VVDATRKGNKIRFANHSVNPNCYAKVMMVNGDHRIGIFAKRAIQT
GEELFFDYRYSQADALKYVGIEREMEIPGGGGSGKRPAATKKAGQ
AKKKKGSYPYDVPDYA
G9A-dCas9-EZH2 protein without HA tag
(SEQ ID NO. 80)
MAPKKKRKVGIHGVPAASGGGGSGSAAIAEVLLNARCDLHAVNYH
GDTPLHIAARESYHDCVLLFLSRGANPELRNKEGDTAWDLTPERS
DVWFALQLNRKLRLGVGNRAIRTEKIICRDVARGYENVPIPCVNG
VDGEPCPEDYKYISENCETSTMNIDRNITHLQHCTCVDDCSSSNC
LCGQLSIRCWYDKDGRLLQEFNKIEPPLIFECNQACSCWRNCKNR
VVQSGIKVRLQLYRTAKMGWGVRALQTIPQGTFICEYVGELISDA
EADVREDDSYLFDLDNKDGEVYCIDARYYGNISRFINHLCDPNII
PVRVFMLHQDLRFPRIAFFSSRDIRTGEELGFDYGDRFWDIKSKY
FTCQCGSEKCKHSAEAIALEQSRLARLDPHPELLPELGSLPPVNT
GGGGSGDKKYSIGLAIGTNSVGWAVITDEYKVPSKKFKVLGNTDR
HSIKKNLIGALLFDSGETAEATRLKRTARRRYTRRKNRICYLQEI
FSNEMAKVDDSFFHRLEESFLVEEDKKHERHPIFGNIVDEVAYHE
KYPTIYHLRKKLVDSTDKADLRLIYLALAHMIKFRGHFLIEGDLN
PDNSDVDKLFIQLVQTYNQLFEENPINASGVDAKAILSARLSKSR
RLENLIAQLPGEKKNGLFGNLIALSLGLTPNFKSNFDLAEDAKLQ
LSKDTYDDDLDNLLAQIGDQYADLFLAAKNLSDAILLSDILRVNT
EITKAPLSASMIKRYDEHHQDLTLLKALVRQQLPEKYKEIFFDQS
KNGYAGYIDGGASQEEFYKFIKPILEKMDGTEELLVKLNREDLLR
KQRTFDNGSIPHQIHLGELHAILRRQEDFYPFLKDNREKIEKILT
FRIPYYVGPLARGNSRFAWMTRKSEETITPWNFEEVVDKGASAQS
FIERMTNFDKNLPNEKVLPKHSLLYEYFTVYNELTKVKYVTEGMR
KPAFLSGEQKKAIVDLLFKTNRKVTVKQLKEDYFKKIECFDSVEI
SGVEDRFNASLGTYHDLLKIIKDKDFLDNEENEDILEDIVLTLTL
FEDREMIEERLKTYAHLFDDKVMKQLKRRRYTGWGRLSRKLINGI
RDKQSGKTILDFLKSDGFANRNFMQLIHDDSLTFKEDIQKAQVSG
QGDSLHEHIANLAGSPAIKKGILQTVKVVDELVKVMGRHKPENIV
IEMARENQTTQKGQKNSRERMKRIEEGIKELGSQILKEHPVENTQ
LQNEKLYLYYLQNGRDMYVDQELDINRLSDYDVAAIVPQSFLKDD
SIDNKVLTRSDKARGKSDNVPSEEVVKKMKNYWRQLLNAKLITQR
KFDNLTKAERGGLSELDKAGFIKRQLVETRQITKHVAQILDSRMN
TKYDENDKLIREVKVITLKSKLVSDFRKDFQFYKVREINNYHHAH
DAYLNAVVGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKRNSDKLI
ARKKDWDPKKYGGFDSPTVAYSVLVVAKVEKGKSKKLKSVKELLG
ITIMERSSFEKNPIDFLEAKGYKEVKKDLIIKLPKYSLFELENGR
KRMLASAGELQKGNELALPSKYVNFLYLASHYEKLKGSPEDNEQK
QLFVEQHKHYLDEIIEQISEFSDATLIHQSITGLYETRIDLSQLG
GDSGGKRPAATKKAGQAKKKKSGGGGSGQTGKKSEKGPVCWRKRV
KSEYMRLRQLKRFRRADEVKSMFSSNRQKILERTEILNQEWKQRR
IQPVHILTSVSSLRGTRECSVTSDLDFPTQVIPLKTLNAVASVPI
MYSWSPLQQNFMVEDETVLHNIPYMGDEVLDQDGTFIEELIKNYD
GKVHGDRECGFINDEIFVELVNALGQYNDDDDDDDGDDPEEREEK
QKDLEDHRDDKESRPPRKFPSDKIFEAISSMFPDKGTAEELKEKY
KELTEQQLPGALPPECTPNIDGPNAKSVQREQSLHSFHTLFCRRC
FKYDCFLHPFHATPNTYKRKNTETALDNKPCGPQCYQHLEGAKEF
AAALTAERIKTPPKRPGGRRRGRLPNNSSRPSTPTINVLESKDTD
SDREAGTETGGENNDKEEEEKKDETSSSSEANSRCQTPIKMKPNI
EPPENVEWSGAEASMFRVLIGTYYDNFCAIARLIGTKTCRQVYEF
RVKESSIIAPAPAEDVDTPPRKKKRKHRLWAAHCRKIQLKKDGSS
NHVYNYQPCDHPRQPCDSSCPCVIAQNFCEKFCQCSSECQNRFPG
CRCKAQCNTKQCPCYLAVRECDPDLCLTCGAADHWDSKNVSCKNC
SIQRGSKKHLLLAPSDVAGWGIFIKDPVQKNEFISEYCGEIISQD
EADRRGKVYDKYMCSFLFNLNNDFVVDATRKGNKIRFANHSVNPN
CYAKVMMVNGDHRIGIFAKRAIQTGEELFFDYRYSQADALKYVGI
EREMEIPGGGGSGKRPAATKKAGQAKKKKGS

In some embodiments, a site-specific disrupting agent comprises a targeting moiety (e.g., comprising dCas9, e.g., an S. aureus dCas9), a first effector moiety comprising G9A; e.g., a G9A domain, and a second effector moiety comprising KRAB, e.g., a KRAB domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising G9A, e.g., a G9A domain; and a second effector moiety comprising KRAB, e.g., a KRAB domain. In some embodiments, the site-specific disrupting agent is encoded by the nucleic acid sequence of SEQ ID NO: 71 (e.g., mRNA encoding the site-specific disrupting agent). In some embodiments, a nucleic acid described herein comprises a nucleic acid sequence of SEQ ID NO: 71 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-KRAB (MR-29942) mRNA sequence
(SEQ ID NO. 71)
AGGAAATAAGAGAGAAAAGAAGAGTAAGAAGAAATATAAGAGCCA
CCATGGCCCCCAAGAAGAAGCGGAAGGTGGGCATCCACGGCGTGC
CCGCCGCCTCGGGCGGGGGTGGCTCAGGAAGCGCCGCTATCGCCG
AAGTGCTGCTGAATGCCAGATGCGATCTGCATGCCGTGAACTACC
ACGGCGACACCCCTCTGCATATCGCCGCTAGAGAGAGCTACCATG
ACTGTGTGCTGCTGTTTCTGAGCAGAGGCGCCAATCCCGAGCTCA
GAAACAAAGAGGGCGACACCGCTTGGGATCTGACACCCGAGAGAT
CCGACGTGTGGTTCGCTCTGCAACTGAATAGAAAACTGAGACTGG
GCGTCGGCAATAGGGCCATTAGAACCGAGAAGATCATCTGTAGGG
ACGTGGCTAGGGGCTACGAGAACGTGCCCATCCCTTGTGTGAACG
GAGTGGATGGAGAGCCTTGCCCCGAGGATTACAAATACATCAGCG
AGAACTGCGAAACCTCCACCATGAATATCGATAGAAACATTACAC
ACCTCCAGCACTGTACATGCGTGGACGATTGCAGCAGCAGCAACT
GTCTGTGCGGCCAACTGAGCATCAGATGCTGGTACGACAAGGATG
GCAGACTGCTGCAAGAGTTCAACAAGATCGAACCCCCTCTGATCT
TCGAGTGTAACCAAGCTTGCAGCTGTTGGAGGAACTGCAAGAATA
GGGTCGTGCAGTCCGGAATCAAGGTGAGACTGCAGCTGTATAGAA
CAGCTAAGATGGGATGGGGAGTCAGAGCTCTGCAGACCATCCCCC
AAGGCACATTCATCTGTGAGTACGTCGGCGAACTCATCAGCGACG
CTGAGGCCGATGTGAGGGAGGACGACAGCTATCTCTTCGACCTCG
ACAACAAGGACGGCGAGGTGTACTGCATCGACGCTAGATATTACG
GCAACATCAGCAGATTCATCAACCACCTCTGCGACCCCAATATCA
TCCCCGTGAGAGTGTTCATGCTCCATCAAGATCTGAGATTCCCTA
GGATCGCCTTCTTCAGCTCTAGAGACATTAGAACCGGCGAGGAGC
TGGGATTCGACTACGGCGACAGGTTCTGGGACATCAAGAGCAAGT
ACTTCACATGCCAATGCGGCAGCGAGAAATGCAAGCATAGCGCCG
AGGCCATTGCTCTGGAGCAGTCTAGACTGGCTAGGCTGGACCCTC
ACCCCGAGCTGCTGCCCGAACTGGGATCTCTGCCTCCCGTGAATA
CCGGAGGTGGCGGATCGGGAGACAAGAAGTACAGCATCGGCCTGG
CCATCGGCACCAACAGCGTGGGCTGGGCCGTGATCACCGACGAGT
ACAAGGTGCCCAGCAAGAAGTTCAAGGTGCTGGGCAACACCGACC
GGCACAGCATCAAGAAGAACCTGATCGGCGCCCTGCTGTTCGACA
GCGGCGAGACCGCCGAGGCCACCCGGCTGAAGCGGACCGCCCGGC
GGCGGTACACCCGGCGGAAGAACCGGATCTGCTACCTGCAGGAGA
TCTTCAGCAACGAGATGGCCAAGGTGGACGACAGCTTCTTCCACC
GGCTGGAGGAGAGCTTCCTGGTGGAGGAGGACAAGAAGCACGAGC
GGCACCCCATCTTCGGCAACATCGTGGACGAGGTGGCCTACCACG
AGAAGTACCCCACCATCTACCACCTGCGGAAGAAGCTGGTGGACA
GCACCGACAAGGCCGACCTGCGGCTGATCTACCTGGCCCTGGCCC
ACATGATCAAGTTCCGGGGCCACTTCCTGATCGAGGGCGACCTGA
ACCCCGACAACAGCGACGTGGACAAGCTGTTCATCCAGCTGGTGC
AGACCTACAACCAGCTGTTCGAGGAGAACCCCATCAACGCCAGCG
GCGTGGACGCCAAGGCCATCCTGAGCGCCCGGCTGAGCAAGAGCC
GGCGGCTGGAGAACCTGATCGCCCAGCTGCCCGGCGAGAAGAAGA
ACGGCCTGTTCGGCAACCTGATCGCCCTGAGCCTGGGCCTGACCC
CCAACTTCAAGAGCAACTTCGACCTGGCCGAGGACGCCAAGCTGC
AGCTGAGCAAGGACACCTACGACGACGACCTGGACAACCTGCTGG
CCCAGATCGGCGACCAGTACGCCGACCTGTTCCTGGCCGCCAAGA
ACCTGAGCGACGCCATCCTGCTGAGCGACATCCTGCGGGTGAACA
CCGAGATCACCAAGGCCCCCCTGAGCGCCAGCATGATCAAGCGGT
ACGACGAGCACCACCAGGACCTGACCCTGCTGAAGGCCCTGGTGC
GGCAGCAGCTGCCCGAGAAGTACAAGGAGATCTTCTTCGACCAGA
GCAAGAACGGCTACGCCGGCTACATCGACGGCGGCGCCAGCCAGG
AGGAGTTCTACAAGTTCATCAAGCCCATCCTGGAGAAGATGGACG
GCACCGAGGAGCTGCTGGTGAAGCTGAACCGGGAGGACCTGCTGC
GGAAGCAGCGGACCTTCGACAACGGCAGCATCCCCCACCAGATCC
ACCTGGGCGAGCTGCACGCCATCCTGCGGCGGCAGGAGGACTTCT
ACCCCTTCCTGAAGGACAACCGGGAGAAGATCGAGAAGATCCTGA
CCTTCCGGATCCCCTACTACGTGGGCCCCCTGGCCCGGGGCAACA
GCCGGTTCGCCTGGATGACCCGGAAATCCGAGGAGACCATCACCC
CCTGGAACTTCGAGGAGGTGGTGGACAAGGGCGCCAGCGCCCAGA
GCTTCATCGAGCGGATGACCAACTTCGACAAGAACCTGCCCAACG
AGAAGGTGCTGCCCAAGCACAGCCTGCTGTACGAGTACTTCACCG
TGTACAACGAGCTGACCAAGGTGAAGTACGTGACCGAGGGCATGC
GGAAGCCCGCCTTCCTGAGCGGCGAGCAGAAGAAGGCCATCGTGG
ACCTGCTGTTCAAGACCAACCGGAAGGTGACCGTGAAGCAGCTGA
AGGAGGACTACTTCAAGAAGATCGAGTGCTTCGACAGCGTGGAGA
TCAGCGGCGTGGAGGACCGGTTCAACGCCAGCCTGGGCACCTACC
ACGACCTGCTGAAGATCATCAAGGACAAGGACTTCCTGGACAACG
AGGAGAACGAGGACATCCTGGAGGACATCGTGCTGACCCTGACCC
TGTTCGAGGACCGGGAGATGATCGAGGAGCGGCTGAAAACCTACG
CCCACCTGTTCGACGACAAGGTGATGAAGCAGCTGAAGCGGCGGC
GGTACACCGGCTGGGGCCGGCTGAGCCGGAAGCTGATCAACGGCA
TCCGGGACAAGCAGAGCGGCAAGACCATCCTGGACTTCCTGAAAT
CCGACGGCTTCGCCAACCGGAACTTCATGCAGCTGATCCACGACG
ACAGCCTGACCTTCAAGGAGGACATCCAGAAGGCCCAGGTGAGCG
GCCAGGGCGACAGCCTGCACGAGCACATCGCCAACCTGGCCGGCA
GCCCCGCCATCAAGAAGGGCATCCTGCAGACCGTGAAGGTGGTGG
ACGAGCTGGTGAAGGTGATGGGCCGGCACAAGCCCGAGAACATCG
TGATCGAGATGGCCCGGGAGAACCAGACCACCCAGAAGGGCCAGA
AGAACAGCCGGGAGCGGATGAAGCGGATCGAGGAGGGCATCAAGG
AGCTGGGCAGCCAGATCCTGAAGGAGCACCCCGTGGAGAACACCC
AGCTGCAGAACGAGAAGCTGTACCTGTACTACCTGCAGAACGGCC
GGGACATGTACGTGGACCAGGAGCTGGACATCAACCGGCTGAGCG
ACTACGACGTGGCCGCCATCGTGCCCCAGAGCTTCCTGAAGGACG
ACAGCATCGACAACAAGGTGCTGACCCGGAGCGACAAGGCCCGGG
GCAAGAGCGACAACGTGCCCAGCGAGGAGGTGGTGAAGAAGATGA
AGAACTACTGGCGGCAGCTGCTGAACGCCAAGCTGATCACCCAGC
GGAAGTTCGACAACCTGACCAAGGCCGAGCGGGGCGGCCTGAGCG
AGCTGGACAAGGCCGGCTTCATCAAGCGGCAGCTGGTGGAGACCC
GGCAGATCACCAAGCACGTGGCCCAGATCCTGGACAGCCGGATGA
ACACCAAGTACGACGAGAACGACAAGCTGATCCGGGAGGTGAAGG
TGATCACCCTGAAATCCAAGCTGGTGAGCGACTTCCGGAAGGACT
TCCAGTTCTACAAGGTGCGGGAGATCAACAACTACCACCACGCCC
ACGACGCCTACCTGAACGCCGTGGTGGGCACCGCCCTGATCAAGA
AGTACCCCAAGCTGGAGAGCGAGTTCGTGTACGGCGACTACAAGG
TGTACGACGTGCGGAAGATGATCGCCAAGAGCGAGCAGGAGATCG
GCAAGGCCACCGCCAAGTACTTCTTCTACAGCAACATCATGAACT
TCTTCAAGACCGAGATCACCCTGGCCAACGGCGAGATCCGGAAGC
GGCCCCTGATCGAGACCAACGGCGAGACCGGCGAGATCGTGTGGG
ACAAGGGCCGGGACTTCGCCACCGTGCGGAAGGTGCTGAGCATGC
CCCAGGTGAACATCGTGAAGAAAACCGAGGTGCAGACCGGCGGCT
TCAGCAAGGAGAGCATCCTGCCCAAGCGGAACAGCGACAAGCTGA
TCGCCCGGAAGAAGGACTGGGACCCCAAGAAGTACGGCGGCTTCG
ACAGCCCCACCGTGGCCTACAGCGTGCTGGTGGTGGCCAAGGTGG
AGAAGGGCAAGAGCAAGAAGCTGAAATCCGTGAAGGAGCTGCTGG
GCATCACCATCATGGAGCGGAGCAGCTTCGAGAAGAACCCCATCG
ACTTCCTGGAGGCCAAGGGCTACAAGGAGGTGAAGAAGGACCTGA
TCATCAAGCTGCCCAAGTACAGCCTGTTCGAGCTGGAGAACGGCC
GGAAGCGGATGCTGGCCAGCGCCGGCGAGCTGCAGAAGGGCAACG
AGCTGGCCCTGCCCAGCAAGTACGTGAACTTCCTGTACCTGGCCA
GCCACTACGAGAAGCTGAAGGGCAGCCCCGAGGACAACGAGCAGA
AGCAGCTGTTCGTGGAGCAGCACAAGCACTACCTGGACGAGATCA
TCGAGCAGATCAGCGAGTTCAGCAAGCGGGTGATCCTGGCCGACG
CCAACCTGGACAAGGTGCTGAGCGCCTACAACAAGCACCGGGACA
AGCCCATCCGGGAGCAGGCCGAGAACATCATCCACCTGTTCACCC
TGACCAACCTGGGCGCCCCCGCCGCCTTCAAGTACTTCGACACCA
CCATCGACCGGAAGCGGTACACCAGCACCAAGGAGGTGCTGGACG
CCACCCTGATCCACCAGAGCATCACCGGCCTGTACGAGACCCGGA
TCGACCTGAGCCAGCTGGGCGGCGACAGCGGCGGCAAGCGGCCCG
CCGCCACCAAGAAGGCCGGCCAGGCCAAGAAGAAGAAGTCGGGCG
GGGGTGGCTCAGACGCTAAGTCTCTGACCGCTTGGAGCAGAACAC
TGGTCACCTTCAAGGACGTGTTCGTCGACTTCACAAGAGAGGAGT
GGAAACTGCTGGACACCGCCCAGCAGATCCTCTATAGAAACGTCA
TGCTGGAGAACTACAAGAATCTGGTGTCTCTGGGCTACCAGCTGA
CCAAGCCCGACGTGATTCTGAGGCTGGAGAAGGGCGAGGAGCCTT
GGCTGGTGGAGAGAGAGATCCACCAAGAAACCCACCCCGACAGCG
AAACCGCCTTCGAGATCAAGAGCAGCGTGGGAGGTGGCGGATCGG
GAAAGCGGCCCGCCGCCACCAAGAAGGCCGGTCAGGCCAAGAAGA
AGAAGGGCAGCTACCCCTACGACGTGCCCGACTACGCCTGAGCGG
CCGCTTAATTAAGCTGCCTTCTGCGGGGCTTGCCTTCTGGCCATG
CCCTTCTTCTCTCCCTTGCACCTGTACCTCTTGGTCTTTGAATAA
AGCCTGAGTAGGAAGTCTAGAAAAAAAAAAAAAAAAAAAAAAAAA
AAAAAAAAAAAAAAAAAAAAAAAAAAAAAAAAAAAAAAAAAAAAA
AAAAAAAAAA

In some embodiments, a site-specific disrupting agent comprises the amino acid sequence of SEQ ID NOs: 72 or 81. In some embodiments, a site-specific disrupting agent described herein comprises an amino acid sequence of SEQ ID NO: 72 or 81 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

G9A-dCas9-KRAB protein
(SEQ ID NO. 72)
MAPKKKRKVGIHGVPAASGGGGSGSAAIAEVLLNARCDLHAVNYH
GDTPLHIAARESYHDCVLLFLSRGANPELRNKEGDTAWDLTPERS
DVWFALQLNRKLRLGVGNRAIRTEKIICRDVARGYENVPIPCVNG
VDGEPCPEDYKYISENCETSTMNIDRNITHLQHCTCVDDCSSSNC
LCGQLSIRCWYDKDGRLLQEFNKIEPPLIFECNQACSCWRNCKNR
VVQSGIKVRLQLYRTAKMGWGVRALQTIPQGTFICEYVGELISDA
EADVREDDSYLFDLDNKDGEVYCIDARYYGNISRFINHLCDPNII
PVRVFMLHQDLRFPRIAFFSSRDIRTGEELGFDYGDRFWDIKSKY
FTCQCGSEKCKHSAEAIALEQSRLARLDPHPELLPELGSLPPVNT
GGGGSGDKKYSIGLAIGTNSVGWAVITDEYKVPSKKFKVLGNTDR
HSIKKNLIGALLFDSGETAEATRLKRTARRRYTRRKNRICYLQEI
FSNEMAKVDDSFFHRLEESFLVEEDKKHERHPIFGNIVDEVAYHE
KYPTIYHLRKKLVDSTDKADLRLIYLALAHMIKFRGHFLIEGDLN
PDNSDVDKLFIQLVQTYNQLFEENPINASGVDAKAILSARLSKSR
RLENLIAQLPGEKKNGLFGNLIALSLGLTPNFKSNFDLAEDAKLQ
LSKDTYDDDLDNLLAQIGDQYADLFLAAKNLSDAILLSDILRVNT
EITKAPLSASMIKRYDEHHQDLTLLKALVRQQLPEKYKEIFFDQS
KNGYAGYIDGGASQEEFYKFIKPILEKMDGTEELLVKLNREDLLR
KQRTFDNGSIPHQIHLGELHAILRRQEDFYPFLKDNREKIEKILT
FRIPYYVGPLARGNSRFAWMTRKSEETITPWNFEEVVDKGASAQS
FIERMTNFDKNLPNEKVLPKHSLLYEYFTVYNELTKVKYVTEGMR
KPAFLSGEQKKAIVDLLFKTNRKVTVKQLKEDYFKKIECFDSVEI
SGVEDRFNASLGTYHDLLKIIKDKDFLDNEENEDILEDIVLTLTL
FEDREMIEERLKTYAHLFDDKVMKQLKRRRYTGWGRLSRKLINGI
RDKQSGKTILDFLKSDGFANRNFMQLIHDDSLTFKEDIQKAQVSG
QGDSLHEHIANLAGSPAIKKGILQTVKVVDELVKVMGRHKPENIV
IEMARENQTTQKGQKNSRERMKRIEEGIKELGSQILKEHPVENTQ
LQNEKLYLYYLQNGRDMYVDQELDINRLSDYDVAAIVPQSFLKDD
SIDNKVLTRSDKARGKSDNVPSEEVVKKMKNYWRQLLNAKLITQR
KFDNLTKAERGGLSELDKAGFIKRQLVETRQITKHVAQILDSRMN
TKYDENDKLIREVKVITLKSKLVSDFRKDFQFYKVREINNYHHAH
DAYLNAVVGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKRNSDKLI
ARKKDWDPKKYGGFDSPTVAYSVLVVAKVEKGKSKKLKSVKELLG
ITIMERSSFEKNPIDFLEAKGYKEVKKDLIIKLPKYSLFELENGR
KRMLASAGELQKGNELALPSKYVNFLYLASHYEKLKGSPEDNEQK
QLFVEQHKHYLDEIIEQISEFSDATLIHQSITGLYETRIDLSQLG
GDSGGKRPAATKKAGQAKKKKSGGGGSDAKSLTAWSRTLVTFKDV
FVDFTREEWKLLDTAQQILYRNVMLENYKNLVSLGYQLTKPDVIL
RLEKGEEPWLVEREIHQETHPDSETAFEIKSSVGGGGSGKRPAAT
KKAGQAKKKKGSYPYDVPDYA
G9A-dCas9-KRAB protein without HA tag
(SEQ ID NO. 81)
MAPKKKRKVGIHGVPAASGGGGSGSAAIAEVLLNARCDLHAVNYH
GDTPLHIAARESYHDCVLLFLSRGANPELRNKEGDTAWDLTPERS
DVWFALQLNRKLRLGVGNRAIRTEKIICRDVARGYENVPIPCVNG
VDGEPCPEDYKYISENCETSTMNIDRNITHLQHCTCVDDCSSSNC
LCGQLSIRCWYDKDGRLLQEFNKIEPPLIFECNQACSCWRNCKNR
VVQSGIKVRLQLYRTAKMGWGVRALQTIPQGTFICEYVGELISDA
EADVREDDSYLFDLDNKDGEVYCIDARYYGNISRFINHLCDPNII
PVRVFMLHQDLRFPRIAFFSSRDIRTGEELGFDYGDRFWDIKSKY
FTCQCGSEKCKHSAEAIALEQSRLARLDPHPELLPELGSLPPVNT
GGGGSGDKKYSIGLAIGTNSVGWAVITDEYKVPSKKFKVLGNTDR
HSIKKNLIGALLFDSGETAEATRLKRTARRRYTRRKNRICYLQEI
FSNEMAKVDDSFFHRLEESFLVEEDKKHERHPIFGNIVDEVAYHE
KYPTIYHLRKKLVDSTDKADLRLIYLALAHMIKFRGHFLIEGDLN
PDNSDVDKLFIQLVQTYNQLFEENPINASGVDAKAILSARLSKSR
RLENLIAQLPGEKKNGLFGNLIALSLGLTPNFKSNFDLAEDAKLQ
LSKDTYDDDLDNLLAQIGDQYADLFLAAKNLSDAILLSDILRVNT
EITKAPLSASMIKRYDEHHQDLTLLKALVRQQLPEKYKEIFFDQS
KNGYAGYIDGGASQEEFYKFIKPILEKMDGTEELLVKLNREDLLR
KQRTFDNGSIPHQIHLGELHAILRRQEDFYPFLKDNREKIEKILT
FRIPYYVGPLARGNSRFAWMTRKSEETITPWNFEEVVDKGASAQS
FIERMTNFDKNLPNEKVLPKHSLLYEYFTVYNELTKVKYVTEGMR
KPAFLSGEQKKAIVDLLFKTNRKVTVKQLKEDYFKKIECFDSVEI
SGVEDRFNASLGTYHDLLKIIKDKDFLDNEENEDILEDIVLTLTL
FEDREMIEERLKTYAHLFDDKVMKQLKRRRYTGWGRLSRKLINGI
RDKQSGKTILDFLKSDGFANRNFMQLIHDDSLTFKEDIQKAQVSG
QGDSLHEHIANLAGSPAIKKGILQTVKVVDELVKVMGRHKPENIV
IEMARENQTTQKGQKNSRERMKRIEEGIKELGSQILKEHPVENTQ
LQNEKLYLYYLQNGRDMYVDQELDINRLSDYDVAAIVPQSFLKDD
SIDNKVLTRSDKARGKSDNVPSEEVVKKMKNYWRQLLNAKLITQR
KFDNLTKAERGGLSELDKAGFIKRQLVETRQITKHVAQILDSRMN
TKYDENDKLIREVKVITLKSKLVSDFRKDFQFYKVREINNYHHAH
DAYLNAVVGTALIKKYPKLESEFVYGDYKVYDVRKMIAKSEQEIG
KATAKYFFYSNIMNFFKTEITLANGEIRKRPLIETNGETGEIVWD
KGRDFATVRKVLSMPQVNIVKKTEVQTGGFSKESILPKRNSDKLI
ARKKDWDPKKYGGFDSPTVAYSVLVVAKVEKGKSKKLKSVKELLG
ITIMERSSFEKNPIDFLEAKGYKEVKKDLIIKLPKYSLFELENGR
KRMLASAGELQKGNELALPSKYVNFLYLASHYEKLKGSPEDNEQK
QLFVEQHKHYLDEIIEQISEFSKRVILADANLDKVLSAYNKHRDK
PIREQAENIIHLFTLTNLGAPAAFKYFDTTIDRKRYTSTKEVLDA
TLIHQSITGLYETRIDLSQLGGDSGGKRPAATKKAGQAKKKKSGG
GGSDAKSLTAWSRTLVTFKDVFVDFTREEWKLLDTAQQILYRNVM
LENYKNLVSLGYQLTKPDVILRLEKGEEPWLVEREIHQETHPDSE
TAFEIKSSVGGGGSGKRPAATKKAGQAKKKKGS

In some embodiments, a site-specific disrupting agent comprises a targeting moiety (e.g., comprising dCas9, e.g., an S. aureus dCas9), a first effector moiety comprising EZH2; e.g., an EZH2 domain, and a second effector moiety comprising KRAB, e.g., a KRAB domain. In some embodiments, a site-specific disrupting agent comprises a targeting moiety comprising dCas9, e.g., an S. pyogenes dCas9, e.g., a mutant S. pyogenes Cas9, e.g., Cas9m4; a first effector moiety comprising EZH2, e.g., an EZH2 domain; and a second effector moiety comprising KRAB, e.g., a KRAB domain. In some embodiments, the site-specific disrupting agent is encoded by the nucleic acid sequence of SEQ ID NO: 85 (e.g., mRNA encoding the site-specific disrupting agent). In some embodiments, a nucleic acid described herein comprises a nucleic acid sequence of SEQ ID NO: 85 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-KRAB (MR-29948) mRNA sequence
(SEQ ID NO. 85)
AGGAAATAAGAGAGAAAAGAAGAGTAAGAAGAAATATAAGAGCCA
CCATGGCCCCCAAGAAGAAGCGGAAGGTGGGCGGCAGCGGCGGCA
GCGGCCAGACCGGCAAGAAGAGCGAGAAGGGCCCCGTGTGCTGGC
GGAAGCGGGTGAAGAGCGAGTACATGCGGCTGCGGCAGCTGAAGC
GGTTCCGGCGGGCCGACGAGGTGAAGAGCATGTTCAGCAGCAACC
GGCAGAAGATCCTGGAGCGGACCGAGATCCTGAACCAGGAGTGGA
AGCAGCGGCGAATCCAGCCCGTGCACATCCTGACCAGCGTGAGCA
GCCTGCGGGGCACCCGGGAGTGCAGCGTGACCAGCGACCTGGACT
TCCCCACCCAGGTGATCCCCCTAAAGACCCTGAACGCCGTGGCCA
GCGTGCCCATCATGTACAGCTGGAGCCCCCTGCAGCAGAACTTCA
TGGTGGAGGACGAGACCGTGCTGCACAACATCCCCTACATGGGCG
ACGAGGTGCTGGACCAGGACGGCACCTTCATCGAGGAGCTGATCA
AGAACTACGACGGCAAGGTGCACGGCGACCGGGAGTGCGGCTTCA
TCAACGACGAGATCTTCGTGGAGCTGGTGAACGCCCTGGGCCAGT
ACAACGACGACGACGACGACGACGACGGCGACGACCCCGAGGAGC
GGGAGGAGAAGCAGAAGGACCTGGAGGACCACCGGGACGACAAGG
AGAGCCGGCCCCCCCGGAAGTTCCCCAGCGACAAGATCTTCGAGG
CCATCAGCAGCATGTTCCCCGACAAGGGCACCGCCGAGGAGCTGA
AGGAGAAGTACAAGGAGCTGACCGAGCAGCAGCTGCCCGGCGCCC
TGCCCCCCGAGTGCACCCCCAACATCGACGGCCCCAACGCCAAGA
GCGTGCAGCGGGAGCAGAGCCTGCACAGCTTCCACACCCTGTTCT
GCCGGCGGTGCTTCAAGTACGACTGCTTCCTGCACCCCTTCCACG
CCACCCCCAACACCTACAAGCGGAAGAACACCGAGACCGCCCTGG
ACAACAAGCCCTGCGGCCCCCAGTGCTACCAGCACCTGGAGGGCG
CCAAGGAGTTCGCCGCCGCCCTGACCGCCGAGCGGATCAAGACCC
CCCCCAAGCGGCCCGGCGGCCGGCGGCGGGGCCGGCTGCCCAACA
ACAGCAGCCGGCCCAGCACCCCCACCATCAACGTGCTGGAGAGCA
AGGACACCGACAGCGACCGGGAGGCCGGCACCGAGACCGGCGGCG
AGAACAACGACAAGGAGGAGGAGGAGAAGAAGGACGAGACCAGCA
GCAGCAGCGAGGCCAACAGCCGGTGCCAGACCCCCATCAAGATGA
AGCCCAACATCGAGCCCCCCGAGAACGTGGAGTGGAGCGGCGCCG
AGGCCAGCATGTTCCGGGTGCTGATCGGCACCTACTACGACAACT
TCTGCGCCATCGCCCGGCTGATCGGCACCAAGACCTGCCGGCAGG
TGTACGAGTTCCGGGTGAAGGAGAGCAGCATCATCGCCCCCGCCC
CCGCCGAGGACGTGGACACCCCCCCCCGGAAGAAGAAGCGGAAGC
ACCGGCTGTGGGCCGCCCACTGCCGGAAGATCCAGCTGAAGAAGG
ACGGCAGCAGCAACCACGTGTACAACTACCAGCCCTGCGACCACC
CCCGGCAGCCCTGCGACAGCAGCTGCCCCTGCGTGATCGCCCAGA
ACTTCTGCGAGAAGTTCTGCCAGTGCAGCAGCGAGTGCCAGAACC
GGTTCCCCGGCTGCCGGTGCAAGGCCCAGTGCAACACCAAGCAGT
GCCCCTGCTACCTGGCCGTGCGGGAGTGCGACCCCGACCTGTGCC
TGACCTGCGGCGCCGCCGACCACTGGGACAGCAAGAACGTGAGCT
GCAAGAACTGCAGCATCCAGCGGGGCAGCAAGAAGCACCTGCTGC
TGGCCCCCAGCGACGTGGCCGGCTGGGGCATCTTCATCAAGGACC
CCGTGCAGAAGAACGAGTTCATCAGCGAGTACTGCGGCGAGATCA
TCAGCCAGGACGAGGCCGACCGGCGGGGCAAGGTGTACGACAAGT
ACATGTGCAGCTTCCTGTTCAACCTGAACAACGACTTCGTGGTGG
ACGCCACCCGGAAGGGCAACAAGATCCGGTTCGCCAACCACAGCG
TGAACCCCAACTGCTACGCCAAGGTGATGATGGTGAACGGCGACC
ACCGGATCGGCATCTTCGCCAAGCGGGCCATCCAGACCGGCGAGG
AGCTGTTCTTCGACTACCGGTACAGCCAGGCCGACGCCCTGAAGT
ACGTGGGCATCGAGCGGGAGATGGAGATCCCCAGCACCGGCGGCA
GCGGCGGCAGCGGCGGCAGCGGCGGCAGCGGCGGCAGCGGCCGAC
CCGACAAGAAGTACAGCATCGGCCTGGCCATCGGCACCAACAGCG
TGGGCTGGGCCGTGATCACCGACGAGTACAAGGTGCCCAGCAAGA
AGTTCAAGGTGCTGGGCAACACCGACCGGCACAGCATCAAGAAGA
ACCTGATCGGCGCCCTGCTGTTCGACAGCGGCGAGACCGCCGAGG
CCACCCGGCTGAAGCGGACCGCCCGGCGGCGGTACACCCGGCGGA
AGAACCGGATCTGCTACCTGCAGGAGATCTTCAGCAACGAGATGG
CCAAGGTGGACGACAGCTTCTTCCACCGGCTGGAGGAGAGCTTCC
TGGTGGAGGAGGACAAGAAGCACGAGCGGCACCCCATCTTCGGCA
ACATCGTGGACGAGGTGGCCTACCACGAGAAGTACCCCACCATCT
ACCACCTGCGGAAGAAGCTGGTGGACAGCACCGACAAGGCCGACC
TGCGGCTGATCTACCTGGCCCTGGCCCACATGATCAAGTTCCGGG
GCCACTTCCTGATCGAGGGCGACCTGAACCCCGACAACAGCGACG
TGGACAAGCTGTTCATCCAGCTGGTGCAGACCTACAACCAGCTGT
TCGAGGAGAACCCCATCAACGCCAGCGGCGTGGACGCCAAGGCCA
TCCTGAGCGCCCGGCTGAGCAAGAGCCGGCGGCTGGAGAACCTGA
TCGCCCAGCTGCCCGGCGAGAAGAAGAACGGCCTGTTCGGCAACC
TGATCGCCCTGAGCCTGGGCCTGACCCCCAACTTCAAGAGCAACT
TCGACCTGGCCGAGGACGCCAAGCTGCAGCTGAGCAAGGACACCT
ACGACGACGACCTGGACAACCTGCTGGCCCAGATCGGCGACCAGT
ACGCCGACCTGTTCCTGGCCGCCAAGAACCTGAGCGACGCCATCC
TGCTGAGCGACATCCTGCGGGTGAACACCGAGATCACCAAGGCCC
CCCTGAGCGCCAGCATGATCAAGCGGTACGACGAGCACCACCAGG
ACCTGACCCTGCTGAAGGCCCTGGTGCGGCAGCAGCTGCCCGAGA
AGTACAAGGAGATCTTCTTCGACCAGAGCAAGAACGGCTACGCCG
GCTACATCGACGGCGGCGCCAGCCAGGAGGAGTTCTACAAGTTCA
TCAAGCCCATCCTGGAGAAGATGGACGGCACCGAGGAGCTGCTGG
TGAAGCTGAACCGGGAGGACCTGCTGCGGAAGCAGCGGACCTTCG
ACAACGGCAGCATCCCCCACCAGATCCACCTGGGCGAGCTGCACG
CCATCCTGCGGCGGCAGGAGGACTTCTACCCCTTCCTGAAGGACA
ACCGGGAGAAGATCGAGAAGATCCTGACCTTCCGGATCCCCTACT
ACGTGGGCCCCCTGGCCCGGGGCAACAGCCGGTTCGCCTGGATGA
CCCGGAAATCCGAGGAGACCATCACCCCCTGGAACTTCGAGGAGG
TGGTGGACAAGGGCGCCAGCGCCCAGAGCTTCATCGAGCGGATGA
CCAACTTCGACAAGAACCTGCCCAACGAGAAGGTGCTGCCCAAGC
ACAGCCTGCTGTACGAGTACTTCACCGTGTACAACGAGCTGACCA
AGGTGAAGTACGTGACCGAGGGCATGCGGAAGCCCGCCTTCCTGA
GCGGCGAGCAGAAGAAGGCCATCGTGGACCTGCTGTTCAAGACCA
ACCGGAAGGTGACCGTGAAGCAGCTGAAGGAGGACTACTTCAAGA
AGATCGAGTGCTTCGACAGCGTGGAGATCAGCGGCGTGGAGGACC
GGTTCAACGCCAGCCTGGGCACCTACCACGACCTGCTGAAGATCA
TCAAGGACAAGGACTTCCTGGACAACGAGGAGAACGAGGACATCC
TGGAGGACATCGTGCTGACCCTGACCCTGTTCGAGGACCGGGAGA
TGATCGAGGAGCGGCTGAAAACCTACGCCCACCTGTTCGACGACA
AGGTGATGAAGCAGCTGAAGCGGCGGCGGTACACCGGCTGGGGCC
GGCTGAGCCGGAAGCTGATCAACGGCATCCGGGACAAGCAGAGCG
GCAAGACCATCCTGGACTTCCTGAAATCCGACGGCTTCGCCAACC
GGAACTTCATGCAGCTGATCCACGACGACAGCCTGACCTTCAAGG
AGGACATCCAGAAGGCCCAGGTGAGCGGCCAGGGCGACAGCCTGC
ACGAGCACATCGCCAACCTGGCCGGCAGCCCCGCCATCAAGAAGG
GCATCCTGCAGACCGTGAAGGTGGTGGACGAGCTGGTGAAGGTGA
TGGGCCGGCACAAGCCCGAGAACATCGTGATCGAGATGGCCCGGG
AGAACCAGACCACCCAGAAGGGCCAGAAGAACAGCCGGGAGCGGA
TGAAGCGGATCGAGGAGGGCATCAAGGAGCTGGGCAGCCAGATCC
TGAAGGAGCACCCCGTGGAGAACACCCAGCTGCAGAACGAGAAGC
TGTACCTGTACTACCTGCAGAACGGCCGGGACATGTACGTGGACC
AGGAGCTGGACATCAACCGGCTGAGCGACTACGACGTGGCCGCCA
TCGTGCCCCAGAGCTTCCTGAAGGACGACAGCATCGACAACAAGG
TGCTGACCCGGAGCGACAAGGCCCGGGGCAAGAGCGACAACGTGC
CCAGCGAGGAGGTGGTGAAGAAGATGAAGAACTACTGGCGGCAGC
TGCTGAACGCCAAGCTGATCACCCAGCGGAAGTTCGACAACCTGA
CCAAGGCCGAGCGGGGCGGCCTGAGCGAGCTGGACAAGGCCGGCT
TCATCAAGCGGCAGCTGGTGGAGACCCGGCAGATCACCAAGCACG
TGGCCCAGATCCTGGACAGCCGGATGAACACCAAGTACGACGAGA
ACGACAAGCTGATCCGGGAGGTGAAGGTGATCACCCTGAAATCCA
AGCTGGTGAGCGACTTCCGGAAGGACTTCCAGTTCTACAAGGTGC
GGGAGATCAACAACTACCACCACGCCCACGACGCCTACCTGAACG
CCGTGGTGGGCACCGCCCTGATCAAGAAGTACCCCAAGCTGGAGA
GCGAGTTCGTGTACGGCGACTACAAGGTGTACGACGTGCGGAAGA
TGATCGCCAAGAGCGAGCAGGAGATCGGCAAGGCCACCGCCAAGT
ACTTCTTCTACAGCAACATCATGAACTTCTTCAAGACCGAGATCA
CCCTGGCCAACGGCGAGATCCGGAAGCGGCCCCTGATCGAGACCA
ACGGCGAGACCGGCGAGATCGTGTGGGACAAGGGCCGGGACTTCG
CCACCGTGCGGAAGGTGCTGAGCATGCCCCAGGTGAACATCGTGA
AGAAAACCGAGGTGCAGACCGGCGGCTTCAGCAAGGAGAGCATCC
TGCCCAAGCGGAACAGCGACAAGCTGATCGCCCGGAAGAAGGACT
GGGACCCCAAGAAGTACGGCGGCTTCGACAGCCCCACCGTGGCCT
ACAGCGTGCTGGTGGTGGCCAAGGTGGAGAAGGGCAAGAGCAAGA
AGCTGAAATCCGTGAAGGAGCTGCTGGGCATCACCATCATGGAGC
GGAGCAGCTTCGAGAAGAACCCCATCGACTTCCTGGAGGCCAAGG
GCTACAAGGAGGTGAAGAAGGACCTGATCATCAAGCTGCCCAAGT
ACAGCCTGTTCGAGCTGGAGAACGGCCGGAAGCGGATGCTGGCCA
GCGCCGGCGAGCTGCAGAAGGGCAACGAGCTGGCCCTGCCCAGCA
AGTACGTGAACTTCCTGTACCTGGCCAGCCACTACGAGAAGCTGA
AGGGCAGCCCCGAGGACAACGAGCAGAAGCAGCTGTTCGTGGAGC
AGCACAAGCACTACCTGGACGAGATCATCGAGCAGATCAGCGAGT
TCAGCAAGCGGGTGATCCTGGCCGACGCCAACCTGGACAAGGTGC
TGAGCGCCTACAACAAGCACCGGGACAAGCCCATCCGGGAGCAGG
CCGAGAACATCATCCACCTGTTCACCCTGACCAACCTGGGCGCCC
CCGCCGCCTTCAAGTACTTCGACACCACCATCGACCGGAAGCGGT
ACACCAGCACCAAGGAGGTGCTGGACGCCACCCTGATCCACCAGA
GCATCACCGGCCTGTACGAGACCCGGATCGACCTGAGCCAGCTGG
GCGGCGACAGCGGCGGCAAGCGGCCCGCCGCCACCAAGAAGGCCG
GCCAGGCCAAGAAGAAGAAGTCGGGCGGGGGTGGCTCAGACGCTA
AGTCTCTGACCGCTTGGAGCAGAACACTGGTCACCTTCAAGGACG
TGTTCGTCGACTTCACAAGAGAGGAGTGGAAACTGCTGGACACCG
CCCAGCAGATCCTCTATAGAAACGTCATGCTGGAGAACTACAAGA
ATCTGGTGTCTCTGGGCTACCAGCTGACCAAGCCCGACGTGATTC
TGAGGCTGGAGAAGGGCGAGGAGCCTTGGCTGGTGGAGAGAGAGA
TCCACCAAGAAACCCACCCCGACAGCGAAACCGCCTTCGAGATCA
AGAGCAGCGTGGGAGGTGGCGGATCGGGAAAGCGGCCCGCCGCCA
CCAAGAAGGCCGGTCAGGCCAAGAAGAAGAAGGGCAGCTACCCCT
ACGACGTGCCCGACTACGCCTGAGCGGCCGCTTAATTAAGCTGCC
TTCTGCGGGGCTTGCCTTCTGGCCATGCCCTTCTTCTCTCCCTTG
CACCTGTACCTCTTGGTCTTTGAATAAAGCCTGAGTAGGAAGTCT
AGAAAAAAAAAAAAAAAAAAAAAAAAAAAAAAAAAAAAAAAAAAA
AAAAAAAAAAAAAAAAAAAAAAAAAAAAAAAAAAAAA

In some embodiments, a site-specific disrupting agent comprises the amino acid sequence of SEQ ID NOs: 86 or 82. In some embodiments, a site-specific disrupting agent described herein comprises an amino acid sequence of SEQ ID NO: 86 or 82 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

EZH2-dCas9-KRAB protein
(SEQ ID NO. 86)
MAPKKKRKVGGSGGSGQTGKKSEKGPVCWRKRVKSEYMRLRQLKR
FRRADEVKSMFSSNRQKLQQNFMVEDETVLHNIPYMGDEVLDQDG
TFIEELIKNYDGKVHGDRECGFINDEIFVELVNALGQYNDDDDDD
DGDDPEEREEKQKDLEDHRDDKESRPPRKFPSDKIFEAISSMFPD
KGTAEELKEKYKELTEQQLPGALPPECTPNIDGPNAKSVQREQSL
HSFHTLFCRRCFKYDCFLHPFHATPNTYKRKNTETALDNKPCGPQ
CYQHLEGAKEFAAALTAERIKTPPKRPGGRRRGRLPNNSSRPSTP
TINVLESKDTDSDREAGTETGGENNDKEEEEKKDETSSSSEANSR
CQTPIKMKPNIEPPENVEWSGAEASMFRVLIGTYYDNFCAIARLI
GTKTCRQVYEFRVKESSIIAPAPAEDVDTPPRKKKRKHRLWAAHC
RKIQLKKDGSSNHVYNYQPCDHPRQPCDSSCPCVIAQNFCEKFCQ
CSSECQNRFPGCRCKAQCNTKQCPCYLAVRECDPDLCLTCGAADH
WDSKNVSCKNCSIQRGSKKHLLLAPSDVAGWGIFIKDPVQKNEFI
SEYCGEIISQDEADRRGKVYDKYMCSFLFNLNNDFVVDATRKGNK
IRFANHSVNPNCYAKVMMVNGDHRIGIFAKRAIQTGEELFFDYRY
SQADALKYVGIEREMEIPSTGGSGGSGGSGGSGGSGRPDKKYSIG
LAIGTNSVGWAVITDEYKVPSKKFKVLGNTDRHSIKKNLIGALLF
DSGETAEATRLKRTARRRYTRRKNRICYLQEIFSNEMAKVDDSFF
HRLEESFLVEEDKKHERHPIFGNIVDEVAYHEKYPTIYHLRKKLV
DSTDKADLRLIYLALAHMIKFRGHFLIEGDLNPDNSDVDKLFIQL
VQTYNQLFEENPINASGVDAKAILSARLSKSRRLENLIAQLPGEK
KNGLFGNLIALSLGLTPNFKSNFDLAEDAKLQLSKDTYDDDLDNL
LAQIGDQYADLFLAAKNLSDAILLSDILRVNTEITKAPLSASMIK
RYDEHHQDLTLLKALVRQQLPEKYKEIFFDQSKNGYAGYIDGGAS
QEEFYKFIKPILEKMDGTEELLVKLNREDLLRKQRTFDNGSIPHQ
IHLGELHAILRRQEDFYPFLKDNREKIEKILTFRIPYYVGPLARG
NSRFAWMTRKSEETITPWNFEEVVDKGASAQSFIERMTNFDKNLP
NEKVLPKHSLLYEYFTVYNELTKVKYVTEGMRKPAFLSGEQKKAI
VDLLFKTNRKVTVKQLKEDYFKKIECFDSVEISGVEDRFNASLGT
YHDLLKIIKDKDFLDNEENEDILEDIVLTLTLFEDREMIEERLKT
YAHLFDDKVMKQLKRRRYTGWGRLSRKLINGIRDKQSGKTILDFL
KSDGFANRNFMQLIHDDSLTFKEDIQKAQVSGQGDSLHEHIANLA
GSPAIKKGILQTVKVVDELVKVMGRHKPENIVIEMARENQTTQKG
QKNSRERMKRIEEGIKELGSQILKEHPVENTQLQNEKLYLYYLQN
GRDMYVDQELDINRLSDYDVAAIVPQSFLKDDSIDNKVLTRSDKA
RGKSDNVPSEEVVKKMKNYWRQLLNAKLITQRKFDNLTKAERGGL
SELDKAGFIKRQLVETRQITKHVAQILDSRMNTKYDENDKLIREV
KVITLKSKLVSDFRKDFQFYKVREINNYHHAHDAYLNAVVGTALI
KKYPKLESEFVYGDYKVYDVRKMIAKSEQEIGKATAKYFFYSNIM
NFFKTEITLANGEIRKRPLIETNGETGEIVWDKGRDFATVRKVLS
MPQVNIVKKTEVQTGGFSKESILPKRNSDKLIARKKDWDPKKYGG
FDSPTVAYSVLVVAKVEKGKSKKLKSVKELLGITIMERSSFEKNP
IDFLEAKGYKEVKKDLIIKLPKYSLFELENGRKRMLASAGELQKG
NELALYNKHRDKPIREQAENIIHLFTLTNLGAPAAFKYFDTTIDR
KRYTSTKEVLDATLIHQSITGLYETRIDLSQLGGDSGGKRPAATK
KAGQAKKKKSGGGGSDAKSLTAWSRTLVTFKDVFVDFTREEWKLL
DTAQQILYRNVMLENYKNLVSLGYQLTKPDVILRLEKGEEPWLVE
REIHQETHPDSETAFEIKSSVGGGGSGKRPAATKKAGQAKKKKGS
YPYDVPDYA
EZH2-dCas9-KRAB protein without HA tag
(SEQ ID NO. 82)
MAPKKKRKVGGSGGSGQTGKKSEKGPVCWRKRVKSEYMRLRQLKR
FRRADEVKSMFSSNRQKILERTEILNQEWKQRRIQPVHILTSVSS
LRGTRECSVTSDLDFPTQVIPLKTLNAVASVPIMYSWSPLQQNFM
VEDETVLHNIPYMGDEVLDQDGTFIEELIKNYDGKVHGDRECGFI
NDEIFVELVNALGQYNDDDDDDDGDDPEEREEKQKDLEDHRDDKE
SRPPRKFPSDKIFEAISSMFPDKGTAEELKEKYKELTEQQLPGAL
PPECTPNIDGPNAKSVQREQSLHSFHTLFCRRCFKYDCFLHPFHA
TPNTYKRKNTETALDNKPCGPQCYQHLEGAKEFAAALTAERIKTP
PKRPGGRRRGRLPNNSSRPSTPTINVLESKDTDSDREAGTETGGE
NNDKEEEEKKDETSSSSEANSRCQTPIKMKPNIEPPENVEWSGAE
ASMFRVLIGTYYDNFCAIARLIGTKTCRQVYEFRVKESSIIAPAP
AEDVDTPPRKKKRKHRLWAAHCRKIQLKKDGSSNHVYNYQPCDHP
RQPCDSSCPCVIAQNFCEKFCQCSSECQNRFPGCRCKAQCNTKQC
PCYLAVRECDPDLCLTCGAADHWDSKNVSCKNCSIQRGSKKHLLL
APSDVAGWGIFIKDPVQKNEFISEYCGEIISQDEADRRGKVYDKY
MCSFLFNLNNDFVVDATRKGNKIRFANHSVNPNCYAKVMMVNGDH
RIGIFAKRAIQTGEELFFDYRYSQADALKYVGIEREMEIPSTGGS
GGSGGSGGSGGSGRPDKKYSIGLAIGTNSVGWAVITDEYKVPSKK
FKVLGNTDRHSIKKNLIGALLFDSGETAEATRLKRTARRRYTRRK
NRICYLQEIFSNEMAKVDDSFFHRLEESFLVEEDKKHERHPIFGN
IVDEVAYHEKYPTIYHLRKKLVDSTDKADLRLIYLALAHMIKFRG
HFLIEGDLNPDNSDVDKLFIQLVQTYNQLFEENPINASGVDAKAI
LSARLSKSRRLENLIAQLPGEKKNGLFGNLIALSLGLTPNFKSNF
DLAEDAKLQLSKDTYDDDLDNLLAQIGDQYADLFLAAKNLSDAIL
LSDILRVNTEITKAPLSASMIKRYDEHHQDLTLLKALVRQQLPEK
YKEIFFDQSKNGYAGYIDGGASQEEFYKFIKPILEKMDGTEELLV
KLNREDLLRKQRTFDNGSIPHQIHLGELHAILRRQEDFYPFLKDN
REKIEKILTFRIPYYVGPLARGNSRFAWMTRKSEETITPWNFEEV
VDKGASAQSFIERMTNFDKNLPNEKVLPKHSLLYEYFTVYNELTK
VKYVTEGMRKPAFLSGEQKKAIVDLLFKTNRKVTVKQLKEDYFKK
IECFDSVEISGVEDRFNASLGTYHDLLKIIKDKDFLDNEENEDIL
EDIVLTLTLFEDREMIEERLKTYAHLFDDKVMKQLKRRRYTGWGR
LSRKLINGIRDKQSGKTILDFLKSDGFANRNFMQLIHDDSLTFKE
DIQKAQVSGQGDSLHEHIANLAGSPAIKKGILQTVKVVDELVKVM
GRHKPENIVIEMARENQTTQKGQKNSRERMKRIEEGIKELGSQIL
KEHPVENTQLQNEKLYLYYLQNGRDMYVDQELDINRLSDYDVAAI
VPQSFLKDDSIDNKVLTRSDKARGKSDNVPSEEVVKKMKNYWRQL
LNAKLITQRKFDNLTKAERGGLSELDKAGFIKRQLVETRQITKHV
AQILDSRMNTKYDENDKLIREVKVITLKSKLVSDFRKDFQFYKVR
EINNYHHAHDAYLNAVVGTALIKKYPKLESEFVYGDYKVYDVRKM
IAKSEQEIGKATAKYFFYSNIMNFFKTEITLANGEIRKRPLIETN
GETGEIVWDKGRDFATVRKVLSMPQVNIVKKTEVQTGGFSKESIL
PKRNSDKLIARKKDWDPKKYGGFDSPTVAYSVLVVAKVEKGKSKK
LKSVKELLGITIMERSSFEKNPIDFLEAKGYKEVKKDLIIKLPKY
SLFELENGRKRMLASAGELQKGNELALYNKHRDKPIREQAENIIH
LFTLTNLGAPAAFKYFDTTIDRKRYTSTKEVLDATLIHQSITGLY
ETRIDLSQLGGDSGGKRPAATKKAGQAKKKKSGGGGSDAKSLTAW
SRTLVTFKDVFVDFTREEWKLLDTAQQILYRNVMLENYKNLVSLG
YQLTKPDVILRLEKGEEPWLVEREIHQETHPDSETAFEIKSSVGG
GGSGKRPAATKKAGQAKKKKGS

In some embodiments, a site-specific disrupting agent comprises a CRISPR/Cas molecule comprising Cas9. In some embodiments, the site-specific disrupting agent is encoded by the nucleic acid sequence of SEQ ID NO: 217 (e.g., mRNA encoding the site-specific disrupting agent). In some embodiments, a nucleic acid described herein comprises a nucleic acid sequence of SEQ ID NO: 217 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Cas9 mRNA (MR-28127)
(SEQ ID NO: 217)
AGGAAAUAAGAGAGAAAAGAAGAGUAAGAAGAAAUAUAAGAGCCA
CCAUGGCCCCCAAGAAGAAGCGGAAGGUGGGCAUCCACGGCGUGC
CCGCCGCCGACAAGAAGUACAGCAUCGGCCUGGACAUCGGCACCA
ACAGCGUGGGCUGGGCCGUGAUCACCGACGAGUACAAGGUGCCCA
GCAAGAAGUUCAAGGUGCUGGGCAACACCGACCGGCACAGCAUCA
AGAAGAACCUGAUCGGCGCCCUGCUGUUCGACAGCGGCGAGACCG
CCGAGGCCACCCGGCUGAAGCGGACCGCCCGGCGGCGGUACACCC
GGCGGAAGAACCGGAUCUGCUACCUGCAGGAGAUCUUCAGCAACG
AGAUGGCCAAGGUGGACGACAGCUUCUUCCACCGGCUGGAGGAGA
GCUUCCUGGUGGAGGAGGACAAGAAGCACGAGCGGCACCCCAUCU
UCGGCAACAUCGUGGACGAGGUGGCCUACCACGAGAAGUACCCCA
CCAUCUACCACCUGCGGAAGAAGCUGGUGGACAGCACCGACAAGG
CCGACCUGCGGCUGAUCUACCUGGCCCUGGCCCACAUGAUCAAGU
UCCGGGGCCACUUCCUGAUCGAGGGCGACCUGAACCCCGACAACA
GCGACGUGGACAAGCUGUUCAUCCAGCUGGUGCAGACCUACAACC
AGCUGUUCGAGGAGAACCCCAUCAACGCCAGCGGCGUGGACGCCA
AGGCCAUCCUGAGCGCCCGGCUGAGCAAGAGCCGGCGGCUGGAGA
ACCUGAUCGCCCAGCUGCCCGGCGAGAAGAAGAACGGCCUGUUCG
GCAACCUGAUCGCCCUGAGCCUGGGCCUGACCCCCAACUUCAAGA
GCAACUUCGACCUGGCCGAGGACGCCAAGCUGCAGCUGAGCAAGG
ACACCUACGACGACGACCUGGACAACCUGCUGGCCCAGAUCGGCG
ACCAGUACGCCGACCUGUUCCUGGCCGCCAAGAACCUGAGCGACG
CCAUCCUGCUGAGCGACAUCCUGCGGGUGAACACCGAGAUCACCA
AGGCCCCCCUGAGCGCCAGCAUGAUCAAGCGGUACGACGAGCACC
ACCAGGACCUGACCCUGCUGAAGGCCCUGGUGCGGCAGCAGCUGC
CCGAGAAGUACAAGGAGAUCUUCUUCGACCAGAGCAAGAACGGCU
ACGCCGGCUACAUCGACGGCGGCGCCAGCCAGGAGGAGUUCUACA
AGUUCAUCAAGCCCAUCCUGGAGAAGAUGGACGGCACCGAGGAGC
UGCUGGUGAAGCUGAACCGGGAGGACCUGCUGCGGAAGCAGCGGA
CCUUCGACAACGGCAGCAUCCCCCACCAGAUCCACCUGGGCGAGC
UGCACGCCAUCCUGCGGCGGCAGGAGGACUUCUACCCCUUCCUGA
AGGACAACCGGGAGAAGAUCGAGAAGAUCCUGACCUUCCGGAUCC
CCUACUACGUGGGCCCCCUGGCCCGGGGCAACAGCCGGUUCGCCU
GGAUGACCCGGAAAUCCGAGGAGACCAUCACCCCCUGGAACUUCG
AGGAGGUGGUGGACAAGGGCGCCAGCGCCCAGAGCUUCAUCGAGC
GGAUGACCAACUUCGACAAGAACCUGCCCAACGAGAAGGUGCUGC
CCAAGCACAGCCUGCUGUACGAGUACUUCACCGUGUACAACGAGC
UGACCAAGGUGAAGUACGUGACCGAGGGCAUGCGGAAGCCCGCCU
UCCUGAGCGGCGAGCAGAAGAAGGCCAUCGUGGACCUGCUGUUCA
AGACCAACCGGAAGGUGACCGUGAAGCAGCUGAAGGAGGACUACU
UCAAGAAGAUCGAGUGCUUCGACAGCGUGGAGAUCAGCGGCGUGG
AGGACCGGUUCAACGCCAGCCUGGGCACCUACCACGACCUGCUGA
AGAUCAUCAAGGACAAGGACUUCCUGGACAACGAGGAGAACGAGG
ACAUCCUGGAGGACAUCGUGCUGACCCUGACCCUGUUCGAGGACC
GGGAGAUGAUCGAGGAGCGGCUGAAAACCUACGCCCACCUGUUCG
ACGACAAGGUGAUGAAGCAGCUGAAGCGGCGGCGGUACACCGGCU
GGGGCCGGCUGAGCCGGAAGCUGAUCAACGGCAUCCGGGACAAGC
AGAGCGGCAAGACCAUCCUGGACUUCCUGAAAUCCGACGGCUUCG
CCAACCGGAACUUCAUGCAGCUGAUCCACGACGACAGCCUGACCU
UCAAGGAGGACAUCCAGAAGGCCCAGGUGAGCGGCCAGGGCGACA
GCCUGCACGAGCACAUCGCCAACCUGGCCGGCAGCCCCGCCAUCA
AGAAGGGCAUCCUGCAGACCGUGAAGGUGGUGGACGAGCUGGUGA
AGGUGAUGGGCCGGCACAAGCCCGAGAACAUCGUGAUCGAGAUGG
CCCGGGAGAACCAGACCACCCAGAAGGGCCAGAAGAACAGCCGGG
AGCGGAUGAAGCGGAUCGAGGAGGGCAUCAAGGAGCUGGGCAGCC
AGAUCCUGAAGGAGCACCCCGUGGAGAACACCCAGCUGCAGAACG
AGAAGCUGUACCUGUACUACCUGCAGAACGGCCGGGACAUGUACG
UGGACCAGGAGCUGGACAUCAACCGGCUGAGCGACUACGACGUGG
ACCACAUCGUGCCCCAGAGCUUCCUGAAGGACGACAGCAUCGACA
ACAAGGUGCUGACCCGGAGCGACAAGAACCGGGGCAAGAGCGACA
ACGUGCCCAGCGAGGAGGUGGUGAAGAAGAUGAAGAACUACUGGC
GGCAGCUGCUGAACGCCAAGCUGAUCACCCAGCGGAAGUUCGACA
ACCUGACCAAGGCCGAGCGGGGCGGCCUGAGCGAGCUGGACAAGG
CCGGCUUCAUCAAGCGGCAGCUGGUGGAGACCCGGCAGAUCACCA
AGCACGUGGCCCAGAUCCUGGACAGCCGGAUGAACACCAAGUACG
ACGAGAACGACAAGCUGAUCCGGGAGGUGAAGGUGAUCACCCUGA
AAUCCAAGCUGGUGAGCGACUUCCGGAAGGACUUCCAGUUCUACA
AGGUGCGGGAGAUCAACAACUACCACCACGCCCACGACGCCUACC
UGAACGCCGUGGUGGGCACCGCCCUGAUCAAGAAGUACCCCAAGC
UGGAGAGCGAGUUCGUGUACGGCGACUACAAGGUGUACGACGUGC
GGAAGAUGAUCGCCAAGAGCGAGCAGGAGAUCGGCAAGGCCACCG
CCAAGUACUUCUUCUACAGCAACAUCAUGAACUUCUUCAAGACCG
AGAUCACCCUGGCCAACGGCGAGAUCCGGAAGCGGCCCCUGAUCG
AGACCAACGGCGAGACCGGCGAGAUCGUGUGGGACAAGGGCCGGG
ACUUCGCCACCGUGCGGAAGGUGCUGAGCAUGCCCCAGGUGAACA
UCGUGAAGAAAACCGAGGUGCAGACCGGCGGCUUCAGCAAGGAGA
GCAUCCUGCCCAAGCGGAACAGCGACAAGCUGAUCGCCCGGAAGA
AGGACUGGGACCCCAAGAAGUACGGCGGCUUCGACAGCCCCACCG
UGGCCUACAGCGUGCUGGUGGUGGCCAAGGUGGAGAAGGGCAAGA
GCAAGAAGCUGAAAUCCGUGAAGGAGCUGCUGGGCAUCACCAUCA
UGGAGCGGAGCAGCUUCGAGAAGAACCCCAUCGACUUCCUGGAGG
CCAAGGGCUACAAGGAGGUGAAGAAGGACCUGAUCAUCAAGCUGC
CCAAGUACAGCCUGUUCGAGCUGGAGAACGGCCGGAAGCGGAUGC
UGGCCAGCGCCGGCGAGCUGCAGAAGGGCAACGAGCUGGCCCUGC
CCAGCAAGUACGUGAACUUCCUGUACCUGGCCAGCCACUACGAGA
AGCUGAAGGGCAGCCCCGAGGACAACGAGCAGAAGCAGCUGUUCG
UGGAGCAGCACAAGCACUACCUGGACGAGAUCAUCGAGCAGAUCA
GCGAGUUCAGCAAGCGGGUGAUCCUGGCCGACGCCAACCUGGACA
AGGUGCUGAGCGCCUACAACAAGCACCGGGACAAGCCCAUCCGGG
AGCAGGCCGAGAACAUCAUCCACCUGUUCACCCUGACCAACCUGG
GCGCCCCCGCCGCCUUCAAGUACUUCGACACCACCAUCGACCGGA
AGCGGUACACCAGCACCAAGGAGGUGCUGGACGCCACCCUGAUCC
ACCAGAGCAUCACCGGCCUGUACGAGACCCGGAUCGACCUGAGCC
AGCUGGGCGGCGACAGCGGCGGCAAGCGGCCCGCCGCCACCAAGA
AGGCCGGCCAGGCCAAGAAGAAGAAGGGCAGCUACCCCUACGACG
UGCCCGACUACGCCUGAGCGGCCGCUUAAUUAAGCUGCCUUCUGC
GGGGCUUGCCUUCUGGCCAUGCCCUUCUUCUCUCCCUUGCACCUG
UACCUCUUGGUCUUUGAAUAAAGCCUGAGUAGGAAGUCUAGAAAA
AAAAAAAAAAAAAAAAAAAAAAAAAAAAAAAAAAAAAAAAAAAAA
AAAAAAAAAAAAAAAAAAAAAAAAAAAAAAA

In some embodiments, a site-specific disrupting agent comprises the amino acid sequence of SEQ ID NOs: 218 or 84. In some embodiments, a site-specific disrupting agent described herein comprises an amino acid sequence of SEQ ID NO: 218 or 84 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

Cas9 Protein Sequence (corresponding
to MR-28127)
(SEQ ID NO: 218)
MAPKKKRKVGIHGVPAADKKYSIGLDIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVD
HIVPQSFLKDDSIDNKVLTRSDKNRGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSGGKRPAATKK
AGQAKKKKGSYPYDVPDYA
Cas9 Protein Sequence (corresponding
to MR-28127) without HA tag
(SEQ ID NO: 84)
MAPKKKRKVGIHGVPAADKKYSIGLDIGTNSVGWAVITDEYKVPS
KKFKVLGNTDRHSIKKNLIGALLFDSGETAEATRLKRTARRRYTR
RKNRICYLQEIFSNEMAKVDDSFFHRLEESFLVEEDKKHERHPIF
GNIVDEVAYHEKYPTIYHLRKKLVDSTDKADLRLIYLALAHMIKF
RGHFLIEGDLNPDNSDVDKLFIQLVQTYNQLFEENPINASGVDAK
AILSARLSKSRRLENLIAQLPGEKKNGLFGNLIALSLGLTPNFKS
NFDLAEDAKLQLSKDTYDDDLDNLLAQIGDQYADLFLAAKNLSDA
ILLSDILRVNTEITKAPLSASMIKRYDEHHQDLTLLKALVRQQLP
EKYKEIFFDQSKNGYAGYIDGGASQEEFYKFIKPILEKMDGTEEL
LVKLNREDLLRKQRTFDNGSIPHQIHLGELHAILRRQEDFYPFLK
DNREKIEKILTFRIPYYVGPLARGNSRFAWMTRKSEETITPWNFE
EVVDKGASAQSFIERMTNFDKNLPNEKVLPKHSLLYEYFTVYNEL
TKVKYVTEGMRKPAFLSGEQKKAIVDLLFKTNRKVTVKQLKEDYF
KKIECFDSVEISGVEDRFNASLGTYHDLLKIIKDKDFLDNEENED
ILEDIVLTLTLFEDREMIEERLKTYAHLFDDKVMKQLKRRRYTGW
GRLSRKLINGIRDKQSGKTILDFLKSDGFANRNFMQLIHDDSLTF
KEDIQKAQVSGQGDSLHEHIANLAGSPAIKKGILQTVKVVDELVK
VMGRHKPENIVIEMARENQTTQKGQKNSRERMKRIEEGIKELGSQ
ILKEHPVENTQLQNEKLYLYYLQNGRDMYVDQELDINRLSDYDVD
HIVPQSFLKDDSIDNKVLTRSDKNRGKSDNVPSEEVVKKMKNYWR
QLLNAKLITQRKFDNLTKAERGGLSELDKAGFIKRQLVETRQITK
HVAQILDSRMNTKYDENDKLIREVKVITLKSKLVSDFRKDFQFYK
VREINNYHHAHDAYLNAVVGTALIKKYPKLESEFVYGDYKVYDVR
KMIAKSEQEIGKATAKYFFYSNIMNFFKTEITLANGEIRKRPLIE
TNGETGEIVWDKGRDFATVRKVLSMPQVNIVKKTEVQTGGFSKES
ILPKRNSDKLIARKKDWDPKKYGGFDSPTVAYSVLVVAKVEKGKS
KKLKSVKELLGITIMERSSFEKNPIDFLEAKGYKEVKKDLIIKLP
KYSLFELENGRKRMLASAGELQKGNELALPSKYVNFLYLASHYEK
LKGSPEDNEQKQLFVEQHKHYLDEIIEQISEFSKRVILADANLDK
VLSAYNKHRDKPIREQAENIIHLFTLTNLGAPAAFKYFDTTIDRK
RYTSTKEVLDATLIHQSITGLYETRIDLSQLGGDSGGKRPAATKK
AGQAKKKKGS

In some embodiments, a site-specific disrupting agent comprises a nuclear localization sequence (NLS). In some embodiments, the site-specific disrupting agent comprises an NLS, e.g., an SV40 NLS at the N-terminus. In some embodiments, the site-specific disrupting agent comprises an NLS, e.g., an SV40 NLS at the C-terminus. In some embodiments, the site-specific disrupting agent comprises an NLS, e.g., a nucleoplasmin NLS at the C-terminus. In some embodiments, the site-specific disrupting agent comprises a first NLS at the N-terminus and a second NLS at the C-terminus. In some embodiments the first and the second NLS have the same sequence. In some embodiments, the first and the second NLS have different sequences. In some embodiments, the site-specific disrupting agent comprises a first NLS at the N-terminus, a second NLS, and a third NLS at the C-terminus. In some embodiments, at least two NLSs have the same sequence. In some embodiments, the first and the second NLS have the same sequence and the third NLS has a different sequence than the first and the second NLS. In some embodiments, the site-specific disrupting agent comprises an SV40 NLS, e.g., the site-specific disrupting agent comprises a sequence according to PKKKRK (SEQ ID NO: 63). In some embodiments, the site-specific disrupting agent comprises a nucleoplasmin NLS, e.g., the site-specific disrupting agent comprises a sequence of KRPAATKKAGQAKKK (SEQ ID NO: 64). In some embodiments, the site-specific disrupting agent comprises a C-terminal sequence comprising one or more of, e.g., any one or both of: a nucleoplasmin nuclear localization sequence and an HA-tag. In some embodiments, the site-specific disrupting agent comprises an epitope tag, e.g., an HA tag: YPYDVPDYA (SEQ ID NO: 65). In some embodiments, the site-specific disrupting agent may comprise two copies of the epitope tag.

While an epitope tag is useful in many research contexts, it is sometimes desirable to omit an epitope tag in a therapeutic context. Accordingly, in some embodiments, the site-specific disrupting agent lacks an epitope tag. In some embodiments, a site-specific disrupting agent described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking the HA tag of SEQ ID NO: 65. In some embodiments, a nucleic acid described herein comprises a sequence provided herein (or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto), but lacking a region encoding the HA tag of SEQ ID NO: 65. In some embodiments, the site-specific disrupting agent does not comprise an NLS. In some embodiments, the site-specific disrupting agent does not comprise an epitope tag. In some embodiments the site-specific disrupting agent does not comprise an HA tag. In some embodiments, the site-specific disrupting agent does not comprise an HA tag sequence according to SEQ ID NO: 65.

In some embodiments, a site-specific disrupting agent comprises a targeting moiety comprising a Zn finger molecule and an effector moiety comprising EZH2 or a functional fragment or variant thereof. In some embodiments, the site-specific disrupting agent is encoded by the nucleic acid sequence of SEQ ID NOs: 219, 220, 222, 223, 233, or 234, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a site-specific disrupting agent comprises a targeting moiety comprising a Zn finger molecule and an effector moiety comprising DNMT3 (e.g., DNMT3a or DNMT3L) or a functional fragment or variant thereof. In some embodiments, the site-specific disrupting agent is encoded by the nucleic acid sequence of SEQ ID NOs: 221, 231, or 236-239, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a site-specific disrupting agent comprises a targeting moiety comprising a Zn finger molecule and an effector moiety comprising G9A or a functional fragment or variant thereof. In some embodiments, the site-specific disrupting agent is encoded by the nucleic acid sequence of SEQ ID NOs: 224, 225, or 227-230, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

In some embodiments, a site-specific disrupting agent comprises a targeting moiety comprising a Zn finger molecule and an effector moiety comprising HDAC8 or a functional fragment or variant thereof. In some embodiments, the site-specific disrupting agent is encoded by the nucleic acid sequence of SEQ ID NOs: 226, 232, 235, or 240-242, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

TABLE 3
Additional Exemplary Site-Specific Disrupting Agent Encoding Sequences

| Ref. | | |
|---|---|---|
| number | | |
| and | | |
| (SEQ | Agent | |
| ID NO) | Description | Sequence |
| MR- | EZH2- | AGGAAATAAGAGAGAAAAGAAGAGTAAGAAGAAATATAAGAGCCACCATG |
| 29950 | CXCL1 ZF6 | GCCCCCAAGAAGAAGCGGAAGGTGGGCGGCAGCGGCGGCAGCGGCCAGAC |
| (SEQ ID | T7 | CGGCAAGAAGAGCGAGAAGGGCCCCGTGTGCTGGCGGAAGCGGGTGAAGA |
| NO: 219) | | GCGAGTACATGCGGCTGCGGCAGCTGAAGCGGTTCCGGCGGGCCGACGAG |
| | | GTGAAGAGCATGTTCAGCAGCAACCGGCAGAAGATCCTGGAGCGGACCGA |
| | | GATCCTGAACCAGGAGTGGAAGCAGCGGCGAATCCAGCCCGTGCACATCC |
| | | TGACCAGCGTGAGCAGCCTGCGGGGCACCCGGGAGTGCAGCGTGACCAGC |
| | | GACCTGGACTTCCCCACCCAGGTGATCCCCCTAAAGACCCTGAACGCCGT |
| | | GGCCAGCGTGCCCATCATGTACAGCTGGAGCCCCCTGCAGCAGAACTTCA |
| | | TGGTGGAGGACGAGACCGTGCTGCACAACATCCCCTACATGGGCGACGAG |
| | | GTGCTGGACCAGGACGGCACCTTCATCGAGGAGCTGATCAAGAACTACGA |
| | | CGGCAAGGTGCACGGCGACCGGGAGTGCGGCTTCATCAACGACGAGATCT |
| | | TCGTGGAGCTGGTGAACGCCCTGGGCCAGTACAACGACGACGACGACGAC |
| | | GACGACGGCGACGACCCCGAGGAGCGGGAGGAGAAGCAGAAGGACCTGGA |
| | | GGACCACCGGGACGACAAGGAGAGCCGGCCCCCCCGGAAGTTCCCCAGCG |
| | | ACAAGATCTTCGAGGCCATCAGCAGCATGTTCCCCGACAAGGGCACCGCC |
| | | GAGGAGCTGAAGGAGAAGTACAAGGAGCTGACCGAGCAGCAGCTGCCCGG |
| | | CGCCCTGCCCCCCGAGTGCACCCCCAACATCGACGGCCCCAACGCCAAGA |
| | | GCGTGCAGCGGGAGCAGAGCCTGCACAGCTTCCACACCCTGTTCTGCCGG |
| | | CGGTGCTTCAAGTACGACTGCTTCCTGCACCCCTTCCACGCCACCCCCAA |
| | | CACCTACAAGCGGAAGAACACCGAGACCGCCCTGGACAACAAGCCCTGCG |
| | | GCCCCCAGTGCTACCAGCACCTGGAGGGCGCCAAGGAGTTCGCCGCCGCC |
| | | CTGACCGCCGAGCGGATCAAGACCCCCCCCAAGCGGCCCGGCGGCCGGCG |
| | | GCGGGGCCGGCTGCCCAACAACAGCAGCCGGCCCAGCACCCCCACCATCA |
| | | ACGTGCTGGAGAGCAAGGACACCGACAGCGACCGGGAGGCCGGCACCGAG |
| | | ACCGGCGGCGAGAACAACGACAAGGAGGAGGAGGAGAAGAAGGACGAGAC |
| | | CAGCAGCAGCAGCGAGGCCAACAGCCGGTGCCAGACCCCCATCAAGATGA |
| | | AGCCCAACATCGAGCCCCCCGAGAACGTGGAGTGGAGCGGCGCCGAGGCC |
| | | AGCATGTTCCGGGTGCTGATCGGCACCTACTACGACAACTTCTGCGCCAT |
| | | CGCCCGGCTGATCGGCACCAAGACCTGCCGGCAGGTGTACGAGTTCCGGG |
| | | TGAAGGAGAGCAGCATCATCGCCCCCGCCCCCGCCGAGGACGTGGACACC |
| | | CCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACC |
| | | AGCCCTGCGACCACCCCCGGCAGCCCTGCGACAGCAGCTGCCCCTGCGTG |
| | | ATCGCCCAGAACTTCTGCGAGAAGTTCTGCCAGTGCAGCAGCGAGTGCCA |
| | | GAACCGGTTCCCCGGCTGCCGGTGCAAGGCCCAGTGCAACACCAAGCAGT |
| | | GCCCCTGCTACCTGGCCGTGCGGGAGTGCGACCCCGACCTGTGCCTGACC |
| | | TGCGGCGCCGCCGACCACTGGGACAGCAAGAACGTGAGCTGCAAGAACTG |
| | | CAGCATCCAGCGGGGCAGCAAGAAGCACCTGCTGCTGGCCCCCAGCGACG |
| | | TGGCCGGCTGGGGCATCTTCATCAAGGACCCCGTGCAGAAGAACGAGTTC |
| | | ATCAGCGAGTACTGCGGCGAGATCATCAGCCAGGACGAGGCCGACCGGCG |
| | | GGGCAAGGTGTACGACAAGTACATGTGCAGCTTCCTGTTCAACCTGAACA |
| | | ACGACTTCGTGGTGGACGCCACCCGGAAGGGCAACAAGATCCGGTTCGCC |
| | | AACCACAGCGTGAACCCCAACTGCTACGCCAAGGTGATGATGGTGAACGG |
| | | CGACCACCGGATCGGCATCTTCGCCAAGCGGGCCATCCAGACCGGCGAGG |
| | | AGCTGTTCTTCGACTACCGGTACAGCCAGGCCGACGCCCTGAAGTACGTG |
| | | GGCATCGAGCGGGAGATGGAGATCCCCGGCAGCAGCGGATCCCTGGAGCC |
| | | CGGCGAAAAGCCTTACAAGTGTCCCGAGTGCGGAAAGAGCTTCAGCAGAG |
| | | CCGATAATCTGACCGAGCACCAAAGGACCCACACCGGAGAGAAGCCTTAT |
| | | AAGTGTCCCGAATGCGGCAAAAGCTTTTCTAGAAGCGATCATCTGACCAA |
| | | CCACCAGAGGACACACACCGGAGAAAAACCTTACAAATGCCCCGAGTGCG |
| | | GCAAAAGCTTCTCCCAGAGCAGCAATCTGGTGAGACACCAAAGGACCCAC |
| | | ACCGGCGAAAAACCCTATAAATGCCCCGAATGTGGCAAGAGCTTTAGCAC |
| | | ATCCGGCGAGCTGGTGAGGCATCAAAGAACACATACCGGCGAGAAGCCCT |
| | | ACAAGTGCCCCGAGTGTGGAAAAAGCTTCAGCACCCACCTCGATCTGATC |
| | | AGACACCAGAGGACCCATACCGGAGAGAAACCCTACAAATGTCCCGAGTG |
| | | CGGAAAGTCCTTTAGCCAGCTGGCCCATCTGAGAGCTCATCAAAGGACAC |
| | | ACACCGGCGAGAAGCCTTACAAGTGTCCCGAGTGCGGAAAATCCTTCTCC |
| | | CAACTGGCCCATCTGAGGGCCCACCAGAGAACCCACACCGGCAAAAAGAC |
| | | CTCCGCTAGCGGCAGCGGCGGCGGCAGCGGCGGCAAGCGGCCCGCCGCCA |
| | | CCAAGAAGGCCGGCCAGGCCAAGAAGAAGAAGGGCAGCTACCCCTACGAC |
| | | GTGCCCGACTACGCCTGAGCGGCCGCTTAATTAAGCTGCCTTCTGCGGGG |
| | | CTTGCCTTCTGGCCATGCCCTTCTTCTCTCCCTTGCACCTGTACCTCTTG |
| | | GTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAA |
| | | AAAAAAAAAAAA |
| MR- | EZH2- | AGGAAATAAGAGAGAAAAGAAGAGTAAGAAGAAATATAAGAGCCACCATG |
| 29949 | CXCL1 ZF1 | GCCCCCAAGAAGAAGCGGAAGGTGGGCGGCAGCGGCGGCAGCGGCCAGAC |
| (SEQ ID | T7 | CGGCAAGAAGAGCGAGAAGGGCCCCGTGTGCTGGCGGAAGCGGGTGAAGA |
| NO: 220) | | GCGAGTACATGCGGCTGCGGCAGCTGAAGCGGTTCCGGCGGGCCGACGAG |
| | | GTGAAGAGCATGTTCAGCAGCAACCGGCAGAAGATCCTGGAGCGGACCGA |
| | | GATCCTGAACCAGGAGTGGAAGCAGCGGCGAATCCAGCCCGTGCACATCC |
| | | TGACCAGCGTGAGCAGCCTGCGGGGCACCCGGGAGTGCAGCGTGACCAGC |
| | | GACCTGGACTTCCCCACCCAGGTGATCCCCCTAAAGACCCTGAACGCCGT |
| | | GGCCAGCGTGCCCATCATGTACAGCTGGAGCCCCCTGCAGCAGAACTTCA |
| | | TGGTGGAGGACGAGACCGTGCTGCACAACATCCCCTACATGGGCGACGAG |
| | | GTGCTGGACCAGGACGGCACCTTCATCGAGGAGCTGATCAAGAACTACGA |
| | | CGGCAAGGTGCACGGCGACCGGGAGTGCGGCTTCATCAACGACGAGATCT |
| | | TCGTGGAGCTGGTGAACGCCCTGGGCCAGTACAACGACGACGACGACGAC |
| | | GACGACGGCGACGACCCCGAGGAGCGGGAGGAGAAGCAGAAGGACCTGGA |
| | | GGACCACCGGGACGACAAGGAGAGCCGGCCCCCCCGGAAGTTCCCCAGCG |
| | | ACAAGATCTTCGAGGCCATCAGCAGCATGTTCCCCGACAAGGGCACCGCC |
| | | GAGGAGCTGAAGGAGAAGTACAAGGAGCTGACCGAGCAGCAGCTGCCCGG |
| | | CGCCCTGCCCCCCGAGTGCACCCCCAACATCGACGGCCCCAACGCCAAGA |
| | | GCGTGCAGCGGGAGCAGAGCCTGCACAGCTTCCACACCCTGTTCTGCCGG |
| | | CGGTGCTTCAAGTACGACTGCTTCCTGCACCCCTTCCACGCCACCCCCAA |
| | | CACCTACAAGCGGAAGAACACCGAGACCGCCCTGGACAACAAGCCCTGCG |
| | | GCCCCCAGTGCTACCAGCACCTGGAGGGCGCCAAGGAGTTCGCCGCCGCC |
| | | CTGACCGCCGAGCGGATCAAGACCCCCCCCAAGCGGCCCGGCGGCCGGCG |
| | | GCGGGGCCGGCTGCCCAACAACAGCAGCCGGCCCAGCACCCCCACCATCA |
| | | ACGTGCTGGAGAGCAAGGACACCGACAGCGACCGGGAGGCCGGCACCGAG |
| | | ACCGGCGGCGAGAACAACGACAAGGAGGAGGAGGAGAAGAAGGACGAGAC |
| | | CAGCAGCAGCAGCGAGGCCAACAGCCGGTGCCAGACCCCCATCAAGATGA |
| | | AGCCCAACATCGAGCCCCCCGAGAACGTGGAGTGGAGCGGCGCCGAGGCC |
| | | AGCATGTTCCGGGTGCTGATCGGCACCTACTACGACAACTTCTGCGCCAT |
| | | CGCCCGGCTGATCGGCACCAAGACCTGCCGGCAGGTGTACGAGTTCCGGG |
| | | TGAAGGAGAGCAGCATCATCGCCCCCGCCCCCGCCGAGGACGTGGACACC |
| | | CCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACC |
| | | AGCCCTGCGACCACCCCCGGCAGCCCTGCGACAGCAGCTGCCCCTGCGTG |
| | | ATCGCCCAGAACTTCTGCGAGAAGTTCTGCCAGTGCAGCAGCGAGTGCCA |
| | | GAACCGGTTCCCCGGCTGCCGGTGCAAGGCCCAGTGCAACACCAAGCAGT |
| | | GCCCCTGCTACCTGGCCGTGCGGGAGTGCGACCCCGACCTGTGCCTGACC |
| | | TGCGGCGCCGCCGACCACTGGGACAGCAAGAACGTGAGCTGCAAGAACTG |
| | | CAGCATCCAGCGGGGCAGCAAGAAGCACCTGCTGCTGGCCCCCAGCGACG |
| | | TGGCCGGCTGGGGCATCTTCATCAAGGACCCCGTGCAGAAGAACGAGTTC |
| | | ATCAGCGAGTACTGCGGCGAGATCATCAGCCAGGACGAGGCCGACCGGCG |
| | | GGGCAAGGTGTACGACAAGTACATGTGCAGCTTCCTGTTCAACCTGAACA |
| | | ACGACTTCGTGGTGGACGCCACCCGGAAGGGCAACAAGATCCGGTTCGCC |
| | | AACCACAGCGTGAACCCCAACTGCTACGCCAAGGTGATGATGGTGAACGG |
| | | CGACCACCGGATCGGCATCTTCGCCAAGCGGGCCATCCAGACCGGCGAGG |
| | | AGCTGTTCTTCGACTACCGGTACAGCCAGGCCGACGCCCTGAAGTACGTG |
| | | GGCATCGAGCGGGAGATGGAGATCCCCGGCAGCAGCGGATCCCTGGAGCC |
| | | CGGCGAAAAGCCTTACAAATGTCCCGAATGCGGAAAGAGCTTCAGCAGAG |
| | | CCGACAATCTGACCGAACATCAGAGAACCCATACCGGAGAAAAACCTTAC |
| | | AAATGTCCCGAGTGCGGCAAAAGCTTCTCCCAAGCCGGACATCTGGCCAG |
| | | CCACCAAAGGACACATACCGGCGAGAAACCCTACAAGTGCCCCGAGTGCG |
| | | GCAAGTCCTTCTCTAGATCCGATGAGCTGGTCAGACATCAGAGAACCCAT |
| | | ACCGGCGAGAAGCCTTATAAGTGCCCCGAATGTGGCAAGTCCTTCAGCCA |
| | | GAGAGCTCATCTGGAGAGGCATCAAAGAACACACACCGGAGAGAAACCTT |
| | | ACAAGTGTCCCGAGTGTGGAAAGAGCTTCTCCAGAAGGGACGAGCTGAAC |
| | | GTCCACCAAAGAACCCATACCGGCGAAAAGCCCTATAAATGCCCCGAGTG |
| | | TGGAAAATCCTTTTCTAGATCCGACCATCTGACAACCCACCAGAGGACCC |
| | | ATACCGGAGAGAAGCCCTACAAATGCCCCGAGTGTGGAAAAAGCTTCTCT |
| | | AGAAACGATGCTCTGACAGAGCACCAAAGGACCCACACCGGCAAAAAGAC |
| | | CAGCGCTAGCGGCAGCGGCGGCGGCAGCGGCGGCAAGCGGCCCGCCGCCA |
| | | CCAAGAAGGCCGGCCAGGCCAAGAAGAAGAAGGGCAGCTACCCCTACGAC |
| | | GTGCCCGACTACGCCTGAGCGGCCGCTTAATTAAGCTGCCTTCTGCGGGG |
| | | CTTGCCTTCTGGCCATGCCCTTCTTCTCTCCCTTGCACCTGTACCTCTTG |
| | | GTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAA |
| | | AAAAAAAAAAAA |
| MR- | CXCL1 ZF5- | AGGAAATAAGAGAGAAAAGAAGAGTAAGAAGAAATATAAGAGCCACCATG |
| 29945 | DNMT3a-3L | GCCCCCAAGAAGAAGCGGAAGGTGGGCATCCACGGCGTGCCCGCCGCCGG |
| (SEQ ID | SP6 | CAGCAGCGGATCCCTGGAGCCCGGCGAAAAGCCTTACAAGTGCCCCGAGT |
| NO: 221) | | GTGGCAAATCCTTTAGCACCACCGGAAATCTGACCGTCCACCAGAGAACA |
| | | CATACCGGCGAGAAACCCTACAAGTGTCCCGAGTGCGGCAAATCCTTCAG |
| | | CCAGCTGGCCCATCTGAGAGCCCATCAAAGGACCCATACCGGCGAGAAAC |
| | | CTTACAAGTGTCCCGAATGTGGAAAGTCCTTTAGCAGCCCCGCCGATCTG |
| | | ACAAGACATCAAAGAACCCACACCGGCGAGAAGCCCTATAAATGTCCCGA |
| | | GTGTGGAAAGTCCTTCAGCCAGAGCGGCAATCTGACCGAGCATCAAAGAA |
| | | CCCATACCGGCGAAAAGCCCTATAAGTGCCCCGAATGCGGAAAAAGCTTC |
| | | TCCACAAGCGGCGAGCTGGTGAGACACCAAAGGACACATACCGGCGAAAA |
| | | GCCTTATAAATGCCCCGAGTGCGGCAAGAGCTTCTCTAGAAAGGACAATC |
| | | TGAAGAACCACCAAAGAACACACACCGGCGAGAAGCCCTACAAATGCCCC |
| | | GAGTGCGGCAAGAGCTTTAGCCAGTCCAGCAACCTCGTGAGACATCAGAG |
| | | GACACATACCGGAAAAAAGACCAGCGCTAGCGGCAGCGGCGGCGGCAGCG |
| | | GCGGCAACCACGACCAGGAGTTCGACCCCCCCAAGGTGTACCCCCCCGTG |
| | | CCCGCCGAGAAGCGGAAGCCCATCCGGGTGCTGAGCCTGTTCGACGGCAT |
| | | CGCCACCGGCCTGCTGGTGCTGAAGGACCTGGGCATCCAGGTGGACCGGT |
| | | ACATCGCCAGCGAGGTGTGCGAGGACAGCATCACCGTGGGCATGGTGCGG |
| | | CACCAGGGCAAGATCATGTACGTGGGCGACGTGCGGAGCGTGACCCAGAA |
| | | GCACATCCAGGAGTGGGGCCCCTTCGACCTGGTGATCGGCGGCAGCCCCT |
| | | GCAACGACCTGAGCATCGTGAACCCCGCCCGGAAGGGCCTGTACGAGGGC |
| | | ACCGGCCGGCTGTTCTTCGAGTTCTACCGGCTGCTGCACGACGCCCGGCC |
| | | CAAGGAGGGCGACGACCGGCCCTTCTTCTGGCTGTTCGAGAACGTGGTGG |
| | | CCATGGGCGTGAGCGACAAGCGGGACATCAGCCGGTTCCTGGAGAGCAAC |
| | | CCCGTGATGATCGACGCCAAGGAGGTGAGCGCCGCCCACCGGGCCCGGTA |
| | | CTTCTGGGGCAACCTGCCCGGCATGAACCGGCCCCTGGCCAGCACCGTGA |
| | | ACGACAAGCTGGAGCTGCAGGAGTGCCTGGAGCACGGCCGGATCGCCAAG |
| | | TTCAGCAAGGTGCGGACCATCACCACCCGGAGCAACAGCATCAAGCAGGG |
| | | CAAGGACCAGCACTTCCCCGTGTTCATGAACGAGAAGGAGGACATCCTGT |
| | | GGTGCACCGAGATGGAGCGGGTGTTCGGCTTCCCCGTGCACTACACCGAC |
| | | GTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCG |
| | | CCTGCGTGAGCAGCGGCAACAGCAACGCCAACAGCCGGGGCCCCAGCTTC |
| | | AGCAGCGGCCTGGTGCCCCTGAGCCTGCGGGGCAGCCACATGAATCCTCT |
| | | GGAGATGTTCGAGACAGTGCCCGTGTGGAGAAGGCAACCCGTGAGGGTGC |
| | | TGAGCCTCTTCGAGGACATTAAGAAGGAGCTGACCTCTCTGGGCTTTCTG |
| | | GAATCCGGCAGCGACCCCGGCCAGCTGAAACACGTGGTGGACGTGACCGA |
| | | CACAGTGAGGAAGGACGTGGAAGAGTGGGGCCCCTTTGACCTCGTGTATG |
| | | GAGCCACACCTCCTCTCGGCCACACATGCGATAGGCCTCCCAGCTGGTAT |
| | | CTCTTCCAGTTCCACAGACTGCTCCAGTACGCCAGACCTAAGCCCGGCAG |
| | | CCCCAGACCCTTCTTCTGGATGTTCGTGGACAATCTGGTGCTGAACAAGG |
| | | AGGATCTGGATGTGGCCAGCAGATTTCTGGAGATGGAACCCGTGACAATC |
| | | CCCGACGTGCATGGCGGCTCTCTGCAGAACGCCGTGAGAGTGTGGTCCAA |
| | | CATCCCCGCCATTAGAAGCAGACACTGGGCTCTGGTGAGCGAGGAGGAAC |
| | | TGTCTCTGCTGGCCCAGAATAAGCAGTCCTCCAAGCTGGCCGCCAAGTGG |
| | | CCCACCAAGCTGGTGAAGAACTGCTTTCTGCCTCTGAGGGAGTATTTCAA |
| | | GTATTTCAGCACCGAACTGACCAGCAGCCTGAGCGGCGGCAAGCGGCCCG |
| | | CCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCT |
| | | GCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAAAA |
| | | AAAAAAAAAAAAAAAAAA |
| MR- | EZH2- | AGGAAATAAGAGAGAAAAGAAGAGTAAGAAGAAATATAAGAGCCACCATG |
| 29908 | CXCL1 ZF5 | GCCCCCAAGAAGAAGCGGAAGGTGGGCGGCAGCGGCGGCAGCGGCCAGAC |
| (SEQ ID | T7 | CGGCAAGAAGAGCGAGAAGGGCCCCGTGTGCTGGCGGAAGCGGGTGAAGA |
| NO: 222) | | GCGAGTACATGCGGCTGCGGCAGCTGAAGCGGTTCCGGCGGGCCGACGAG |
| | | GTGAAGAGCATGTTCAGCAGCAACCGGCAGAAGATCCTGGAGCGGACCGA |
| | | GATCCTGAACCAGGAGTGGAAGCAGCGGCGAATCCAGCCCGTGCACATCC |
| | | TGACCAGCGTGAGCAGCCTGCGGGGCACCCGGGAGTGCAGCGTGACCAGC |
| | | GACCTGGACTTCCCCACCCAGGTGATCCCCCTAAAGACCCTGAACGCCGT |
| | | GGCCAGCGTGCCCATCATGTACAGCTGGAGCCCCCTGCAGCAGAACTTCA |
| | | TGGTGGAGGACGAGACCGTGCTGCACAACATCCCCTACATGGGCGACGAG |
| | | GTGCTGGACCAGGACGGCACCTTCATCGAGGAGCTGATCAAGAACTACGA |
| | | CGGCAAGGTGCACGGCGACCGGGAGTGCGGCTTCATCAACGACGAGATCT |
| | | TCGTGGAGCTGGTGAACGCCCTGGGCCAGTACAACGACGACGACGACGAC |
| | | GACGACGGCGACGACCCCGAGGAGCGGGAGGAGAAGCAGAAGGACCTGGA |
| | | GGACCACCGGGACGACAAGGAGAGCCGGCCCCCCCGGAAGTTCCCCAGCG |
| | | ACAAGATCTTCGAGGCCATCAGCAGCATGTTCCCCGACAAGGGCACCGCC |
| | | GAGGAGCTGAAGGAGAAGTACAAGGAGCTGACCGAGCAGCAGCTGCCCGG |
| | | CGCCCTGCCCCCCGAGTGCACCCCCAACATCGACGGCCCCAACGCCAAGA |
| | | GCGTGCAGCGGGAGCAGAGCCTGCACAGCTTCCACACCCTGTTCTGCCGG |
| | | CGGTGCTTCAAGTACGACTGCTTCCTGCACCCCTTCCACGCCACCCCCAA |
| | | CACCTACAAGCGGAAGAACACCGAGACCGCCCTGGACAACAAGCCCTGCG |
| | | GCCCCCAGTGCTACCAGCACCTGGAGGGCGCCAAGGAGTTCGCCGCCGCC |
| | | CTGACCGCCGAGCGGATCAAGACCCCCCCCAAGCGGCCCGGCGGCCGGCG |
| | | GCGGGGCCGGCTGCCCAACAACAGCAGCCGGCCCAGCACCCCCACCATCA |
| | | ACGTGCTGGAGAGCAAGGACACCGACAGCGACCGGGAGGCCGGCACCGAG |
| | | ACCGGCGGCGAGAACAACGACAAGGAGGAGGAGGAGAAGAAGGACGAGAC |
| | | CAGCAGCAGCAGCGAGGCCAACAGCCGGTGCCAGACCCCCATCAAGATGA |
| | | AGCCCAACATCGAGCCCCCCGAGAACGTGGAGTGGAGCGGCGCCGAGGCC |
| | | AGCATGTTCCGGGTGCTGATCGGCACCTACTACGACAACTTCTGCGCCAT |
| | | CGCCCGGCTGATCGGCACCAAGACCTGCCGGCAGGTGTACGAGTTCCGGG |
| | | TGAAGGAGAGCAGCATCATCGCCCCCGCCCCCGCCGAGGACGTGGACACC |
| | | CCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACC |
| | | AGCCCTGCGACCACCCCCGGCAGCCCTGCGACAGCAGCTGCCCCTGCGTG |
| | | ATCGCCCAGAACTTCTGCGAGAAGTTCTGCCAGTGCAGCAGCGAGTGCCA |
| | | GAACCGGTTCCCCGGCTGCCGGTGCAAGGCCCAGTGCAACACCAAGCAGT |
| | | GCCCCTGCTACCTGGCCGTGCGGGAGTGCGACCCCGACCTGTGCCTGACC |
| | | TGCGGCGCCGCCGACCACTGGGACAGCAAGAACGTGAGCTGCAAGAACTG |
| | | CAGCATCCAGCGGGGCAGCAAGAAGCACCTGCTGCTGGCCCCCAGCGACG |
| | | TGGCCGGCTGGGGCATCTTCATCAAGGACCCCGTGCAGAAGAACGAGTTC |
| | | ATCAGCGAGTACTGCGGCGAGATCATCAGCCAGGACGAGGCCGACCGGCG |
| | | GGGCAAGGTGTACGACAAGTACATGTGCAGCTTCCTGTTCAACCTGAACA |
| | | ACGACTTCGTGGTGGACGCCACCCGGAAGGGCAACAAGATCCGGTTCGCC |
| | | AACCACAGCGTGAACCCCAACTGCTACGCCAAGGTGATGATGGTGAACGG |
| | | CGACCACCGGATCGGCATCTTCGCCAAGCGGGCCATCCAGACCGGCGAGG |
| | | AGCTGTTCTTCGACTACCGGTACAGCCAGGCCGACGCCCTGAAGTACGTG |
| | | GGCATCGAGCGGGAGATGGAGATCCCCGGCAGCAGCGGATCCCTGGAGCC |
| | | CGGCGAAAAGCCTTACAAGTGCCCCGAGTGTGGCAAATCCTTTAGCACCA |
| | | CCGGAAATCTGACCGTCCACCAGAGAACACATACCGGCGAGAAACCCTAC |
| | | AAGTGTCCCGAGTGCGGCAAATCCTTCAGCCAGCTGGCCCATCTGAGAGC |
| | | CCATCAAAGGACCCATACCGGCGAGAAACCTTACAAGTGTCCCGAATGTG |
| | | GAAAGTCCTTTAGCAGCCCCGCCGATCTGACAAGACATCAAAGAACCCAC |
| | | ACCGGCGAGAAGCCCTATAAATGTCCCGAGTGTGGAAAGTCCTTCAGCCA |
| | | GAGCGGCAATCTGACCGAGCATCAAAGAACCCATACCGGCGAAAAGCCCT |
| | | ATAAGTGCCCCGAATGCGGAAAAAGCTTCTCCACAAGCGGCGAGCTGGTG |
| | | AGACACCAAAGGACACATACCGGCGAAAAGCCTTATAAATGCCCCGAGTG |
| | | CGGCAAGAGCTTCTCTAGAAAGGACAATCTGAAGAACCACCAAAGAACAC |
| | | ACACCGGCGAGAAGCCCTACAAATGCCCCGAGTGCGGCAAGAGCTTTAGC |
| | | CAGTCCAGCAACCTCGTGAGACATCAGAGGACACATACCGGAAAAAAGAC |
| | | CAGCGCTAGCGGCAGCGGCGGCGGCAGCGGCGGCAAGCGGCCCGCCGCCA |
| | | CCAAGAAGGCCGGCCAGGCCAAGAAGAAGAAGGGCAGCTACCCCTACGAC |
| | | GTGCCCGACTACGCCTGAGCGGCCGCTTAATTAAGCTGCCTTCTGCGGGG |
| | | CTTGCCTTCTGGCCATGCCCTTCTTCTCTCCCTTGCACCTGTACCTCTTG |
| | | GTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAA |
| | | AAAAAAAAAAAA |
| MR- | EZH2- | AGGAAATAAGAGAGAAAAGAAGAGTAAGAAGAAATATAAGAGCCACCATG |
| 29907 | CXCL1 ZF2 | GCCCCCAAGAAGAAGCGGAAGGTGGGCGGCAGCGGCGGCAGCGGCCAGAC |
| (SEQ ID | T7 | CGGCAAGAAGAGCGAGAAGGGCCCCGTGTGCTGGCGGAAGCGGGTGAAGA |
| NO: 223) | | GCGAGTACATGCGGCTGCGGCAGCTGAAGCGGTTCCGGCGGGCCGACGAG |
| | | GTGAAGAGCATGTTCAGCAGCAACCGGCAGAAGATCCTGGAGCGGACCGA |
| | | GATCCTGAACCAGGAGTGGAAGCAGCGGCGAATCCAGCCCGTGCACATCC |
| | | TGACCAGCGTGAGCAGCCTGCGGGGCACCCGGGAGTGCAGCGTGACCAGC |
| | | GACCTGGACTTCCCCACCCAGGTGATCCCCCTAAAGACCCTGAACGCCGT |
| | | GGCCAGCGTGCCCATCATGTACAGCTGGAGCCCCCTGCAGCAGAACTTCA |
| | | TGGTGGAGGACGAGACCGTGCTGCACAACATCCCCTACATGGGCGACGAG |
| | | GTGCTGGACCAGGACGGCACCTTCATCGAGGAGCTGATCAAGAACTACGA |
| | | CGGCAAGGTGCACGGCGACCGGGAGTGCGGCTTCATCAACGACGAGATCT |
| | | TCGTGGAGCTGGTGAACGCCCTGGGCCAGTACAACGACGACGACGACGAC |
| | | GACGACGGCGACGACCCCGAGGAGCGGGAGGAGAAGCAGAAGGACCTGGA |
| | | GGACCACCGGGACGACAAGGAGAGCCGGCCCCCCCGGAAGTTCCCCAGCG |
| | | ACAAGATCTTCGAGGCCATCAGCAGCATGTTCCCCGACAAGGGCACCGCC |
| | | GAGGAGCTGAAGGAGAAGTACAAGGAGCTGACCGAGCAGCAGCTGCCCGG |
| | | CGCCCTGCCCCCCGAGTGCACCCCCAACATCGACGGCCCCAACGCCAAGA |
| | | GCGTGCAGCGGGAGCAGAGCCTGCACAGCTTCCACACCCTGTTCTGCCGG |
| | | CGGTGCTTCAAGTACGACTGCTTCCTGCACCCCTTCCACGCCACCCCCAA |
| | | CACCTACAAGCGGAAGAACACCGAGACCGCCCTGGACAACAAGCCCTGCG |
| | | GCCCCCAGTGCTACCAGCACCTGGAGGGCGCCAAGGAGTTCGCCGCCGCC |
| | | CTGACCGCCGAGCGGATCAAGACCCCCCCCAAGCGGCCCGGCGGCCGGCG |
| | | GCGGGGCCGGCTGCCCAACAACAGCAGCCGGCCCAGCACCCCCACCATCA |
| | | ACGTGCTGGAGAGCAAGGACACCGACAGCGACCGGGAGGCCGGCACCGAG |
| | | ACCGGCGGCGAGAACAACGACAAGGAGGAGGAGGAGAAGAAGGACGAGAC |
| | | CAGCAGCAGCAGCGAGGCCAACAGCCGGTGCCAGACCCCCATCAAGATGA |
| | | AGCCCAACATCGAGCCCCCCGAGAACGTGGAGTGGAGCGGCGCCGAGGCC |
| | | AGCATGTTCCGGGTGCTGATCGGCACCTACTACGACAACTTCTGCGCCAT |
| | | CGCCCGGCTGATCGGCACCAAGACCTGCCGGCAGGTGTACGAGTTCCGGG |
| | | TGAAGGAGAGCAGCATCATCGCCCCCGCCCCCGCCGAGGACGTGGACACC |
| | | CCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACC |
| | | AGCCCTGCGACCACCCCCGGCAGCCCTGCGACAGCAGCTGCCCCTGCGTG |
| | | ATCGCCCAGAACTTCTGCGAGAAGTTCTGCCAGTGCAGCAGCGAGTGCCA |
| | | GAACCGGTTCCCCGGCTGCCGGTGCAAGGCCCAGTGCAACACCAAGCAGT |
| | | GCCCCTGCTACCTGGCCGTGCGGGAGTGCGACCCCGACCTGTGCCTGACC |
| | | TGCGGCGCCGCCGACCACTGGGACAGCAAGAACGTGAGCTGCAAGAACTG |
| | | CAGCATCCAGCGGGGCAGCAAGAAGCACCTGCTGCTGGCCCCCAGCGACG |
| | | TGGCCGGCTGGGGCATCTTCATCAAGGACCCCGTGCAGAAGAACGAGTTC |
| | | ATCAGCGAGTACTGCGGCGAGATCATCAGCCAGGACGAGGCCGACCGGCG |
| | | GGGCAAGGTGTACGACAAGTACATGTGCAGCTTCCTGTTCAACCTGAACA |
| | | ACGACTTCGTGGTGGACGCCACCCGGAAGGGCAACAAGATCCGGTTCGCC |
| | | AACCACAGCGTGAACCCCAACTGCTACGCCAAGGTGATGATGGTGAACGG |
| | | CGACCACCGGATCGGCATCTTCGCCAAGCGGGCCATCCAGACCGGCGAGG |
| | | AGCTGTTCTTCGACTACCGGTACAGCCAGGCCGACGCCCTGAAGTACGTG |
| | | GGCATCGAGCGGGAGATGGAGATCCCCGGCAGCAGCGGATCCCTGGAGCC |
| | | CGGCGAAAAACCCTATAAGTGCCCCGAGTGCGGCAAGAGCTTTAGCGATC |
| | | CCGGCCATCTGGTGAGGCATCAGAGGACCCACACCGGCGAAAAGCCTTAC |
| | | AAATGCCCCGAGTGTGGAAAAAGCTTCAGCAGAAGCGATCATCTGACCAC |
| | | CCATCAGAGGACACATACCGGCGAGAAGCCTTATAAATGCCCCGAATGTG |
| | | GAAAGAGCTTCTCCAGAAGCGACCATCTGACCAACCACCAGAGGACCCAT |
| | | ACCGGAGAAAAACCTTACAAATGCCCCGAGTGTGGAAAGTCCTTCAGCTC |
| | | CCCCGCCGATCTGACAAGACATCAGAGAACCCACACCGGCGAAAAACCTT |
| | | ATAAATGTCCCGAGTGTGGCAAAAGCTTCTCCGACAAGAAGGATCTGACA |
| | | AGACACCAAAGGACCCACACCGGCGAGAAACCTTATAAATGTCCCGAATG |
| | | CGGAAAAAGCTTTAGCAGAAACGACGCTCTGACCGAACACCAGAGAACAC |
| | | ATACCGGAGAGAAACCCTATAAATGTCCCGAGTGCGGAAAATCCTTCAGC |
| | | ACCACCGGCGCTCTGACAGAGCATCAGAGGACACACACCGGCAAAAAGAC |
| | | CTCCGCTAGCGGCAGCGGCGGCGGCAGCGGCGGCAAGCGGCCCGCCGCCA |
| | | CCAAGAAGGCCGGCCAGGCCAAGAAGAAGAAGGGCAGCTACCCCTACGAC |
| | | GTGCCCGACTACGCCTGAGCGGCCGCTTAATTAAGCTGCCTTCTGCGGGG |
| | | CTTGCCTTCTGGCCATGCCCTTCTTCTCTCCCTTGCACCTGTACCTCTTG |
| | | GTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAA |
| | | AAAAAAAAAAAA |
| MR- | G9A-CXCL1 | AGGAAATAAGAGAGAAAAGAAGAGTAAGAAGAAATATAAGAGCCACCATG |
| 29906 | ZF4 SP6 | GCCCCCAAGAAGAAGCGGAAGGTGGGCATCCACGGCGTGCCCGCCGCCTC |
| (SEQ ID | | GGGCGGGGGTGGCTCAGGAAATAGGGCTATCAGAACCGAGAAGATCATCT |
| NO: 224) | | GTAGGGACGTGGCTAGAGGCTACGAGAACGTGCCCATTCCTTGCGTGAAT |
| | | GGCGTGGATGGCGAACCTTGCCCCGAGGACTACAAATACATCTCCGAGAA |
| | | CTGCGAAACCAGCACAATGAACATCGACAGAAACATCACCCACCTCCAGC |
| | | ACTGCACATGTGTGGATGACTGCTCCTCCAGCAACTGTCTGTGCGGCCAG |
| | | CTCTCCATCAGATGCTGGTACGACAAGGACGGCAGACTGCTGCAAGAGTT |
| | | CAACAAGATCGAACCCCCTCTCATCTTCGAGTGTAACCAAGCTTGCAGCT |
| | | GCTGGAGAAACTGCAAGAATAGAGTGGTCCAGAGCGGCATCAAGGTGAGA |
| | | CTGCAACTGTACAGAACCGCCAAGATGGGATGGGGAGTGAGGGCTCTGCA |
| | | AACCATTCCCCAAGGCACCTTCATCTGCGAATACGTGGGCGAACTGATCT |
| | | CCGACGCCGAAGCTGACGTGAGAGAGGACGACAGCTATCTCTTCGATCTG |
| | | GACAATAAGGACGGCGAGGTGTACTGCATCGACGCTAGATATTACGGCAA |
| | | CATCTCTAGATTCATCAACCACCTCTGCGATCCCAACATCATTCCCGTGA |
| | | GGGTGTTCATGCTGCACCAAGATCTGAGGTTCCCTAGAATCGCCTTCTTC |
| | | AGCTCTAGAGACATCAGAACCGGCGAGGAGCTGGGCTTCGATTACGGCGA |
| | | TAGATTCTGGGACATCAAGTCCAAGTACTTCACATGCCAGTGCGGCAGCG |
| | | AGAAGTGTAAGCACAGCGCTGAGGCCATTGCTCTGGAGCAGTCTAGACTG |
| | | GCCAGACTGGATGGCAGCAGCGGATCCCTGGAGCCCGGAGAAAAGCCTTA |
| | | CAAATGCCCCGAGTGCGGCAAGTCCTTCAGCCAGCTGGCTCATCTGAGAG |
| | | CTCATCAAAGGACCCACACCGGCGAGAAGCCCTATAAGTGCCCCGAGTGC |
| | | GGAAAATCCTTCTCCCAGAGCAGCAATCTCGTCAGACACCAGAGGACCCA |
| | | CACCGGCGAGAAACCTTACAAGTGTCCCGAATGTGGAAAGTCCTTCTCCC |
| | | AAAAGAGCTCTCTGATCGCCCATCAGAGAACACATACCGGCGAAAAACCC |
| | | TACAAGTGCCCCGAGTGTGGCAAAAGCTTTTCCACCACCGGCAATCTGAC |
| | | CGTGCATCAAAGAACCCACACCGGCGAAAAACCCTACAAATGCCCCGAGT |
| | | GTGGCAAATCCTTCTCCGACCCCGGCCATCTGGTGAGGCACCAGAGGACA |
| | | CACACCGGCGAGAAACCTTATAAATGTCCCGAATGCGGCAAGTCCTTTAG |
| | | CACCAGCGGCTCTCTGGTGAGACATCAGAGGACACATACCGGCGAAAAGC |
| | | CTTACAAGTGTCCCGAGTGTGGCAAAAGCTTCAGCCAGAACAGCACACTG |
| | | ACAGAGCATCAGAGAACCCATACCGGCAAAAAGACCAGCGCTAGCGGCAG |
| | | CGGCGGCGGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCC |
| | | TGAGCGGCCGCTTAATTAAGCTGCCTTCTGCGGGGCTTGCCTTCTGGCCA |
| | | TGCCCTTCTTCTCTCCCTTGCACCTGTACCTCTTGGTCTTTGAATAAAGC |
| | | CTGAGTAGGAAGTCTAGAAAAAAAAAAAAAAAAAAAAAAAAAAAAAAAAA |
| | | AAAAAAAAAAAAAAAAAAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATG |
| 29904 | ZF5 SP6 | GCCCCCAAGAAGAAGCGGAAGGTGGGCATCCACGGCGTGCCCGCCGCCTC |
| (SEQ ID | | GGGCGGGGGTGGCTCAGGAAATAGGGCTATCAGAACCGAGAAGATCATCT |
| NO: 225) | | GTAGGGACGTGGCTAGAGGCTACGAGAACGTGCCCATTCCTTGCGTGAAT |
| | | GGCGTGGATGGCGAACCTTGCCCCGAGGACTACAAATACATCTCCGAGAA |
| | | CTGCGAAACCAGCACAATGAACATCGACAGAAACATCACCCACCTCCAGC |
| | | ACTGCACATGTGTGGATGACTGCTCCTCCAGCAACTGTCTGTGCGGCCAG |
| | | CTCTCCATCAGATGCTGGTACGACAAGGACGGCAGACTGCTGCAAGAGTT |
| | | CAACAAGATCGAACCCCCTCTCATCTTCGAGTGTAACCAAGCTTGCAGCT |
| | | GCTGGAGAAACTGCAAGAATAGAGTGGTCCAGAGCGGCATCAAGGTGAGA |
| | | CTGCAACTGTACAGAACCGCCAAGATGGGATGGGGAGTGAGGGCTCTGCA |
| | | AACCATTCCCCAAGGCACCTTCATCTGCGAATACGTGGGCGAACTGATCT |
| | | CCGACGCCGAAGCTGACGTGAGAGAGGACGACAGCTATCTCTTCGATCTG |
| | | GACAATAAGGACGGCGAGGTGTACTGCATCGACGCTAGATATTACGGCAA |
| | | CATCTCTAGATTCATCAACCACCTCTGCGATCCCAACATCATTCCCGTGA |
| | | GGGTGTTCATGCTGCACCAAGATCTGAGGTTCCCTAGAATCGCCTTCTTC |
| | | AGCTCTAGAGACATCAGAACCGGCGAGGAGCTGGGCTTCGATTACGGCGA |
| | | TAGATTCTGGGACATCAAGTCCAAGTACTTCACATGCCAGTGCGGCAGCG |
| | | AGAAGTGTAAGCACAGCGCTGAGGCCATTGCTCTGGAGCAGTCTAGACTG |
| | | GCCAGACTGGATGGCAGCAGCGGATCCCTGGAGCCCGGCGAAAAGCCTTA |
| | | CAAGTGCCCCGAGTGTGGCAAATCCTTTAGCACCACCGGAAATCTGACCG |
| | | TCCACCAGAGAACACATACCGGCGAGAAACCCTACAAGTGTCCCGAGTGC |
| | | GGCAAATCCTTCAGCCAGCTGGCCCATCTGAGAGCCCATCAAAGGACCCA |
| | | TACCGGCGAGAAACCTTACAAGTGTCCCGAATGTGGAAAGTCCTTTAGCA |
| | | GCCCCGCCGATCTGACAAGACATCAAAGAACCCACACCGGCGAGAAGCCC |
| | | TATAAATGTCCCGAGTGTGGAAAGTCCTTCAGCCAGAGCGGCAATCTGAC |
| | | CGAGCATCAAAGAACCCATACCGGCGAAAAGCCCTATAAGTGCCCCGAAT |
| | | GCGGAAAAAGCTTCTCCACAAGCGGCGAGCTGGTGAGACACCAAAGGACA |
| | | CATACCGGCGAAAAGCCTTATAAATGCCCCGAGTGCGGCAAGAGCTTCTC |
| | | TAGAAAGGACAATCTGAAGAACCACCAAAGAACACACACCGGCGAGAAGC |
| | | CCTACAAATGCCCCGAGTGCGGCAAGAGCTTTAGCCAGTCCAGCAACCTC |
| | | GTGAGACATCAGAGGACACATACCGGAAAAAAGACCAGCGCTAGCGGCAG |
| | | CGGCGGCGGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCC |
| | | TGAGCGGCCGCTTAATTAAGCTGCCTTCTGCGGGGCTTGCCTTCTGGCCA |
| | | TGCCCTTCTTCTCTCCCTTGCACCTGTACCTCTTGGTCTTTGAATAAAGC |
| | | CTGAGTAGGAAGTCTAGAGAAAAAAAAAAAAAAAAAAAAAAAAAAAAAAA |
| | | AAAAAAAAAAAAAAAAAAAAAAAAAAAAAAAAAAAAAAAAAAAAAAAAA |
| MR- | CXCL1 ZF4- | AGGAAATAAGAGAGAAAAGAAGAGTAAGAAGAAATATAAGAGCCACCATG |
| 29890 | HDAC8 SP6 | GCCCCCAAGAAGAAGCGGAAGGTGGGCATCCACGGCGTGCCCGCCGCCGG |
| (SEQ ID | | CAGCAGCGGATCCCTGGAGCCCGGAGAAAAGCCTTACAAATGCCCCGAGT |
| NO: 226) | | GCGGCAAGTCCTTCAGCCAGCTGGCTCATCTGAGAGCTCATCAAAGGACC |
| | | CACACCGGCGAGAAGCCCTATAAGTGCCCCGAGTGCGGAAAATCCTTCTC |
| | | CCAGAGCAGCAATCTCGTCAGACACCAGAGGACCCACACCGGCGAGAAAC |
| | | CTTACAAGTGTCCCGAATGTGGAAAGTCCTTCTCCCAAAAGAGCTCTCTG |
| | | ATCGCCCATCAGAGAACACATACCGGCGAAAAACCCTACAAGTGCCCCGA |
| | | GTGTGGCAAAAGCTTTTCCACCACCGGCAATCTGACCGTGCATCAAAGAA |
| | | CCCACACCGGCGAAAAACCCTACAAATGCCCCGAGTGTGGCAAATCCTTC |
| | | TCCGACCCCGGCCATCTGGTGAGGCACCAGAGGACACACACCGGCGAGAA |
| | | ACCTTATAAATGTCCCGAATGCGGCAAGTCCTTTAGCACCAGCGGCTCTC |
| | | TGGTGAGACATCAGAGGACACATACCGGCGAAAAGCCTTACAAGTGTCCC |
| | | GAGTGTGGCAAAAGCTTCAGCCAGAACAGCACACTGACAGAGCATCAGAG |
| | | AACCCATACCGGCAAAAAGACCAGCGCTAGCGGCAGCGGCGGCGGCAGCG |
| | | GCGGCGAGGAGCCCGAGGAGCCCGCCGATAGCGGACAATCTCTGGTGCCC |
| | | GTCTACATCTACAGCCCCGAATATGTGAGCATGTGTGATTCCCTCGCCAA |
| | | GATCCCTAAGAGAGCCAGCATGGTGCATTCTCTGATCGAGGCCTACGCTC |
| | | TGCATAAGCAAATGAGGATCGTGAAGCCCAAGGTCGCCAGCATGGAAGAG |
| | | ATGGCCACCTTTCACACCGATGCCTACCTCCAACATCTCCAGAAGGTGTC |
| | | CCAAGAGGGCGACGACGACCACCCCGACTCCATTGAGTACGGACTGGGCT |
| | | ATGATTGCCCCGCCACCGAGGGCATCTTTGACTATGCCGCCGCTATCGGC |
| | | GGAGCTACCATCACAGCCGCCCAGTGTCTGATTGATGGCATGTGCAAGGT |
| | | CGCCATCAACTGGTCCGGAGGCTGGCATCATGCCAAGAAGGATGAGGCCT |
| | | CCGGCTTCTGTTATCTGAATGACGCCGTGCTGGGCATTCTGAGACTGAGG |
| | | AGGAAATTCGAGAGGATTCTGTACGTGGATCTGGATCTGCATCACGGAGA |
| | | TGGAGTCGAAGATGCCTTCAGCTTCACCAGCAAGGTGATGACAGTCTCTC |
| | | TGCACAAGTTCTCCCCCGGCTTCTTTCCCGGAACCGGCGACGTGTCCGAC |
| | | GTGGGACTGGGCAAGGGAAGGTACTACAGCGTGAACGTGCCCATTCAAGA |
| | | CGGCATCCAAGACGAGAAGTACTACCAGATCTGCGAGTCCGTGCTCAAGG |
| | | AGGTCTACCAAGCCTTCAATCCTAAGGCTGTCGTGCTCCAACTGGGAGCT |
| | | GATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCA |
| | | TTCTCGGAGGAGGCGGATACAATCTGGCTAATACCGCCAGATGCTGGACC |
| | | TATCTGACCGGCGTGATTCTGGGCAAAACACTGAGCAGCGAAATCCCCGA |
| | | CCACGAGTTTTTCACCGCTTACGGCCCCGACTACGTGCTGGAGATCACCC |
| | | CCAGCTGCAGACCCGATAGAAACGAACCCCATAGAATCCAGCAAATTCTG |
| | | AACTATATCAAGGGCAACCTCAAGCACGTCGTGGGAGGTGGCGGATCGGG |
| | | AAAGCGGCCCGCCGCCACCAAGAAGGCCGGTCAGGCCAAGAAGAAGAAGG |
| | | GCAGCTACCCCTACGACGTGCCCGACTACGCCTGAGCGGCCGCTTAATTA |
| | | AGCTGCCTTCTGCGGGGCTTGCCTTCTGGCCATGCCCTTCTTCTCTCCCT |
| | | TGCACCTGTACCTCTTGGTCTTTGAATAAAGCCTGAGTAGGAAGTCTAGA |
| | | AAAAAAAAAAAAAAAAAAAAAAAAAAAAAAAAAAAAAAAAAAAAAAAAAA |
| | | AAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATG |
| 29886 | ZF6 SP6 | GCCCCCAAGAAGAAGCGGAAGGTGGGCATCCACGGCGTGCCCGCCGCCTC |
| (SEQ ID | | GGGCGGGGGTGGCTCAGGAAATAGGGCTATCAGAACCGAGAAGATCATCT |
| NO: 227) | | GTAGGGACGTGGCTAGAGGCTACGAGAACGTGCCCATTCCTTGCGTGAAT |
| | | GGCGTGGATGGCGAACCTTGCCCCGAGGACTACAAATACATCTCCGAGAA |
| | | CTGCGAAACCAGCACAATGAACATCGACAGAAACATCACCCACCTCCAGC |
| | | ACTGCACATGTGTGGATGACTGCTCCTCCAGCAACTGTCTGTGCGGCCAG |
| | | CTCTCCATCAGATGCTGGTACGACAAGGACGGCAGACTGCTGCAAGAGTT |
| | | CAACAAGATCGAACCCCCTCTCATCTTCGAGTGTAACCAAGCTTGCAGCT |
| | | GCTGGAGAAACTGCAAGAATAGAGTGGTCCAGAGCGGCATCAAGGTGAGA |
| | | CTGCAACTGTACAGAACCGCCAAGATGGGATGGGGAGTGAGGGCTCTGCA |
| | | AACCATTCCCCAAGGCACCTTCATCTGCGAATACGTGGGCGAACTGATCT |
| | | CCGACGCCGAAGCTGACGTGAGAGAGGACGACAGCTATCTCTTCGATCTG |
| | | GACAATAAGGACGGCGAGGTGTACTGCATCGACGCTAGATATTACGGCAA |
| | | CATCTCTAGATTCATCAACCACCTCTGCGATCCCAACATCATTCCCGTGA |
| | | GGGTGTTCATGCTGCACCAAGATCTGAGGTTCCCTAGAATCGCCTTCTTC |
| | | AGCTCTAGAGACATCAGAACCGGCGAGGAGCTGGGCTTCGATTACGGCGA |
| | | TAGATTCTGGGACATCAAGTCCAAGTACTTCACATGCCAGTGCGGCAGCG |
| | | AGAAGTGTAAGCACAGCGCTGAGGCCATTGCTCTGGAGCAGTCTAGACTG |
| | | GCCAGACTGGATGGCAGCAGCGGATCCCTGGAGCCCGGCGAAAAGCCTTA |
| | | CAAGTGTCCCGAGTGCGGAAAGAGCTTCAGCAGAGCCGATAATCTGACCG |
| | | AGCACCAAAGGACCCACACCGGAGAGAAGCCTTATAAGTGTCCCGAATGC |
| | | GGCAAAAGCTTTTCTAGAAGCGATCATCTGACCAACCACCAGAGGACACA |
| | | CACCGGAGAAAAACCTTACAAATGCCCCGAGTGCGGCAAAAGCTTCTCCC |
| | | AGAGCAGCAATCTGGTGAGACACCAAAGGACCCACACCGGCGAAAAACCC |
| | | TATAAATGCCCCGAATGTGGCAAGAGCTTTAGCACATCCGGCGAGCTGGT |
| | | GAGGCATCAAAGAACACATACCGGCGAGAAGCCCTACAAGTGCCCCGAGT |
| | | GTGGAAAAAGCTTCAGCACCCACCTCGATCTGATCAGACACCAGAGGACC |
| | | CATACCGGAGAGAAACCCTACAAATGTCCCGAGTGCGGAAAGTCCTTTAG |
| | | CCAGCTGGCCCATCTGAGAGCTCATCAAAGGACACACACCGGCGAGAAGC |
| | | CTTACAAGTGTCCCGAGTGCGGAAAATCCTTCTCCCAACTGGCCCATCTG |
| | | AGGGCCCACCAGAGAACCCACACCGGCAAAAAGACCTCCGCTAGCGGCAG |
| | | CGGCGGCGGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCC |
| | | TGAGCGGCCGCTTAATTAAGCTGCCTTCTGCGGGGCTTGCCTTCTGGCCA |
| | | TGCCCTTCTTCTCTCCCTTGCACCTGTACCTCTTGGTCTTTGAATAAAGC |
| | | CTGAGTAGGAAGTCTAGAAAAAAAAAAAAAAAAAA |
| MR- | G9A-CXCL1 | AGGAAATAAGAGAGAAAAGAAGAGTAAGAAGAAATATAAGAGCCACCATG |
| 29873 | ZF3 SP6 | GCCCCCAAGAAGAAGCGGAAGGTGGGCATCCACGGCGTGCCCGCCGCCTC |
| (SEQ ID | | GGGCGGGGGTGGCTCAGGAAATAGGGCTATCAGAACCGAGAAGATCATCT |
| NO: 228) | | GTAGGGACGTGGCTAGAGGCTACGAGAACGTGCCCATTCCTTGCGTGAAT |
| | | GGCGTGGATGGCGAACCTTGCCCCGAGGACTACAAATACATCTCCGAGAA |
| | | CTGCGAAACCAGCACAATGAACATCGACAGAAACATCACCCACCTCCAGC |
| | | ACTGCACATGTGTGGATGACTGCTCCTCCAGCAACTGTCTGTGCGGCCAG |
| | | CTCTCCATCAGATGCTGGTACGACAAGGACGGCAGACTGCTGCAAGAGTT |
| | | CAACAAGATCGAACCCCCTCTCATCTTCGAGTGTAACCAAGCTTGCAGCT |
| | | GCTGGAGAAACTGCAAGAATAGAGTGGTCCAGAGCGGCATCAAGGTGAGA |
| | | CTGCAACTGTACAGAACCGCCAAGATGGGATGGGGAGTGAGGGCTCTGCA |
| | | AACCATTCCCCAAGGCACCTTCATCTGCGAATACGTGGGCGAACTGATCT |
| | | CCGACGCCGAAGCTGACGTGAGAGAGGACGACAGCTATCTCTTCGATCTG |
| | | GACAATAAGGACGGCGAGGTGTACTGCATCGACGCTAGATATTACGGCAA |
| | | CATCTCTAGATTCATCAACCACCTCTGCGATCCCAACATCATTCCCGTGA |
| | | GGGTGTTCATGCTGCACCAAGATCTGAGGTTCCCTAGAATCGCCTTCTTC |
| | | AGCTCTAGAGACATCAGAACCGGCGAGGAGCTGGGCTTCGATTACGGCGA |
| | | TAGATTCTGGGACATCAAGTCCAAGTACTTCACATGCCAGTGCGGCAGCG |
| | | AGAAGTGTAAGCACAGCGCTGAGGCCATTGCTCTGGAGCAGTCTAGACTG |
| | | GCCAGACTGGATGGCAGCAGCGGATCCCTGGAGCCCGGCGAAAAACCCTA |
| | | TAAATGCCCCGAGTGTGGCAAGAGCTTTTCCGACCCCGGACACCTCGTGA |
| | | GGCATCAGAGAACACATACCGGCGAGAAACCCTACAAGTGCCCCGAATGC |
| | | GGCAAATCCTTCTCTAGAAAGGACAATCTGAAAAACCATCAAAGAACCCA |
| | | TACCGGCGAGAAGCCCTATAAATGTCCCGAGTGTGGAAAGAGCTTCAGCC |
| | | ACAAGAACGCTCTGCAGAACCATCAGAGGACCCATACCGGCGAAAAGCCT |
| | | TATAAGTGCCCCGAGTGCGGAAAATCCTTTTCTAGAAGGGACGAGCTGAA |
| | | TGTGCACCAAAGGACACATACCGGAGAGAAACCCTACAAATGCCCCGAGT |
| | | GCGGCAAGTCCTTCAGCACCTCCGGCAATCTGGTGAGGCACCAAAGGACA |
| | | CACACCGGCGAAAAACCTTACAAGTGTCCCGAGTGCGGAAAAAGCTTTTC |
| | | CCAGAACAGCACACTGACCGAACACCAAAGGACCCACACCGGAGAGAAAC |
| | | CTTATAAATGTCCCGAGTGTGGAAAGTCCTTTAGCCAGTCCGGCAATCTG |
| | | ACAGAGCATCAAAGAACCCACACCGGCAAAAAGACCTCCGCTAGCGGCAG |
| | | CGGCGGCGGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCC |
| | | TGAGCGGCCGCTTAATTAAGCTGCCTTCTGCGGGGCTTGCCTTCTGGCCA |
| | | TGCCCTTCTTCTCTCCAAAAAAAAAAAAAAAAAAAAAAAAAAAAAAAAAA |
| | | AAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATG |
| 29872 | ZF2 SP6 | GCCCCCAAGAAGAAGCGGAAGGTGGGCATCCACGGCGTGCCCGCCGCCTC |
| (SEQ ID | | GGGCGGGGGTGGCTCAGGAAATAGGGCTATCAGAACCGAGAAGATCATCT |
| NO: 229) | | GTAGGGACGTGGCTAGAGGCTACGAGAACGTGCCCATTCCTTGCGTGAAT |
| | | GGCGTGGATGGCGAACCTTGCCCCGAGGACTACAAATACATCTCCGAGAA |
| | | CTGCGAAACCAGCACAATGAACATCGACAGAAACATCACCCACCTCCAGC |
| | | ACTGCACATGTGTGGATGACTGCTCCTCCAGCAACTGTCTGTGCGGCCAG |
| | | CTCTCCATCAGATGCTGGTACGACAAGGACGGCAGACTGCTGCAAGAGTT |
| | | CAACAAGATCGAACCCCCTCTCATCTTCGAGTGTAACCAAGCTTGCAGCT |
| | | GCTGGAGAAACTGCAAGAATAGAGTGGTCCAGAGCGGCATCAAGGTGAGA |
| | | CTGCAACTGTACAGAACCGCCAAGATGGGATGGGGAGTGAGGGCTCTGCA |
| | | AACCATTCCCCAAGGCACCTTCATCTGCGAATACGTGGGCGAACTGATCT |
| | | CCGACGCCGAAGCTGACGTGAGAGAGGACGACAGCTATCTCTTCGATCTG |
| | | GACAATAAGGACGGCGAGGTGTACTGCATCGACGCTAGATATTACGGCAA |
| | | CATCTCTAGATTCATCAACCACCTCTGCGATCCCAACATCATTCCCGTGA |
| | | GGGTGTTCATGCTGCACCAAGATCTGAGGTTCCCTAGAATCGCCTTCTTC |
| | | AGCTCTAGAGACATCAGAACCGGCGAGGAGCTGGGCTTCGATTACGGCGA |
| | | TAGATTCTGGGACATCAAGTCCAAGTACTTCACATGCCAGTGCGGCAGCG |
| | | AGAAGTGTAAGCACAGCGCTGAGGCCATTGCTCTGGAGCAGTCTAGACTG |
| | | GCCAGACTGGATGGCAGCAGCGGATCCCTGGAGCCCGGCGAAAAACCCTA |
| | | TAAGTGCCCCGAGTGCGGCAAGAGCTTTAGCGATCCCGGCCATCTGGTGA |
| | | GGCATCAGAGGACCCACACCGGCGAAAAGCCTTACAAATGCCCCGAGTGT |
| | | GGAAAAAGCTTCAGCAGAAGCGATCATCTGACCACCCATCAGAGGACACA |
| | | TACCGGCGAGAAGCCTTATAAATGCCCCGAATGTGGAAAGAGCTTCTCCA |
| | | GAAGCGACCATCTGACCAACCACCAGAGGACCCATACCGGAGAAAAACCT |
| | | TACAAATGCCCCGAGTGTGGAAAGTCCTTCAGCTCCCCCGCCGATCTGAC |
| | | AAGACATCAGAGAACCCACACCGGCGAAAAACCTTATAAATGTCCCGAGT |
| | | GTGGCAAAAGCTTCTCCGACAAGAAGGATCTGACAAGACACCAAAGGACC |
| | | CACACCGGCGAGAAACCTTATAAATGTCCCGAATGCGGAAAAAGCTTTAG |
| | | CAGAAACGACGCTCTGACCGAACACCAGAGAACACATACCGGAGAGAAAC |
| | | CCTATAAATGTCCCGAGTGCGGAAAATCCTTCAGCACCACCGGCGCTCTG |
| | | ACAGAGCATCAGAGGACACACACCGGCAAAAAGACCTCCGCTAGCGGCAG |
| | | CGGCGGCGGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCC |
| | | TGAGCGGCCGCTTAATTAAGCTGCCTTCTGCGGGGCTTGCCTTCTGGCCA |
| | | TGCCCTTCTTCTCTCCCTTGCACCTGTACCTCTTGGTCTTTGAATAAAGC |
| | | CTGAGTAGGAAGTCTAGAAAAAAAAAAAAAAAAAAAAAAAAAAAAAAAAA |
| | | AAAAAAAAAAAAAAAAAAAAAAAAAAAAAAAAAAAAAAAAAAAAAAA |
| MR- | G9A-CXCL1 | AGGAAATAAGAGAGAAAAGAAGAGTAAGAAGAAATATAAGAGCCACCATG |
| 29871 | ZF1 SP6 | GCCCCCAAGAAGAAGCGGAAGGTGGGCATCCACGGCGTGCCCGCCGCCTC |
| (SEQ ID | | GGGCGGGGGTGGCTCAGGAAATAGGGCTATCAGAACCGAGAAGATCATCT |
| NO: 230) | | GTAGGGACGTGGCTAGAGGCTACGAGAACGTGCCCATTCCTTGCGTGAAT |
| | | GGCGTGGATGGCGAACCTTGCCCCGAGGACTACAAATACATCTCCGAGAA |
| | | CTGCGAAACCAGCACAATGAACATCGACAGAAACATCACCCACCTCCAGC |
| | | ACTGCACATGTGTGGATGACTGCTCCTCCAGCAACTGTCTGTGCGGCCAG |
| | | CTCTCCATCAGATGCTGGTACGACAAGGACGGCAGACTGCTGCAAGAGTT |
| | | CAACAAGATCGAACCCCCTCTCATCTTCGAGTGTAACCAAGCTTGCAGCT |
| | | GCTGGAGAAACTGCAAGAATAGAGTGGTCCAGAGCGGCATCAAGGTGAGA |
| | | CTGCAACTGTACAGAACCGCCAAGATGGGATGGGGAGTGAGGGCTCTGCA |
| | | AACCATTCCCCAAGGCACCTTCATCTGCGAATACGTGGGCGAACTGATCT |
| | | CCGACGCCGAAGCTGACGTGAGAGAGGACGACAGCTATCTCTTCGATCTG |
| | | GACAATAAGGACGGCGAGGTGTACTGCATCGACGCTAGATATTACGGCAA |
| | | CATCTCTAGATTCATCAACCACCTCTGCGATCCCAACATCATTCCCGTGA |
| | | GGGTGTTCATGCTGCACCAAGATCTGAGGTTCCCTAGAATCGCCTTCTTC |
| | | AGCTCTAGAGACATCAGAACCGGCGAGGAGCTGGGCTTCGATTACGGCGA |
| | | TAGATTCTGGGACATCAAGTCCAAGTACTTCACATGCCAGTGCGGCAGCG |
| | | AGAAGTGTAAGCACAGCGCTGAGGCCATTGCTCTGGAGCAGTCTAGACTG |
| | | GCCAGACTGGATGGCAGCAGCGGATCCCTGGAGCCCGGCGAAAAGCCTTA |
| | | CAAATGTCCCGAATGCGGAAAGAGCTTCAGCAGAGCCGACAATCTGACCG |
| | | AACATCAGAGAACCCATACCGGAGAAAAACCTTACAAATGTCCCGAGTGC |
| | | GGCAAAAGCTTCTCCCAAGCCGGACATCTGGCCAGCCACCAAAGGACACA |
| | | TACCGGCGAGAAACCCTACAAGTGCCCCGAGTGCGGCAAGTCCTTCTCTA |
| | | GATCCGATGAGCTGGTCAGACATCAGAGAACCCATACCGGCGAGAAGCCT |
| | | TATAAGTGCCCCGAATGTGGCAAGTCCTTCAGCCAGAGAGCTCATCTGGA |
| | | GAGGCATCAAAGAACACACACCGGAGAGAAACCTTACAAGTGTCCCGAGT |
| | | GTGGAAAGAGCTTCTCCAGAAGGGACGAGCTGAACGTCCACCAAAGAACC |
| | | CATACCGGCGAAAAGCCCTATAAATGCCCCGAGTGTGGAAAATCCTTTTC |
| | | TAGATCCGACCATCTGACAACCCACCAGAGGACCCATACCGGAGAGAAGC |
| | | CCTACAAATGCCCCGAGTGTGGAAAAAGCTTCTCTAGAAACGATGCTCTG |
| | | ACAGAGCACCAAAGGACCCACACCGGCAAAAAGACCAGCGCTAGCGGCAG |
| | | CGGCGGCGGCAGCGGCGGCAAGCGGCCCGCCGCCACCAAGAAGGCCGGCC |
| | | AGGCCAAGAAGAAGAAGGGCAGCTACCCCTACGACGTGCCCGACTACGCC |
| | | TGAGCGGCCGCTTAATTAAGCTGCCTTCTGCGGGGCTTGCCTTCTGGCCA |
| | | TGCCCTTCTTCTCTCCCTTGCACCTGTACCTCTTGGTCTTTGAATAAAGC |
| | | CTGAGTAGGAAGTCTAGAAAAAAAAAAAAAAAAAAAAAAAAAAAAAAAAA |
| | | AAAAAAAAAAAAAAAAAAAAAAAAAAAAAAAAAAAAAAAAAAAAAAA |
| MR- | CXCL1 ZF2- | AGGAAATAAGAGAGAAAAGAAGAGTAAGAAGAAATATAAGAGCCACCATG |
| 29870 | DNMT3a-3L | GCCCCCAAGAAGAAGCGGAAGGTGGGCATCCACGGCGTGCCCGCCGCCGG |
| (SEQ ID | SP6 | CAGCAGCGGATCCCTGGAGCCCGGCGAAAAACCCTATAAGTGCCCCGAGT |
| NO: 231) | | GCGGCAAGAGCTTTAGCGATCCCGGCCATCTGGTGAGGCATCAGAGGACC |
| | | CACACCGGCGAAAAGCCTTACAAATGCCCCGAGTGTGGAAAAAGCTTCAG |
| | | CAGAAGCGATCATCTGACCACCCATCAGAGGACACATACCGGCGAGAAGC |
| | | CTTATAAATGCCCCGAATGTGGAAAGAGCTTCTCCAGAAGCGACCATCTG |
| | | ACCAACCACCAGAGGACCCATACCGGAGAAAAACCTTACAAATGCCCCGA |
| | | GTGTGGAAAGTCCTTCAGCTCCCCCGCCGATCTGACAAGACATCAGAGAA |
| | | CCCACACCGGCGAAAAACCTTATAAATGTCCCGAGTGTGGCAAAAGCTTC |
| | | TCCGACAAGAAGGATCTGACAAGACACCAAAGGACCCACACCGGCGAGAA |
| | | ACCTTATAAATGTCCCGAATGCGGAAAAAGCTTTAGCAGAAACGACGCTC |
| | | TGACCGAACACCAGAGAACACATACCGGAGAGAAACCCTATAAATGTCCC |
| | | GAGTGCGGAAAATCCTTCAGCACCACCGGCGCTCTGACAGAGCATCAGAG |
| | | GACACACACCGGCAAAAAGACCTCCGCTAGCGGCAGCGGCGGCGGCAGCG |
| | | GCGGCAACCACGACCAGGAGTTCGACCCCCCCAAGGTGTACCCCCCCGTG |
| | | CCCGCCGAGAAGCGGAAGCCCATCCGGGTGCTGAGCCTGTTCGACGGCAT |
| | | CGCCACCGGCCTGCTGGTGCTGAAGGACCTGGGCATCCAGGTGGACCGGT |
| | | ACATCGCCAGCGAGGTGTGCGAGGACAGCATCACCGTGGGCATGGTGCGG |
| | | CACCAGGGCAAGATCATGTACGTGGGCGACGTGCGGAGCGTGACCCAGAA |
| | | GCACATCCAGGAGTGGGGCCCCTTCGACCTGGTGATCGGCGGCAGCCCCT |
| | | GCAACGACCTGAGCATCGTGAACCCCGCCCGGAAGGGCCTGTACGAGGGC |
| | | ACCGGCCGGCTGTTCTTCGAGTTCTACCGGCTGCTGCACGACGCCCGGCC |
| | | CAAGGAGGGCGACGACCGGCCCTTCTTCTGGCTGTTCGAGAACGTGGTGG |
| | | CCATGGGCGTGAGCGACAAGCGGGACATCAGCCGGTTCCTGGAGAGCAAC |
| | | CCCGTGATGATCGACGCCAAGGAGGTGAGCGCCGCCCACCGGGCCCGGTA |
| | | CTTCTGGGGCAACCTGCCCGGCATGAACCGGCCCCTGGCCAGCACCGTGA |
| | | ACGACAAGCTGGAGCTGCAGGAGTGCCTGGAGCACGGCCGGATCGCCAAG |
| | | TTCAGCAAGGTGCGGACCATCACCACCCGGAGCAACAGCATCAAGCAGGG |
| | | CAAGGACCAGCACTTCCCCGTGTTCATGAACGAGAAGGAGGACATCCTGT |
| | | GGTGCACCGAGATGGAGCGGGTGTTCGGCTTCCCCGTGCACTACACCGAC |
| | | GTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCG |
| | | CCTGCGTGAGCAGCGGCAACAGCAACGCCAACAGCCGGGGCCCCAGCTTC |
| | | AGCAGCGGCCTGGTGCCCCTGAGCCTGCGGGGCAGCCACATGAATCCTCT |
| | | GGAGATGTTCGAGACAGTGCCCGTGTGGAGAAGGCAACCCGTGAGGGTGC |
| | | TGAGCCTCTTCGAGGACATTAAGAAGGAGCTGACCTCTCTGGGCTTTCTG |
| | | GAATCCGGCAGCGACCCCGGCCAGCTGAAACACGTGGTGGACGTGACCGA |
| | | CACAGTGAGGAAGGACGTGGAAGAGTGGGGCCCCTTTGACCTCGTGTATG |
| | | GAGCCACACCTCCTCTCGGCCACACATGCGATAGGCCTCCCAGCTGGTAT |
| | | CTCTTCCAGTTCCACAGACTGCTCCAGTACGCCAGACCTAAGCCCGGCAG |
| | | CCCCAGACCCTTCTTCTGGATGTTCGTGGACAATCTGGTGCTGAACAAGG |
| | | AGGATCTGGATGTGGCCAGCAGATTTCTGGAGATGGAACCCGTGACAATC |
| | | CCCGACGTGCATGGCGGCTCTCTGCAGAACGCCGTGAGAGTGTGGTCCAA |
| | | CATCCCCGCCATTAGAAGCAGACACTGGGCTCTGGTGAGCGAGGAGGAAC |
| | | TGTCTCTGCTGGCCCAGAATAAGCAGTCCTCCAAGCTGGCCGCCAAGTGG |
| | | CCCACCAAGCTGGTGAAGAACTGCTTTCTGCCTCTGAGGGAGTATTTCAA |
| | | GTATTTCAGCACCGAACTGACCAGCAGCCTGAGCGGCGGCAAGCGGCCCG |
| | | CCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCT |
| | | GCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAAAA |
| | | AAAAAAAAAAAAAAAAAA |
| MR- | CXCL1 ZF6- | AGGAAATAAGAGAGAAAAGAAGAGTAAGAAGAAATATAAGAGCCACCATG |
| 29867 | HDAC8 SP6 | GCCCCCAAGAAGAAGCGGAAGGTGGGCATCCACGGCGTGCCCGCCGCCGG |
| (SEQ ID | | CAGCAGCGGATCCCTGGAGCCCGGCGAAAAGCCTTACAAGTGTCCCGAGT |
| NO: 232) | | GCGGAAAGAGCTTCAGCAGAGCCGATAATCTGACCGAGCACCAAAGGACC |
| | | CACACCGGAGAGAAGCCTTATAAGTGTCCCGAATGCGGCAAAAGCTTTTC |
| | | TAGAAGCGATCATCTGACCAACCACCAGAGGACACACACCGGAGAAAAAC |
| | | CTTACAAATGCCCCGAGTGCGGCAAAAGCTTCTCCCAGAGCAGCAATCTG |
| | | GTGAGACACCAAAGGACCCACACCGGCGAAAAACCCTATAAATGCCCCGA |
| | | ATGTGGCAAGAGCTTTAGCACATCCGGCGAGCTGGTGAGGCATCAAAGAA |
| | | CACATACCGGCGAGAAGCCCTACAAGTGCCCCGAGTGTGGAAAAAGCTTC |
| | | AGCACCCACCTCGATCTGATCAGACACCAGAGGACCCATACCGGAGAGAA |
| | | ACCCTACAAATGTCCCGAGTGCGGAAAGTCCTTTAGCCAGCTGGCCCATC |
| | | TGAGAGCTCATCAAAGGACACACACCGGCGAGAAGCCTTACAAGTGTCCC |
| | | GAGTGCGGAAAATCCTTCTCCCAACTGGCCCATCTGAGGGCCCACCAGAG |
| | | AACCCACACCGGCAAAAAGACCTCCGCTAGCGGCAGCGGCGGCGGCAGCG |
| | | GCGGCGAGGAGCCCGAGGAGCCCGCCGATAGCGGACAATCTCTGGTGCCC |
| | | GTCTACATCTACAGCCCCGAATATGTGAGCATGTGTGATTCCCTCGCCAA |
| | | GATCCCTAAGAGAGCCAGCATGGTGCATTCTCTGATCGAGGCCTACGCTC |
| | | TGCATAAGCAAATGAGGATCGTGAAGCCCAAGGTCGCCAGCATGGAAGAG |
| | | ATGGCCACCTTTCACACCGATGCCTACCTCCAACATCTCCAGAAGGTGTC |
| | | CCAAGAGGGCGACGACGACCACCCCGACTCCATTGAGTACGGACTGGGCT |
| | | ATGATTGCCCCGCCACCGAGGGCATCTTTGACTATGCCGCCGCTATCGGC |
| | | GGAGCTACCATCACAGCCGCCCAGTGTCTGATTGATGGCATGTGCAAGGT |
| | | CGCCATCAACTGGTCCGGAGGCTGGCATCATGCCAAGAAGGATGAGGCCT |
| | | CCGGCTTCTGTTATCTGAATGACGCCGTGCTGGGCATTCTGAGACTGAGG |
| | | AGGAAATTCGAGAGGATTCTGTACGTGGATCTGGATCTGCATCACGGAGA |
| | | TGGAGTCGAAGATGCCTTCAGCTTCACCAGCAAGGTGATGACAGTCTCTC |
| | | TGCACAAGTTCTCCCCCGGCTTCTTTCCCGGAACCGGCGACGTGTCCGAC |
| | | GTGGGACTGGGCAAGGGAAGGTACTACAGCGTGAACGTGCCCATTCAAGA |
| | | CGGCATCCAAGACGAGAAGTACTACCAGATCTGCGAGTCCGTGCTCAAGG |
| | | AGGTCTACCAAGCCTTCAATCCTAAGGCTGTCGTGCTCCAACTGGGAGCT |
| | | GATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCA |
| | | TTCTCGGAGGAGGCGGATACAATCTGGCTAATACCGCCAGATGCTGGACC |
| | | TATCTGACCGGCGTGATTCTGGGCAAAACACTGAGCAGCGAAATCCCCGA |
| | | CCACGAGTTTTTCACCGCTTACGGCCCCGACTACGTGCTGGAGATCACCC |
| | | CCAGCTGCAGACCCGATAGAAACGAACCCCATAGAATCCAGCAAATTCTG |
| | | AACTATATCAAGGGCAACCTCAAGCACGTCGTGGGAGGTGGCGGATCGGG |
| | | AAAGCGGCCCGCCGCCACCAAGAAGGCCGGTCAGGCCAAGAAGAAGAAGG |
| | | GCAGCTACCCCTACGACGTGCCCGACTACGCCTGAGCGGCCGCTTAATTA |
| | | AGCTGCCTTCTGCGGGGCTTGCCTTCTGGCCATGCCCTTCTTCTCTCCCT |
| | | TGCACCTGTACCTCTTGGTCTTTGAATAAAGCCTGAGTAGGAAGTCTAGA |
| | | AAAAAAAAAAAAAAAAAAAAAAAAAAAAAAAAAAAAAAAAAAAAAAAAAA |
| | | AAAAAAAAAAAAAAAAAAAAAAAAAAAAA |
| MR- | EZH2- | AGGAAATAAGAGAGAAAAGAAGAGTAAGAAGAAATATAAGAGCCACCATG |
| 29850 | CXCL1 ZF4 | GCCCCCAAGAAGAAGCGGAAGGTGGGCGGCAGCGGCGGCAGCGGCCAGAC |
| (SEQ ID | T7 | CGGCAAGAAGAGCGAGAAGGGCCCCGTGTGCTGGCGGAAGCGGGTGAAGA |
| NO: 233) | | GCGAGTACATGCGGCTGCGGCAGCTGAAGCGGTTCCGGCGGGCCGACGAG |
| | | GTGAAGAGCATGTTCAGCAGCAACCGGCAGAAGATCCTGGAGCGGACCGA |
| | | GATCCTGAACCAGGAGTGGAAGCAGCGGCGAATCCAGCCCGTGCACATCC |
| | | TGACCAGCGTGAGCAGCCTGCGGGGCACCCGGGAGTGCAGCGTGACCAGC |
| | | GACCTGGACTTCCCCACCCAGGTGATCCCCCTAAAGACCCTGAACGCCGT |
| | | GGCCAGCGTGCCCATCATGTACAGCTGGAGCCCCCTGCAGCAGAACTTCA |
| | | TGGTGGAGGACGAGACCGTGCTGCACAACATCCCCTACATGGGCGACGAG |
| | | GTGCTGGACCAGGACGGCACCTTCATCGAGGAGCTGATCAAGAACTACGA |
| | | CGGCAAGGTGCACGGCGACCGGGAGTGCGGCTTCATCAACGACGAGATCT |
| | | TCGTGGAGCTGGTGAACGCCCTGGGCCAGTACAACGACGACGACGACGAC |
| | | GACGACGGCGACGACCCCGAGGAGCGGGAGGAGAAGCAGAAGGACCTGGA |
| | | GGACCACCGGGACGACAAGGAGAGCCGGCCCCCCCGGAAGTTCCCCAGCG |
| | | ACAAGATCTTCGAGGCCATCAGCAGCATGTTCCCCGACAAGGGCACCGCC |
| | | GAGGAGCTGAAGGAGAAGTACAAGGAGCTGACCGAGCAGCAGCTGCCCGG |
| | | CGCCCTGCCCCCCGAGTGCACCCCCAACATCGACGGCCCCAACGCCAAGA |
| | | GCGTGCAGCGGGAGCAGAGCCTGCACAGCTTCCACACCCTGTTCTGCCGG |
| | | CGGTGCTTCAAGTACGACTGCTTCCTGCACCCCTTCCACGCCACCCCCAA |
| | | CACCTACAAGCGGAAGAACACCGAGACCGCCCTGGACAACAAGCCCTGCG |
| | | GCCCCCAGTGCTACCAGCACCTGGAGGGCGCCAAGGAGTTCGCCGCCGCC |
| | | CTGACCGCCGAGCGGATCAAGACCCCCCCCAAGCGGCCCGGCGGCCGGCG |
| | | GCGGGGCCGGCTGCCCAACAACAGCAGCCGGCCCAGCACCCCCACCATCA |
| | | ACGTGCTGGAGAGCAAGGACACCGACAGCGACCGGGAGGCCGGCACCGAG |
| | | ACCGGCGGCGAGAACAACGACAAGGAGGAGGAGGAGAAGAAGGACGAGAC |
| | | CAGCAGCAGCAGCGAGGCCAACAGCCGGTGCCAGACCCCCATCAAGATGA |
| | | AGCCCAACATCGAGCCCCCCGAGAACGTGGAGTGGAGCGGCGCCGAGGCC |
| | | AGCATGTTCCGGGTGCTGATCGGCACCTACTACGACAACTTCTGCGCCAT |
| | | CGCCCGGCTGATCGGCACCAAGACCTGCCGGCAGGTGTACGAGTTCCGGG |
| | | TGAAGGAGAGCAGCATCATCGCCCCCGCCCCCGCCGAGGACGTGGACACC |
| | | CCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACC |
| | | AGCCCTGCGACCACCCCCGGCAGCCCTGCGACAGCAGCTGCCCCTGCGTG |
| | | ATCGCCCAGAACTTCTGCGAGAAGTTCTGCCAGTGCAGCAGCGAGTGCCA |
| | | GAACCGGTTCCCCGGCTGCCGGTGCAAGGCCCAGTGCAACACCAAGCAGT |
| | | GCCCCTGCTACCTGGCCGTGCGGGAGTGCGACCCCGACCTGTGCCTGACC |
| | | TGCGGCGCCGCCGACCACTGGGACAGCAAGAACGTGAGCTGCAAGAACTG |
| | | CAGCATCCAGCGGGGCAGCAAGAAGCACCTGCTGCTGGCCCCCAGCGACG |
| | | TGGCCGGCTGGGGCATCTTCATCAAGGACCCCGTGCAGAAGAACGAGTTC |
| | | ATCAGCGAGTACTGCGGCGAGATCATCAGCCAGGACGAGGCCGACCGGCG |
| | | GGGCAAGGTGTACGACAAGTACATGTGCAGCTTCCTGTTCAACCTGAACA |
| | | ACGACTTCGTGGTGGACGCCACCCGGAAGGGCAACAAGATCCGGTTCGCC |
| | | AACCACAGCGTGAACCCCAACTGCTACGCCAAGGTGATGATGGTGAACGG |
| | | CGACCACCGGATCGGCATCTTCGCCAAGCGGGCCATCCAGACCGGCGAGG |
| | | AGCTGTTCTTCGACTACCGGTACA |
| | | GCCAGGCCGACGCCCTGAAGTACGTGGGCATCGAGCGGGAGATGGAGATC |
| | | CCCGGCAGCAGCGGATCCCTGGAGCCCGGAGAAAAGCCTTACAAATGCCC |
| | | CGAGTGCGGCAAGTCCTTCAGCCAGCTGGCTCATCTGAGAGCTCATCAAA |
| | | GGACCCACACCGGCGAGAAGCCCTATAAGTGCCCCGAGTGCGGAAAATCC |
| | | TTCTCCCAGAGCAGCAATCTCGTCAGACACCAGAGGACCCACACCGGCGA |
| | | GAAACCTTACAAGTGTCCCGAATGTGGAAAGTCCTTCTCCCAAAAGAGCT |
| | | CTCTGATCGCCCATCAGAGAACACATACCGGCGAAAAACCCTACAAGTGC |
| | | CCCGAGTGTGGCAAAAGCTTTTCCACCACCGGCAATCTGACCGTGCATCA |
| | | AAGAACCCACACCGGCGAAAAACCCTACAAATGCCCCGAGTGTGGCAAAT |
| | | CCTTCTCCGACCCCGGCCATCTGGTGAGGCACCAGAGGACACACACCGGC |
| | | GAGAAACCTTATAAATGTCCCGAATGCGGCAAGTCCTTTAGCACCAGCGG |
| | | CTCTCTGGTGAGACATCAGAGGACACATACCGGCGAAAAGCCTTACAAGT |
| | | GTCCCGAGTGTGGCAAAAGCTTCAGCCAGAACAGCACACTGACAGAGCAT |
| | | CAGAGAACCCATACCGGCAAAAAGACCAGCGCTAGCGGCAGCGGCGGCGG |
| | | CAGCGGCGGCAAGCGGCCCGCCGCCACCAAGAAGGCCGGCCAGGCCAAGA |
| | | AGAAGAAGGGCAGCTACCCCTACGACGTGCCCGACTACGCCTGAGCGGCC |
| | | GCTTAATTAAGCTGCCTTCTGCGGGGCTTGCCTTCTGGCCATGCCCTTCT |
| | | TCTCTCCCTTGCACCTGTACCTCTTGGTCTTTGAATAAAGCCTGAGTAGG |
| | | AAGTCTAGAAAAAAAAAAAAAAAAAAAAAAAAAAAAAAAAAAAAAAAAAA |
| | | AAAAAAAAAAAAAAAAAAAAAAAAAAAAAAAAAAAAAA |
| MR- | EZH2- | AGGAAATAAGAGAGAAAAGAAGAGTAAGAAGAAATATAAGAGCCACCATG |
| 29849 | CXCL1 ZF3 | GCCCCCAAGAAGAAGCGGAAGGTGGGCGGCAGCGGCGGCAGCGGCCAGAC |
| (SEQ ID | T7 | CGGCAAGAAGAGCGAGAAGGGCCCCGTGTGCTGGCGGAAGCGGGTGAAGA |
| NO: 234) | | GCGAGTACATGCGGCTGCGGCAGCTGAAGCGGTTCCGGCGGGCCGACGAG |
| | | GTGAAGAGCATGTTCAGCAGCAACCGGCAGAAGATCCTGGAGCGGACCGA |
| | | GATCCTGAACCAGGAGTGGAAGCAGCGGCGAATCCAGCCCGTGCACATCC |
| | | TGACCAGCGTGAGCAGCCTGCGGGGCACCCGGGAGTGCAGCGTGACCAGC |
| | | GACCTGGACTTCCCCACCCAGGTGATCCCCCTAAAGACCCTGAACGCCGT |
| | | GGCCAGCGTGCCCATCATGTACAGCTGGAGCCCCCTGCAGCAGAACTTCA |
| | | TGGTGGAGGACGAGACCGTGCTGCACAACATCCCCTACATGGGCGACGAG |
| | | GTGCTGGACCAGGACGGCACCTTCATCGAGGAGCTGATCAAGAACTACGA |
| | | CGGCAAGGTGCACGGCGACCGGGAGTGCGGCTTCATCAACGACGAGATCT |
| | | TCGTGGAGCTGGTGAACGCCCTGGGCCAGTACAACGACGACGACGACGAC |
| | | GACGACGGCGACGACCCCGAGGAGCGGGAGGAGAAGCAGAAGGACCTGGA |
| | | GGACCACCGGGACGACAAGGAGAGCCGGCCCCCCCGGAAGTTCCCCAGCG |
| | | ACAAGATCTTCGAGGCCATCAGCAGCATGTTCCCCGACAAGGGCACCGCC |
| | | GAGGAGCTGAAGGAGAAGTACAAGGAGCTGACCGAGCAGCAGCTGCCCGG |
| | | CGCCCTGCCCCCCGAGTGCACCCCCAACATCGACGGCCCCAACGCCAAGA |
| | | GCGTGCAGCGGGAGCAGAGCCTGCACAGCTTCCACACCCTGTTCTGCCGG |
| | | CGGTGCTTCAAGTACGACTGCTTCCTGCACCCCTTCCACGCCACCCCCAA |
| | | CACCTACAAGCGGAAGAACACCGAGACCGCCCTGGACAACAAGCCCTGCG |
| | | GCCCCCAGTGCTACCAGCACCTGGAGGGCGCCAAGGAGTTCGCCGCCGCC |
| | | CTGACCGCCGAGCGGATCAAGACCCCCCCCAAGCGGCCCGGCGGCCGGCG |
| | | GCGGGGCCGGCTGCCCAACAACAGCAGCCGGCCCAGCACCCCCACCATCA |
| | | ACGTGCTGGAGAGCAAGGACACCGACAGCGACCGGGAGGCCGGCACCGAG |
| | | ACCGGCGGCGAGAACAACGACAAGGAGGAGGAGGAGAAGAAGGACGAGAC |
| | | CAGCAGCAGCAGCGAGGCCAACAGCCGGTGCCAGACCCCCATCAAGATGA |
| | | AGCCCAACATCGAGCCCCCCGAGAACGTGGAGTGGAGCGGCGCCGAGGCC |
| | | AGCATGTTCCGGGTGCTGATCGGCACCTACTACGACAACTTCTGCGCCAT |
| | | CGCCCGGCTGATCGGCACCAAGACCTGCCGGCAGGTGTACGAGTTCCGGG |
| | | TGAAGGAGAGCAGCATCATCGCCCCCGCCCCCGCCGAGGACGTGGACACC |
| | | CCCCCCCGGAAGAAGAAGCGGAAGCACCGGCTGTGGGCCGCCCACTGCCG |
| | | GAAGATCCAGCTGAAGAAGGACGGCAGCAGCAACCACGTGTACAACTACC |
| | | AGCCCTGCGACCACCCCCGGCAGCCCTGCGACAGCAGCTGCCCCTGCGTG |
| | | ATCGCCCAGAACTTCTGCGAGAAGTTCTGCCAGTGCAGCAGCGAGTGCCA |
| | | GAACCGGTTCCCCGGCTGCCGGTGCAAGGCCCAGTGCAACACCAAGCAGT |
| | | GCCCCTGCTACCTGGCCGTGCGGGAGTGCGACCCCGACCTGTGCCTGACC |
| | | TGCGGCGCCGCCGACCACTGGGACAGCAAGAACGTGAGCTGCAAGAACTG |
| | | CAGCATCCAGCGGGGCAGCAAGAAGCACCTGCTGCTGGCCCCCAGCGACG |
| | | TGGCCGGCTGGGGCATCTTCATCAAGGACCCCGTGCAGAAGAACGAGTTC |
| | | ATCAGCGAGTACTGCGGCGAGATCATCAGCCAGGACGAGGCCGACCGGCG |
| | | GGGCAAGGTGTACGACAAGTACATGTGCAGCTTCCTGTTCAACCTGAACA |
| | | ACGACTTCGTGGTGGACGCCACCCGGAAGGGCAACAAGATCCGGTTCGCC |
| | | AACCACAGCGTGAACCCCAACTGCTACGCCAAGGTGATGATGGTGAACGG |
| | | CGACCACCGGATCGGCATCTTCGCCAAGCGGGCCATCCAGACCGGCGAGG |
| | | AGCTGTTCTTCGACTACCGGTACAGCCAGGCCGACGCCCTGAAGTACGTG |
| | | GGCATCGAGCGGGAGATGGAGATCCCCGGCAGCAGCGGATCCCTGGAGCC |
| | | CGGCGAAAAACCCTATAAATGCCCCGAGTGTGGCAAGAGCTTTTCCGACC |
| | | CCGGACATCTCGTGAGGCATCAGAGAACACATACCGGCGAGAAACCCTAC |
| | | AAGTGCCCCGAATGCGGCAAATCCTTCTCTAGAAAGGACAATCTGAAAAA |
| | | CCATCAAAGAACCCATACCGGCGAGAAGCCCTATAAATGTCCCGAGTGTG |
| | | GAAAGAGCTTCAGCCACAAGAACGCTCTGCAGAACCATCAGAGGACCCAT |
| | | ACCGGCGAAAAGCCTTATAAGTGCCCCGAGTGCGGAAAATCCTTTTCTAG |
| | | AAGGGACGAGCTGAATGTGCACCAAAGGACACATACCGGAGAGAAACCCT |
| | | ACAAATGCCCCGAGTGCGGCAAGTCCTTCAGCACCTCCGGCAATCTGGTG |
| | | AGGCACCAAAGGACACACACCGGCGAAAAACCTTACAAGTGTCCCGAGTG |
| | | CGGAAAAAGCTTTTCCCAGAACAGCACACTGACCGAACACCAAAGGACCC |
| | | ACACCGGAGAGAAACCTTATAAATGTCCCGAGTGTGGAAAGTCCTTTAGC |
| | | CAGTCCGGCAATCTGACAGAGCATCAAAGAACCCACACCGGCAAAAAGAC |
| | | CTCCGCTAGCGGCAGCGGCGGCGGCAGCGGCGGCAAGCGGCCCGCCGCCA |
| | | CCAAGAAGGCCGGCCAGGCCAAGAAGAAGAAGGGCAGCTACCCCTACGAC |
| | | GTGCCCGACTACGCCTGAGCGGCCGCTTAATTAAGCTGCCTTCTGCGGGG |
| | | CTTGCCTTCTGGCCATGCCCTTCTTCTCTCCCTTGCACCTGTACCTCTTG |
| | | GTCTTTGAATAAAGCCTGAGTAGGAAGTCTAGAAAAAAAAAAAAAAAAAA |
| | | AAAAAAAAAAAAAAAAAAAAAAAAAAAAAAAAAAAAAAAAAAAAAAAAAA |
| | | AAAAAAAAAAAA |
| MR- | CXCL1 ZF3- | AGGAAATAAGAGAGAAAAGAAGAGTAAGAAGAAATATAAGAGCCACCATG |
| 29848 | HDAC8 SP6 | GCCCCCAAGAAGAAGCGGAAGGTGGGCATCCACGGCGTGCCCGCCGCCGG |
| (SEQ ID | | CAGCAGCGGATCCCTGGAGCCCGGCGAAAAACCCTATAAATGCCCCGAGT |
| NO: 235) | | GTGGCAAGAGCTTTTCCGACCCCGGACACCTCGTGAGGCATCAGAGAACA |
| | | CATACCGGCGAGAAACCCTACAAGTGCCCCGAATGCGGCAAATCCTTCTC |
| | | TAGAAAGGACAATCTGAAAAACCATCAAAGAACCCATACCGGCGAGAAGC |
| | | CCTATAAATGTCCCGAGTGTGGAAAGAGCTTCAGCCACAAGAACGCTCTG |
| | | CAGAACCATCAGAGGACCCATACCGGCGAAAAGCCTTATAAGTGCCCCGA |
| | | GTGCGGAAAATCCTTTTCTAGAAGGGACGAGCTGAATGTGCACCAAAGGA |
| | | CACATACCGGAGAGAAACCCTACAAATGCCCCGAGTGCGGCAAGTCCTTC |
| | | AGCACCTCCGGCAATCTGGTGAGGCACCAAAGGACACACACCGGCGAAAA |
| | | ACCTTACAAGTGTCCCGAGTGCGGAAAAAGCTTTTCCCAGAACAGCACAC |
| | | TGACCGAACACCAAAGGACCCACACCGGAGAGAAACCTTATAAATGTCCC |
| | | GAGTGTGGAAAGTCCTTTAGCCAGTCCGGCAATCTGACAGAGCATCAAAG |
| | | AACCCACACCGGCAAAAAGACCTCCGCTAGCGGCAGCGGCGGCGGCAGCG |
| | | GCGGCGAGGAGCCCGAGGAGCCCGCCGATAGCGGACAATCTCTGGTGCCC |
| | | GTCTACATCTACAGCCCCGAATATGTGAGCATGTGTGATTCCCTCGCCAA |
| | | GATCCCTAAGAGAGCCAGCATGGTGCATTCTCTGATCGAGGCCTACGCTC |
| | | TGCATAAGCAAATGAGGATCGTGAAGCCCAAGGTCGCCAGCATGGAAGAG |
| | | ATGGCCACCTTTCACACCGATGCCTACCTCCAACATCTCCAGAAGGTGTC |
| | | CCAAGAGGGCGACGACGACCACCCCGACTCCATTGAGTACGGACTGGGCT |
| | | ATGATTGCCCCGCCACCGAGGGCATCTTTGACTATGCCGCCGCTATCGGC |
| | | GGAGCTACCATCACAGCCGCCCAGTGTCTGATTGATGGCATGTGCAAGGT |
| | | CGCCATCAACTGGTCCGGAGGCTGGCATCATGCCAAGAAGGATGAGGCCT |
| | | CCGGCTTCTGTTATCTGAATGACGCCGTGCTGGGCATTCTGAGACTGAGG |
| | | AGGAAATTCGAGAGGATTCTGTACGTGGATCTGGATCTGCATCACGGAGA |
| | | TGGAGTCGAAGATGCCTTCAGCTTCACCAGCAAGGTGATGACAGTCTCTC |
| | | TGCACAAGTTCTCCCCCGGCTTCTTTCCCGGAACCGGCGACGTGTCCGAC |
| | | GTGGGACTGGGCAAGGGAAGGTACTACAGCGTGAACGTGCCCATTCAAGA |
| | | CGGCATCCAAGACGAGAAGTACTACCAGATCTGCGAGTCCGTGCTCAAGG |
| | | AGGTCTACCAAGCCTTCAATCCTAAGGCTGTCGTGCTCCAACTGGGAGCT |
| | | GATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCA |
| | | TTCTCGGAGGAGGCGGATACAATCTGGCTAATACCGCCAGATGCTGGACC |
| | | TATCTGACCGGCGTGATTCTGGGCAAAACACTGAGCAGCGAAATCCCCGA |
| | | CCACGAGTTTTTCACCGCTTACGGCCCCGACTACGTGCTGGAGATCACCC |
| | | CCAGCTGCAGACCCGATAGAAACGAACCCCATAGAATCCAGCAAATTCTG |
| | | AACTATATCAAGGGCAACCTCAAGCACGTCGTGGGAGGTGGCGGATCGGG |
| | | AAAGCGGCCCGCCGCCACCAAGAAGGCCGGTCAGGCCAAGAAGAAGAAGG |
| | | GCAGCTACCCCTACGACGTGCCCGACTACGCCTGAGCGGCCGCTTAATTA |
| | | AGCTGCCTTCTGCGGGGCTTGCCTTCTGGCCATGCCCTTCTTCTCTCCCT |
| | | TGCACCTGTACCTCTTGGTCTTTGAATAAAGCCTGAGTAGGAAGTCTAGA |
| | | AAAAAAAAAAAAAAAAAAAAAAAAAAAAAAAAAAAAAAAAAAAAAAAAAA |
| | | AAAAAAAAAAAAAAAAAAAAAAAAAAAAA |
| MR- | CXCL1 ZF6- | AGGAAATAAGAGAGAAAAGAAGAGTAAGAAGAAATATAAGAGCCACCATG |
| 29847 | DNMT3a-3L | GCCCCCAAGAAGAAGCGGAAGGTGGGCATCCACGGCGTGCCCGCCGCCGG |
| (SEQ ID | SP6 | CAGCAGCGGATCCCTGGAGCCCGGCGAAAAGCCTTACAAGTGTCCCGAGT |
| NO: 236) | | GCGGAAAGAGCTTCAGCAGAGCCGATAATCTGACCGAGCACCAAAGGACC |
| | | CACACCGGAGAGAAGCCTTATAAGTGTCCCGAATGCGGCAAAAGCTTTTC |
| | | TAGAAGCGATCATCTGACCAACCACCAGAGGACACACACCGGAGAAAAAC |
| | | CTTACAAATGCCCCGAGTGCGGCAAAAGCTTCTCCCAGAGCAGCAATCTG |
| | | GTGAGACACCAAAGGACCCACACCGGCGAAAAACCCTATAAATGCCCCGA |
| | | ATGTGGCAAGAGCTTTAGCACATCCGGCGAGCTGGTGAGGCATCAAAGAA |
| | | CACATACCGGCGAGAAGCCCTACAAGTGCCCCGAGTGTGGAAAAAGCTTC |
| | | AGCACCCACCTCGATCTGATCAGACACCAGAGGACCCATACCGGAGAGAA |
| | | ACCCTACAAATGTCCCGAGTGCGGAAAGTCCTTTAGCCAGCTGGCCCATC |
| | | TGAGAGCTCATCAAAGGACACACACCGGCGAGAAGCCTTACAAGTGTCCC |
| | | GAGTGCGGAAAATCCTTCTCCCAACTGGCCCATCTGAGGGCCCACCAGAG |
| | | AACCCACACCGGCAAAAAGACCTCCGCTAGCGGCAGCGGCGGCGGCAGCG |
| | | GCGGCAACCACGACCAGGAGTTCGACCCCCCCAAGGTGTACCCCCCCGTG |
| | | CCCGCCGAGAAGCGGAAGCCCATCCGGGTGCTGAGCCTGTTCGACGGCAT |
| | | CGCCACCGGCCTGCTGGTGCTGAAGGACCTGGGCATCCAGGTGGACCGGT |
| | | ACATCGCCAGCGAGGTGTGCGAGGACAGCATCACCGTGGGCATGGTGCGG |
| | | CACCAGGGCAAGATCATGTACGTGGGCGACGTGCGGAGCGTGACCCAGAA |
| | | GCACATCCAGGAGTGGGGCCCCTTCGACCTGGTGATCGGCGGCAGCCCCT |
| | | GCAACGACCTGAGCATCGTGAACCCCGCCCGGAAGGGCCTGTACGAGGGC |
| | | ACCGGCCGGCTGTTCTTCGAGTTCTACCGGCTGCTGCACGACGCCCGGCC |
| | | CAAGGAGGGCGACGACCGGCCCTTCTTCTGGCTGTTCGAGAACGTGGTGG |
| | | CCATGGGCGTGAGCGACAAGCGGGACATCAGCCGGTTCCTGGAGAGCAAC |
| | | CCCGTGATGATCGACGCCAAGGAGGTGAGCGCCGCCCACCGGGCCCGGTA |
| | | CTTCTGGGGCAACCTGCCCGGCATGAACCGGCCCCTGGCCAGCACCGTGA |
| | | ACGACAAGCTGGAGCTGCAGGAGTGCCTGGAGCACGGCCGGATCGCCAAG |
| | | TTCAGCAAGGTGCGGACCATCACCACCCGGAGCAACAGCATCAAGCAGGG |
| | | CAAGGACCAGCACTTCCCCGTGTTCATGAACGAGAAGGAGGACATCCTGT |
| | | GGTGCACCGAGATGGAGCGGGTGTTCGGCTTCCCCGTGCACTACACCGAC |
| | | GTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCG |
| | | CCTGCGTGAGCAGCGGCAACAGCAACGCCAACAGCCGGGGCCCCAGCTTC |
| | | AGCAGCGGCCTGGTGCCCCTGAGCCTGCGGGGCAGCCACATGAATCCTCT |
| | | GGAGATGTTCGAGACAGTGCCCGTGTGGAGAAGGCAACCCGTGAGGGTGC |
| | | TGAGCCTCTTCGAGGACATTAAGAAGGAGCTGACCTCTCTGGGCTTTCTG |
| | | GAATCCGGCAGCGACCCCGGCCAGCTGAAACACGTGGTGGACGTGACCGA |
| | | CACAGTGAGGAAGGACGTGGAAGAGTGGGGCCCCTTTGACCTCGTGTATG |
| | | GAGCCACACCTCCTCTCGGCCACACATGCGATAGGCCTCCCAGCTGGTAT |
| | | CTCTTCCAGTTCCACAGACTGCTCCAGTACGCCAGACCTAAGCCCGGCAG |
| | | CCCCAGACCCTTCTTCTGGATGTTCGTGGACAATCTGGTGCTGAACAAGG |
| | | AGGATCTGGATGTGGCCAGCAGATTTCTGGAGATGGAACCCGTGACAATC |
| | | CCCGACGTGCATGGCGGCTCTCTGCAGAACGCCGTGAGAGTGTGGTCCAA |
| | | CATCCCCGCCATTAGAAGCAGACACTGGGCTCTGGTGAGCGAGGAGGAAC |
| | | TGTCTCTGCTGGCCCAGAATAAGCAGTCCTCCAAGCTGGCCGCCAAGTGG |
| | | CCCACCAAGCTGGTGAAGAACTGCTTTCTGCCTCTGAGGGAGTATTTCAA |
| | | GTATTTCAGCACCGAACTGACCAGCAGCCTGAGCGGCGGCAAGCGGCCCG |
| | | CCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCT |
| | | GCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAAAA |
| | | AAAAAAAAAAAAAAAAAA |
| MR- | CXCL1 ZF4- | AGGAAATAAGAGAGAAAAGAAGAGTAAGAAGAAATATAAGAGCCACCATG |
| 29846 | DNMT3a-3L | GCCCCCAAGAAGAAGCGGAAGGTGGGCATCCACGGCGTGCCCGCCGCCGG |
| (SEQ ID | SP6 | CAGCAGCGGATCCCTGGAGCCCGGAGAAAAGCCTTACAAATGCCCCGAGT |
| NO: 237) | | GCGGCAAGTCCTTCAGCCAGCTGGCTCATCTGAGAGCTCATCAAAGGACC |
| | | CACACCGGCGAGAAGCCCTATAAGTGCCCCGAGTGCGGAAAATCCTTCTC |
| | | CCAGAGCAGCAATCTCGTCAGACACCAGAGGACCCACACCGGCGAGAAAC |
| | | CTTACAAGTGTCCCGAATGTGGAAAGTCCTTCTCCCAAAAGAGCTCTCTG |
| | | ATCGCCCATCAGAGAACACATACCGGCGAAAAACCCTACAAGTGCCCCGA |
| | | GTGTGGCAAAAGCTTTTCCACCACCGGCAATCTGACCGTGCATCAAAGAA |
| | | CCCACACCGGCGAAAAACCCTACAAATGCCCCGAGTGTGGCAAATCCTTC |
| | | TCCGACCCCGGCCATCTGGTGAGGCACCAGAGGACACACACCGGCGAGAA |
| | | ACCTTATAAATGTCCCGAATGCGGCAAGTCCTTTAGCACCAGCGGCTCTC |
| | | TGGTGAGACATCAGAGGACACATACCGGCGAAAAGCCTTACAAGTGTCCC |
| | | GAGTGTGGCAAAAGCTTCAGCCAGAACAGCACACTGACAGAGCATCAGAG |
| | | AACCCATACCGGCAAAAAGACCAGCGCTAGCGGCAGCGGCGGCGGCAGCG |
| | | GCGGCAACCACGACCAGGAGTTCGACCCCCCCAAGGTGTACCCCCCCGTG |
| | | CCCGCCGAGAAGCGGAAGCCCATCCGGGTGCTGAGCCTGTTCGACGGCAT |
| | | CGCCACCGGCCTGCTGGTGCTGAAGGACCTGGGCATCCAGGTGGACCGGT |
| | | ACATCGCCAGCGAGGTGTGCGAGGACAGCATCACCGTGGGCATGGTGCGG |
| | | CACCAGGGCAAGATCATGTACGTGGGCGACGTGCGGAGCGTGACCCAGAA |
| | | GCACATCCAGGAGTGGGGCCCCTTCGACCTGGTGATCGGCGGCAGCCCCT |
| | | GCAACGACCTGAGCATCGTGAACCCCGCCCGGAAGGGCCTGTACGAGGGC |
| | | ACCGGCCGGCTGTTCTTCGAGTTCTACCGGCTGCTGCACGACGCCCGGCC |
| | | CAAGGAGGGCGACGACCGGCCCTTCTTCTGGCTGTTCGAGAACGTGGTGG |
| | | CCATGGGCGTGAGCGACAAGCGGGACATCAGCCGGTTCCTGGAGAGCAAC |
| | | CCCGTGATGATCGACGCCAAGGAGGTGAGCGCCGCCCACCGGGCCCGGTA |
| | | CTTCTGGGGCAACCTGCCCGGCATGAACCGGCCCCTGGCCAGCACCGTGA |
| | | ACGACAAGCTGGAGCTGCAGGAGTGCCTGGAGCACGGCCGGATCGCCAAG |
| | | TTCAGCAAGGTGCGGACCATCACCACCCGGAGCAACAGCATCAAGCAGGG |
| | | CAAGGACCAGCACTTCCCCGTGTTCATGAACGAGAAGGAGGACATCCTGT |
| | | GGTGCACCGAGATGGAGCGGGTGTTCGGCTTCCCCGTGCACTACACCGAC |
| | | GTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCG |
| | | CCTGCGTGAGCAGCGGCAACAGCAACGCCAACAGCCGGGGCCCCAGCTTC |
| | | AGCAGCGGCCTGGTGCCCCTGAGCCTGCGGGGCAGCCACATGAATCCTCT |
| | | GGAGATGTTCGAGACAGTGCCCGTGTGGAGAAGGCAACCCGTGAGGGTGC |
| | | TGAGCCTCTTCGAGGACATTAAGAAGGAGCTGACCTCTCTGGGCTTTCTG |
| | | GAATCCGGCAGCGACCCCGGCCAGCTGAAACACGTGGTGGACGTGACCGA |
| | | CACAGTGAGGAAGGACGTGGAAGAGTGGGGCCCCTTTGACCTCGTGTATG |
| | | GAGCCACACCTCCTCTCGGCCACACATGCGATAGGCCTCCCAGCTGGTAT |
| | | CTCTTCCAGTTCCACAGACTGCTCCAGTACGCCAGACCTAAGCCCGGCAG |
| | | CCCCAGACCCTTCTTCTGGATGTTCGTGGACAATCTGGTGCTGAACAAGG |
| | | AGGATCTGGATGTGGCCAGCAGATTTCTGGAGATGGAACCCGTGACAATC |
| | | CCCGACGTGCATGGCGGCTCTCTGCAGAACGCCGTGAGAGTGTGGTCCAA |
| | | CATCCCCGCCATTAGAAGCAGACACTGGGCTCTGGTGAGCGAGGAGGAAC |
| | | TGTCTCTGCTGGCCCAGAATAAGCAGTCCTCCAAGCTGGCCGCCAAGTGG |
| | | CCCACCAAGCTGGTGAAGAACTGCTTTCTGCCTCTGAGGGAGTATTTCAA |
| | | GTATTTCAGCACCGAACTGACCAGCAGCCTGAGCGGCGGCAAGCGGCCCG |
| | | CCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCT |
| | | GCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAAAA |
| | | AAAAAAAAAAAAAAAAAA |
| MR- | CXCL1 ZF3- | AGGAAATAAGAGAGAAAAGAAGAGTAAGAAGAAATATAAGAGCCACCATG |
| 29845 | DNMT3a-3L | GCCCCCAAGAAGAAGCGGAAGGTGGGCATCCACGGCGTGCCCGCCGCCGG |
| (SEQ ID | SP6 | CAGCAGCGGATCCCTGGAGCCCGGCGAAAAACCCTATAAATGCCCCGAGT |
| NO: 238) | | GTGGCAAGAGCTTTTCCGACCCCGGACACCTCGTGAGGCATCAGAGAACA |
| | | CATACCGGCGAGAAACCCTACAAGTGCCCCGAATGCGGCAAATCCTTCTC |
| | | TAGAAAGGACAATCTGAAAAACCATCAAAGAACCCATACCGGCGAGAAGC |
| | | CCTATAAATGTCCCGAGTGTGGAAAGAGCTTCAGCCACAAGAACGCTCTG |
| | | CAGAACCATCAGAGGACCCATACCGGCGAAAAGCCTTATAAGTGCCCCGA |
| | | GTGCGGAAAATCCTTTTCTAGAAGGGACGAGCTGAATGTGCACCAAAGGA |
| | | CACATACCGGAGAGAAACCCTACAAATGCCCCGAGTGCGGCAAGTCCTTC |
| | | AGCACCTCCGGCAATCTGGTGAGGCACCAAAGGACACACACCGGCGAAAA |
| | | ACCTTACAAGTGTCCCGAGTGCGGAAAAAGCTTTTCCCAGAACAGCACAC |
| | | TGACCGAACACCAAAGGACCCACACCGGAGAGAAACCTTATAAATGTCCC |
| | | GAGTGTGGAAAGTCCTTTAGCCAGTCCGGCAATCTGACAGAGCATCAAAG |
| | | AACCCACACCGGCAAAAAGACCTCCGCTAGCGGCAGCGGCGGCGGCAGCG |
| | | GCGGCAACCACGACCAGGAGTTCGACCCCCCCAAGGTGTACCCCCCCGTG |
| | | CCCGCCGAGAAGCGGAAGCCCATCCGGGTGCTGAGCCTGTTCGACGGCAT |
| | | CGCCACCGGCCTGCTGGTGCTGAAGGACCTGGGCATCCAGGTGGACCGGT |
| | | ACATCGCCAGCGAGGTGTGCGAGGACAGCATCACCGTGGGCATGGTGCGG |
| | | CACCAGGGCAAGATCATGTACGTGGGCGACGTGCGGAGCGTGACCCAGAA |
| | | GCACATCCAGGAGTGGGGCCCCTTCGACCTGGTGATCGGCGGCAGCCCCT |
| | | GCAACGACCTGAGCATCGTGAACCCCGCCCGGAAGGGCCTGTACGAGGGC |
| | | ACCGGCCGGCTGTTCTTCGAGTTCTACCGGCTGCTGCACGACGCCCGGCC |
| | | CAAGGAGGGCGACGACCGGCCCTTCTTCTGGCTGTTCGAGAACGTGGTGG |
| | | CCATGGGCGTGAGCGACAAGCGGGACATCAGCCGGTTCCTGGAGAGCAAC |
| | | CCCGTGATGATCGACGCCAAGGAGGTGAGCGCCGCCCACCGGGCCCGGTA |
| | | CTTCTGGGGCAACCTGCCCGGCATGAACCGGCCCCTGGCCAGCACCGTGA |
| | | ACGACAAGCTGGAGCTGCAGGAGTGCCTGGAGCACGGCCGGATCGCCAAG |
| | | TTCAGCAAGGTGCGGACCATCACCACCCGGAGCAACAGCATCAAGCAGGG |
| | | CAAGGACCAGCACTTCCCCGTGTTCATGAACGAGAAGGAGGACATCCTGT |
| | | GGTGCACCGAGATGGAGCGGGTGTTCGGCTTCCCCGTGCACTACACCGAC |
| | | GTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCG |
| | | CCTGCGTGAGCAGCGGCAACAGCAACGCCAACAGCCGGGGCCCCAGCTTC |
| | | AGCAGCGGCCTGGTGCCCCTGAGCCTGCGGGGCAGCCACATGAATCCTCT |
| | | GGAGATGTTCGAGACAGTGCCCGTGTGGAGAAGGCAACCCGTGAGGGTGC |
| | | TGAGCCTCTTCGAGGACATTAAGAAGGAGCTGACCTCTCTGGGCTTTCTG |
| | | GAATCCGGCAGCGACCCCGGCCAGCTGAAACACGTGGTGGACGTGACCGA |
| | | CACAGTGAGGAAGGACGTGGAAGAGTGGGGCCCCTTTGACCTCGTGTATG |
| | | GAGCCACACCTCCTCTCGGCCACACATGCGATAGGCCTCCCAGCTGGTAT |
| | | CTCTTCCAGTTCCACAGACTGCTCCAGTACGCCAGACCTAAGCCCGGCAG |
| | | CCCCAGACCCTTCTTCTGGATGTTCGTGGACAATCTGGTGCTGAACAAGG |
| | | AGGATCTGGATGTGGCCAGCAGATTTCTGGAGATGGAACCCGTGACAATC |
| | | CCCGACGTGCATGGCGGCTCTCTGCAGAACGCCGTGAGAGTGTGGTCCAA |
| | | CATCCCCGCCATTAGAAGCAGACACTGGGCTCTGGTGAGCGAGGAGGAAC |
| | | TGTCTCTGCTGGCCCAGAATAAGCAGTCCTCCAAGCTGGCCGCCAAGTGG |
| | | CCCACCAAGCTGGTGAAGAACTGCTTTCTGCCTCTGAGGGAGTATTTCAA |
| | | GTATTTCAGCACCGAACTGACCAGCAGCCTGAGCGGCGGCAAGCGGCCCG |
| | | CCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCT |
| | | GCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAAAA |
| | | AAAAAAAAAAAAAAAAAA |
| MR- | CXCL1 ZF1- | AGGAAATAAGAGAGAAAAGAAGAGTAAGAAGAAATATAAGAGCCACCATG |
| 29787 | DNMT3a-3L | GCCCCCAAGAAGAAGCGGAAGGTGGGCATCCACGGCGTGCCCGCCGCCGG |
| (SEQ ID | SP6 | CAGCAGCGGATCCCTGGAGCCCGGCGAAAAGCCTTACAAATGTCCCGAAT |
| NO: 239) | | GCGGAAAGAGCTTCAGCAGAGCCGACAATCTGACCGAACATCAGAGAACC |
| | | CATACCGGAGAAAAACCTTACAAATGTCCCGAGTGCGGCAAAAGCTTCTC |
| | | CCAAGCCGGACATCTGGCCAGCCACCAAAGGACACATACCGGCGAGAAAC |
| | | CCTACAAGTGCCCCGAGTGCGGCAAGTCCTTCTCTAGATCCGATGAGCTG |
| | | GTCAGACATCAGAGAACCCATACCGGCGAGAAGCCTTATAAGTGCCCCGA |
| | | ATGTGGCAAGTCCTTCAGCCAGAGAGCTCATCTGGAGAGGCATCAAAGAA |
| | | CACACACCGGAGAGAAACCTTACAAGTGTCCCGAGTGTGGAAAGAGCTTC |
| | | TCCAGAAGGGACGAGCTGAACGTCCACCAAAGAACCCATACCGGCGAAAA |
| | | GCCCTATAAATGCCCCGAGTGTGGAAAATCCTTTTCTAGATCCGACCATC |
| | | TGACAACCCACCAGAGGACCCATACCGGAGAGAAGCCCTACAAATGCCCC |
| | | GAGTGTGGAAAAAGCTTCTCTAGAAACGATGCTCTGACAGAGCACCAAAG |
| | | GACCCACACCGGCAAAAAGACCAGCGCTAGCGGCAGCGGCGGCGGCAGCG |
| | | GCGGCAACCACGACCAGGAGTTCGACCCCCCCAAGGTGTACCCCCCCGTG |
| | | CCCGCCGAGAAGCGGAAGCCCATCCGGGTGCTGAGCCTGTTCGACGGCAT |
| | | CGCCACCGGCCTGCTGGTGCTGAAGGACCTGGGCATCCAGGTGGACCGGT |
| | | ACATCGCCAGCGAGGTGTGCGAGGACAGCATCACCGTGGGCATGGTGCGG |
| | | CACCAGGGCAAGATCATGTACGTGGGCGACGTGCGGAGCGTGACCCAGAA |
| | | GCACATCCAGGAGTGGGGCCCCTTCGACCTGGTGATCGGCGGCAGCCCCT |
| | | GCAACGACCTGAGCATCGTGAACCCCGCCCGGAAGGGCCTGTACGAGGGC |
| | | ACCGGCCGGCTGTTCTTCGAGTTCTACCGGCTGCTGCACGACGCCCGGCC |
| | | CAAGGAGGGCGACGACCGGCCCTTCTTCTGGCTGTTCGAGAACGTGGTGG |
| | | CCATGGGCGTGAGCGACAAGCGGGACATCAGCCGGTTCCTGGAGAGCAAC |
| | | CCCGTGATGATCGACGCCAAGGAGGTGAGCGCCGCCCACCGGGCCCGGTA |
| | | CTTCTGGGGCAACCTGCCCGGCATGAACCGGCCCCTGGCCAGCACCGTGA |
| | | ACGACAAGCTGGAGCTGCAGGAGTGCCTGGAGCACGGCCGGATCGCCAAG |
| | | TTCAGCAAGGTGCGGACCATCACCACCCGGAGCAACAGCATCAAGCAGGG |
| | | CAAGGACCAGCACTTCCCCGTGTTCATGAACGAGAAGGAGGACATCCTGT |
| | | GGTGCACCGAGATGGAGCGGGTGTTCGGCTTCCCCGTGCACTACACCGAC |
| | | GTGAGCAACATGAGCCGGCTGGCCCGGCAGCGGCTGCTGGGCCGGAGCTG |
| | | GAGCGTGCCCGTGATCCGGCACCTGTTCGCCCCCCTGAAGGAGTACTTCG |
| | | CCTGCGTGAGCAGCGGCAACAGCAACGCCAACAGCCGGGGCCCCAGCTTC |
| | | AGCAGCGGCCTGGTGCCCCTGAGCCTGCGGGGCAGCCACATGAATCCTCT |
| | | GGAGATGTTCGAGACAGTGCCCGTGTGGAGAAGGCAACCCGTGAGGGTGC |
| | | TGAGCCTCTTCGAGGACATTAAGAAGGAGCTGACCTCTCTGGGCTTTCTG |
| | | GAATCCGGCAGCGACCCCGGCCAGCTGAAACACGTGGTGGACGTGACCGA |
| | | CACAGTGAGGAAGGACGTGGAAGAGTGGGGCCCCTTTGACCTCGTGTATG |
| | | GAGCCACACCTCCTCTCGGCCACACATGCGATAGGCCTCCCAGCTGGTAT |
| | | CTCTTCCAGTTCCACAGACTGCTCCAGTACGCCAGACCTAAGCCCGGCAG |
| | | CCCCAGACCCTTCTTCTGGATGTTCGTGGACAATCTGGTGCTGAACAAGG |
| | | AGGATCTGGATGTGGCCAGCAGATTTCTGGAGATGGAACCCGTGACAATC |
| | | CCCGACGTGCATGGCGGCTCTCTGCAGAACGCCGTGAGAGTGTGGTCCAA |
| | | CATCCCCGCCATTAGAAGCAGACACTGGGCTCTGGTGAGCGAGGAGGAAC |
| | | TGTCTCTGCTGGCCCAGAATAAGCAGTCCTCCAAGCTGGCCGCCAAGTGG |
| | | CCCACCAAGCTGGTGAAGAACTGCTTTCTGCCTCTGAGGGAGTATTTCAA |
| | | GTATTTCAGCACCGAACTGACCAGCAGCCTGAGCGGCGGCAAGCGGCCCG |
| | | CCGCCACCAAGAAGGCCGGCCAGGCCAAGAAGAAGAAGGGCAGCTACCCC |
| | | TACGACGTGCCCGACTACGCCTGAGCGGCCGCTTAATTAAGCTGCCTTCT |
| | | GCGGGGCTTGCCTTCTGGCCATGCCCTTCTTCTCTCCCTTGCACCTGTAC |
| | | CTCTTGGTCTTTGAATAAAGCCTGAGTAGGAAGTCTAGAAAAAAAAAAAA |
| | | AAAAAAAAAAAAAAAAAAAAAAAAAAAAAAAAAAAAAAAAAAAAAAAAAA |
| | | AAAAAAAAAAAAAAAAAA |
| MR- | CXCL1 ZF5- | AGGAAATAAGAGAGAAAAGAAGAGTAAGAAGAAATATAAGAGCCACCATG |
| 29783 | HDAC8 SP6 | GCCCCCAAGAAGAAGCGGAAGGTGGGCATCCACGGCGTGCCCGCCGCCGG |
| (SEQ ID | | CAGCAGCGGATCCCTGGAGCCCGGCGAAAAGCCTTACAAGTGCCCCGAGT |
| NO: 240) | | GTGGCAAATCCTTTAGCACCACCGGAAATCTGACCGTCCACCAGAGAACA |
| | | CATACCGGCGAGAAACCCTACAAGTGTCCCGAGTGCGGCAAATCCTTCAG |
| | | CCAGCTGGCCCATCTGAGAGCCCATCAAAGGACCCATACCGGCGAGAAAC |
| | | CTTACAAGTGTCCCGAATGTGGAAAGTCCTTTAGCAGCCCCGCCGATCTG |
| | | ACAAGACATCAAAGAACCCACACCGGCGAGAAGCCCTATAAATGTCCCGA |
| | | GTGTGGAAAGTCCTTCAGCCAGAGCGGCAATCTGACCGAGCATCAAAGAA |
| | | CCCATACCGGCGAAAAGCCCTATAAGTGCCCCGAATGCGGAAAAAGCTTC |
| | | TCCACAAGCGGCGAGCTGGTGAGACACCAAAGGACACATACCGGCGAAAA |
| | | GCCTTATAAATGCCCCGAGTGCGGCAAGAGCTTCTCTAGAAAGGACAATC |
| | | TGAAGAACCACCAAAGAACACACACCGGCGAGAAGCCCTACAAATGCCCC |
| | | GAGTGCGGCAAGAGCTTTAGCCAGTCCAGCAACCTCGTGAGACATCAGAG |
| | | GACACATACCGGAAAAAAGACCAGCGCTAGCGGCAGCGGCGGCGGCAGCG |
| | | GCGGCGAGGAGCCCGAGGAGCCCGCCGATAGCGGACAATCTCTGGTGCCC |
| | | GTCTACATCTACAGCCCCGAATATGTGAGCATGTGTGATTCCCTCGCCAA |
| | | GATCCCTAAGAGAGCCAGCATGGTGCATTCTCTGATCGAGGCCTACGCTC |
| | | TGCATAAGCAAATGAGGATCGTGAAGCCCAAGGTCGCCAGCATGGAAGAG |
| | | ATGGCCACCTTTCACACCGATGCCTACCTCCAACATCTCCAGAAGGTGTC |
| | | CCAAGAGGGCGACGACGACCACCCCGACTCCATTGAGTACGGACTGGGCT |
| | | ATGATTGCCCCGCCACCGAGGGCATCTTTGACTATGCCGCCGCTATCGGC |
| | | GGAGCTACCATCACAGCCGCCCAGTGTCTGATTGATGGCATGTGCAAGGT |
| | | CGCCATCAACTGGTCCGGAGGCTGGCATCATGCCAAGAAGGATGAGGCCT |
| | | CCGGCTTCTGTTATCTGAATGACGCCGTGCTGGGCATTCTGAGACTGAGG |
| | | AGGAAATTCGAGAGGATTCTGTACGTGGATCTGGATCTGCATCACGGAGA |
| | | TGGAGTCGAAGATGCCTTCAGCTTCACCAGCAAGGTGATGACAGTCTCTC |
| | | TGCACAAGTTCTCCCCCGGCTTCTTTCCCGGAACCGGCGACGTGTCCGAC |
| | | GTGGGACTGGGCAAGGGAAGGTACTACAGCGTGAACGTGCCCATTCAAGA |
| | | CGGCATCCAAGACGAGAAGTACTACCAGATCTGCGAGTCCGTGCTCAAGG |
| | | AGGTCTACCAAGCCTTCAATCCTAAGGCTGTCGTGCTCCAACTGGGAGCT |
| | | GATACCATTGCTGGCGATCCCATGTGCAGCTTCAATATGACACCCGTCGG |
| | | AATCGGCAAGTGCCTCAAGTACATCCTCCAGTGGCAGCTCGCCACCCTCA |
| | | TTCTCGGAGGAGGCGGATACAATCTGGCTAATACCGCCAGATGCTGGACC |
| | | TATCTGACCGGCGTGATTCTGGGCAAAACACTGAGCAGCGAAATCCCCGA |
| | | CCACGAGTTTTTCACCGCTTACGGCCCCGACTACGTGCTGGAGATCACCC |
| | | CCAGCTGCAGACCCGATAGAAACGAACCCCATAGAATCCAGCAAATTCTG |
| | | AACTATATCAAGGGCAACCTCAAGCACGTCGTGGGAGGTGGCGGATCGGG |
| | | AAAGCGGCCCGCCGCCACCAAGAAGGCCGGTCAGGCCAAGAAGAAGAAGG |
| | | GCAGCTACCCCTACGACGTGCCCGACTACGCCTGAGCGGCCGCTTAATTA |
| | | AGCTGCCTTCTGCGGGGCTTGCCTTCTGGCCATGCCCTTCTTCTCTCCCT |
| | | TGCACCTGTACCTCTTGGTCTTTGAATAAAGCCTGAGTAGGAAGTCTAGA |
| | | AAAAAAAAAAAAAAAAAAAAAAAAAAAAAAAAAAAAAAAAAAAAAAAAAA |
| | | AAAAAAAAAAAAAAAAAAAAAAAAAAAAA |
| MR- | CXCL1 ZF2- | AGGAAATAAGAGAGAAAAGAAGAGTAAGAAGAAATATAAGAGCCACCATG |
| 29782 | HDAC8 SP6 | GCCCCCAAGAAGAAGCGGAAGGTGGGCATCCACGGCGTGCCCGCCGCCGG |
| (SEQ ID | | CAGCAGCGGATCCCTGGAGCCCGGCGAAAAACCCTATAAGTGCCCCGAGT |
| NO: 241) | | GCGGCAAGAGCTTTAGCGATCCCGGCCATCTGGTGAGGCATCAGAGGACC |
| | | CACACCGGCGAAAAGCCTTACAAATGCCCCGAGTGTGGAAAAAGCTTCAG |
| | | CAGAAGCGATCATCTGACCACCCATCAGAGGACACATACCGGCGAGAAGC |
| | | CTTATAAATGCCCCGAATGTGGAAAGAGCTTCTCCAGAAGCGACCATCTG |
| | | ACCAACCACCAGAGGACCCATACCGGAGAAAAACCTTACAAATGCCCCGA |
| | | GTGTGGAAAGTCCTTCAGCTCCCCCGCCGATCTGACAAGACATCAGAGAA |
| | | CCCACACCGGCGAAAAACCTTATAAATGTCCCGAGTGTGGCAAAAGCTTC |
| | | TCCGACAAGAAGGATCTGACAAGACACCAAAGGACCCACACCGGCGAGAA |
| | | ACCTTATAAATGTCCCGAATGCGGAAAAAGCTTTAGCAGAAACGACGCTC |
| | | TGACCGAACACCAGAGAACACATACCGGAGAGAAACCCTATAAATGTCCC |
| | | GAGTGCGGAAAATCCTTCAGCACCACCGGCGCTCTGACAGAGCATCAGAG |
| | | GACACACACCGGCAAAAAGACCTCCGCTAGCGGCAGCGGCGGCGGCAGCG |
| | | GCGGCGAGGAGCCCGAGGAGCCCGCCGATAGCGGACAATCTCTGGTGCCC |
| | | GTCTACATCTACAGCCCCGAATATGTGAGCATGTGTGATTCCCTCG |
| | | CCAAGATCCCTAAGAGAGCCAGCATGGTGCATTCTCTGATCGAGGCCTAC |
| | | GCTCTGCATAAGCAAATGAGGATCGTGAAGCCCAAGGTCGCCAGCATGGA |
| | | AGAGATGGCCACCTTTCACACCGATGCCTACCTCCAACATCTCCAGAAGG |
| | | TGTCCCAAGAGGGCGACGACGACCACCCCGACTCCATTGAGTACGGACTG |
| | | GGCTATGATTGCCCCGCCACCGAGGGCATCTTTGACTATGCCGCCGCTAT |
| | | CGGCGGAGCTACCATCACAGCCGCCCAGTGTCTGATTGATGGCATGTGCA |
| | | AGGTCGCCATCAACTGGTCCGGAGGCTGGCATCATGCCAAGAAGGATGAG |
| | | GCCTCCGGCTTCTGTTATCTGAATGACGCCGTGCTGGGCATTCTGAGACT |
| | | GAGGAGGAAATTCGAGAGGATTCTGTACGTGGATCTGGATCTGCATCACG |
| | | GAGATGGAGTCGAAGATGCCTTCAGCTTCACCAGCAAGGTGATGACAGTC |
| | | TCTCTGCACAAGTTCTCCCCCGGCTTCTTTCCCGGAACCGGCGACGTGTC |
| | | CGACGTGGGACTGGGCAAGGGAAGGTACTACAGCGTGAACGTGCCCATTC |
| | | AAGACGGCATCCAAGACGAGAAGTACTACCAGATCTGCGAGTCCGTGCTC |
| | | AAGGAGGTCTACCAAGCCTTCAATCCTAAGGCTGTCGTGCTCCAACTGGG |
| | | AGCTGATACCATTGCTGGCGATCCCATGTGCAGCTTCAATATGACACCCG |
| | | TCGGAATCGGCAAGTGCCTCAAGTACATCCTCCAGTGGCAGCTCGCCACC |
| | | CTCATTCTCGGAGGAGGCGGATACAATCTGGCTAATACCGCCAGATGCTG |
| | | GACCTATCTGACCGGCGTGATTCTGGGCAAAACACTGAGCAGCGAAATCC |
| | | CCGACCACGAGTTTTTCACCGCTTACGGCCCCGACTACGTGCTGGAGATC |
| | | ACCCCCAGCTGCAGACCCGATAGAAACGAACCCCATAGAATCCAGCAAAT |
| | | TCTGAACTATATCAAGGGCAACCTCAAGCACGTCGTGGGAGGTGGCGGAT |
| | | CGGGAAAGCGGCCCGCCGCCACCAAGAAGGCCGGTCAGGCCAAGAAGAAG |
| | | AAGGGCAGCTACCCCTACGACGTGCCCGACTACGCCTGAGCGGCCGCTTA |
| | | ATTAAGCTGCCTTCTGCGGGGCTTGCCTTCTGGCCATGCCCTTCTTCTCT |
| | | CCCTTGCACCTGTACCTCTTGGTCTTTGAATAAAGCCTGAGTAGGAAGTC |
| | | TAGAAAAAAAAAAAAAAAAAAAAAAAAAAAAAAAAAAAAAAAAAAAAAAA |
| | | AAAAAAAAAAAAAAAAAAAAAAAAAAAAAAAAA |
| MR- | CXCL1 ZF1- | AGGAAATAAGAGAGAAAAGAAGAGTAAGAAGAAATATAAGAGCCACCATG |
| 29780 | HDAC8 SP6 | GCCCCCAAGAAGAAGCGGAAGGTGGGCATCCACGGCGTGCCCGCCGCCGG |
| (SEQ ID | | CAGCAGCGGATCCCTGGAGCCCGGCGAAAAGCCTTACAAATGTCCCGAAT |
| NO: 242) | | GCGGAAAGAGCTTCAGCAGAGCCGACAATCTGACCGAACATCAGAGAACC |
| | | CATACCGGAGAAAAACCTTACAAATGTCCCGAGTGCGGCAAAAG |
| | | CTTCTCCCAAGCCGGACATCTGGCCAGCCACCAAAGGACACATACCGGCG |
| | | AGAAACCCTACAAGTGCCCCGAGTGCGGCAAGTCCTTCTCTAGATCCGAT |
| | | GAGCTGGTCAGACATCAGAGAACCCATACCGGCGAGAAGCCTTATAAGTG |
| | | CCCCGAATGTGGCAAGTCCTTCAGCCAGAGAGCTCATCTGGAGAGGCATC |
| | | AAAGAACACACACCGGAGAGAAACCTTACAAGTGTCCCGAGTGTGGAAAG |
| | | AGCTTCTCCAGAAGGGACGAGCTGAACGTCCACCAAAGAACCCATACCGG |
| | | CGAAAAGCCCTATAAATGCCCCGAGTGTGGAAAATCCTTTTCTAGATCCG |
| | | ACCATCTGACAACCCACCAGAGGACCCATACCGGAGAGAAGCCCTACAAA |
| | | TGCCCCGAGTGTGGAAAAAGCTTCTCTAGAAACGATGCTCTGACAGAGCA |
| | | CCAAAGGACCCACACCGGCAAAAAGACCAGCGCTAGCGGCAGCGGCGGCG |
| | | GCAGCGGCGGCGAGGAGCCCGAGGAGCCCGCCGATAGCGGACAATCTCTG |
| | | GTGCCCGTCTACATCTACAGCCCCGAATATGTGAGCATGTGTGATTCCCT |
| | | CGCCAAGATCCCTAAGAGAGCCAGCATGGTGCATTCTCTGATCGAGGCCT |
| | | ACGCTCTGCATAAGCAAATGAGGATCGTGAAGCCCAAGGTCGCCAGCATG |
| | | GAAGAGATGGCCACCTTTCACACCGATGCCTACCTCCAACATCTCCAGAA |
| | | GGTGTCCCAAGAGGGCGACGACGACCACCCCGACTCCATTGAGTACGGAC |
| | | TGGGCTATGATTGCCCCGCCACCGAGGGCATCTTTGACTATGCCGCCGCT |
| | | ATCGGCGGAGCTACCATCACAGCCGCCCAGTGTCTGATTGATGGCATGTG |
| | | CAAGGTCGCCATCAACTGGTCCGGAGGCTGGCATCATGCCAAGAAGGATG |
| | | AGGCCTCCGGCTTCTGTTATCTGAATGACGCCGTGCTGGGCATTCTGAGA |
| | | CTGAGGAGGAAATTCGAGAGGATTCTGTACGTGGATCTGGATCTGCATCA |
| | | CGGAGATGGAGTCGAAGATGCCTTCAGCTTCACCAGCAAGGTGATGACAG |
| | | TCTCTCTGCACAAGTTCTCCCCCGGCTTCTTTCCCGGAACCGGCGACGTG |
| | | TCCGACGTGGGACTGGGCAAGGGAAGGTACTACAGCGTGAACGTGCCCAT |
| | | TCAAGACGGCATCCAAGACGAGAAGTACTACCAGATCTGCGAGTCCGTGC |
| | | TCAAGGAGGTCTACCAAGCCTTCAATCCTAAGGCTGTCGTGCTCCAACTG |
| | | GGAGCTGATACCATTGCTGGCGATCCCATGTGCAGCTTCAATATGACACC |
| | | CGTCGGAATCGGCAAGTGCCTCAAGTACATCCTCCAGTGGCAGCTCGCCA |
| | | CCCTCATTCTCGGAGGAGGCGGATACAATCTGGCTAATACCGCCAGATGC |
| | | TGGACCTATCTGACCGGCGTGATTCTGGGCAAAACACTGAGCAGCGAAAT |
| | | CCCCGACCACGAGTTTTTCACCGCTTACGGCCCCGACTACGTGCTGGAGA |
| | | TCACCCCCAGCTGCAGACCCGATAGAAACGAACCCCATAGAATCCAGCAA |
| | | ATTCTGAACTATATCAAGGGCAACCTCAAGCACGTCGTGGGAGGTGGCGG |
| | | ATCGGGAAAGCGGCCCGCCGCCACCAAGAAGGCCGGTCAGGCCAAGAAGA |
| | | AGAAGGGCAGCTACCCCTACGACGTGCCCGACTACGCCTGAGCGGCCGCT |
| | | TAATTAAGCTGCCTTCTGCGGGGCTTGCCTTCTGGCCATGCCCTTCTTCT |
| | | CTCCCTTGCACCTGTACCTCTTGGTCTTTGAATAAAGCCTGAGTAGGAAG |
| | | TCTAGAAAAAAAAAAAAAAAAAAAAAAAAAAAAAAAAAAAAAAAAAAAAA |
| | | AAAAAAAAAAAAAAAAAAAAAAAAAAAAAAAAAAA |

In some embodiments, a nucleic acid for use in a method or composition described herein (e.g., a nucleic acid encoding a site-specific disrupting agent) comprises a nucleic acid sequence of any one of SEQ ID NOs: 69, 71, 85, 201, 202, 204, 205, 207, 209, 211, 213, 215, 217, or 219-242, a complementary or reverse complementary sequence of any thereof, or comprises a sequence with at least 80, 85, 90, 91, 92, 93, 94, 95, 96, 97, 98, or 99% identity to any thereof. In some embodiments, a site-specific disrupting agent for use in a method or composition described herein comprises an amino acid sequence of any one of SEQ ID NOs: 70, 72-82, 84, 86, 203, 206, 208, 210, 212, 214, 216, or 218, or comprises a sequence with at least 80, 85, 90, 91, 92, 93, 94, 95, 96, 97, 98, or 99% identity to any thereof. In some embodiments, a site-specific disrupting agent for use in a method or composition described herein comprises an amino acid sequence encoded by any one of SEQ ID NOs: 69, 71, 85, 201, 202, 204, 205, 207, 209, 211, 213, 215, 217, or 219-242, or an amino acid sequence with at least 80, 85, 90, 91, 92, 93, 94, 95, 96, 97, 98, or 99% identity to any thereof.

Functional Characteristics

A site-specific disrupting agent or a system of the present disclosure can be used to modulate, e.g., decrease, expression of a target plurality of genes in a cell. In some embodiments, modulating expression comprises decreasing the level of RNA, e.g., mRNA, encoded by each of the target plurality of genes. In some embodiments, modulating expression comprises decreasing the level of protein encoded by each of the target plurality of genes. In some embodiments, modulating expression comprises both decreasing the level of mRNA and protein encoded by each of the target plurality of genes. In some embodiments, the expression of a gene of the target plurality of genes in a cell contacted by or comprising the site-specific disrupting agent is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of expression of the gene in a cell not contacted by or comprising the site-specific disrupting agent or system. In some embodiments, the expression of each gene of the target plurality of genes in a cell contacted by or comprising the site-specific disrupting agent or system is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× lower than the level of expression of the gene in a cell not contacted by or comprising the site-specific disrupting agent or system. Expression of a gene may be assayed by methods known to those of skill in the art, including RT-PCR, ELISA, Western blot, and the methods of Examples 2 or 4-19.

A site-specific disrupting agent or a system of the present disclosure can be used to decrease binding of a nucleating polypeptide, e.g., CTCF, to an anchor sequence. In some embodiments, contacting a cell or administering a site-specific disrupting agent or a system results in a decrease in binding of a nucleating polypeptide, e.g., CTCF, to an anchor sequence (e.g., an anchor sequence of an ASMC comprising a target plurality of genes). In some embodiments, contacting a cell or administering a site-specific disrupting agent or a system results in a complete loss of binding or a decrease of at least 50, 60, 70, 80, 90, 95, or 99%, e.g., as measured by ChIP and/or quantitative PCR, relative to the binding of the nucleating polypeptide (e.g., CTCF) to the anchor sequence prior to treatment with the site-specific disrupting agent or a system or in the absence of the site-specific disrupting agent or a system.

A site-specific disrupting agent or a system of the present disclosure can be used to disrupt a genomic complex (e.g., ASMC) comprising a target plurality of cells. In some embodiments, contacting a cell or administering a site-specific disrupting agent or a system results in a decrease in the level of a genomic complex (e.g., ASMC) comprising the target plurality of genes relative to the level of the complex prior to treatment with the site-specific disrupting agent or a system or in the absence of the site-specific disrupting agent or a system. In some embodiments, contacting a cell or administering a site-specific disrupting agent or a system results in a complete loss of the genomic complex, e.g., ASMC, or a decrease of at least 25, 30, 35, 40, 45, 50, 60, 70, 80, 90, 95, or 99%, e.g., as measured by ChIA-PET, ELISA (e.g., to assess gene expression changes), CUT&RUN, ATAC-SEQ, ChIP and/or quantitative PCR, relative to the level of the complex prior to treatment with the site-specific disrupting agent or a system or in the absence of the site-specific disrupting agent or a system.

A site-specific disrupting agent or a system of the present disclosure can be used to modulate, e.g., decrease, expression of a target plurality of genes in a cell for a time period. In some embodiments, the expression of a gene of the target plurality of genes in a cell contacted by or comprising the site-specific disrupting agent or a system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). In some embodiments, the expression of each gene of the target plurality of genes in a cell contacted by or comprising the site-specific disrupting agent or a system is appreciably decreased for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, the expression of a gene of the target plurality of genes in a cell contacted by or comprising the site-specific disrupting agent or a system is appreciably decreased for no more than 10, 9, 8, 7, 6, 5, 4, 3, 2, or 1 years. Optionally, the expression of each gene of the target plurality of genes in a cell contacted by or comprising the site-specific disrupting agent or a system is appreciably decreased for no more than 10, 9, 8, 7, 6, 5, 4, 3, 2, or 1 years.

A site-specific disrupting agent or a system may comprise a plurality of effector moieties, where each effector moiety has a different functionality from each other effector moiety. For example, a site-specific disrupting agent or a system may comprise a first effector moiety comprising histone deacetylase functionality and a second effector moiety comprising DNA methyltransferase functionality. In some embodiments, a site-specific disrupting agent comprises a combination of effector moieties whose functionalities are complementary to one another with regard to modulating, e.g., decreasing, expression of a target plurality of genes, e.g., where the functionalities together decrease expression and, optionally, do not decrease or negligibly decrease expression when present individually.

In some embodiments, a site-specific disrupting agent or a system comprises a combination of effector moieties whose functionalities synergize with one another with regard to modulating, e.g., decreasing, expression of a target plurality of genes. Without wishing to be bound by theory, it is thought that epigenetic modifications to a genomic locus are cumulative, in that multiple repressive epigenetic markers (e.g., multiple different types of epigenetic markers and/or more extensive marking of a given type) together reduce expression more effectively than individual modifications alone (e.g., producing a greater decrease in expression and/or a longer-lasting decrease in expression). In some embodiments, a site-specific disrupting agent or a system comprises a plurality of effector moieties that synergize with each other, e.g., each effector moiety decreases expression of a target gene. In some embodiments, a site-specific disrupting agent comprising a plurality of different effector moieties which synergize with one another is more effective at modulating, e.g., decreasing, expression of a target plurality of genes than a site-specific disrupting agent comprising only one of the plurality of different effector moieties or a non-synergistic combination of effector moieties. In some embodiments, such a site-specific disrupting agent is at least 1.05× (i.e., 1.05 times), 1.1×, 1.15×, 1.2×, 1.25×, 1.3×, 1.35×, 1.4×, 1.45×, 1.5×, 1.55×, 1.6×, 1.65×, 1.7×, 1.75×, 1.8×, 1.85×, 1.9×, 1.95×, 2×, 3×, 4×, 5×, 6×, 7×, 8×, 9×, 10×, 20×, 30×, 40×, 50×, 60×, 70×, 80×, 90×, or 100× as effective at modulating, e.g., decreasing, expression of a target plurality of genes than a site-specific disrupting agent or a system comprising only one of the plurality of different effector moieties or a non-synergistic combination of effector moieties.

### Target Sites

A site-specific disrupting agent or a system disclosed herein is useful for modulating, e.g., decreasing, expression of a target plurality of genes in cell, e.g., in a subject or patient. A target plurality of genes may include any gene known to those of skill in the art. A target plurality of genes comprises at least two genes. In some embodiments, a targeted plurality of genes comprises at least 2, 3, 4, 5, 6, 7, 8, 9, 10, 11, 12, 13, 14, 15, 16, 17, 18, 19, or 20 genes (and optionally, no more than 2, 3, 4, 5, 6, 7, 8, 9, 10, 11, 12, 13, 14, 15, 16, 17, 18, 19, 20, or 30 genes), e.g., a first gene and a second gene, and optionally a third gene, a fourth gene, a fifth gene, a sixth gene, a seventh gene, an eighth gene, a ninth gene, a tenth gene, an eleventh gene, a twelfth gene, a thirteenth gene, a fourteenth gene, a fifteenth gene, a sixteenth gene, a seventeenth gene, an eighteenth gene, a nineteenth gene, and/or a twentieth gene. In some embodiments, a targeted plurality of genes comprises 2-20, 2-18, 2-16, 2-14, 2-12, 2-10, 2-9, 2-8, 2-7, 2-6, 2-5, 2-4, 2-3, 3-20, 3-18, 3-16, 3-14, 3-12, 3-10, 3-9, 3-8, 3-7, 3-6, 3-5, 3-4, 4-20, 4-18, 4-16, 4-14, 4-12, 4-10, 4-9, 4-8, 4-7, 4-6, 4-5, 5-20, 5-18, 5-16, 5-14, 5-12, 5-10, 5-9, 5-8, 5-7, 5-6, 6-20, 6-18, 6-16, 6- 14, 6-12, 6-10, 6-9, 6-8, 6-7, 7-20, 7-18, 7-16, 7-14, 7-12, 7-10, 7-9, 7-8, 8-20, 8-18, 8-16, 8-14, 8-12, 8-10, 8-9, 9-20, 9-18, 9-16, 9-14, 9-12, 9-10, 10-20, 10-18, 10-16, 10-14, 10-12, 12-20, 12-18, 12-16, 12-14, 14-20, 14-18, 14-16, 16-20, 16-18, or 18-20 genes.

In some embodiments, two or more (e.g., all) genes of a target plurality of genes are associated with a disease or condition in a subject, e.g., a mammal, e.g., a human, bovine, horse, sheep, chicken, rat, mouse, cat, or dog. In some embodiments, the disease or condition is an inflammatory disease, e.g., an immune mediated inflammatory disease. In some embodiments, the disease or condition is one or more of rheumatoid arthritis, inflammatory, arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a virus, or a fungus), external injury (e.g., scrapes or foreign objects), effects of radiation or chemical injury, osteoarthritis, osteoarthritic joint pain, joint pain, inflammatory pain, acute pain, chronic pain, cystisis, bronchitis, dermatitis, cardiovascular disease, neurodegenerative disease, liver disease, lung disease, kidney disease, pain, swelling, stiffness, tenderness, redness, warmth, or elevated biomarkers related to disease states (e.g., cytokines, immune receptors, or inflammatory markers). In some embodiments, the inflammatory disorder is associated with an infection, e.g., by a virus, e.g., Sars-Cov-2 virus. In some embodiments, the inflammatory disorder is an autoimmune disorder. In some embodiments, the inflammatory disorder is associated with hypoxia. In some embodiments, the inflammatory disorder is associated with ARDS, hypoxia, and/or sepsis. In some embodiments, the infection is a super infection, e.g., caused by more than one pathogen, e.g., a first virus or a bacterium, or a fungus, and a second virus, or a bacterium, or a fungus. In some embodiments, the inflammatory disorder may change lung cell composition, e.g., decreased AT2 cells and/or increased dendritic cell, macrophages, neutrophils, NK cells, fibroblasts, leukocytes, lymphatic endothelial cells and/or vascular endothelial cells. In some embodiments, the disorder is associated with one or more comorbidities, e.g., respiratory infections, obesity, gastroesophageal reflux disease, skin lesions, and/or obstructive sleep apnea.

In some embodiments, two or more (e.g., all) genes of a target plurality of genes are aberrantly expressed, e.g., over-expressed, in a cell, e.g., in a subject, e.g., a human subject.

In some embodiments, two or more (e.g., all) genes of a target plurality of genes have related functionalities. Without wishing to be bound by theory, it is thought that genes with related functionalities are frequently positioned in close proximity to one another in the genome and are also frequently found within (wholly or in part) common genomic complexes, e.g., ASMCs. Modulating, e.g., decreasing, expression of a target plurality of genes where two or more (e.g., all) of the genes of the plurality have related functionalities may be accomplished efficiently and effectively by targeting a genomic complex, e.g., ASMC, comprising said interrelated genes.

In some embodiments, one, two, three, or more (e.g., all) genes of a target plurality of genes are cytokines, e.g., chemokines, an interleukin, a transcription factor (e.g., an interferon regulatory transcription factor), intercellular adhesion molecule (ICAM), or an interferon receptor. In some embodiments, two or more (e.g., all) genes of a target plurality of genes are cytokines, an interleukin, a transcription factor (e.g., an interferon regulatory transcription factor), intercellular adhesion molecule (ICAM), or an interferon receptor. In some embodiments, the target plurality of genes are mammalian gene, e.g., mouse genes, human genes.

In some embodiments, two or more (e.g., all) genes of a target plurality of genes have pro-inflammatory functionality. In some embodiments, two or more (e.g., all) genes of a target plurality of genes may act as a chemoattractant for immune cells, e.g., neutrophils. For example, genes having pro-inflammatory functionality (also referred to herein as pro-inflammatory genes) include CXCL1, CXCL2, CXCL3, CXCL4, CXCL5, CXCL6, CXCL7, IL8, CXCL15, CCL2, CCL7, CCL9, IL1A, IL1B, CSF2, IRF1, ICAM1, ICAM4, ICAM5, IFNAR2, IL10RB, or IFNGR2. In some embodiments, a target plurality of genes comprises two or more of CXCL1, CXCL2, CXCL3, CXCL4, CXCL5, CXCL6, CXCL7, IL8, CXCL15, CCL2, CCL7, CCL9, IL1A, IL1B, CSF2, IRF1, ICAM1, ICAM4, ICAM5, IFNAR2, IL10RB, or IFNGR2. In some embodiments, the plurality of genes comprises one or more genes more human CXCL family. In some embodiments, a target plurality of genes comprises CXCL1 (e.g., nucleic acid sequence encoding an RNA according to NM_002089 or a nucleic acid encoding a polypeptide according to P09341, or a mutant thereof), CXCL2 (e.g., nucleic acid sequence encoding an RNA according to NM_001511 or a nucleic acid encoding a polypeptide according to P19875, or a mutant thereof), CXCL3 (e.g., nucleic acid sequence encoding an RNA according to NM_002090 or a nucleic acid encoding a polypeptide according to P19876, or a mutant thereof), CXCL4 (e.g., nucleic acid sequence encoding an RNA according to NM_002619 or NM_001363352, or a nucleic acid encoding a polypeptide according to P02776, or a mutant thereof), CXCL5 (e.g., nucleic acid sequence encoding an RNA according to NM_002994 or a nucleic acid encoding a polypeptide according to P42830, or a mutant thereof), CXCL6 (e.g., nucleic acid sequence encoding an RNA according to NM_002993 or a nucleic acid encoding a polypeptide according to P80162, or a mutant thereof), CXCL7 (e.g., nucleic acid sequence encoding an RNA according to NM_002704 or a nucleic acid encoding a polypeptide according to P02775, or a mutant thereof), and IL8 (e.g., nucleic acid sequence encoding an RNA according to NM_000584 or NM_001354840, or a nucleic acid encoding a polypeptide according to P10145, or a mutant thereof). In some embodiments, the plurality of genes comprises one or more genes more mouse CXCL family. In some embodiments, a target plurality of genes comprises CXCL1 (e.g., nucleic acid sequence encoding an RNA according to NM_008176.3 or a nucleic acid encoding a polypeptide according to P12850, or a mutant thereof), CXCL2 (e.g., nucleic acid sequence encoding an RNA according to NM_009140.2 or a nucleic acid encoding a polypeptide according to P10889, or a mutant thereof), CXCL3 (e.g., nucleic acid sequence encoding an RNA according to NM_203320.3 or a nucleic acid encoding a polypeptide according to Q6W5C0, or a mutant thereof), CXCL4 (e.g., nucleic acid sequence encoding an RNA according to NM_019932 or a nucleic acid encoding a polypeptide according to Q9Z126, or a mutant thereof), CXCL5 (e.g., nucleic acid sequence encoding an RNA according to NM_009141.3 or a nucleic acid encoding a polypeptide according to P50228, or a mutant thereof), CXCL7 (e.g., nucleic acid sequence encoding an RNA according to NM_023785.3 or a nucleic acid encoding a polypeptide according to Q9EQI5, or a mutant thereof), and CXCL15 (e.g., nucleic acid sequence encoding an RNA according to NM_011339 or a nucleic acid encoding a polypeptide according to Q9WVL7, or a mutant thereof). In some embodiments, a target plurality of genes comprises CCL2, CCL7, CCL9, IL1A, and IL1B. In some embodiments, a target plurality of genes comprises CSF2, IRF1, ICAM1, ICAM4, and ICAM5. In some embodiments, a target plurality of genes comprises IFNAR2, IL10RB, and IFNGR2.

In some embodiments, inhibition expression of two or more (e.g., all) genes of a target plurality of genes may modulate expression of other genes encoding a protein, e.g., cytokines, e.g., decreasing CXCL expression and cellular recruitment of CXCL to the site of inflammation, reduces presence of GM-CSF, and/or IL-6 in the site of inflammation.

In some embodiments, a target plurality of genes is part of a genomic complex, e.g., ASMC. As used herein, referring to a target plurality of genes being part of a genomic complex, e.g., ASMC, means that each of the genes of the plurality are at least partly comprised within the genomic complex, e.g., ASMC. Referring to a target plurality of genes as part of a genomic complex, e.g., ASMC, is used interchangeably with reference to a genomic complex, e.g., ASMC, comprising a target plurality of genes. For example, a target plurality of genes may consist of two genes positioned adjacent one another in the genome wherein a first anchor sequence is disposed within the first of the genes and a second anchor sequence is disposed outside of the second of the genes distal to the first gene. An ASMC formed by association of said first and second anchor sequence would wholly comprise the second of the genes and partly comprise the first of the genes; a plurality of genes consisting of these two genes would be part of this ASMC. In some embodiments, each gene of a target plurality of genes is wholly within a genomic complex, e.g., ASMC, (e.g., no portion of the transcript encoding gene sequence is outside of the genomic complex, e.g., ASMC). In some embodiments, each gene of a target plurality of genes is partly within a genomic complex, e.g., ASMC, (e.g., some portion of the transcript encoding gene sequence is outside of the genomic complex, e.g., ASMC). In some embodiments, at least one gene of a target plurality of genes is wholly within a genomic complex, e.g., ASMC, (e.g., no portion of the transcript encoding gene sequence is outside of the genomic complex, e.g., ASMC), and at least one gene of a target plurality of genes is partly within a genomic complex, e.g., ASMC, (e.g., some portion of the transcript encoding gene sequence is outside of the genomic complex, e.g., ASMC).

A gene of a target plurality of genes may include coding sequences, e.g., exons, and/or non-coding sequences, e.g., introns, 3′UTR, or 5′UTR. In some embodiments, a gene of a target plurality of genes is operably linked to a transcription control element. In some embodiments, a transcription control element of a gene of a target plurality of genes is also part of the genomic complex, e.g., ASMC, that the gene is part of. Referring to a transcription control element operably linked to a gene as part of a genomic complex, e.g., ASMC, can be understood in the same sense as described above in reference to the target plurality of genes. In some embodiments, each transcription control element operably linked to a gene of a target plurality of genes is wholly within a genomic complex, e.g., ASMC, (e.g., no portion of the transcription control element sequence is outside of the genomic complex, e.g., ASMC). In some embodiments, each transcription control element of a target plurality of genes is partly within a genomic complex, e.g., ASMC, (e.g., some portion of the transcription control element sequence is outside of the genomic complex, e.g., ASMC). In some embodiments, each transcription control element of a target plurality of genes is completely outside of the genomic complex, e.g., ASMC, (e.g., each transcription control element sequence is outside of the genomic complex, e.g., ASMC). In some embodiments, at least one transcription control element operably linked to a gene of a target plurality of genes is wholly within a genomic complex, e.g., ASMC (e.g., no portion of the transcription control element sequence is outside of the genomic complex, e.g., ASMC). In some embodiments, at least one transcription control element operably linked to another gene of the target plurality of genes is partly within the genomic complex, e.g., ASMC, (e.g., some portion of the transcript encoding gene sequence is outside of the genomic complex, e.g., ASMC). In some embodiments, at least one transcription control element operably linked to a gene of a target plurality of genes is completely outside of the genomic complex, e.g., ASMC.

In some embodiments, a site-specific disrupting agent or a system targets a target plurality of genes by binding to an anchor sequence, e.g., an anchor sequence that is part of a genomic complex (e.g., ASMC) comprising the target plurality of genes. In some embodiments, a targeting moiety binds to the anchor sequence. In some embodiments, binding of a genomic complex component, e.g., nucleating polypeptide, to an anchor sequence nucleates complex formation, e.g., anchor sequence-mediated conjunction formation. Each anchor sequence-mediated conjunction comprises one or more anchor sequences, e.g., a plurality of anchor sequences. In some embodiments, an anchor sequence-mediated conjunction can be disrupted to alter, e.g., inhibit, expression of a target plurality of genes. Such disruptions may modulate gene expression by, e.g., changing topological structure of DNA, e.g., by modulating the ability of a gene of the plurality to interact with a transcription control element (e.g., enhancing and silencing/repressive sequences).

A targeting moiety suitable for use in a site-specific disrupting agent or a system may bind, e.g., specifically bind, to a site that is proximal to an anchor sequence, e.g., an anchor sequence that is part of a genomic complex (e.g., ASMC) comprising the target plurality of genes. As used herein, proximal refers to a closeness of two sites, e.g., nucleic acid sites, such that binding of a site-specific disrupting agent or a system at the first site and/or modification of the first site by the site-specific disrupting agent will produce the same or substantially the same effect as binding and/or modification of the other site. For example, a targeting moiety may bind to a first site that is proximal to an anchor sequence that is part of a genomic complex (e.g., ASMC) comprising the target plurality of genes (the second site), and an effector moiety associated with said targeting moiety may epigenetically modify the first site such that genomic complex (e.g., ASMC) comprising the anchor sequence is modified, substantially the same as if the second site had been bound and/or modified. In some embodiments, a site proximal to a target gene (e.g., an exon, intron, or splice site within the target gene), proximal to a transcription control element operably linked to the target gene, or proximal to an anchor sequence is within 5000, 4000, 3000, 2000, 1000, 900, 800, 700, 600, 500, 400, 300, 200, 100, 50, 40, 30, 25, 20, 15, 10, or 5 base pairs from the target gene (e.g., an exon, intron, or splice site within the target gene), transcription control element, or anchor sequence (and optionally at least 5, 10, 20, 25, 50, 100, 200, or 300 base pairs from the target gene (e.g., an exon, intron, or splice site within the target gene), transcription control element, or anchor sequence). In some embodiments, a site proximal to an anchor sequence is a site that is less than 1000, 900, 800, 700, 600, 500, 400, 300, 200, 100, 50, 40, 30, 20, 10, or 5 base pairs from the anchor sequence (and optionally at least 5, 10, 20, 25, 50, 100, 200, or 300 base pairs). In some embodiments, a site proximal to an anchor sequence is a site that is less than 800, 700, 600, 500, 400, or 300 base pairs from the anchor sequence (and optionally at least 5, 10, 20, 25, 50, 100, 200, or 300 base pairs).

A targeting moiety suitable for use in a site-specific disrupting agent or a system described herein may bind, e.g., specifically bind, to a site comprising at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs (and optionally no more 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 nucleotides or base pairs). In some embodiments, a targeting moiety binds to a site comprising 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs.

### Genomic Complexes

Genomic complexes relevant to the present disclosure include stable structures that comprise a plurality of polypeptide and/or nucleic acid (particularly ribonucleic acid) components and that co-localize two or more genomic sequence elements (e.g., anchor sequences, promoter and/or enhancer elements). In some embodiments, relevant genomic complexes comprise anchor-sequence-mediated conjunctions (e.g., genomic loops). In some embodiments, genomic sequence elements that are (i.e., in three-dimensional space) in genomic complexes include transcriptional promoter and/or regulatory (e.g., enhancer or repressor) sequences. Alternatively, or additionally, in some embodiments, genomic sequence elements that are in genomic complexes include binding sites for one or more of CTCF, YY1, etc. In some embodiments, a genomic complex comprises a target plurality of genes. In some embodiments, two or more (e.g., all) genes of a target plurality of genes are located in a single loop of an ASMC. In some embodiments, two or more (e.g., all) genes of a target plurality of genes are located in different loops of an ASMC.

In some embodiments, a genomic complex whose incidence is decreased in accordance with the present disclosure comprises, or consists of, one or more components chosen from: a genomic sequence element (e.g., an anchor sequence, e.g., a CTCF binding motif, a YY1 binding motif, etc., that may, in some embodiments, be recognized by a nucleating component), one or more polypeptide components (e.g., one or more nucleating polypeptides, one or more transcriptional machinery proteins, and/or one or more transcriptional regulatory proteins), and/or one or more non-genomic nucleic acid components (e.g., non-coding RNA and/or an mRNA, for example, transcribed from a gene associated with the genomic complex).

In some embodiments, a genomic complex component is part of a genomic complex, wherein the genomic complex brings together two genomic sequence elements that are spaced apart from one another on a chromosome, e.g., via an interaction between and among a plurality of protein and/or other components.

In some embodiments, a genomic sequence element is an anchor sequences to which one or more protein components of the complex binds; thus, in some embodiments, a genomic complex comprises an anchor-sequence-mediated conjunction. In some embodiments, a genomic sequence element comprises a CTCF binding motif, a promoter and/or an enhancer. In some embodiments, a genomic sequence element includes at least one or both of a promoter and/or regulatory site (e.g., an enhancer). In some embodiments, complex formation is nucleated at the genomic sequence element(s) and/or by binding of one or more of the protein component(s) to the genomic sequence element(s).

Genomic sequence elements involved in genomic complexes as described herein, may be non-contiguous with one another. In some embodiments with noncontiguous genomic sequence elements (e.g., anchor sequences, promoters, and/or transcriptional regulatory sequences), a first genomic sequence element (e.g., anchor sequence, promoter, or transcriptional regulatory sequence) may be separated from a second genomic sequence element (e.g., anchor sequence, promoter, or transcriptional regulatory sequence) by about 500 bp to about 500 Mb, about 750 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a first genomic sequence element (e.g., anchor sequence, promoter, or transcriptional, regulatory sequence) is separated from a second genomic sequence element (e.g., anchor sequence, promoter, or transcriptional regulatory sequence) by about 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

### Anchor Sequence-Mediated Conjunction

In some embodiments, a genomic complex relevant to the present disclosure is or comprises an anchor sequence-mediated conjunction (ASMC). In some embodiments, an anchor-sequence-mediated conjunction is formed when nucleating polypeptide(s) bind to anchor sequences in the genome and interactions between and among these proteins and, optionally, one or more other components, forms a conjunction in which the anchor sequences are physically co-localized. In many embodiments described herein, one or more genes is associated with an anchor-sequence-mediated conjunction; in such embodiments, the anchor sequence-mediated conjunction typically includes one or more anchor sequences, one or more genes, and one or more transcriptional control sequences, such as an enhancing or silencing sequence. In some embodiments, a transcriptional control sequence is within, partially within, or outside an anchor sequence-mediated conjunction. In some embodiments, the ASMC comprises an internal enhancing sequence, e.g., an enhancer. In some embodiments, an ASMC comprises a target plurality of genes.

In some embodiments, a genomic complex as described herein (e.g., an anchor sequence-mediated conjunction) is or comprises a genomic loop, such as an intra-chromosomal loop. In certain embodiments, genomic complex as described herein (e.g., an anchor sequence-mediated conjunction) comprises a plurality of genomic loops. One or more genomic loops may include a first anchor sequence, a nucleic acid sequence, a transcriptional control sequence, and a second anchor sequence. In some embodiments, at least one genomic loop includes, in order, a first anchor sequence, a transcriptional control sequence, and a second anchor sequence; or a first anchor sequence, a nucleic acid sequence, and a second anchor sequence. In yet some embodiments, either one or both of nucleic acid sequences and transcriptional control sequence is located within a genomic loop. In yet some embodiments, either one or both of nucleic acid sequences and transcriptional control sequence is located outside a genomic loop. In some embodiments, one or more genomic loops comprise a transcriptional control sequence. In some embodiments, genomic complex (e.g., an anchor sequence-mediated conjunction) includes a TATA box, a CAAT box, a GC box, or a CAP site.

In some embodiments, an anchor sequence-mediated conjunction comprises a plurality of genomic loops; in some such embodiments, an anchor sequence-mediated conjunction comprises at least one of an anchor sequence, a nucleic acid sequence, and a transcriptional control sequence in one or more genomic loops.

Types of Loops

In some embodiments, a genomic loop comprises one or more, e.g., 2, 3, 4, 5, or more, genes, e.g., a target plurality of genes. In some embodiments, two or more, e.g., 2, 3, 4, 5, or more, genes of the target plurality of genes are transcribed in the same direction. In some embodiments, all genes of the target plurality of genes are transcribed in the same direction.

In some embodiments, the present disclosure provides methods of modulating (e.g., decreasing) expression of a target plurality of genes in a loop comprising inhibiting, dissociating, degrading, and/or modifying a genomic complex that achieves co-localization of genomic sequences that are outside of, not part of, or comprised within (i) a gene whose expression is modulated (e.g. of a target plurality of genes); and/or (ii) one or more associated transcriptional control sequences that influence transcription of a gene whose expression is modulated.

In some embodiments, the present disclosure provides methods of modulating (e.g., decreasing) transcription of a target plurality of genes comprising inhibiting formation of and/or destabilizing a complex that achieves co-localization of genomic sequences that are non-contiguous with (i) a gene whose expression is modulated (e.g., of a target plurality of genes); and/or (ii) associated transcriptional control sequences that influence transcription of a gene whose expression is modulated.

In some embodiments, an anchor sequence-mediated conjunction is associated with one or more, e.g., 2, 3, 4, 5, or more, transcriptional control sequences. In some embodiments, a gene of a target plurality of genes (e.g., one, two, or more, e.g., all of the target plurality of genes) is non-contiguous with one or more transcriptional control sequences. In some embodiments where a gene is non-contiguous with its transcriptional control sequence(s), a gene may be separated from one or more transcriptional control sequences by about 100 bp to about 500 Mb, about 500 bp to about 200 Mb, about 1 kb to about 100 Mb, about 25 kb to about 50 Mb, about 50 kb to about 1 Mb, about 100 kb to about 750 kb, about 150 kb to about 500 kb, or about 175 kb to about 500 kb. In some embodiments, a gene is separated from a transcriptional control sequence by about 100 bp, 300 bp, 500 bp, 600 bp, 700 bp, 800 bp, 900 bp, 1 kb, 5 kb, 10 kb, 15 kb, 20 kb, 25 kb, 30 kb, 35 kb, 40 kb, 45 kb, 50 kb, 55 kb, 60 kb, 65 kb, 70 kb, 75 kb, 80 kb, 85 kb, 90 kb, 95 kb, 100 kb, 125 kb, 150 kb, 175 kb, 200 kb, 225 kb, 250 kb, 275 kb, 300 kb, 350 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 15 Mb, 20 Mb, 25 Mb, 50 Mb, 75 Mb, 100 Mb, 200 Mb, 300 Mb, 400 Mb, 500 Mb, or any size therebetween.

### Anchor Sequences

In general, an anchor sequence is a genomic sequence element to which a genomic complex component, e.g., nucleating polypeptide, binds specifically. In some embodiments, binding of a genomic complex component to an anchor sequence nucleates complex formation.

Each anchor sequence-mediated conjunction comprises one or more anchor sequences, e.g., a plurality. In some embodiments, anchor sequences can be manipulated or altered to form and/or stabilize naturally occurring loops, to form one or more new loops (e.g., to form exogenous loops or to form non-naturally occurring loops with exogenous or altered anchor sequences), or to inhibit formation of or destabilize naturally occurring or exogenous loops. Such alterations may modulate gene expression by, e.g., changing topological structure of DNA, e.g., by thereby modulating ability of a target gene to interact with gene regulation and control factors (e.g., enhancing and silencing/repressor sequences). In some embodiments, chromatin structure is modified by substituting, adding or deleting one or more nucleotides within an anchor sequence-mediated conjunction. In some embodiments, chromatin structure is modified by substituting, adding, or deleting one or more nucleotides within an anchor sequence of an anchor sequence-mediated conjunction.

In some embodiments, an anchor sequence comprises a common nucleotide sequence, e.g., a CTCF-binding motif: N(T/C/G)N(G/A/T)CC(A/T/G)(C/G)(C/T/A)AG(G/A)(G/T)GG(C/A/T)(G/A)(C/G)(C/T/A)(G/A/C) (SEQ ID NO:1), where N is any nucleotide.

A CTCF-binding motif may also be in an opposite orientation, e.g.,

(G/A/C)(C/T/A)(C/G)(G/A)(C/A/T)GG(G/T)(G/A)GA(C/T/A)(C/G)(A/T/G)CC(G/A/T)N(T/C/G)N (SEQ ID NO:2). In some embodiments, an anchor sequence comprises SEQ ID NO:1 or SEQ ID NO:2 or a sequence at least 75%, at least 80%, at least 90%, at least 95%, at least 96%, at least 97%, at least 98%, at least 99% identical to either SEQ ID NO:1 or SEQ ID NO:2.

In some embodiments, an anchor sequence comprises a CTCF binding motif, a USF1 binding motif, a YY1 binding motif, a TAF3 binding motif, or a ZNF143 binding motif. In some embodiments, an anchor sequence comprises a nucleating polypeptide binding motif, e.g., a YY1-binding motif: CCGCCATNTT (SEQ ID NO: 3), where N is any nucleotide. A YY1-binding motif may also be in an opposite orientation, e.g., AANATGGCGG (SEQ ID NO: 4), where N is any nucleotide. In some embodiments, an anchor sequence comprises SEQ ID NO:3 or SEQ ID NO:4 or a sequence at least 75%, at least 80%, at least 90%, at least 95%, at least 96%, at least 97%, at least 98%, at least 99% identical to either SEQ ID NO:3 or SEQ ID NO:4.

In some embodiments, an anchor sequence-mediated conjunction comprises at least a first anchor sequence and a second anchor sequence. For example, in some embodiments, a first anchor sequence and a second anchor sequence may each comprise a common nucleotide sequence, e.g., each comprises a CTCF binding motif. In some embodiments, a first anchor sequence and a second anchor sequence may each comprise a USF1 binding motif. In some embodiments, a first anchor sequence and a second anchor sequence may each comprise a YY1 binding motif. In some embodiments, a first anchor sequence and a second anchor sequence may each comprise a TAF3 binding motif. In some embodiments, a first anchor sequence and a second anchor sequence may each comprise a ZNF143 binding motif.

In some embodiments, a first anchor sequence and second anchor sequence comprise different sequences, e.g., a first anchor sequence comprises a CTCF binding motif, and a second anchor sequence comprises an anchor sequence other than a CTCF binding motif. In some embodiments, a first anchor sequence comprises a CTCF binding motif, a USF1 binding motif, a YY1 binding motif, a TAF3 binding motif, or a ZNF143 binding motif, and a second anchor sequence comprises a CTCF binding motif, a USF1 binding motif, a YY1 binding motif, a TAF3 binding motif, or a ZNF143 binding motif, wherein the first and second anchor sequences do not both comprise a CTCF binding motif, a USF1 binding motif, a YY1 binding motif, a TAF3 binding motif, or a ZNF143 binding motif. In some embodiments, each anchor sequence comprises a common nucleotide sequence (e.g., a CTCF binding motif, a USF1 binding motif, a YY1 binding motif, a TAF3 binding motif, or a ZNF143 binding motif) and one or more flanking nucleotides on one or both sides of a common nucleotide sequence.

Two anchor sequences (e.g., each comprising a CTCF binding motif, a USF1 binding motif, a YY1 binding motif, a TAF3 binding motif, or a ZNF143 binding motif) that can form a conjunction may be present in a genome in any orientation, e.g., in the same orientation (tandem) either 5′-3′ (left tandem, or 3′-5′ (right tandem), or convergent orientation, where one anchor sequence is oriented 5′-3′ and the other is oriented 3′-5′.

Two CTCF-binding motifs (e.g., contiguous or non-contiguous CTCF binding motifs) that can form a conjunction may be present in a genome in any orientation, e.g., in the same orientation (tandem) either 5′-3′ (left tandem, e.g., the two CTCF-binding motifs that comprise SEQ ID NO:1) or 3′-5′ (right tandem, e.g., the two CTCF-binding motifs comprise SEQ ID NO:2), or convergent orientation, where one CTCF-binding motif comprises SEQ ID NO:1 and another other comprises SEQ ID NO:2. CTCFBSDB 2.0: Database For CTCF binding motifs And Genome Organization (on the world wide web at insulatordb.uthsc.edu/) can be used to identify CTCF binding motifs associated with a target gene. In some embodiments, an anchor sequence comprises a CTCF binding motif associated with a target plurality of genes, wherein the target plurality of genes is associated with a disease, disorder and/or condition. In some embodiments, an anchor sequence comprises a CTCF binding motif associated with a target plurality of genes, wherein the genes of the target plurality of genes have related functionalities. In some embodiments, an anchor sequence comprises a CTCF binding motif associated with a target plurality of genes, wherein the target plurality of genes (e.g., two or more, e.g., all, of the plurality) are aberrantly expressed in a cell of a subject.

In some embodiments, chromatin structure may be modified by substituting, adding, or deleting one or more nucleotides within at least one anchor sequence, e.g., a nucleating polypeptide binding motif. One or more nucleotides may be specifically targeted, e.g., a targeted alteration, for substitution, addition or deletion within an anchor sequence, e.g., a nucleating polypeptide binding motif.

In some embodiments, an anchor sequence-mediated conjunction may be altered by changing an orientation of at least one common nucleotide sequence, e.g., a nucleating polypeptide binding motif. In some embodiments, an anchor sequence comprises a nucleating polypeptide binding motif, e.g., CTCF binding motif, and a targeting moiety introduces an alteration in at least one nucleating polypeptide binding motif, e.g., altering binding affinity for a nucleating polypeptide.
In some embodiments, an anchor sequence-mediated conjunction may be altered by introducing an exogenous anchor sequence. In some embodiments, addition of a non-naturally occurring or exogenous anchor sequence to destabilize or inhibit formation of a naturally occurring anchor sequence-mediated conjunction, e.g., by inducing a non-naturally occurring loop to form, alters (e.g., decreases) transcription of a nucleic acid sequence.

### Other Compositions

Nucleic Acids and Vectors

The present disclosure is further directed, in part, to nucleic acids encoding a site-specific disrupting agent or a system described herein. In some embodiments, a site-specific disrupting agent may be provided via a composition comprising a nucleic acid encoding a site-specific disrupting agent, e.g., a targeting moiety and/or effector moiety of the site-specific disrupting agent, wherein the nucleic acid is associated with sufficient other sequences to achieve expression of the site-specific disrupting agent in a system of interest (e.g., in a particular cell, tissue, organism, etc). In some embodiments, system may be provided via a composition comprising a first nucleic acid encoding a first site-specific disrupting agent, e.g., a first targeting moiety and/or a first effector moiety of the first site-specific disrupting agent, and a second nucleic acid encoding a second site-specific disrupting agent, e.g., a second targeting moiety and/or a second effector moiety of the second site-specific disrupting agent wherein the first and/or the second nucleic acid is associated with sufficient other sequences to achieve expression of the site-specific disrupting agents in a system of interest (e.g., in a particular cell, tissue, organism, etc).

In some particular embodiments, the present disclosure provides compositions of nucleic acids that encode a site-specific disrupting agent or polypeptide or nucleic acid portion thereof (e.g., a targeting moiety and/or effector moiety comprising a polypeptide and/or nucleic acid). In some particular embodiments, the present disclosure provides compositions of nucleic acids that encode a first site-specific disrupting agent and a second site-specific disrupting agent, or polypeptides or nucleic acid portions thereof (e.g., a targeting moiety and/or effector moiety comprising a polypeptide and/or nucleic acid). In some such embodiments, provided nucleic acids may include DNA, RNA, or any other nucleic acid moiety or entity as described herein, and may be prepared by any technology described herein or otherwise available in the art (e.g., synthesis, cloning, amplification, in vitro or in vivo transcription, etc). In some embodiments, provided nucleic acids that encode one or more site-specific disrupting agents, or polypeptides or nucleic acid portions thereof may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, a composition for delivering a site-specific disrupting agent or a system described herein comprises a vector, e.g., a viral vector, comprising one or more nucleic acids encoding a site-specific disrupting agent or polypeptide or nucleic acid portion thereof. In some embodiments, a first vector comprises a first nucleic acid encoding a first site-specific disrupting agent, and second vector comprises a second nucleic acid encoding a second site-specific disrupting agent. In some embodiments a single vector comprises a first nucleic acid encoding a first site-specific disrupting agent and a second nucleic acid encoding a second site-specific disrupting agent.

In some embodiments, a composition for delivering a site-specific disrupting agent or a system described herein is or comprises RNA, e.g., mRNA, comprising one or more nucleic acids encoding one or more components of a site-specific disrupting agent or polypeptide or nucleic acid portion thereof.

Nucleic acids as described herein or nucleic acids encoding a protein described herein, may be incorporated into a vector. Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. An expression vector may be provided to a cell in the form of a viral vector. Viral vector technology is well known in the art and described in a variety of virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

Expression of natural or synthetic nucleic acids is typically achieved by operably linking a nucleic acid encoding the gene of interest to a promoter and incorporating the construct into an expression vector. Vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, may regulate frequency of transcriptional initiation. Typically, these sequences are located in a region 30-110 bp upstream of a transcription start site, although a number of promoters have recently been shown to contain functional elements downstream of transcription start sites as well. Spacing between promoter elements frequently is flexible, so that promoter function is preserved when elements are inverted or moved relative to one another. In a thymidine kinase (tk) promoter, spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. In some embodiments of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, an actin promoter, a myosin promoter, a hemoglobin promoter, and a creatine kinase promoter. The present disclosure should not be interpreted to be limited to use of any particular promoter or category of promoters (e.g., constitutive promoters). For example, in some embodiments, inducible promoters are contemplated as part of the present disclosure. In some embodiments, use of an inducible promoter provides a molecular switch capable of turning on expression of a polynucleotide sequence to which it is operatively linked, when such expression is desired. In some embodiments, use of an inducible promoter provides a molecular switch capable of turning off expression when expression is not desired. Examples of inducible promoters include, but are not limited to a metallothionine promoter, a glucocorticoid promoter, a progesterone promoter, and a tetracycline promoter.

In some embodiments, an expression vector to be introduced can also contain either a selectable marker gene or a reporter gene or both to facilitate identification and selection of expressing cells from the population of cells sought to be transfected or infected through viral vectors. In some aspects, a selectable marker may be carried on a separate piece of DNA and used in a co-transfection procedure. Both selectable markers and reporter genes may be flanked with appropriate transcriptional control sequences to enable expression in the host cells. Useful selectable markers may include, for example, antibiotic-resistance genes, such as neo, etc.

In some embodiments, reporter genes may be used for identifying potentially transfected cells and/or for evaluating the functionality of transcriptional control sequences. In general, a reporter gene is a gene that is not present in or expressed by a recipient source (of a reporter gene) and that encodes a polypeptide whose expression is manifested by some easily detectable property, e.g., enzymatic activity or visualizable fluorescence. Expression of a reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, a construct with a minimal 5′ flanking region that shows highest level of expression of reporter gene is identified as a promoter. Such promoter regions may be linked to a reporter gene and used to evaluate agents for ability to modulate promoter-driven transcription.

Cells

The present disclosure is further directed, in part, to cells comprising a site-specific disrupting agent or a system described herein. Any cell, e.g., cell line, e.g., a cell line suitable for expression of a recombinant polypeptide, known to one of skill in the art is suitable to comprise a site-specific disrupting agent described herein. In some embodiments, a cell, e.g., cell line, may be used to express a site-specific disrupting agent, a system comprising one or more site-specific agents, or nucleic acid or polypeptide portion thereof. In some embodiments, a cell, e.g., cell line, may be used to express or amplify a nucleic acid, e.g., a vector, encoding a site-specific disrupting agent. In some embodiments, a cell, e.g., cell line, may be used to express or amplify one or more nucleic acids, e.g., a vector, encoding a first site-specific disrupting agent and a vector encoding a second site specific disrupting agent. In some embodiments, a cell comprises a nucleic acid encoding a site-specific disrupting agent described herein. In some embodiments, a cell comprises a first nucleic acid encoding a first site-specific disrupting agent and a second nucleic acid encoding a second site-specific disrupting agent described herein.

In some embodiments, a cell comprises a nucleic acid encoding a site-specific disrupting agent or nucleic acid or polypeptide portion thereof, and the nucleic acid is integrated into the genomic DNA of the cell. In some embodiments, a cell comprises a nucleic acid encoding a site-specific disrupting agent or nucleic acid or polypeptide portion thereof, and the nucleic acid is disposed on a vector. In some embodiments, a cell comprises a first nucleic acid encoding a first site-specific disrupting agent and a second nucleic acid encoding a second site-specific disrupting agent or nucleic acid or polypeptide portion thereof, and the first and the second nucleic acid are integrated into the genomic DNA of the cell. In some embodiments, a cell comprises a first nucleic acid encoding a first site-specific disrupting agent and a second nucleic acid encoding a second site-specific disrupting agent or nucleic acid or polypeptide portion thereof, and the first and the second nucleic acid are disposed on a vector.

Examples of cells that may comprise and/or express a site-specific disrupting agent or a system herein include, but are not limited to, hepatocytes, stellate cells, Kupffer cells, neuronal cells, endothelial cells, alveolar cells, epithelial cells, myocytes, synovial layer, chondrocytes, immune cells, and lymphocytes.

The present disclosure is further directed, in part, to a cell made by a method or process described herein. In some embodiments, the disclosure provides a cell produced by, providing a site-specific disrupting agent described herein, providing the cell, and contacting the cell with the site-specific disrupting agent (or a nucleic acid encoding the site-specific disrupting agent, or a composition comprising said site-specific disrupting agent or nucleic acid). In some embodiments, the disclosure provides a cell produced by, providing system described herein, providing the cell, and contacting the cell with the system (or a first nucleic acid encoding the first site-specific disrupting agent and second nucleic acid encoding the second site-specific disrupting agent, or a composition comprising said system or nucleic acids). Without wishing to be bound by theory, a cell contacted with a site-specific disrupting agent or a system described herein may exhibit: a decrease in expression of a target plurality of genes; a modification of epigenetic markers associated with the target plurality of genes, a transcription control element operably linked to the target plurality of genes, or an anchor sequence proximal to the target plurality of genes or associated with an anchor sequence-mediated conjunction comprising the target plurality of genes; a genetic modification of a gene of the target plurality of genes, a transcription control element operably linked to the target plurality of genes, or an anchor sequence proximal to the target plurality of genes or associated with an anchor sequence-mediated conjunction comprising the target plurality of genes; and/or a decrease (e.g., the absence of) in the level of a genomic complex, e.g., ASMC, comprising a target plurality of genes, compared to a similar cell that has not been contacted by the site-specific disrupting agent. In some embodiments, a cell exhibiting said decrease in expression of a target plurality of genes, modification of epigenetic markers, and/or genetic modification does not comprise the site-specific disrupting agent. The decrease in expression of a target plurality of genes, modification of epigenetic markers, and/or genetic modification may persist, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after contact with the site-specific disrupting agent. In some embodiments, a cell previously contacted by a site-specific disrupting agent retains the decrease in expression of a target plurality of genes, modification of epigenetic markers, and/or genetic modification after the site-specific disrupting agent is no longer present in the cell, e.g., for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, 7, 10, or 14 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely) after the site-specific disrupting agent is no longer present in the cell. In some embodiments, the cell is a mammalian, e.g., human, cell. In some embodiments, the cell is a somatic cell and/or a primary cell.

Kits

The present disclosure further directed, in part, to a kit comprising a site-specific disrupting agent, a system, nucleic acid encoding a site-specific disrupting agent, or a first nucleic acid encoding the first site-specific disrupting agent and a second nucleic acid encoding the second site-specific disrupting agent described herein. In some embodiments, a kit comprises a site-specific disrupting agent, a system, or nucleic acid encoding the same and instructions for the use of said site-specific disrupting agent or the system. In some embodiments, a kit comprises a nucleic acid encoding the site-specific disrupting agent or a component thereof (e.g., a polypeptide or nucleic acid portion of the site-specific disrupting agent) and instructions for the use of said nucleic acid and/or said site-specific disrupting agent. In some embodiments, a kit comprises a cell comprising a nucleic acid encoding the site-specific disrupting agent or a component thereof (e.g., a polypeptide or nucleic acid portion of the site-specific disrupting agent) and instructions for the use of said cell, nucleic acid, and/or said site-specific disrupting agent. In some embodiments, a kit comprises or a first nucleic acid encoding the first site-specific disrupting agent and a second nucleic acid encoding the second site-specific disrupting agent or a component thereof (e.g., a polypeptide or nucleic acid portion of the first and the second site-specific disrupting agent) and instructions for the use of said nucleic acids and/or said system. In some embodiments, a kit comprises a cell comprising a nucleic acid encoding or a first nucleic acid encoding the first site-specific disrupting agent and a second nucleic acid encoding the second site-specific disrupting agent or a component thereof (e.g., a polypeptide or nucleic acid portion of the first and the second site-specific disrupting agent) and instructions for the use of said cell, nucleic acid, and/or said system.

In some embodiments, a kit comprises a unit dosage of a site-specific disrupting agent, or a unit dosage of a nucleic acid, e.g., a vector, encoding a site-specific disrupting agent described herein. In some embodiments, a kit comprises a unit dosage of a system, or a unit dosage of a first and a second nucleic acid, e.g., a vector, encoding a first site-specific disrupting agent and a second site-specific disrupting agent described herein.

### Methods of Making a Site-Specific Disrupting Agent

In some embodiments, a site-specific disrupting agent or a system comprises one or more proteins and may thus be produced by methods of making proteins. As will be appreciated by one of skill, methods of making proteins or polypeptides (which may be included in modulating agents as described herein) are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

A protein or polypeptide of compositions of the present disclosure can be biochemically synthesized by employing standard solid phase techniques. Such methods include exclusive solid phase synthesis, partial solid phase synthesis methods, fragment condensation, classical solution synthesis. These methods can be used when a peptide is relatively short (e.g., 10 kDa) and/or when it cannot be produced by recombinant techniques (i.e., not encoded by a nucleic acid sequence) and therefore involves different chemistry.

Solid phase synthesis procedures are well known in the art and further described by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

For longer peptides, recombinant methods may be used. Methods of making a recombinant therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Exemplary methods for producing a therapeutic pharmaceutical protein or polypeptide involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

In cases where large amounts of the protein or polypeptide are desired, it can be generated using techniques such as described by Brian Bray, Nature Reviews Drug Discovery, 2:587-593, 2003; and Weissbach & Weissbach, 1988, Methods for Plant Molecular Biology, Academic Press, NY, Section VIII, pp 421-463.

Various mammalian cell culture systems can be employed to express and manufacture recombinant protein. Examples of mammalian expression systems include CHO cells, COS cells, HeLA and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein. Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012). Proteins comprise one or more amino acids. Amino acids include any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

### Pharmaceutical Compositions, Formulation, Delivery, and Administration

The present disclosure is further directed, in part, to pharmaceutical compositions comprising a site-specific disrupting agent described herein, and to pharmaceutical compositions comprising nucleic acids encoding a site-specific disrupting agent or a system described herein.

As used herein, the term “pharmaceutical composition” refers to an active agent (e.g., a site-specific disrupting agent or a system, or nucleic acid encoding the same), formulated together with one or more pharmaceutically acceptable carriers (e.g., pharmaceutically acceptable carriers known to those of skill in the art). In some embodiments, active agent is present in unit dose amount appropriate for administration in a therapeutic regimen that shows a statistically significant probability of achieving a predetermined therapeutic effect when administered to a relevant population. In some embodiments, a pharmaceutical composition comprises a site-specific disrupting agent or a system of the present disclosure.

In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; trans-dermally; or nasally, pulmonary, and/or to other mucosal surfaces, for example, as aerosols, aqueous solutions, or suspensions. In some embodiments, the composition may be lyophilized or spray dried. In some embodiments, the composition may be formulated for pulmonary administration and/or intravenous administration.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” means a pharmaceutically acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In some embodiments, for example, materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt”, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid, or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate, and aryl sulfonate.

In various embodiments, the present disclosure provides pharmaceutical compositions described herein with a pharmaceutically acceptable excipient. Pharmaceutically acceptable excipient includes an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

Pharmaceutical preparations may be made following conventional techniques of pharmacy involving milling, mixing, granulation, and compressing, when necessary, for tablet forms; or milling, mixing, and filling for hard gelatin capsule forms. When a liquid carrier is used, a preparation can be in the form of a syrup, elixir, emulsion or an aqueous or non-aqueous solution or suspension. Such a liquid formulation may be administered directly per os.

In some embodiments, pharmaceutical compositions may be formulated for delivery to a cell and/or to a subject via any route of administration. Modes of administration to a subject may include injection, infusion, inhalation, intranasal, intraocular, topical delivery, inter-cannular delivery, or ingestion. Injection includes, without limitation, intravenous, intramuscular, intra-arterial, intrathecal, intraventricular, intracapsular, intra-orbital, intracardiac, intradermal, intraperitoneal, transtracheal, subcutaneous, subcuticular, intraarticular, bronchial, sub-capsular, subarachnoid, intraspinal, intra-cerebrospinal, and intra-sternal injection and infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), or local (e.g., local application on the skin, intravitreal injection). In some embodiments, one or more compositions is administered systemically. In some embodiments, administration is non-parenteral and a therapeutic is a parenteral therapeutic. In some embodiments, administration may be bronchial (e.g., by bronchial instillation), buccal, dermal (which may be or comprise, for example, one or more of topical to the dermis, intradermal, inter-dermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be a single dose. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, six, eight, ten, 12, 15 or 20 or more administrations may be given to the subject during one treatment or over a period of time as a treatment regimen. In some embodiments, administrations may be given as needed, e.g., for as long as symptoms associated with the disease, disorder or condition persist. In some embodiments, repeated administrations may be indicated for the remainder of the subject's life. Treatment periods may vary and could be, e.g., one day, two days, three days, one week, two weeks, one month, two months, three months, six months, a year, or longer.

In some embodiments, administration is provided using a respiratory delivery device, e.g., nebulizer, e.g., metered-dose inhaler, e.g., dry powder inhaler. Some of the commercially available dry powder inhalers include Spinhaler (Fisons Pharmaceuticals, Rochester, NY) and Rotahaler (GSK, RTP, NC). In some embodiments, the nebulizer may include a jet nebulizer, an ultrasonic nebulizer, and/or a vibrating mesh nebulizer.

### Dosage

Pharmaceutical compositions according to the present disclosure may be delivered in a therapeutically effective amount. A precise therapeutically effective amount is an amount of a composition that will yield the most effective results in terms of efficacy of treatment in a given subject. This amount will vary depending upon a variety of factors, including but not limited to characteristics of a therapeutic compound (including activity, pharmacokinetics, pharmacodynamics, and bioavailability), physiological condition of a subject (including age, sex, disease type and stage, general physical condition, responsiveness to a given dosage, and type of medication), nature of a pharmaceutically acceptable carrier or carriers in a formulation, and/or route of administration.

In some aspects, the present disclosure provides methods of delivering a therapeutic comprising administering a composition as described herein to a subject, wherein a genomic complex modulating agent is a therapeutic and/or wherein delivery of a therapeutic causes changes in gene expression relative to gene expression in absence of a therapeutic.

Methods as provided in various embodiments herein may be utilized in any some aspects delineated herein. In some embodiments, one or more compositions is/are targeted to specific cells, or one or more specific tissues.

For example, in some embodiments one or more compositions is/are targeted to epithelial, connective, muscular, and/or nervous tissue or cells. In some embodiments a composition is targeted to a cell or tissue of a particular organ system, e.g., respiratory system (pharynx, larynx, trachea, bronchi, lungs, diaphragm), cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); skeletal system (bone, cartilage); and/or combinations thereof. In some embodiments, a composition is targeted to a cell, e.g., endothelial, alveolar, epithelial, hepatocytes, stellate cells, Kupffer cells, synovial layer, chondrocytes, fibroblast cells, ductal epithelial cells, epithelial enterocytes, goblet cells, basal cells, and/or immune cells. In some embodiments, a composition is targeted to a cell of an organ, e.g., nasal cells, lung cells, ileum cells, cardiac cells, optic cells, liver cells, bladder cells, pancreatic cells, kidney cells, neural cells, prostrate cells, testis cells, In some embodiments, a composition of the present disclosure crosses a blood-brain-barrier, a placental membrane, or a blood-testis barrier. In some embodiments, a composition is targeted to a cell expressing an ACE-2 receptor.

In some embodiments, a pharmaceutical composition as provided herein is administered systemically.

In some embodiments, administration is non-parenteral and a therapeutic is a parenteral therapeutic.

In some embodiments, a pharmaceutical composition of the present disclosure has improved PK/PD, e.g., increased pharmacokinetics or pharmacodynamics, such as improved targeting, absorption, or transport (e.g., at least 5%, 10%, 15%, 20%, 30%, 40%, 50%, 60%, 75%, 80%, 90% improved or more) as compared to a active agent alone. In some embodiments, a pharmaceutical composition has reduced undesirable effects, such as reduced diffusion to a nontarget location, off-target activity, or toxic metabolism, as compared to a therapeutic alone (e.g., at least 5%, 10%, 15%, 20%, 30%, 40%, 50%, 60%, 75%, 80%, 90% or more reduced, as compared to an active agent alone). In some embodiments, a composition increases efficacy and/or decreases toxicity of a therapeutic (e.g., at least 5%, 10%, 15%, 20%, 30%, 40%, 50%, 60%, 75%, 80%, 90% or more) as compared to an active agent alone.

Pharmaceutical compositions described herein may be formulated for example including a carrier, such as a pharmaceutical carrier and/or a polymeric carrier, e.g., a liposome or vesicle, and delivered by known methods to a subject in need thereof (e.g., a human or non-human agricultural or domestic animal, e.g., cattle, dog, cat, horse, poultry). Such methods include transfection (e.g., lipid-mediated, cationic polymers, calcium phosphate); electroporation or other methods of membrane disruption (e.g., nucleofection) and viral delivery (e.g., lentivirus, retrovirus, adenovirus, AAV). Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

Lipid Nanoparticles

Site-specific disrupting agents as described herein can be delivered using any biological delivery system/formulation including a particle, for example, a nanoparticle delivery system. Nanoparticles include particles with a dimension (e.g. diameter) between about 1 and about 1000 nanometers, between about 1 and about 500 nanometers in size, between about 1 and about 100 nm, between about 30 nm and about 200 nm, between about 50 nm and about 300 nm, between about 75 nm and about 200 nm, between about 100 nm and about 200 nm, and any range therebetween. A nanoparticle has a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In some embodiments, nanoparticles have a greatest dimension ranging between 25 nm and 200 nm. Nanoparticles as described herein comprise delivery systems that may be provided in any form, including but not limited to solid, semi-solid, emulsion, or colloidal nanoparticles. A nanoparticle delivery system may include but not limited to lipid-based systems, liposomes, micelles, microvesicles, exosomes, or gene gun. In one embodiment, the nanoparticle is a lipid nanoparticle (LNP). In some embodiments, the LNP is a particle that comprises a plurality of lipid molecules physically associated with each other by intermolecular forces. In some embodiments, an LNP may comprise multiple components, e.g., 3-4 components. In one embodiment, the site-specific disrupting agent or a pharmaceutical composition comprising said site-specific disrupting agent (or a nucleic acid encoding the same, or pharmaceutical composition comprising a nucleic acid encoding said site specific disrupting agent) is encapsulated in an LNP. In one embodiment, the system or a pharmaceutical composition comprising said system (or a nucleic acid encoding the same, or pharmaceutical composition comprising nucleic acid encoding the said system) is encapsulated in an LNP. In some embodiments, the nucleic acid encoding the first site-specific disrupting agent and the nucleic acid encoding the second site-specific disrupting agent are present in same LNP. In some embodiments, the nucleic acid encoding the first site-specific disrupting agent and the nucleic acid encoding the second site-specific disrupting agent are present in different LNPs. Preparation of LNPs and the modulating agent encapsulation may be used/and or adapted from Rosin et al, Molecular Therapy, vol. 19, no. 12, pages 1286-2200, December 2011). In some embodiments, lipid nanoparticle compositions disclosed herein are useful for expression of protein encoded by mRNA. In some embodiments, nucleic acids, when present in the lipid nanoparticles, are resistant in aqueous solution to degradation with a nuclease.

In some embodiments, the LNP formulations may include a CCD lipid, a neutral lipid, and/or a helper lipid. In some embodiments, the LNP formulation comprises an ionizable lipid. In some embodiments, an ionizable lipid may be a cationic lipid, an ionizable cationic lipid, or an amine-containing lipid that can be readily protonated. In some embodiments, the lipid is a cationic lipid that can exist in a positively charged or neutral form depending on pH. In some embodiments, the cationic lipid is a lipid capable of being positively charged, e.g., under physiological conditions. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, LNP formulation (e.g., MC3 and/or SSOP) includes cholesterol, PEG, and/or a helper lipid. The LNPs may be, e.g., microspheres (including uni-lamellar and multi-lamellar vesicles, lamellar phase lipid bilayers that, in some embodiments, are substantially spherical. In some embodiments, the LNP can comprise an aqueous core, e.g., comprising a nucleic acid encoding a site-specific disrupting agent or a system as disclosed herein. In some embodiments of the present disclosure, the cargo for the LNP formulation includes at least one guide RNA. In some embodiments, the cargo, e.g., a nucleic acid encoding a site-specific disrupting agent, or a system as disclosed herein, may be adsorbed to the surface of an LNP, e.g., an LNP comprising a cationic lipid. In some embodiments, the cargo, e.g., a nucleic acid encoding a site-specific disrupting agent, or a system as disclosed herein may be associated with the LNP. In some embodiments, the cargo, e.g., a nucleic acid encoding a site-specific disrupting agent, or a system as disclosed herein, may be encapsulated, e.g., fully encapsulated and/or partially encapsulated in an LNP.

In some embodiments, an LNP comprising a cargo may be administered for systemic delivery, e.g., delivery of a therapeutically effective dose of cargo that can result in a broad exposure of an active agent within an organism. Systemic delivery of lipid nanoparticles can be for example, intravenous, pulmonary, bronchial, intraarterial, subcutaneous, and intraperitoneal delivery. In some embodiments, systemic delivery of lipid nanoparticles is by intravenous delivery. In some embodiments, an LNP comprising a cargo may be administered for local delivery, e.g., delivery of an active agent directly to a target site within an organism. In some embodiments, an LNP may be locally delivered into a disease site, e.g., a tumor, other target site, e.g., a site of inflammation, or to a target organ, e.g., the liver, lung, stomach, colon, pancreas, uterus, breast, lymph nodes, and the like. In some embodiments, an LNP as disclosed herein may be locally delivered to a specific cell, e.g., hepatocytes, stellate cells, Kupffer cells, endothelial, alveolar, and/or epithelial cells. In some embodiments, an LNP as disclosed herein may be locally delivered to a specific tumor site, e.g., subcutaneous, orthotopic.

The LNPs may be formulated as a dispersed phase in an emulsion, micelles, or an internal phase in a suspension. In some embodiments, the LNPs are biodegradable. In some embodiments, the LNPs do not accumulate to cytotoxic levels or cause toxicity in vivo at a therapeutically effective dose. In some embodiments, the LNPs do not accumulate to cytotoxic levels or cause toxicity in vivo after repeat administrations at a therapeutically effective dose. In some embodiments, the LNPs do not cause an innate immune response that leads to a substantially adverse effect at a therapeutically effective dose.

In some embodiments, the LNP used, comprises the formula (6Z,9Z,28Z,31Z)-heptatriacont-6,9,28,31-tetraene-19-yl 4-(dimethylamino) butanoate or ssPalmO-phenyl-P4C2 (ssPalmO-Phe, SS-OP). In some embodiments, the LNP formulation comprises the formula, (6Z,9Z,28Z,31Z)-heptatriacont-6,9,28,31-tetraene-19-yl 4-(dimethylamino)butanoate (MC3), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., MC3 LNP or ssPalmO-phenyl-P4C2 (ssPalmO-Phe, SS-OP), 1,2-dioleoyl-sn-glycero-3-phosphocholine (DOPC), Cholesterol, 1,2-dimyristoyl-rac-glycero-3-methoxypolyethylene glycol-2000 (PEG2k-DMG), e.g., SSOP-LNP.

Liposomes are spherical vesicle structures composed of a uni- or multi-lamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Methods and compositions provided herein may comprise a pharmaceutical composition administered by a regimen sufficient to alleviate a symptom of a disease, disorder, and/or condition. In some aspects, the present disclosure provides methods of delivering a therapeutic by administering compositions as described herein.

### Uses

The present disclosure is further directed to uses of the site-specific disrupting agents or systems disclosed herein. Among other things, in some embodiments such provided technologies may be used to achieve modulation, e.g., repression, of expression of a target plurality of genes and, for example, enable control of the activity, delivery, and penetrance of one or more products of a target plurality of genes, e.g., in a cell. In some embodiments, a cell is a mammalian, e.g., human, cell. In some embodiments, a cell is a somatic cell. In some embodiments, a cell is a primary cell. For example, in some embodiments, a cell is a mammalian somatic cell. In some embodiments, a mammalian somatic cell is a primary cell. In some embodiments, a mammalian somatic cell is a non-embryonic cell.

### Modulating Gene Expression

The present disclosure is further directed, in part, to a method of modulating, e.g., decreasing, expression of a target plurality of genes, comprising providing a site-specific disrupting agent or a system described herein (or a nucleic acid encoding the same, or pharmaceutical composition comprising said site-specific disrupting agent or nucleic acid), and contacting the target plurality of genes, an anchor sequence associated with the target plurality of genes, and/or a genomic complex (e.g., ASMC) comprising the target plurality of genes with the site-specific disrupting agent or a system. In some embodiments, modulating, e.g., decreasing, expression of a target plurality of genes comprises modulation of transcription of a gene of the target plurality of genes as compared with a reference value, e.g., transcription of the gene in the absence of the site-specific disrupting agent or a system. In some embodiments, the method of modulating, e.g., decreasing, expression of a target plurality of genes is used ex vivo, e.g., on a cell from a subject, e.g., a mammalian subject, e.g., a human subject. In some embodiments, the cell is a mammalian, e.g., human, cell. In some embodiments, the cell is a somatic cell. In some embodiments, the cell is a primary cell. In some embodiments, the method of modulating, e.g., decreasing, expression of a target plurality of genes is used in vivo, e.g., on a mammalian subject, e.g., a human subject. In some embodiments, the method of modulating, e.g., decreasing, expression of a target plurality of genes is used in vitro, e.g., on a cell or cell line described herein.

Without wishing to be bound by theory, in some embodiments it is thought that a site-specific disrupting agent or a system may modulate the expression of a target plurality of genes by binding to an anchor sequence of a genomic complex, e.g., ASMC, comprising the target plurality of genes, and having one, two, or all of the following effects: physically or sterically blocking (e.g., competitively inhibiting) binding of a genomic complex component (e.g., nucleating polypeptide) to the anchor sequence; epigenetically modifying the target plurality of genes, a transcription control element operably linked to the target plurality of genes, or an anchor sequence proximal to the target plurality of genes or associated with an anchor sequence-mediated conjunction comprising the target plurality of genes (e.g., thereby decreasing and/or eliminating binding of a genomic complex component, e.g., nucleating polypeptide, to the anchor sequence); or genetically modifying the target plurality of genes, a transcription control element operably linked to the target plurality of genes, or an anchor sequence proximal to the target plurality of genes or associated with an anchor sequence-mediated conjunction comprising the target plurality of genes (e.g., thereby decreasing and/or eliminating binding of a genomic complex component, e.g., nucleating polypeptide, to the anchor sequence).

In some embodiments, a method described herein modulates, e.g., decreases, the expression of two or more genes of a target plurality of genes. In some embodiments, a method described herein modulates, e.g., decreases, the expression of at least 2, 3, 4, 5, 6, 7, 8, 9, 10, 11, 12, 13, 14, 15, 16, 17, 18, 19, or 20 (and optionally, no more than 2, 3, 4, 5, 6, 7, 8, 9, 10, 11, 12, 13, 14, 15, 16, 17, 18, 19, 20, or 30) genes of a target plurality of genes. In some embodiments, a method described herein modulates, e.g., decreases, the expression of 2-20, 2-18, 2-16, 2-14, 2-12, 2-10, 2-9, 2-8, 2-7, 2-6, 2-5, 2-4, 2-3, 3-20, 3-18, 3-16, 3-14, 3-12, 3-10, 3-9, 3-8, 3-7, 3-6, 3-5, 3-4, 4-20, 4-18, 4-16, 4-14, 4-12, 4-10, 4-9, 4-8, 4-7, 4- 6, 4-5, 5-20, 5-18, 5-16, 5-14, 5-12, 5-10, 5-9, 5-8, 5-7, 5-6, 6-20, 6-18, 6-16, 6-14, 6-12, 6-10, 6-9, 6-8, 6-7, 7-20, 7-18, 7-16, 7-14, 7-12, 7-10, 7-9, 7-8, 8-20, 8-18, 8-16, 8-14, 8-12, 8-10, 8-9, 9-20, 9-18, 9-16, 9-14, 9-12, 9-10, 10-20, 10-18, 10-16, 10-14, 10-12, 12-20, 12-18, 12-16, 12-14, 14-20, 14-18, 14-16, 16-20, 16-18, or 18-20 genes of a target plurality of genes. In some embodiments, a method described herein modulates, e.g., decreases, the expression of each gene (e.g., all genes) of a target plurality of genes.

In some embodiments, a method described herein modulates, e.g., decreases, the expression of a gene of a target plurality of genes, wherein one or more (e.g., 1, 2, 3, 4, 5, 6, 7, 8, 9, 10 or more, e.g., all) of the genes is a cytokine, an interleukin, a transcription factor (e.g., an interferon regulatory transcription factor), intercellular adhesion molecule (ICAM), or an interferon receptor. In some embodiments, a method described herein modulates, e.g., decreases, the level of RNA, e.g., mRNA, produced from one or more (e.g., 1, 2, 3, 4, 5, 6, 7, 8, 9, 10 or more, e.g., all) genes of the target plurality of genes. In some embodiments, modulating expression comprises decreasing the level of protein produced from one or more (e.g., 1, 2, 3, 4, 5, 6, 7, 8, 9, 10 or more, e.g., all) genes of the target plurality of genes. In some embodiments, modulating expression comprises both decreasing the level of mRNA and protein produced from one or more (e.g., 1, 2, 3, 4, 5, 6, 7, 8, 9, 10 or more, e.g., all) genes of the target plurality of genes. In some embodiments, the decrease is a decrease of at least 10%, 20%, 30%, 40%, 50%, 60%, 70%, 80%, or 90% compared to pre-treatment levels or levels in the absence of the site-specific disrupting agent.

In some embodiments, a method described herein modulates, e.g., decreases, the expression of one or more of (e.g., 1, 2, 3, or all of) human CXCL1, CXCL2, CXCL3, CXCL4, CXCL5, CXCL6, CXCL7, and IL8, e.g., upon stimulation of the cell with TNF-alpha, e.g., using an assay of any of Examples 2 or 4-11. In some embodiments, a method described herein modulates, e.g., decreases, the expression of one or more of (e.g., 1, 2, 3, or all of) mice CXCL1, CXCL2, CXCL3, CXCL4, CXCL5, CXCL7, and CXCL15, e.g., upon stimulation of the cell with TNF-alpha, e.g., using an assay of Example 14.

In some embodiments, a method described herein decreases binding of a nucleating polypeptide, e.g., CTCF, to an anchor sequence. In some embodiments, contacting a cell or administering a site-specific disrupting agent results in a decrease in binding of a nucleating polypeptide, e.g., CTCF, to an anchor sequence (e.g., an anchor sequence of an ASMC comprising a target plurality of genes). In some embodiments, contacting a cell or administering a site-specific disrupting agent results in a complete loss of binding or a decrease of at least 50, 60, 70, 80, 90, 95, or 99%, e.g., as measured by ChIP and/or quantitative PCR, relative to the binding of the nucleating polypeptide (e.g., CTCF) to the anchor sequence prior to treatment with the site-specific disrupting agent or the system or in the absence of the site-specific disrupting agent or the system.

The present disclosure is further directed, in part, to a method of treating a condition associated with over-expression of a target plurality of genes in a subject, comprising administering to the subject a site-specific disrupting agent, a system, a nucleic acid, a vector, a cell, or a pharmaceutical composition described herein. Conditions associated with over-expression of particular genes are known to those of skill in the art. Such conditions include, but are not limited to, metabolic disorders, neuromuscular disorders, cancer (e.g., solid tumors), fibrosis, diabetes, urea disorders, immune disorders, inflammation, and arthritis. In some embodiments, the disorder is an auto-immune disorder. In some embodiments, the disorder is associated with or caused by an infection, e.g., a viral infection, e.g., SARS-Cov2 viral infection.

The present disclosure is further directed, in part, to a method of modulating, e.g., decreasing, expression of a target plurality of genes, in a cell in a subject, e.g., a human subject. In some embodiments, the subject has a disease or condition. In some embodiments, the disease is an inflammatory disease, e.g., an immune mediated inflammatory disease. In some embodiments, the disease or condition is one or more of rheumatoid arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a viruses, or a fungus), external injury (e.g., scrapes or foreign objects), effects of radiation or chemical injury, osteoarthritis, osteoarthritic joint pain, joint pain, inflammatory pain, acute pain, chronic pain, cystisis, bronchitis, dermatitis, cardiovascular disease, neurodegenerative disease, liver disease, lung disease, kidney disease, pain, swelling, stiffness, tenderness, redness, warmth, or elevated biomarkers related to disease states (e.g., cytokines, immune receptors, or inflammatory markers). In some embodiments, the inflammatory disorder is associated with an infection, e.g., by a virus, e.g., Sars-Cov-2 virus. In some embodiments, the inflammatory disorder is an autoimmune disorder.

Methods and compositions as provided herein may treat a condition associated with over-expression of a target plurality of genes by stably or transiently altering (e.g., decreasing) transcription of a target plurality of genes. In some embodiments, such a modulation persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, a method or composition provided herein may decrease expression of a gene of a target plurality of genes in a cell by at least 10, 20, 30, 40, 50, 60, 70, 80, 90, or 100% (and optionally up to 100%) relative to expression of the gene of the target plurality of genes in a cell not contacted by the composition or treated with the method. In some embodiments, a method or composition provided herein may decrease expression of each gene of a target plurality of genes in a cell by at least 10, 20, 30, 40, 50, 60, 70, 80, 90, or 100% (and optionally up to 100%) relative to expression of each gene of the target plurality of genes in a cell not contacted by the composition or treated with the method.

In some embodiments, a method provided herein may modulate, e.g., decrease, expression of a target plurality of genes by disrupting a genomic complex, e.g., an anchor sequence-mediated conjunction, comprising said target plurality of genes. In some embodiments, a method described herein disrupts a genomic complex (e.g., ASMC). In some embodiments, contacting a cell or administering a site-specific disrupting agent results in a decrease in the level of a genomic complex (e.g., ASMC) comprising the target plurality of genes relative to the level of the complex prior to treatment with the site-specific disrupting agent or the system or in the absence of the site-specific disrupting agent or the system. In some embodiments, contacting a cell or administering a site-specific disrupting agent results in a complete loss of the genomic complex, e.g., ASMC, or a decrease of at least 25, 30, 35, 40, 45, 50, 60, 70, 80, 90, 95, or 99%, e.g., as measured by ChIA-PET, ELISA (e.g., to assess gene expression changes), CUT&RUN, ATAC-SEQ, ChIP and/or quantitative PCR, relative to the level of the complex prior to treatment with the site-specific disrupting agent or the system or in the absence of the site-specific disrupting agent or the system.

In some embodiments, methods and compositions as provided herein may treat a condition associated with cascade of inflammation or cytokine storm by decreasing recruitment of cytokines in the site of inflammation. In some embodiments, the cascade of inflammation and/or cytokine storm is associated with an inflammatory disorder, e.g., a viral mediated inflammatory disorder, e.g., COVID-19 infection. In some embodiments, the inflammatory disorder is associated with an infection, e.g., by a virus, e.g., Sars-Cov-2 virus. In some embodiments, the inflammatory disorder is an autoimmune disorder. In some embodiments, the inflammatory disorder is associated with hypoxia. In some embodiments, the inflammatory disorder is associated with ARDS, hypoxia, and/or sepsis. In some embodiments, the infection is a super infection, e.g., caused by more than one pathogen, e.g., a first virus or a bacterium, or a fungus, and a second virus, or a second bacterium, or a second fungus.

### Epigenetic Modification

The present disclosure is further directed, in part, to a method of epigenetically modifying: one or more (e.g., all) genes of a target plurality of genes; a transcription control element operably linked to the target plurality of genes; an anchor sequence proximal to the target plurality of genes or associated with an anchor sequence-mediated conjunction comprising the target plurality of genes; or a site proximal to said anchor sequence, the method comprising providing a site-specific disrupting agent, a system, a nucleic acid encoding the site-specific disrupting agent, nucleic acids encoding the components of the system, or pharmaceutical composition comprising said site-specific disrupting agent, system or nucleic acid; and contacting the one or more (e.g., all) genes of the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence with the site-specific disrupting agent or the system, thereby epigenetically modifying the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence.

In some embodiments, a method of epigenetically a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence comprises increasing or decreasing DNA methylation of the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence. In some embodiments, a method of epigenetically modifying target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence comprises increasing or decreasing histone methylation of a histone associated with the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence. In some embodiments, a method of epigenetically modifying a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence comprises decreasing histone acetylation of a histone associated with the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence. In some embodiments, a method of epigenetically modifying a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence comprises increasing or decreasing histone sumoylation of a histone associated with the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence. In some embodiments, a method of epigenetically modifying a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence comprises increasing or decreasing histone phosphorylation of a histone associated with the target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence.

In some embodiments, a method of epigenetically modifying a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence may decrease the level of the epigenetic modification by at least 10, 20, 30, 40, 50, 60, 70, 80, 90, or 100% (and optionally up to 100%) relative to the level of the epigenetic modification at that site in a cell not contacted by the composition or treated with the method. In some embodiments, a method of epigenetically modifying a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence may increase the level of the epigenetic modification by at least 10, 20, 30, 40, 50, 60, 70, 80, 90, 100, 120, 140, 160, 180, 200, 300, 400, 500, 600, 700, 800, 900, or 1000% (and optionally up to 200, 300, 400, 500, 600, 700, 800, 900, 1000, or 2000%) relative to the level of the epigenetic modification at that site in a cell not contacted by the composition or treated with the method. In some embodiments epigenetic modification of a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence may modify the level of expression of the target plurality of genes, e.g., as described herein.

In some embodiments, an epigenetic modification produced by a method described herein persists for at least about 1 hour to about 30 days, or at least about 2 hours, 6 hours, 12 hours, 18 hours, 24 hours, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time there between. In some embodiments, such a modulation persists for at least 1, 2, 3, 4, 5, 6, 7, 8, 9, 10, 11, 12, 13, 14, 15, 16, 17, 18, 19, 20, 21, 22, 23, or 24 hours, or at least 1, 2, 3, 4, 5, 6, or 7 days, or at least 1, 2, 3, 4, or 5 weeks, or at least 1, 2, 3, 4, 5, 6, 7, 8, 9, 10, 11, or 12 months, or at least 1, 2, 3, 4, or 5 years (e.g., indefinitely). Optionally, such a modulation persists for no more than 10, 9, 8, 7, 6, 5, 4, 3, 2, or 1 years.

In some embodiments, a site-specific disrupting agent or a system for use in a method of epigenetically modifying a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence comprises an effector moiety that comprises an epigenetic modifying moiety. For example, an effector moiety may comprise an epigenetic modifying moiety with DNA methyltransferase activity, and an endogenous or naturally occurring target sequence (e.g., a gene of a target plurality of genes, a transcription control element operably linked to the target plurality of genes, an anchor sequence proximal to the target plurality of genes or associated with an anchor sequence-mediated conjunction comprising the target plurality of genes, or a site proximal to said anchor sequence) may be altered to increase its methylation (e.g., decreasing interaction of a transcription factor with a portion of a gene of a target plurality of genes or transcription control element, decreasing binding of a nucleating protein to an anchor sequence, and/or disrupting or preventing an anchor sequence-mediated conjunction), or may be altered to decrease its methylation (e.g., increasing interaction of a transcription factor with a portion of a gene of a target plurality of genes or transcription control element, increasing binding of a nucleating protein to an anchor sequence, and/or promoting or increasing strength of an anchor sequence-mediated conjunction).

### Genetic Modification

The present disclosure is further directed, in part, to a method of genetically modifying one or more (e.g., one, two, three, or all) genes of a target plurality of genes, a transcription control element operably linked to the target plurality of genes, or an anchor sequence proximal to the target plurality of genes or associated with an anchor sequence-mediated conjunction comprising the target plurality of genes, the method comprising providing a site-specific disrupting agent or a system or nucleic acid encoding the same or pharmaceutical composition comprising said site-specific disrupting agent, system or nucleic acid; and contacting the one or more (e.g., one, two, three, or all) genes of the target plurality of genes, a transcription control element operably linked to the target plurality of genes, or an anchor sequence proximal to the target plurality of genes or associated with an anchor sequence-mediated conjunction comprising the target plurality of genes with the site-specific disrupting agent, thereby genetically modifying the target plurality of genes, a transcription control element operably linked to the target plurality of genes, or an anchor sequence proximal to the target plurality of genes or associated with an anchor sequence-mediated conjunction comprising the target plurality of genes.

Genetic modification may comprise introducing one or more of an insertion, deletion, or substitution into a gene of a target plurality of genes, a transcription control element operably linked to the target plurality of genes, or an anchor sequence proximal to the target plurality of genes or associated with an anchor sequence-mediated conjunction comprising the target plurality of genes. In some embodiments, an insertion comprises addition of at least 150, 200, 250, 300, 400, 500, 600, 700, 800, 900, 1000, 1200, 1400, 1600, 1800, or 2000 nucleotides (and optionally no more than 3000, 2500, 2000, 1500, 1000, 900, 800, 700, 600, 500, 400, 300, or 200 nucleotides). In some embodiments, an insertion comprises addition of at least 1, 2, 3, 4, 5, 6, 7, 8, 9, 10, 15, 20, 25, 30, 35, 40, 50, 60, 70, 80, 90, or 100 nucleotides (and optionally no more than 200, 150, 100, 90, 80, 70, 60, 50, 40, 35, 30, 25, 20, 15, 10, 9, 8, 7, 6, 5, 4, 3, 2, or 1 nucleotide). In some embodiments, the insertion comprises addition of 1-10, 1-9, 1-8, 1-7, 1-6, 1-5, 1-4, 1-3, or 1-2 nucleotides. In some embodiments, a deletion comprises removal of at least 150, 200, 250, 300, 400, 500, 600, 700, 800, 900, 1000, 1200, 1400, 1600, 1800, or 2000 nucleotides (and optionally no more than 3000, 2500, 2000, 1500, 1000, 900, 800, 700, 600, 500, 400, 300, or 200 nucleotides). In some embodiments, a deletion comprises removal of at least 1, 2, 3, 4, 5, 6, 7, 8, 9, 10, 15, 20, 25, 30, 35, 40, 50, 60, 70, 80, 90, or 100 nucleotides (and optionally no more than 200, 150, 100, 90, 80, 70, 60, 50, 40, 35, 30, 25, 20, 15, 10, 9, 8, 7, 6, 5, 4, 3, 2, or 1 nucleotide). In some embodiments, the deletion comprises removal of 1-10, 1-9, 1-8, 1-7, 1-6, 1-5, 1-4, 1-3, or 1-2 nucleotides. In some embodiments, a substitution comprises alteration of at least 1, 2, 3, 4, 5, 6, 7, 8, 9, 10, 15, 20, 25, 30, 35, 40, 50, 60, 70, 80, 90, or 100 nucleotides (and optionally no more than 200, 150, 100, 90, 80, 70, 60, 50, 40, 35, 30, 25, 20, 15, 10, 9, 8, 7, 6, 5, 4, 3, 2, or 1 nucleotide). In some embodiments, the substitution comprises alteration of 1-10, 1-9, 1-8, 1-7, 1-6, 1-5, 1-4, 1-3, or 1-2 nucleotides.

In some embodiments, a genetic modification comprises an insertion, deletion, or substitution to an anchor sequence, e.g., associated with an ASMC comprising the target plurality of genes. In some embodiments, the genetic modification alters (e.g., decreases or increases) the binding of a genomic complex component, e.g., a nucleating polypeptide, to the anchor sequence. In some embodiments, the genetic modification abrogates (e.g., via an insertion, deletion, or substitution), wholly or in part, an anchor sequence, thereby decreasing or abolishing the binding of a nucleating polypeptide to the anchor sequence, e.g., and decreasing the presence of or abolishing an ASMC comprising said anchor sequence. Without wishing to be bound by theory, the disclosure contemplates use of a site-specific disrupting agent with genetic modification functionality to introduce an insertion, deletion, or substitution into an anchor sequence to decrease or eliminate the anchor sequence's participation in a genomic complex, e.g., ASMC, that comprises a target plurality of genes. As described elsewhere herein, such an alteration is expected to disrupt the genomic complex, e.g., ASMC, and may decrease expression of the target plurality of genes.

In some embodiments, the genetic modification comprises insertion of a sequence comprising an anchor sequence. Without wishing to be bound by theory, the disclosure contemplates use of a site-specific disrupting agent or a system with genetic modification functionality to introduce an exogenous anchor sequence into a gene of a target plurality of genes, a transcription control element operably linked to the target plurality of genes, or an anchor sequence proximal to the target plurality of genes or associated with an anchor sequence-mediated conjunction comprising the target plurality of genes. It is thought that the presence of a new anchor sequence may disrupt the formation and/or maintenance of a genomic complex, e.g., ASMC, comprising the target plurality of genes, thereby modulating, e.g., decreasing, expression of the target plurality of genes.

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### EXAMPLES

### Example 1: Decreasing Expression of an Exemplary Plurality of Genes

This example describes, in part, experiments demonstrating decreasing expression of a target plurality of genes comprising CXCL1, CXCL2, CXCL3, and IL8 using a site-specific disrupting agent comprising a CRISPR/Cas molecule and guide RNAs comprising the given guide sequences.

Transfection of RNPs comprising a site-specific disrupting agent comprising mRNA encoding CRISPR/Cas molecule (Cas9) and sgRNA Cas9/guide RNP complex was carried out by electroporation into THP-1 cells. Cells were cultured in RPMI+10% FBS. A parental line was also analyzed for comparison.

350 k cells were plated in quadruplicate for each edited cell line and the parental control into 24 well plates. One hour later 10 ng/ml of TNF alpha (Sigma Cat #654205) was added to 2 wells for each cell line. The remaining 2 wells are untreated as a control.

The edited and parental cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific). CXCL1-3 & IL7 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells.

The CTCF anchors at both boundaries of the Insulated Genomic Domain (IGD) were located using ChIP-seq data, and the CTCF anchor sequences were identified computationally using the known CTCF position weight matrix (JASPAR). CRISPR (Sp Cas9) guides were chosen to target the CTCF anchor sequence.

The guides sequences are listed in the table below.

TABLE 4

| Guide Sequence | Genomic Coordinates | Guide |
|---|---|---|
| GGGGCCACTAGGGACAGGAT | chr19:55627120- | GD-28662 |
| (SEQ ID NO: 20) | 55627139 | |
| (non-target control) | | |
| AGCCCCACCTTGTGGTCAGA | chr4:74595464- | GD-28481 |
| (SEQ ID NO: 21) | 74595486 | |
| AGTGCTGCCTTCTGACCACA | chr4:74595457- | GD-28482 |
| (SEQ ID NO: 22) | 74595479 | |
| GCTGCCTTCTGACCACAAGG | chr4:74595460- | GD-28483 |
| (SEQ ID NO: 23) | 74595482 | |
| CCAGTATAAGCCCCACCTTG | chr4:74595472- | GD-28484 |
| (SEQ ID NO: 24) | 74595494 | |
| CTGCCTGTCCCATAAGGAGG | chr4:75000088- | GD-28485 |
| (SEQ ID NO: 25) | 75000110 | |
| GCACTGCCTGTCCCATAAGG | chr4:75000091- | GD-28486 |
| (SEQ ID NO: 26) | 75000113 | |
| GGTCCTCCTCCTTATGGGAC | chr4:75000085- | GD-28487 |
| (SEQ ID NO: 27) | 75000107 | |
| GCCTTGTTTTCGGCTCTAGA | chr4:75000157- | GD-28488 |
| (SEQ ID NO: 28) | 75000179 | |
| GCCATCTAGAGCCGAAAACA | chr4:75000156- | GD-28489 |
| (SEQ ID NO: 29) | 75000178 | |

### Example 2: Cytokine Expression Decrease in THP-1 Cells at 72 Hours

This example describes, in part, experiments demonstrating decreasing expression of a target plurality of genes comprising CXCL1, CXCL2, CXCL3, and IL8 using a site-specific disrupting agent comprising a CRISPR/Cas molecule and guide RNAs targeted to the anchor sequences of the ASMC comprising the target plurality of genes.

sgRNA and mRNA encoding Cas9 RNPs were electroporated into THP-1 cells. sgRNA sequences (from Example 1) were chosen to target one of the CTCF sites of the ASMC comprising CXCL1, CXCL2, CXCL3, and IL8. The transfected cells were incubated with 10 ng/ml TNF alpha for 24 hrs. After that time, DNA and RNA were isolated using the AllPrep DNA/RNA Kit (Qiagen), following the manufacturer's protocol. RNA samples were retrotranscribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific).

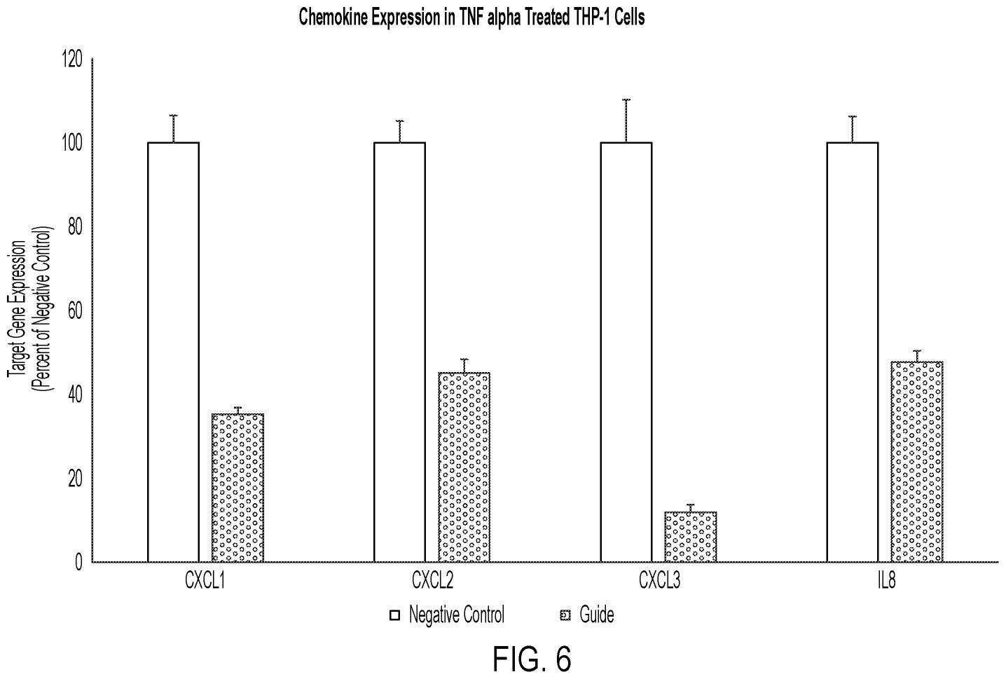

CXCL1-3 & IL8 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results in FIG. 6 show that a site-specific disrupting agent comprising a CRISPR/Cas molecule and an sgRNA can be used to decrease CXCL1, CXCL2, CXCL3, and IL8 expression in THP-1 cells, and that expression is decreased at 72 hours post-treatment. The results also show that LNP delivery of said site-specific disrupting agent can be used to deliver effective amounts of the agent to the target cells.

Similar results were seen when the experiment was repeated with sgRNAs targeting the other CTCF site of the ASMC comprising CXCL1, CXCL2, CXCL3, and IL8 (data not shown).

### Example 3: Cytokine Protein Secretion of THP-1 Cells Decreased by Site-Specific Modulating Agent

This example describes, in part, experiments demonstrating decreasing secretion of CXCL1 and IL-8, two genes of a target plurality of genes, by treating cells using a site-specific disrupting agent comprising a CRISPR/Cas molecule and guide RNAs targeted to the anchor sequences of the ASMC comprising the target plurality of genes.

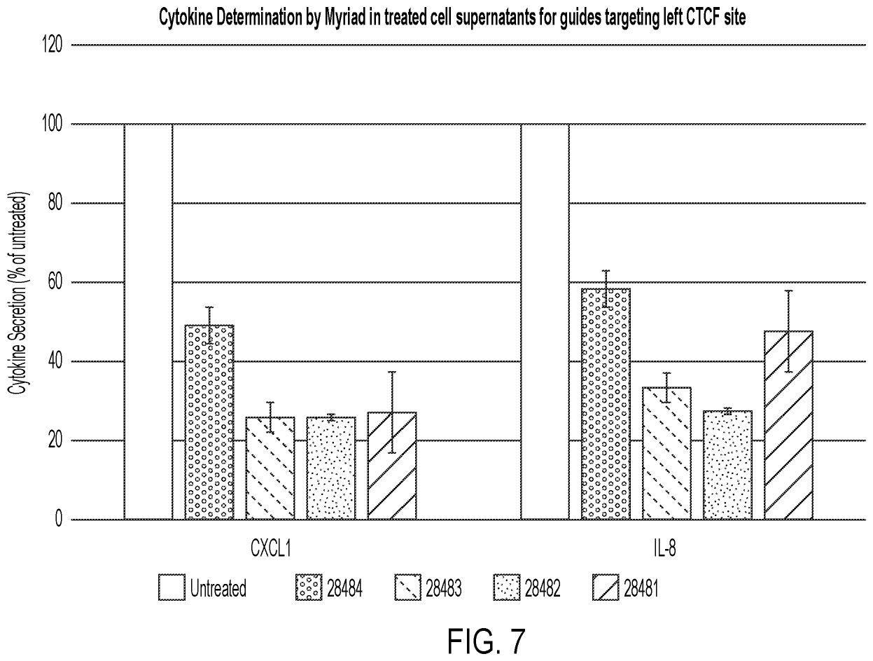

THP-1 cells were electroporated with RNPs comprising sgRNAs and mRNA encoding a site-specific disrupting agent comprising an exemplary CRISPR/Cas molecule (Cas9) as in previous Examples. sgRNAs (from Example 1) were targeted to one of the CTCF sites of the ASMC comprising CXCL1 and IL-8. Cells were stimulated with 10 ng/ml TNF alpha for 24 hours. After that time, cell supernatants were collected and frozen at −80 degrees ° C. Supernatants from cells contacted with 4 different sgRNAs, in addition to the mRNA encoding the CRISPR/Cas molecule, as well as an un-transfected positive control were screened for CXCL1 and IL-8 protein levels on a cytokine panel by Myriad Genetics Inc. FIG. 7 shows diminished levels of CXCL1 and IL8 were seen for each supernatant obtained from cells treated with sgRNA and CRISPR/Cas molecule RNPs, demonstrating a phenotypic response to ASMC disruption (e.g., by disrupting anchor sequence and nucleating polypeptide interactions, e.g., disrupting CTCF binding). This data is in agreement with the decreased mRNA expression seen by qPCR in Example 2.

Similar results were seen when the experiment was repeated with sgRNAs targeting the other CTCF site of the ASMC comprising CXCL1 and IL8 (data not shown).

### Example 4: CXCL3 Expression Decrease as Measured by qPCR

This example describes, in part, experiments demonstrating decreasing expression of CXCL3 by treating THP-1 cells using a site-specific disrupting agent comprising a CRISPR/Cas molecule and a variety of guide RNAs targeted to the anchor sequences of the ASMC comprising the target plurality of genes.

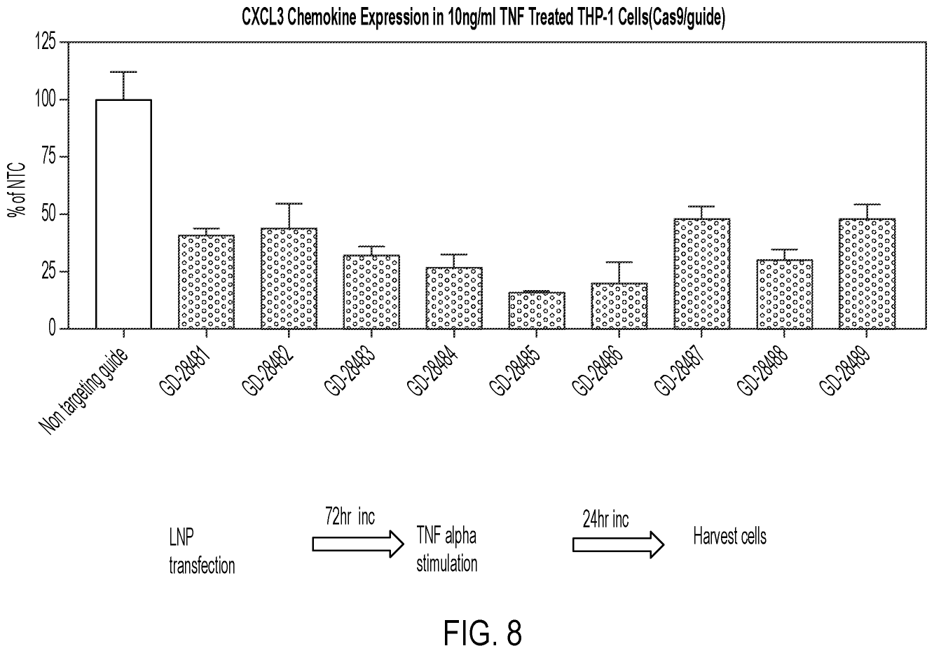

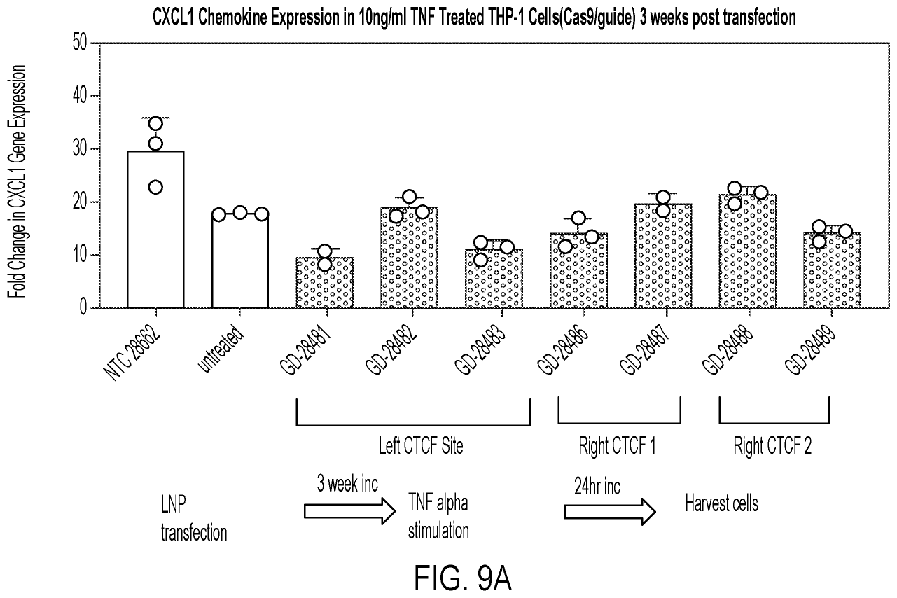

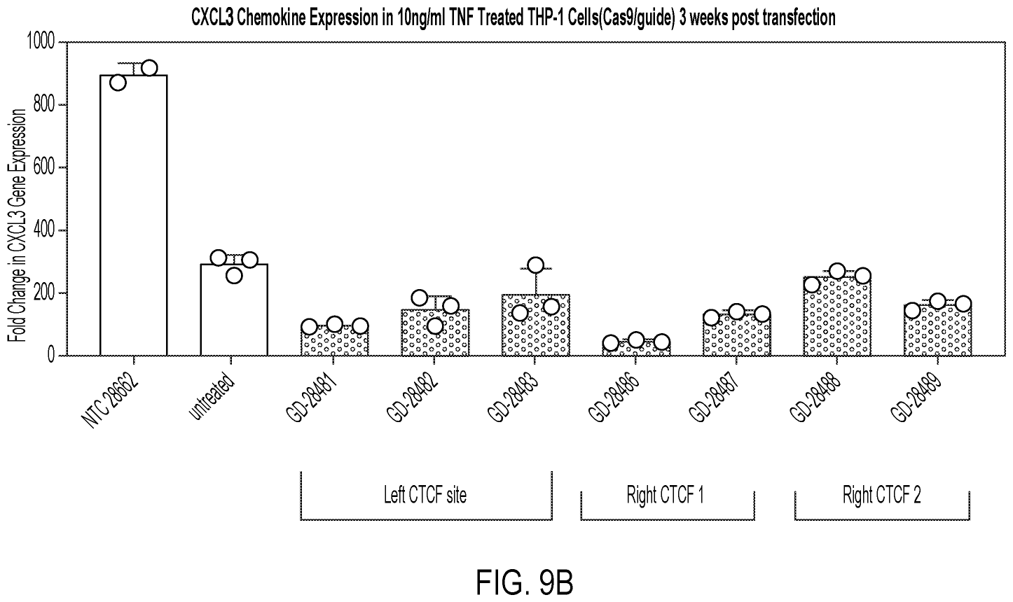

THP-1 cells were grown in RPMI+10% FBS. Cells were transfected with mRNA encoding a CRISPR/Cas molecule (Cas9) and sgRNA targeted to either of the CTCF sites of the ASMC comprising the target plurality of genes using LNPs. sgRNAs (from Example 1) used target the left or right CTCF site as indicated in FIG. 9A. Lipid nanoparticle (LNP) formulation using SSOP lipid mixture was carried out by using the NanoAssemblr® Spark™ from Precision NanoSystems Inc. For experimental conditions, 350 k cells/well were plated 72 hrs after LNP transfection for each transfected condition and the parental control into 24 well plates (see FIG. 8 flow chart). One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL3 TaqMan primer/probe sets with the TaqMan® Fast Advanced Master Mix (Thermo Fisher Scientific).

CXCL3 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 8 graph) that a site-specific disrupting agent comprising a CRISPR/Cas molecule and several different sgRNAs can be used to decrease CXCL3 expression in THP-1 cells. The results also show that LNP delivery of said site-specific disrupting agent can be used to deliver effective amounts of the agent to the target cells. The results also demonstrate that targeting the anchor sequence on either side of the ASMC comprising the target plurality of genes can decrease expression of the target plurality of genes.

Similar results were seen when the experiment was repeated with sgRNAs targeting the other CTCF site of the ASMC comprising CXCL1, CXCL2, CXCL3, and IL8 (data not shown).

### Example 5: CXCL1 and CXCL3 Expression is Decreased 3 Weeks Post-Transfection

This example describes, in part, experiments demonstrating a stable decrease in expression of CXCL1 and CXCL3 in THP-1 cells three weeks post-transfection with a site-specific disrupting agent comprising a CRISPR/Cas molecule and a variety of guide RNAs targeted to the anchor sequences of the ASMC comprising the target plurality of genes.

Cells and LNP were prepared, and samples analyzed as in Example 4, except that transfected cells were incubated for 3 weeks before TNF alpha stimulation (see FIG. 9A flow chart).

The results show (FIGS. 9A and 9B) that a site-specific disrupting agent comprising a CRISPR/Cas molecule and several different sgRNAs can be used to stably decrease CXCL1 and CXCL3 expression in THP-1 cells up to and including 3 weeks after treatment with LNPs comprising the agent(s). The results also demonstrate that targeting the anchor sequence on either side of the ASMC comprising the target plurality of genes can stably decrease expression of the target plurality of genes.

### Example 6: Agents Comprising KRAB Effector Moieties Decrease CXCL1 Expression

This example describes, in part, experiments demonstrating a decrease in expression of CXCL1 in THP-1 cells after transfection with a site-specific disrupting agent comprising a CRISPR/Cas molecule fused to a transcriptional repressor and a variety of guide RNAs targeted to the anchor sequences of the ASMC comprising the target plurality of genes.

THP-1 cells were grown in RPMI+10% FBS. Cells were transfected with mRNA encoding a CRISPR/Cas molecule fused to a transcriptional repressor, dCas9-KRAB, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350 k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha. Transfection with mRNA encoding a CRISPR/Cas molecule (Cas9) and the sgRNAs (per the Examples 2, 4, and 5) was performed as a positive control.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 specific TaqMan primer/probe sets with the TaqMan® Fast Advanced Master Mix (Thermo Fisher Scientific).

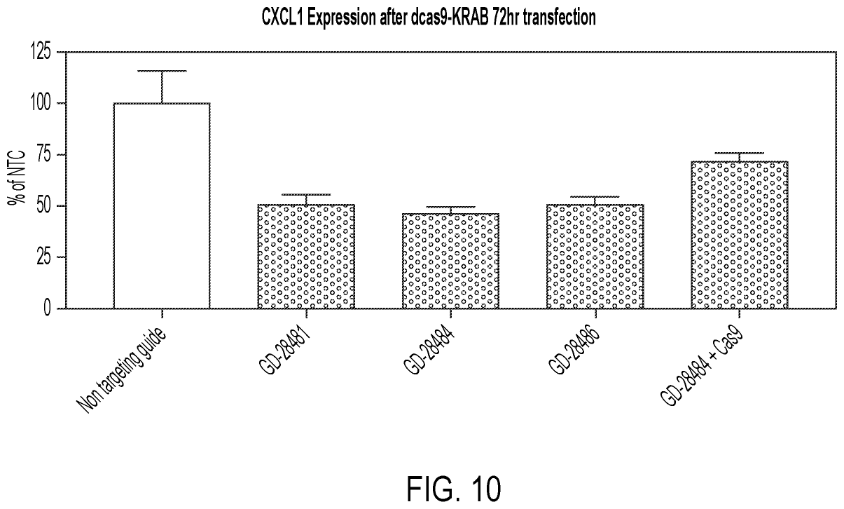

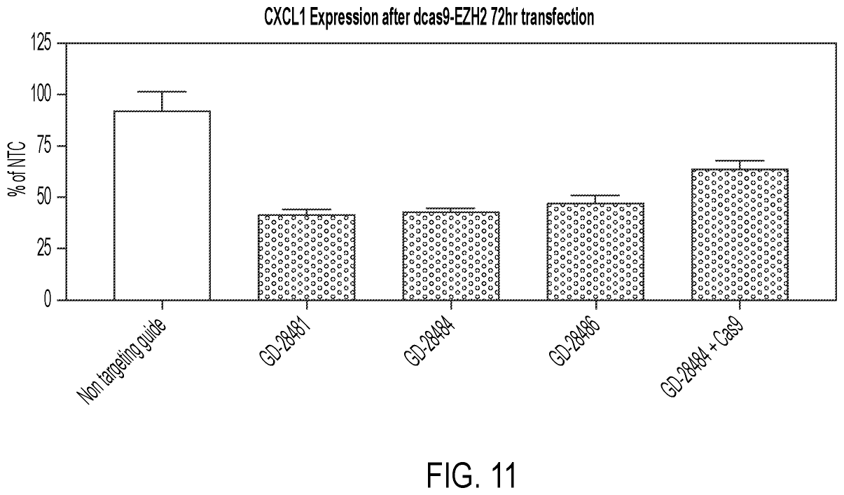

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 10) that a site-specific disrupting agent comprising a catalytically inactive CRISPR/Cas molecule fused to a transcriptional repressor, KRAB, and targeted to CTCF sites by different sgRNAs can be used to decrease CXCL1 expression in THP-1 cells. The decrease in expression was seen 72 hours post-transfection.

### Example 7: Agents Comprising EZH2 Effector Moieties Decrease CXCL1 Expression

This example describes, in part, experiments demonstrating a decrease in expression of CXCL1 in THP-1 cells after transfection with a site-specific disrupting agent comprising a CRISPR/Cas molecule fused to a histone methyltransferase (EZH2) and a variety of guide RNAs targeted to the anchor sequences of the ASMC comprising the target plurality of genes.

THP-1 cells were grown in RPMI+10% FBS. Cells were transfected with mRNA encoding a catalytically inactive CRISPR/Cas molecule (dCas9) fused to a histone deacetylase, dCas9-EZH2, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350 k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 specific TaqMan primer/probe sets with the TaqMan® Fast Advanced Master Mix (Thermo Fisher Scientific).

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 11) that a site-specific disrupting agent comprising a catalytically inactive CRISPR/Cas molecule fused to a histone methyltransferase (EZH2) and targeted to CTCF sites by different sgRNAs can be used to decrease CXCL1 expression in THP-1 cells. The decrease in expression was seen 72 hours post-transfection.

### Example 8: Agents Comprising MQ1 Effector Moieties Decrease CXCL1 Expression

This example describes, in part, experiments demonstrating a decrease in expression of CXCL1 in THP-1 cells after transfection with a site-specific disrupting agent comprising a CRISPR/Cas molecule fused to a DNA methyltransferase (MQ1) and a variety of guide RNAs targeted to the anchor sequences of the ASMC comprising the target plurality of genes.

THP-1 cells were grown in RPMI+10% FBS. Cells were transfected with mRNA encoding a catalytically inactive CRISPR/Cas molecule (dCas9) fused to MQ1, dCas9-MQ1, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350 k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 and CXCL3 specific TaqMan primer/probe sets with the TaqMan® Fast Advanced Master Mix (Thermo Fisher Scientific).

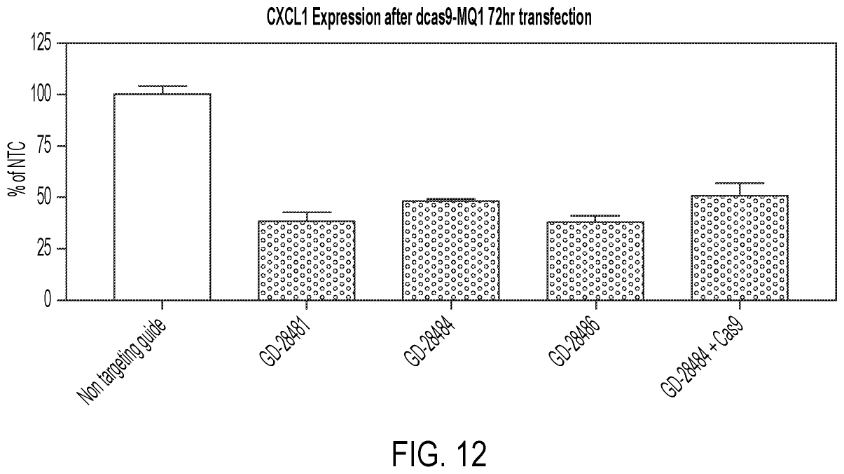

CXCL1 and 3 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 12) that a site-specific disrupting agent comprising a catalytically inactive CRISPR/Cas molecule fused to DNA methyltransferase (MQ1), and targeted to CTCF sites by different sgRNAs can be used to decrease CXCL1 expression in THP-1 cells. The decrease in expression was seen 72 hours post-transfection. Similar results were seen measuring CXCL3 expression (data not shown).

### Example 9: Durable CXCL1 Decrease in Expression After Cas9 or dCas9-EZH2 Treatment

This example describes, in part, experiments demonstrating a stable decrease in expression of CXCL1 in THP-1 cells up to 4 weeks after transfection with a site-specific disrupting agent comprising either a CRISPR/Cas molecule or a catalytically inactive CRISPR/Cas molecule fused to a histone deacetylase (EZH2) and an sgRNA targeted to an anchor sequence of the ASMC comprising the target plurality of genes comprising CXCL1.

Using the ATx™ Scalable Transfection System (MaxCyte), THP-1 cells grown in RPMI+10% FBS were electroporated with mRNA encoding either of the site-specific disrupting agents (Cas9 or dCas9-EZH2) and sgRNA (from Example 1) at 5 million cells per condition in processing assemblies. Samples of the transfected cells were harvested and incubated with TNF alpha for 24 hrs. This was repeated each week carried out to 4 weeks. After that time, DNA and RNA were isolated using the AllPrep DNA/RNA Kit (Qiagen) following the Manufacture's protocol. RNA samples were retrotranscribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific).

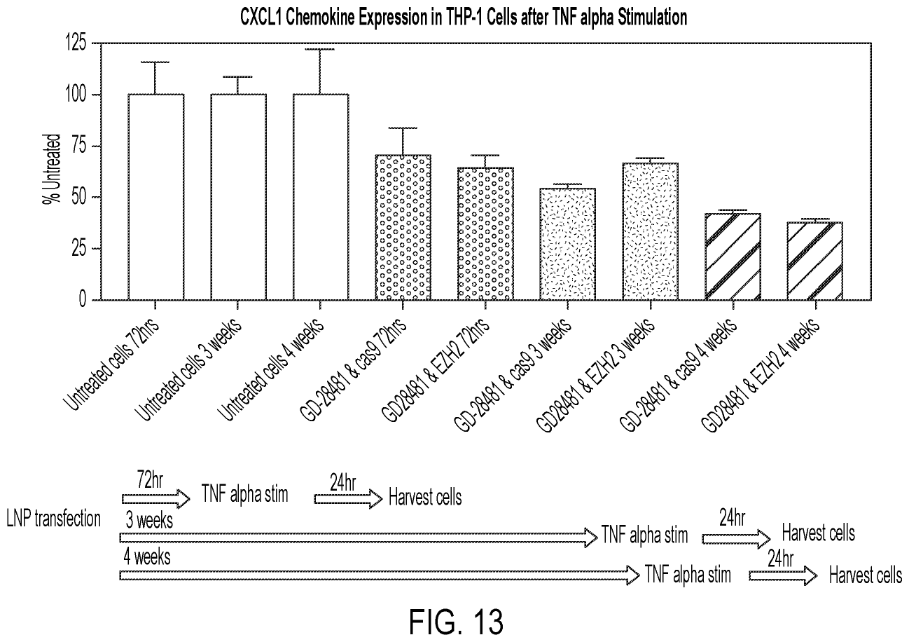

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show that (FIG. 13) a site-specific disrupting agent comprising a CRISPR/Cas molecule, or a catalytically inactive CRISPR/Cas molecule fused to a histone methyltransferase (EZH2) and targeted to a CTCF site by an sgRNA can be used to decrease CXCL1 expression in THP-1 cells. The decrease in expression is durable up to at least 4 weeks, and is also observed at 72 hours and 3 weeks post-transfection.

### Example 10: CXCL3 Expression Decreases Upon Treatment with EZH2-dCas9-KRAB and sgRNA

This example describes, in part, experiments demonstrating a decrease in expression of CXCL3 in THP-1 cells after transfection with a site-specific disrupting agent comprising a catalytically inactive CRISPR/Cas molecule fused to a histone methyltransferase (EZH2) and a transcriptional repressor (KRAB) and a variety of guide RNAs targeted to an anchor sequence of the ASMC comprising the target plurality of genes.

THP-1 cells were grown in RPMI+10% FBS. Several different site-specific disrupting agents were tested: G9A-dCas9-EZH2 (G9A fused to dCas9 fused to EZH2), G9A-dCas9-KRAB, and EZH2-dCas9-KRAB. Cells were transfected with mRNA encoding the site-specific disrupting agent, and sgRNA targeted to a CTCF site of the ASMC comprising the target plurality of genes using LNPs. The sgRNA was chosen to target a genomic DNA site proximal to the left CTCF site but some distance removed from the left CTCF site (e.g., 80, 160, 235, or 300 nucleotides from the CTCF site). Exemplary guide sequences targeting genomic DNA sites proximal to the left CTCF site, but some distance removed from the left CTCF site are given in Table 5.

TABLE 5

| | | Genomic | SEQ |
|---|---|---|---|
| Guide | Guide Sequence | Coordinates | ID No. |
| GD-29251 | CCAATGAAGATGAAACTGGG | chr4:74595215- | 30 |
| | | 74595237 | |
| GD-29252 | AACGTGCTTGCCTAAGATTC | chr4:74595370- | 31 |
| | | 74595392 | |
| GD-29253 | AGCCCTTAATCATATCTAGT | chr4:74595560- | 32 |
| | | 74595582 | |
| GD-29254 | CAGAGCTTAAGACCTGTACT | chr4:74595642- | 33 |
| | | 74595664 | |
| GD-29255 | GCCCACCTTGACCTTCACAA | chr4:74595787- | 34 |
| | | 74595809 | |

Lipid nanoparticle (LNP) formulation using SSOP lipid mixture was carried out by using the NanoAssemblr® Spark™ from Precision NanoSystems Inc. For experimental conditions, 350 k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 and CXCL3 specific TaqMan primer/probe sets with the TaqMan® Fast Advanced Master Mix (Thermo Fisher Scientific).

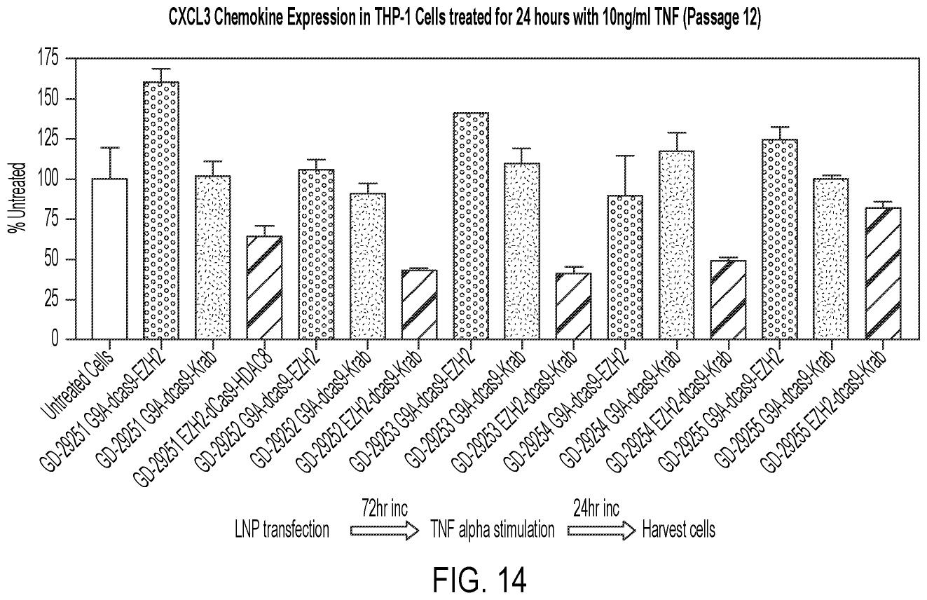

CXCL1 and 3 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 14) that a site-specific disrupting agent comprising a catalytically inactive CRISPR/Cas molecule fused to a histone methyltransferase (EZH2) and a transcriptional repressor (KRAB), and targeted to a site proximal to a CTCF an sgRNA can be used to decrease CXCL3 expression in THP-1 cells. Similar results were seen measuring CXCL1 expression (data not shown).

### Example 11: CXCL1 Expression Decreases Upon Treatment with Site-Specific Disrupting Agents and sgRNA

This example describes, in part, experiments demonstrating a decrease in expression of CXCL1 in THP-1 cells after transfection with various exemplary site-specific disrupting agents including: a catalytically inactive CRISPR/Cas molecule fused to a DNA methyltransferase (DNMT33a/31); a catalytically inactive CRISPR/Cas molecule fused to a histone deacetylase (HDAC8); or a catalytically inactive CRISPR/Cas molecule fused to a histone deacetylase (HDAC8) and a histone methyltransferase (EZH2), and a variety of guide RNAs targeted to an anchor sequence of the ASMC comprising the target plurality of genes.

THP-1 cells were grown in RPMI+10% FBS. Several different site-specific disrupting agents were tested: dCas9-DNMT3a/31 (DNMT3a/31 fused to dCas9), dCas9-HDAC8, and EZH2-dCas9-HDAC8. Cells were transfected with mRNA encoding the site-specific disrupting agent, and sgRNA (from Example 1) targeted to either CTCF site of the ASMC comprising the target plurality of genes using LNPs. Lipid nanoparticle (LNP) formulation using SSOP lipid mixture was carried out by using the NanoAssemblr® Spark™ from Precision NanoSystems Inc. For experimental conditions, 350 k cells/well were plated 72 hrs after LNP transfection for each transfected condition and the parental control into 24 well plates. One hour later 10 ng/ml of TNF alpha (Sigma Cat #654205) was added to each well. Untreated parental cells were plated with and without 10 ng/ml TNF alpha.

The transfected cells were incubated with TNF alpha for 24 hrs. After that time, DNA and RNA were isolated using the AllPrep DNA/RNA Kit (Qiagen), following the Manufacture's protocol. RNA samples were retrotranscribed to cDNA using LunaScript® RT SuperMix Kit (New England Biolabs) and analyzed by quantitative PCR using individual CXCL1 specific TaqMan primer/probe sets with the TaqMan® Fast Advanced Master Mix (Thermo Fisher Scientific).

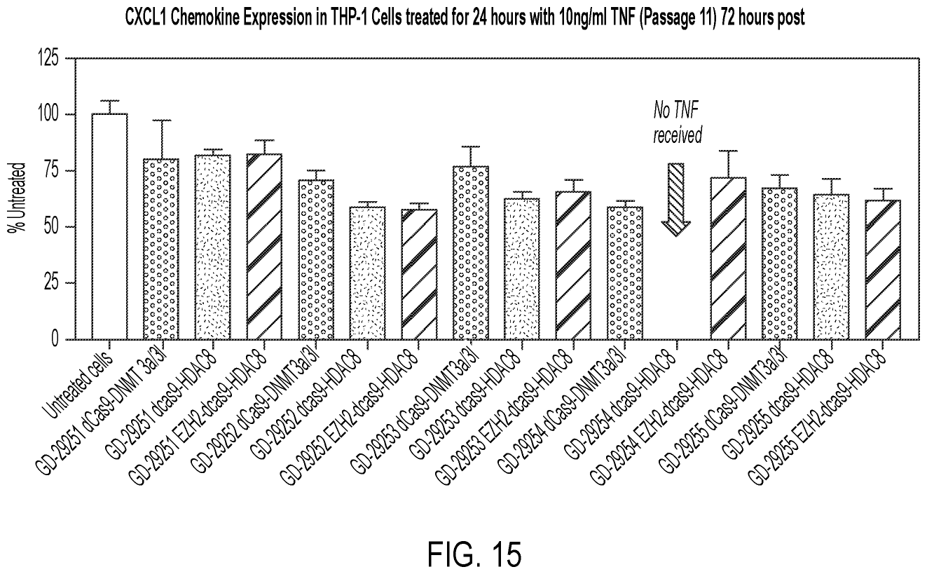

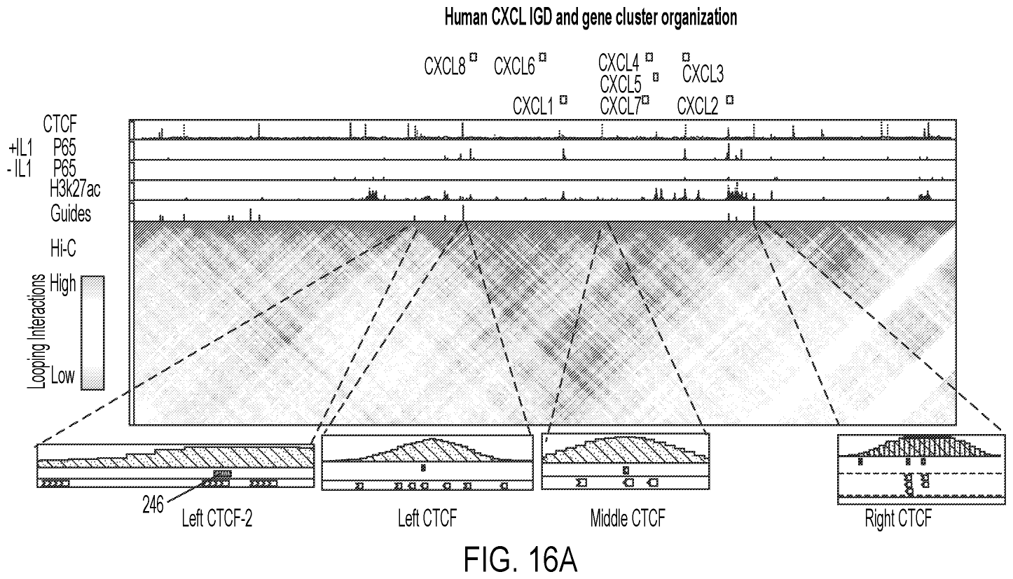

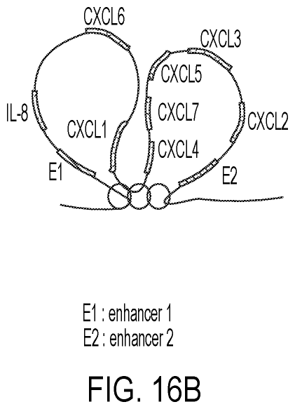

CXCL1 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells. The results show (FIG. 15) that a site-specific disrupting agent comprising dCas9-DNMT3a/31, dCas9-HDAC8, or EZH2-dCas9-HDAC8 can be used to decrease CXCL1 expression in THP-1 cells and that these agents were effective at decreasing cytokine expression when targeted to CTCF sites by several different sgRNAs.

### Example 12: CXCL Gene Cluster Expression Decreases Upon Treatment with dCas9-EZH2 and Guide 30183 in Human A549 Lung Cancer Epithelial Cells and IMR-90 Cells

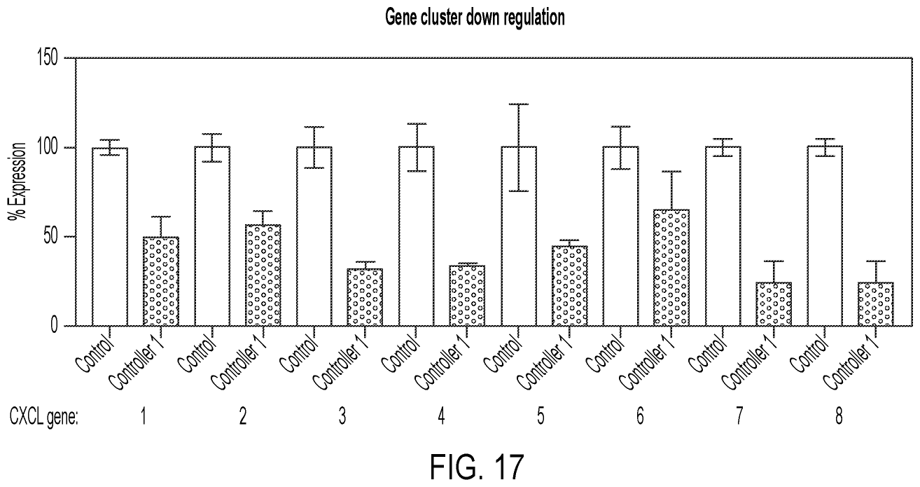

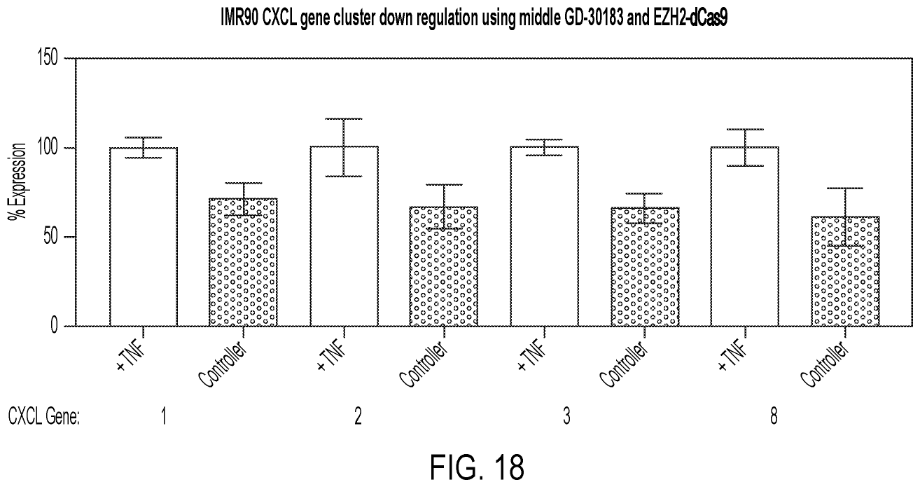

This example demonstrates CXCL gene cluster expression decreases in Human A549 lung cancer epithelial cells and IMR-90 cells when treated with dCas9-EZH2 and guide 30183 (Controller 1). Human A549 cells (ATCC® CCL-185) & IMR-90 cells (ATCC®-CCL-186) were plated at 15,000 cells per well in a flat bottom cell culture treated plate in 100 μl of media. A549 cells received F12/K ATCC®-30-2004 media and IMR-90 cells received EMEM ATCC®-30-2003 media. Both complete medias were made with 10% FBS (VWR cat #97068-085). After 24 hours adhering to the plate, LNPs containing guide 30183 and EZH2-dCas9 controller were added to the media at a final concentration of 2 g/ml SSOP lipid mix. After 6 hours, media was replaced with 100 μl of appropriate media and cells were incubated for 72 hours. After completion of 72-hour incubation, TNF alpha (Sigma Cat #654205) was added to designated wells at 10 ng/ml final concentration and incubated for 24 hours. After 24 hours, RNA was isolated using the NucleoSpin® 96 RNA Core Kit (Macherey-Nagel Inc, cat #740466.4) following the Manufacture's protocol. RNA samples were retrotranscribed to cDNA using LunaScript® RT SuperMix Kit (New England Biolabs) and analyzed by quantitative PCR using individual specific TaqMan primer/probe sets with the TaqMan® Fast Advanced Master Mix (Thermo Fisher Scientific). Gene expression was quantified relative to the human ABL1 reference gene using the ΔΔCt method. Changes in gene expression were further quantified by measuring the fold increase in gene expression after TNF alpha treatment directly compared to the levels of gene expression in the untreated cells. Data showed that expression of genes in CXCL gene cluster (specifically, CXCL1, CXCL2, CXCL3, CXCL4, CXCL5, CXCL6, CXCL7, and IL-8) levels were down between 40-70% in Human A549 lung cancer epithelial cells when treated with dCas9-EZH2 (FIG. 17). The expression of genes in CXCL gene cluster, (specifically, CXCL1, CXCL2, CXCL3, and IL-8) levels were down about 50% in IMR-90 cells when the middle CTCF was target with dCas9-EZH2 and GD-30183 (FIG. 18).

### Example 13: CXCL Gene Cluster Expression Decreases Upon Treatment with dCas9-EZH2 and Guide GD-28481 in Human Monocytes

This example demonstrates CXCL gene cluster expression decreases in Human monocyte cells when treated with dCas based effector (Controller A).

Transfection of the Cas9/guide RNP complex was carried out by electroporation into THP-1 cells (ATCC-TIB-202) by Synthego.

Upon receiving edited cell lines, vials were thawed, and cells were cultured in RPMI+10% FBS (VWR cat #97068-085) for one week to allow cells to recover from freezing and thawing. A parental unedited THP1 cell line was also analyzed for comparison. 350,000 cells were plated in quadruplicate for the edited cell line and the parental control into 24 well plates. One hour later 10 ng/ml of TNF alpha (Sigma Cat #654205) was added. Untreated control wells were also used to compare fold increase in chemokine expression.

The edited and parental cells were incubated with TNF alpha for 24 hrs. Afterwards, DNA and RNA were isolated using the DNA/RNA All Prep Kit (Qiagen) following the Manufacture's protocol. RNA samples were reverse transcribed to cDNA using LunaScript® RT SuperMix Kit (New England Biolabs) and analyzed by quantitative PCR using individual CXCL1, CXCL2, CXCL3 & IL8 specific TaqMan primer/probe sets with the TaqMan® Fast Advanced Master Mix (Thermo Fisher Scientific).

CXCL1-3 & IL8 gene expression was quantified relative to the human GAPDH reference gene using the ΔΔCt method. Changes in gene expression were further quantified by measuring the fold increase in gene expression after TNF alpha treatment directly compared to the levels of gene expression in the untreated cells.

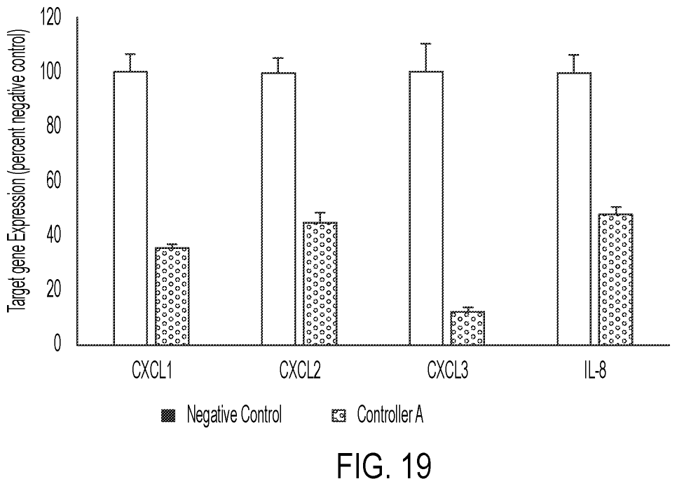

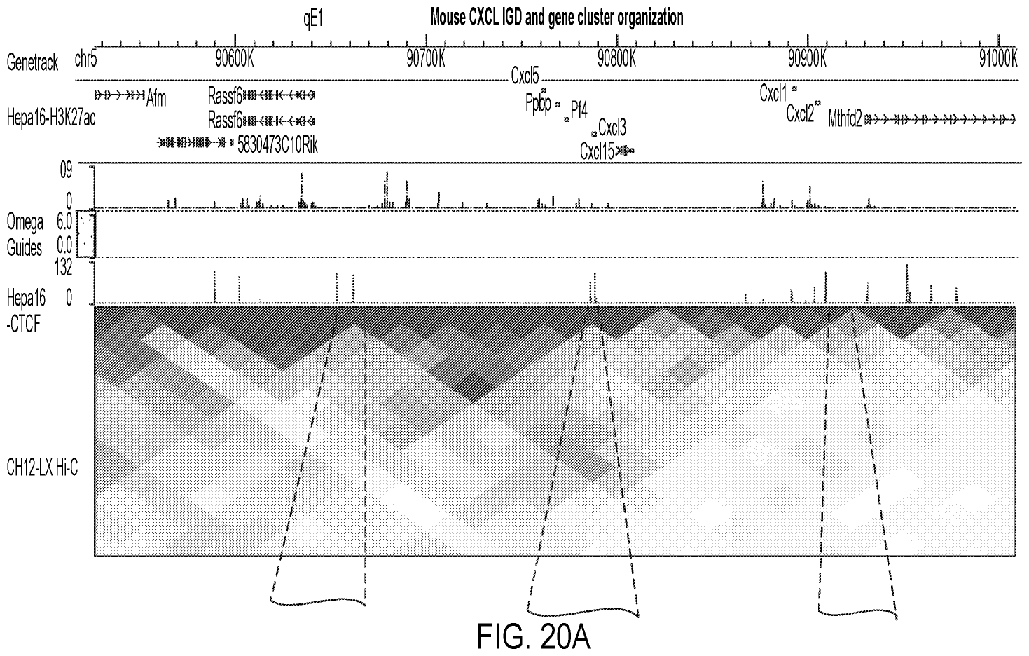

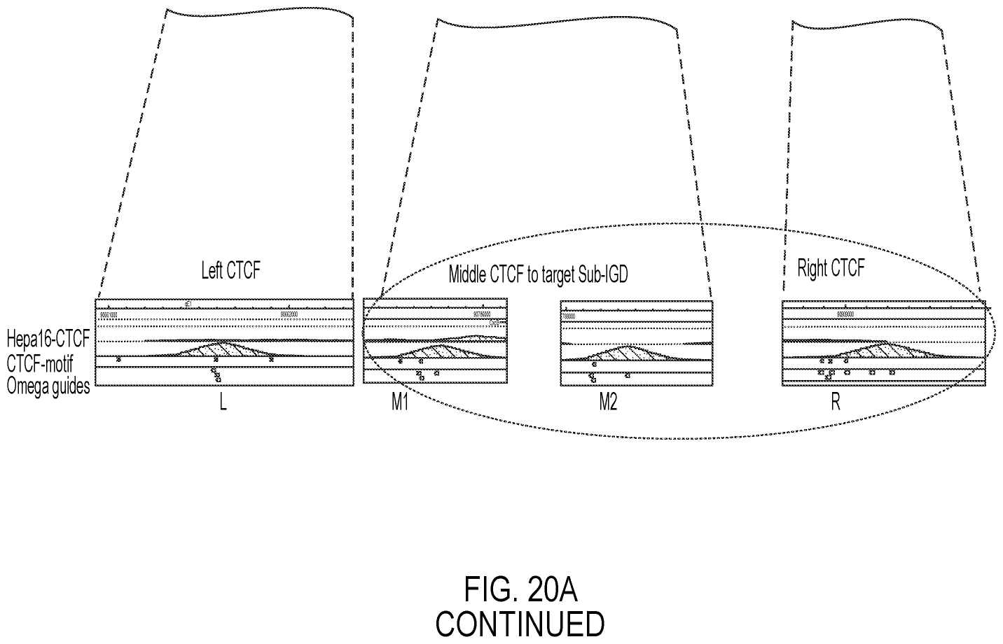

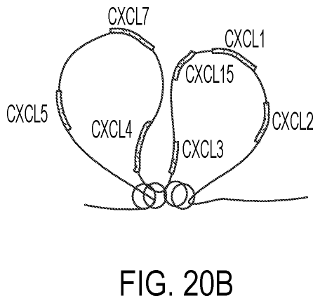

Data showed, at 24 hours post-dosing gene expression of CXCL1, CXCL2, CXCL3, and IL-8, decreased 65%, 55%, 88%, and 52% in monocytes treated with controller A compared to the CXCL1, CXCL2, CXCL3, and IL-8 gene expression respectively in untreated monocytes (FIG. 19).

### Example 14: Mouse CXCL Gene Cluster Expression Decreases Upon Treatment with dCas9-MQ1 and sgRNA Targeting Three Anchor Sequences in Hep 1.6 Cells

This example demonstrates mouse CXCL gene cluster expression downregulates when treated with dCas9-MQ1 and sgRNA targeting three anchor sequences in Hep 1.6 cells.

Mouse cells HEPA 1.6 (ATCC® CRL-1830) were plated at 10 k cells per well in a flat bottom cell culture treated plate in 100 μl of media (DMEM Gibco Cat #11995-065, 10% FBS VWR cat #97068-085). After 24 hours adhering to the plate, the cultures were divided in four treatment groups and three control groups. LNPs containing (i) guides GD-30594 and dCas9-MQ1 controller targeting Right CTCF, (ii) guide GD-30592 with dCas9-MQ1 effector targeting middle CTCF 1, (iii) guide GD-30593 with dCas9-MQ1 effector targeting middle CTCF and (iv) a combination of GD-30594, GD-30592 and GD-30593 with dCas9-MQ1 targeting both middle and right CTCF were added to the cell cultures under treatment group at a final concentration of 2 g/mL SSOP lipid mix. Untreated cells, cells treated with LNP, and cells treated with TNF and a LNP containing a transfection control guide were used as controls. After 6 hours, media was replaced with 100 μl of DMEM and cells were incubated for 72 hours. After completion of 72 hr incubation, TNF alpha (Sigma Cat #654245) is added to designated wells at 10 ng/ml final concentration and incubated for 24 hours. After 24 hrs, RNA was isolated using the NucleoSpin® 96 RNA Core Kit (Macherey-Nagel, cat #740466.4) following the Manufacture's protocol. RNA samples were retrotranscribed to cDNA using LunaScript® RT SuperMix Kit (New England Biolabs) and analyzed by quantitative PCR using individual specific TaqMan primer/probe sets with the TaqMan® Fast Advanced Master Mix (Thermo Fisher Scientific). Gene expression was quantified relative to the mouse HPRT reference gene using the ΔΔCt method. Changes in gene expression were further quantified by measuring the fold increase in gene expression after TNF alpha treatment directly compared to the levels of gene expression in the untreated cells.

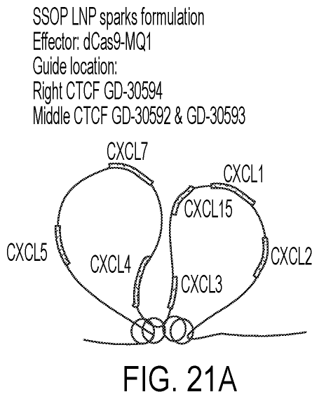

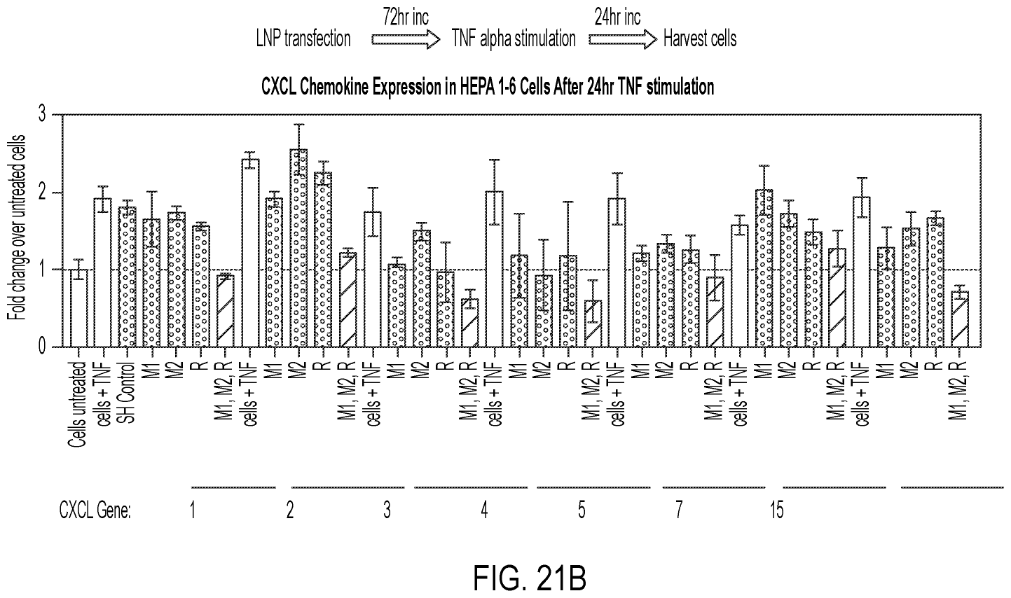

Data demonstrated, cells treated with dCas9-MQ1 transfected using guides targeting the right or one of the two middle CTCF motifs in the CXCL gene cluster, showed some down regulation in all of the seven CXCL genes after TNF alpha stimulation (FIG. 21B). However, the entire CXCL gene cluster was significantly more downregulated when cells were treated with dCas9-MQ1 transfected using a combination of guides targeting both middle and right CTCF (FIG. 21B).

### Example 15: Systemic Administration of dCas9-MQ1 Demonstrates a Significant Decrease in Leukocyte Infiltration in the Inflamed Lungs

This example demonstrates that systemic administration of dCas9-MQ1 decrease leukocyte infiltration in vivo in mouse lungs.

Murine lipopolysaccharide (LPS) lung inflammation model was used to study acute inflammation in the lungs. LNP comprising DOTAP 1% PEG short ncRNA was used as control. Each mouse received 3 mg/kg dose of LNP-DOTAP or dCas9-MQ1 at −2 hour time via intravenous administration point. The mice were stimulated with 5 mg/kg of LPS via oral aspiration at 0 hours. A second dose of LNP-DOTAP or dCas9-MQ1 at 3 mg/kg was administered at +8 hour time point. Dexamethasone was administered intraperitoneally at 10 mg/kg dose at times 0, 24, and 48 hours. The animals were terminated at 72 hours and bronchiolar lavage fluid were collected from the lungs for flow staining. Reduction in neutrophil infiltration in BALF was used a measure to understand the severity of inflammatory response.

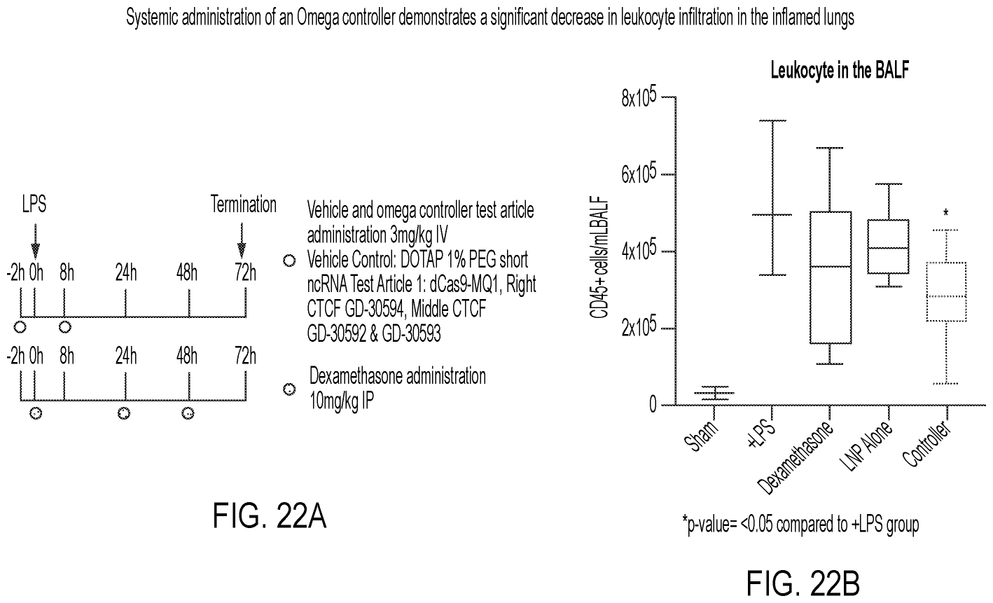

The bronchiolar lavage fluid collected from the lungs of dCas9-MQ1 treated animals showed about 5.0×10⁵ leukocyte count/mL. The sham group, i.e., healthy mice receiving no treatment did not have any significant presence of leukocyte in bronchiolar lavage fluid (BALF). The LPS treated mice, Dexamethasone treated mice, and LNP-DOTAP treated mice showed, about 8.0×10⁵ leukocyte count/mL, about 7.2×10⁵ leukocyte count/mL, and about 6.0×10⁵ leukocyte count/mL in the bronchiolar lavage fluid respectively (FIG. 22B). 56% decrease in neutrophils infiltration in broncho-alveolar lavage fluid (BALF) was also observed in mice 72 hours after treatment with dCas9-MQ1 compared to the disease control.

### Example 16: Systemic Administration of dCas9-MQ1 Demonstrates a Significant Decrease in Neutrophil Infiltration in BALF

This example analyzes BALF obtained in Example 15 to assess the cell population.

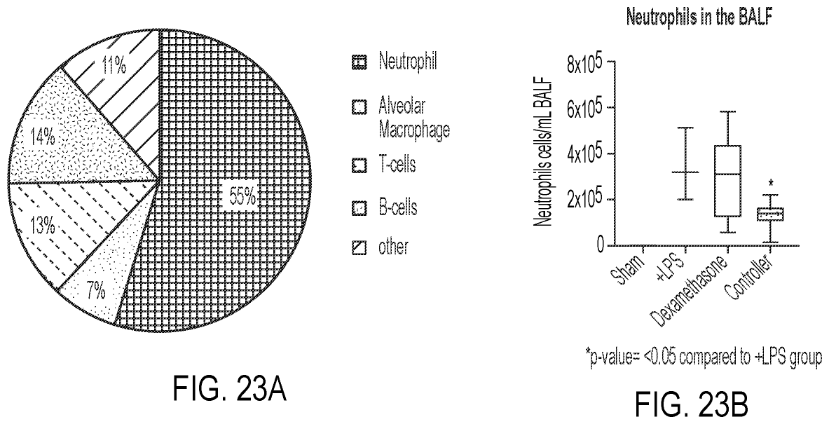

Flow cytometry analysis using the following staining panels below were used to assess the cell population in the BALF obtained in example 15 and the percentage of cells present in the BALF at the time of termination were documented (FIG. 23A). Neutrophil count in the BALF were also graphed using the antibody staining panel below.
alveolar macrophages: CD45⁺, Siglec F⁺, CD11b⁻, CD11c⁺
Neutrophils: CD45⁺, Siglec F⁻, CD11b⁺, CD11c⁻, Ly-6G⁺
T cells: CD45⁺, Siglec F⁻, CD11c⁻, CD3⁺
B cells: CD45⁺, Siglec F⁻, CD11c⁻, B220⁺ (FIG. 23B)
   Analysis showed, the leukocyte cell types that make up the majority of the infiltrating cells are neutrophils, followed by B cells, T cells, macrophages and other types of hematopoietic cells (FIG. 23A). The controller decreased the number of neutrophils infiltrating the lungs with significant difference compared to the +LPS disease group (FIG. 23B).

### Example 17: The Decrease of Leukocyte Cells in the BALF is Lung Specific

This example demonstrates that the reduction of Leukocyte cells in the BALF were lung specific suggesting the decrease was resulted from dCas9-MQ1 treatment.

Murine lipopolysaccharide (LPS) lung inflammation model was used to study acute inflammation in the lungs. LNP comprising DOTAP 1% PEG short ncRNA was used as control. Each mouse received 3 mg/kg dose of LNP-DOTAP or dCas9-MQ1 at −2 hour time via intravenous administration point. The mice were stimulated with 5 mg/kg of LPS via oral aspiration at 0 hours. A second dose of LNP-DOTAP or dCas9-MQ1 at 3 mg/kg was administered at +8 hour time point. Dexamethasone was administered intraperitoneally at 10 mg/kg dose at times 0, 24, and 48 hours. The animals were terminated at 72 hours and bronchiolar lavage fluid were collected from the lungs for flow staining. Peripheral blood was collected at 72h termination. Flow analysis using CD45⁺ antibody staining was used to determine the leukocyte population in the peripheral blood in each group. The leukocyte count obtained for each group were plotted in a graph.

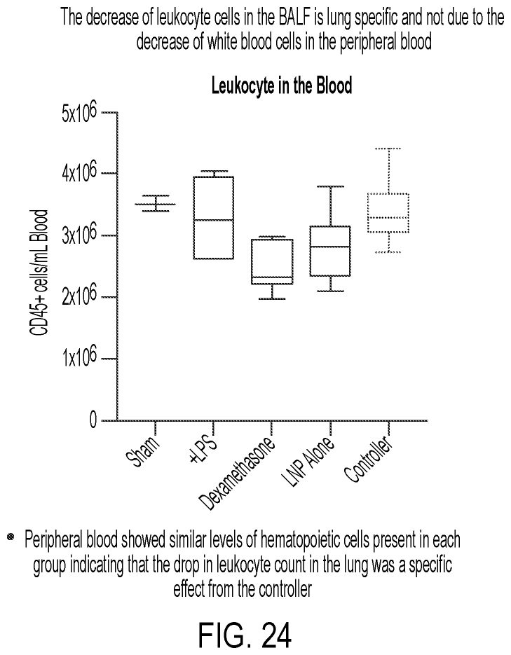

The graph illustrated that the effect of decreasing leukocyte count in the BALF with the controller treatment was lung specific suggesting the decrease in leukocyte count was due to dCas9-MQ1 treatment instead of the mouse itself had a decrease in leukocyte population which would have shown lower leukocyte count in peripheral blood as well. The hematopoietic cell population in the peripheral blood was found to be similar across all groups (FIG. 24).

### Example 18: Systemic Administration of dCas9-MQ1 Demonstrates CXCL Gene Expression is Decreased in the Lung Tissue

This example demonstrates CXCL gene cluster expression downregulates in lung tissue upon systemic administration of dCas9-MQ1.

BALF was collected using the method described in example 15. Following BALF collection, half of the left lung lobe was snapped frozen to store for qPCR analysis. The lung tissue was homogenized, and RNA was extracted for qPCR analysis quantifying specifically for CXCL1-7 and CXCL15. Gene expression was quantified relative to the mouse GAPDH reference gene using the ΔΔCt method.

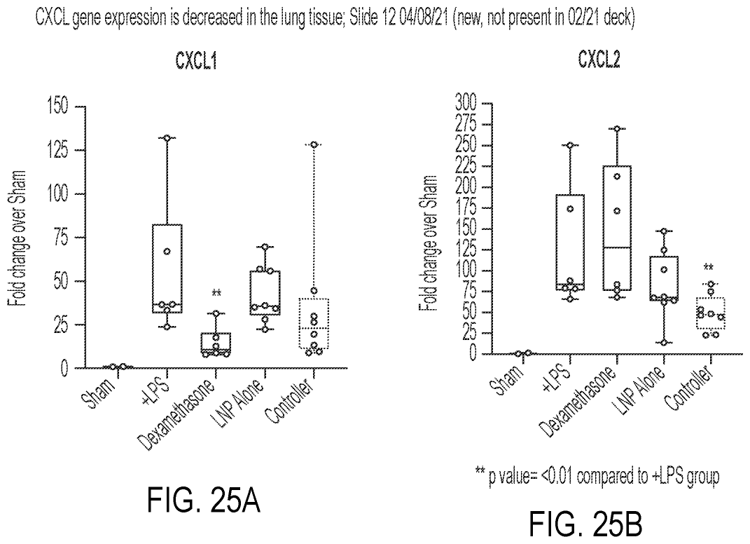

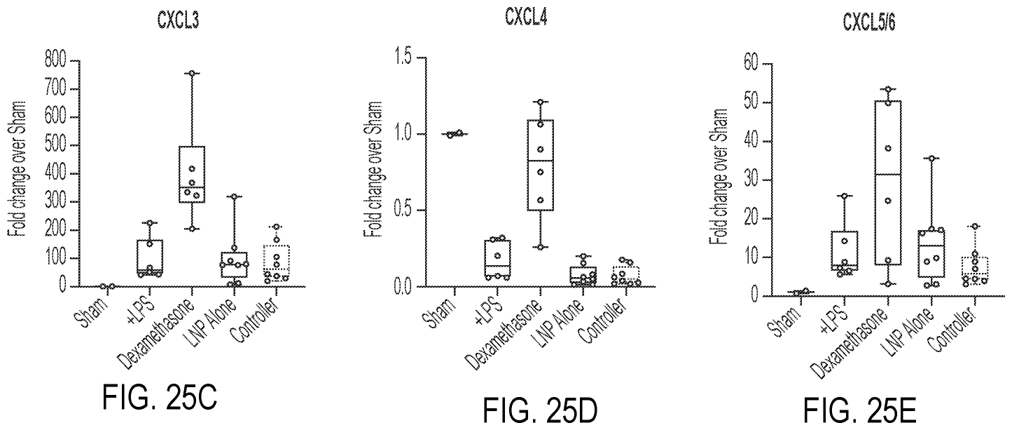

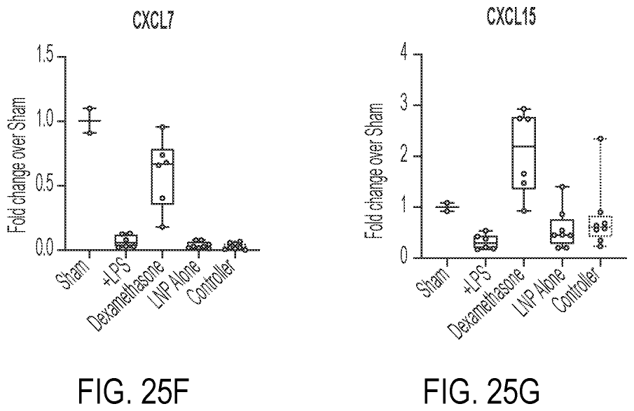

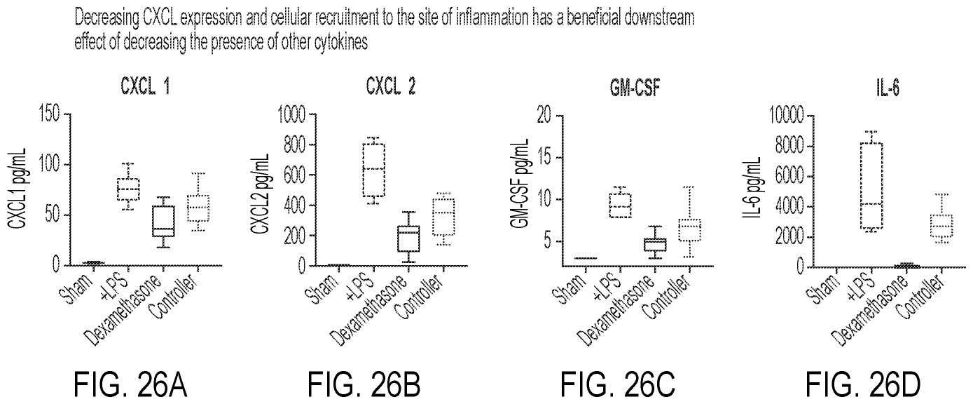

Data showed that the CXCL gene cluster expression was downregulated to varying extent upon in the lung tissue samples obtained from mice treated with dCas9-MQ1 compared to CXCL gene cluster expression in lung tissue samples obtained from mice that were not treated with dCas9-MQ1 (FIG. 25).

### Example 19: Decreasing CXCL Expression has a Beneficial Downstream Effect of Decreasing Cellular Recruitment and the Presence of Other Cytokines to the Site of Inflammation

Over-expression of the CXCL gene cluster produces chemokines that attract neutrophils. Chemokines that recruit inflammatory cells to the lung promote local inflammation, leading to severe pathogenesis. This example demonstrates downregulating CXCL expression has a beneficial downstream effect of reducing cellular recruitment leading to a reduction in the presence of other cytokines at the site of inflammation, suggesting downregulating CXCL expression is a promising method to reduce to the severity of inflammation pathogenesis.

BALF was collected using the method described in Example 15. Following BALF collection, half of the left lung lobe was snapped frozen to store for qPCR analysis. The lung tissue was homogenized, and RNA was extracted for qPCR analysis quantifying specifically for the total count of CXCL1, CXCL2, GM-CSF, and IL-6 protein in the BALF using multiplexing Luminex® instrument.

Data demonstrated that the lung tissues obtained from mice treated with dCas9-MQ1 showed a lower expression of CXCL1, CXCL2, GM-CSF, and IL-6 compared to the CXCL1, CXCL2, GM-CSF, and IL-6 expression found in the lung tissues obtained from mice that were not treated with dCas9-MQ1.

### EQUIVALENTS

It is to be understood that while the invention has been described in conjunction with the detailed description thereof, the foregoing description is intended to illustrate and not limit the scope of the invention, which is defined by the scope of the appended claims. Some aspects, advantages, and modifications are within the scope of the following claims.

We claim:

1. A method of decreasing expression of a first gene and a second gene in a cell, comprising:
contacting the cell with a site-specific disrupting agent that comprises a targeting moiety that binds specifically to a first anchor sequence or a site proximal to a first anchor sequence, in an amount sufficient to decrease expression of the first and second genes,
the first and second genes being within an anchor sequence-mediated conjunction that comprises the first anchor sequence and a second anchor sequence,
wherein optionally the first gene and the second gene are proinflammatory genes;
thereby decreasing expression of the first and second genes.

2. A site-specific disrupting agent, comprising:
a DNA-binding, e.g., a targeting moiety that binds specifically to or proximal to a first anchor sequence within a cell,
wherein the first anchor sequence is part of an anchor sequence-mediated conjunction that further comprises a second anchor sequence, a first gene, and a second gene,
wherein optionally the first gene and the second gene are proinflammatory genes.

3. The site-specific disrupting agent of claim 2, wherein the first or second anchor sequence is located between IL-8 and RASSF6; between the IL-8 enhancer and RASSF6; between CXCL1 and CXCL4; between CXCL2 and EPGN; or between the E2 enhancer and EPGN.

4. The site-specific disrupting agent of claim 2 or 3, wherein the site-specific disrupting agent further comprises an effector moiety.

5. The site-specific disrupting agent of any of claims 2-4 wherein the targeting moiety comprises a TAL effector molecule, a CRISPR/Cas molecule (e.g., a catalytically inactive CRISPR/Cas protein), a zinc finger domain, a tetR domain, a meganuclease, or an oligonucleotide.

6. The site-specific disrupting agent of any of claims 2-5, wherein the effector moiety comprises an effector described herein, e.g., MQ1, DNMT1, DNMT3A1, DNMT3A2, DNMT3B1, DNMT3B2, DNMT3B3, DNMT3B4, DNMT3B5, DNMT3B6, DNMT3L, EZH2, HDAC8, KRAB, MeCP2, HP1, RBBP4, REST, FOG1, SUZ12, SETDB1, SETDB2, EHMT2 (i.e., G9A), EHMT1 (i.e., GLP), SUV39H1, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, EZH1, SUV39H2, SETD8, SUV420H1, SUV420H2 or DNMT3, or a functional variant or fragment of any thereof.

7. The site-specific disrupting agent of any of claims 2-6, wherein the effector moiety is linked to the targeting moiety via a linker.

8. The site-specific disrupting agent of any of claims 2-7, wherein the effector moiety is C-terminal of the targeting moiety.

9. The site-specific disrupting agent of any of claims 2-7, wherein the effector moiety is N-terminal of the targeting moiety.

10. The site-specific disrupting agent of any of claims 2-9, wherein the effector moiety is encoded by a nucleotide sequence chosen from any of SEQ ID NOs: 10, 14, 16, 18, 66, 68, or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

11. The site-specific disrupting agent of any of claims 2-10, wherein the effector moiety comprises an amino acid sequence according to any of SEQ ID NOs: 11, 12, 13, 15, 17, 19, 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

12. The site-specific disrupting agent of any of claims 2-11, wherein the effector moiety is MQ1 or a functional variant or fragment thereof, e.g., wherein the effector moiety comprises an amino acid sequence of SEQ ID NO: 11 or 12 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.

13. The site-specific disrupting agent of any of claims 2-11, wherein the effector moiety is KRAB, or a functional variant or fragment thereof, e.g., wherein the effector moiety comprises an amino acid sequence of SEQ ID NO: 13 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.

14. The site-specific disrupting agent of any of claims 2-11, wherein the effector moiety is DNMT3a/3L, or a functional variant or fragment thereof, e.g., wherein the effector moiety comprises an amino acid sequence of SEQ ID NO: 15 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.

15. The site-specific disrupting agent of any of claims 2-11, wherein the effector moiety is EZH2, or a functional variant or fragment thereof, e.g., wherein the effector moiety comprises an amino acid sequence of SEQ ID NO: 17 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto.

16. The site-specific disrupting agent of any of claims 2-11, wherein the effector moiety is HDAC8, or a functional variant or fragment thereof, e.g., wherein the effector moiety comprises an amino acid sequence of SEQ ID NO: 19 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is C-terminal of the targeting moiety.

17. The site-specific disrupting agent of any of claims 2-11, wherein the effector moiety is G9A, or a functional variant or fragment thereof, e.g., wherein the effector moiety comprises an amino acid sequence of SEQ ID NO: 67 or a sequence with at least 80, 85, 90, 95, 99, or 100% identity thereto, or having no more than 20, 19, 18, 17, 16, 15, 14, 13, 12, 11, 10, 9, 8, 7, 6, 5, 4, 3, 2, or 1 positions of difference thereto, wherein optionally the effector moiety is N-terminal of the targeting moiety.

18. The site-specific disrupting agent of any of claims 2-17, which further comprises a second effector moiety.

19. The site-specific disrupting agent of claim 18, wherein the targeting moiety is situated between the first effector moiety and the second effector moiety.

20. The site-specific disrupting agent of any of claims 2-19, wherein the effector moiety comprises a polymer e.g., an oligonucleotide; e.g., a gRNA.

21. The site-specific disrupting agent of claim 20, wherein the oligonucleotide has a sequence that comprises a complement of the anchor sequence or to a sequence proximal to the anchor sequence.

22. The site-specific disrupting agent of any of claims 2-21, wherein the targeting moiety further comprises a gRNA, e.g., a gRNA that binds a genomic locus comprising at least 14, 15, 16, 17, 18, 19, or 20 nucleotides of the sequence of any of SEQ ID NOs: 20-62, e.g., wherein the gRNA comprises a sequence that comprises at least 14, 15, 16, 17, 18, 19, or 20 nucleotides of the sequence of any of SEQ ID NOs: 20-62.

23. The site-specific disrupting agent of any of claims 2-22, wherein the targeting domain comprises a CRISPR/Cas molecule, e.g., a catalytically inactive CRISPR/Cas protein, and a gRNA, e.g., a gRNA that binds a genomic locus comprising at least 14, 15, 16, 17, 18, 19, or 20 nucleotides of the sequence of any of SEQ ID NOs: 20-62, e.g., wherein the gRNA comprises a sequence that comprises at least 14, 15, 16, 17, 18, 19, or 20 nucleotides of the sequence of any of SEQ ID NOs: 20-62 and the effector moiety comprises an effector chosen from DNMT3a/31, MQ1, KRAB, G9A, HDAC8, or EZH2.

24. The site-specific disrupting agent of claim 23, wherein the targeting domain comprises a CRISPR/Cas molecule, e.g., a catalytically inactive CRISPR/Cas protein, and a gRNA, e.g., a gRNA that binds a genomic locus comprising at least 14, 15, 16, 17, 18, 19, or 20 nucleotides of the sequence of any of SEQ ID NOs: 20-62, e.g., wherein the gRNA comprises a sequence that comprises at least 14, 15, 16, 17, 18, 19, or 20 nucleotides of the sequence of any of SEQ ID NOs: 20-62, the first effector moiety comprises an effector chosen from DNMT3a/31, MQ1, KRAB, G9A, HDAC8, or EZH2, and the second effector moiety comprises an effector chosen from DNMT3a/31, MQ1, KRAB, G9A, HDAC8, or EZH2.

25. The site-specific disrupting agent of any of claims 2-24, wherein the targeting domain binds a genomic locus comprising at least 14, 15, 16, 17, 18, 19, or 20 nucleotides of the sequence of any of SEQ ID NOs: 20-62.

26. The site-specific disrupting agent of any of claims 2-25, wherein the targeting domain binds a genomic locus that is within 50 nucleotides (e.g., upstream or downstream) of the sequence of any of SEQ ID NOs: 20-62.

27. The site-specific disrupting agent of any of claims 2-26, wherein the targeting domain binds a genomic locus that is within 100 nucleotides (e.g., upstream or downstream) of the sequence of any of SEQ ID NOs: 20-62.

28. The site-specific disrupting agent of any of claims 2-27, wherein the targeting domain binds a genomic locus that is within 200 (e.g., upstream or downstream) nucleotides of the sequence of any of SEQ ID NOs: 20-62.

29. The site-specific disrupting agent of any of claims 2-28, wherein the targeting domain binds a genomic locus that is within 300 nucleotides (e.g., upstream or downstream) of the sequence of any of SEQ ID NOs: 20-62.

30. The site-specific disrupting agent of any of claims 2-29, which: (i) comprises one or more nuclear localization signal sequences (NLS), or (ii) does not comprise an NLS, optionally wherein the NLS comprises an amino acid sequence of SEQ ID NO: 63 and/or 64.

31. The site-specific disrupting agent of any of claims 18-30, wherein the first and/or second effector moiety comprises a DNA methyltransferase, a histone methyltransferase, a histone deacetylase, a histone demethylase, or a recruiter of a histone modifying complex.

32. The site-specific disrupting agent of claim 2-31, wherein the ASMC comprises two loops.

33. The site-specific disrupting agent of any of claims 2-32 or the method of claim 1, wherein the first gene is situated in a first loop of the ASMC, and the second gene is situated in a second loop of the ASMC.

34. The site-specific disrupting agent or method of claim 33, wherein the first anchor sequence is situated between the first and second loops.

35. A nucleic acid encoding a site-specific disrupting agent of any of claims 2-34.

36. The method of claim 1 or site-specific disrupting agent of any of claims 2-36, wherein the anchor sequence-mediated conjunction further comprises a third gene, and optionally wherein the method results in decreased expression of the third gene.

37. The method or site-specific disrupting agent of claim 36, wherein the anchor sequence-mediated conjunction further comprises a fourth gene, and optionally wherein the method results in decreased expression of the fourth gene.

38. The method or site-specific disrupting agent of claim 37, wherein the anchor sequence-mediated conjunction further comprises a fifth gene, and optionally wherein the method results in decreased expression of the fifth gene.

39. The method or site-specific disrupting agent of claim 38, wherein the anchor sequence-mediated conjunction further comprises a sixth gene, and optionally wherein the method results in decreased expression of the sixth gene.

40. The method or site-specific disrupting agent of claim 39, wherein the anchor sequence-mediated conjunction further comprises a seventh gene, and optionally wherein the method results in decreased expression of the seventh gene.

41. The method or site-specific disrupting agent of claim 40, wherein the anchor sequence-mediated conjunction further comprises an eighth gene, and optionally wherein the method results in decreased expression of the eighth gene.

42. A human cell having decreased expression of a first gene and a second gene, wherein the first gene and the second gene are proinflammatory genes,
wherein the cell comprises a disrupted (e.g., fully disrupted) anchor sequence-mediated conjunction that comprises the first and second genes.

43. The human cell of claim 42, which has reduced CTCF binding to an anchor sequence that is comprised by the anchor sequence-mediated conjunction, e.g., reduced by at least 20, 30, 40, 50, 60, 70, 80, 90, or 100%.

44. The human cell of either of claim 42 or 43, wherein the human cell has decreased expression of a third proinflammatory gene.

45. The human cell of claim 44, wherein the human cell has decreased expression of a fourth proinflammatory gene.

46. The human cell of claim 45, wherein the human cell has decreased expression of a fifth proinflammatory gene.

47. The human cell of claim 46, wherein the human cell has decreased expression of a sixth proinflammatory gene.

48. The human cell of claim 47, wherein the human cell has decreased expression of a seventh proinflammatory gene.

49. The human cell of claim 48, wherein the human cell has decreased expression of an eighth proinflammatory gene.

50. The human cell of any of claims 42-49, wherein the human cell comprises a mutation at chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, or chr4:74570526-74570548, or within 5, 10, 15, 20, 30, 40, or 50 nucleotides of said region.

51. A human cell comprising a mutation at chr4:74595464-74595486, chr4:74595457-74595479, chr4:74595460-74595482, chr4:74595472-74595494, chr4:75000088-75000110, chr4:75000091-75000113, chr4:75000085-75000107, chr4:75000157-75000179, chr4:75000156-75000178, chr4:74595215-74595237, chr4:74595370-74595392, chr4:74595560-74595582, chr4:74595642-74595664, chr4:74595787-74595809, chr4:74528428-74528450, chr4:74528567-74528589, chr4:74528609-74528631, chr4:74789132-74789154, chr4:74789250-74789272, chr4:74789312-74789334, chr4:74964853-74964875, chr4:74964906-74964928, chr4:74965538-74965560, chr4:74965737-74965759, chr4:75000031-75000053, chr4:75000115-75000137, chr4:75000231-75000253, chr4:74975146-74975168, chr4:74975369-74975391, chr4:74976318-74976340, chr4:74570348-74570370, chr4:74570503-74570525, or chr4:74570526-74570548, or within 5, 10, 15, 20, 30, 40, or 50 nucleotides of said region.

52. The human cell of either of claim 27 or 28, wherein the mutation comprises a deletion, substitution, or insertion (e.g., of 1-10, 1-9, 1-8, 1-7, 1-6, 1-5, 1-4, 1-3, or 1-2 nucleotides).

53. The human cell of any of claims 50-52, which has reduced CTCF binding to the mutation, e.g., reduced by at least 20, 30, 40, 50, 60, 70, 80, 90, or 100% compared to a human cell with an undisrupted ASMC.

54. The human cell of any of claims 42-53, wherein expression of the first and second genes is reduced by at least 10%, 20%, 30%, 40%, 50%, 60%, 70%, 80%, or 90% compared to a human cell with an undisrupted ASMC.

55. A system comprising:
a first site-specific disrupting agent comprising a first targeting moiety and optionally a first effector moiety, wherein the first site-specific disrupting agent binds specifically to a first anchor sequence of an anchor sequence mediated conjunction (ASMC), wherein the ASMC comprises a first gene and a second gene, and
a second site-specific disrupting agent comprising a second targeting moiety and optionally a second effector moiety, wherein the second site-specific disrupting agent binds to a second anchor sequence of the ASMC.

56. The system of claim 55, wherein the first anchor sequence is between IL-8 and RASSF6; between the IL-8 enhancer and RASSF6; between CXCL1 and CXCL4; between CXCL2 and EPGN; or between the E2 enhancer and EPGN.

57. The system of claim 55 or 56, wherein the second anchor sequence is between IL-8 and RASSF6; between the IL-8 enhancer and RASSF6; between CXCL1 and CXCL4; between CXCL2 and EPGN; or between the E2 enhancer and EPGN.

58. The system of any of claims 55-57, wherein the first anchor sequence is between the IL-8 enhancer and RASSF6 and the second anchor sequence is between CXCL1 and CXCL4.

59. The system of any of claims 55-58, wherein the first anchor sequence is between the IL-8 enhancer and RASSF6 and the second anchor sequence is between the E2 enhancer and EPGN.

60. The system of any of claims 55-59, wherein the first anchor sequence is between CXCL1 and CXCL4 and the second anchor sequence is between the E2 enhancer and EPGN.

61. The system of any of claims 55-60, wherein the first site-specific disrupting agent is a site-specific disrupting agent described herein, e.g., a site-specific disrupting agent of any of claims 2-9.

62. The system of any of claims 55-61, wherein the second site-specific disrupting agent is a site-specific disrupting agent described herein, e.g., a site-specific disrupting agent of any of claims 2-9.

63. The system of any of claims 55-62, wherein the first targeting moiety and the second targeting moiety each independent comprises a TAL effector molecule, a CRISPR/Cas molecule, a zinc finger domain, a tetR domain, a meganuclease, or an oligonucleotide.

64. The system of any of claims 55-63, wherein the first effector and the second effector each independently comprises an effector described herein, e.g., MQ1, EZH2, HDAC8, KRAB, G9A, or DNMT3a/31, or a functional variant or fragment of any thereof.

65. The system of any of claims 55-62, wherein the first effector and the second effector each independently comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof.

66. The system of any of claims 55-65, wherein the first effector and the second effector each independently comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

67. The system of any of claims 55-43, wherein the first effector and the second effector each independently comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, DNMT3a/31, or a functional variant or fragment of any thereof.

68. The system of any of claims 55-67, wherein the first effector and the second effector each independently comprises a protein chosen from KRAB, MeCP2, HP1, RBBP4, REST, FOG1, SUZ12, or a functional variant or fragment of any thereof.

69. The system of any of claims 55-68, wherein the first effector and the second effector each independently comprises a polymer e.g., an oligonucleotide.

70. The system of any of claims 55-69, wherein the first oligonucleotide and the second oligonucleotide are identical.

71. The system of any of claims 55-70, wherein the first oligonucleotide and the second oligonucleotide are different.

72. The system of any of claims 55-71, wherein the first oligonucleotide has a sequence that comprises a complement of the first anchor sequence or to a sequence proximal to the first anchor sequence and the second oligonucleotide has a sequence that comprises a complement of the second anchor sequence or to a sequence proximal to the second anchor sequence.

73. The system of any of claims 55-72, wherein the anchor sequence-mediated conjunction further comprises a third gene.

74. The system of any of claims 55-73, wherein the anchor sequence-mediated conjunction further comprises a fourth gene.

75. The system of any of claims 55-74, wherein the anchor sequence-mediated conjunction further comprises a fifth gene.

76. The system of any of claims 55-75, wherein the anchor sequence-mediated conjunction further comprises a sixth gene.

77. The system of any of claims 55-76, wherein the anchor sequence-mediated conjunction further comprises a seventh gene.

78. The system of any of claims 55-77, wherein the anchor sequence-mediated conjunction further comprises an eighth gene.

79. The system of any of claims 55-78, wherein the ASMC comprises two loops.

80. A nucleic acid composition encoding the system of any of claims 55-79.

81. The nucleic acid of claim 80, wherein a single nucleic acid encodes both of the first site-specific disrupting agent and the second site-specific disrupting agent.

82. The nucleic acid of claim 81, wherein a first nucleic acid encodes the first site-specific disrupting agent and a second nucleic acid encodes the second site-specific disrupting agent.

83. A method of decreasing expression of a first gene and a second gene in a cell, comprising contacting the cell with a system according to any of claims 55-79 of a nucleic acid composition according to any of claims 80-82.

84. The method of claim 83, wherein the cell is simultaneously contacted with the first site-specific disrupting agent and the second site-specific disrupting agent.

85. The method of claim 83, wherein the cell is sequentially contacted with the first site-specific disrupting agent and the second site-specific disrupting agent.

86. The method, human cell, site-specific disrupting agent, or system of any of claims 1-85, wherein the first gene is CXCL1 and the second gene is CXCL2.

87. The method, human cell, site-specific disrupting agent, or system of any of claims 1-85, wherein the first gene is CXCL1 and the second gene is CXCL3.

88. The method, human cell, site-specific disrupting agent, or system of any of claims 1-85, wherein the first gene is CXCL1 and the second gene is IL-8.

89. The method, human cell, site-specific disrupting agent, or system of any of claims 1-85, wherein the first gene is CXCL1 and the second gene is CXCL4.

90. The method, human cell, site-specific disrupting agent, or system of any of claims 1-85, wherein the first gene is CXCL1 and the second gene is CXCL5.

91. The method, human cell, site-specific disrupting agent, or system of any of claims 1-85, wherein the first gene is CXCL1 and the second gene is CXCL6.

92. The method, human cell, site-specific disrupting agent, or system of any of claims 1-85, wherein the first gene is CXCL1 and the second gene is CXCL7.

93. The method, human cell, site-specific disrupting agent, or system of any of claims 1-85, wherein the first gene is CXCL2 and the second gene is CXCL3.

94. The method, human cell, site-specific disrupting agent, or system of any of claims 1-85, wherein the first gene is CXCL2 and the second gene is IL-8.

95. The method, human cell, site-specific disrupting agent, or system of any of claims 1-85, wherein the first gene is CXCL2 and the second gene is CXCL4.

96. The method, human cell, site-specific disrupting agent, or system of any of claims 1-85, wherein the first gene is CXCL2 and the second gene is CXCL4.

97. The method, human cell, site-specific disrupting agent, or system of any of claims 1-85, wherein the first gene is CXCL2 and the second gene is CXCL5.

98. The method, human cell, site-specific disrupting agent, or system of any of claims 1-85, wherein the first gene is CXCL2 and the second gene is CXCL6.

99. The method, human cell, site-specific disrupting agent, or system of any of claims 1-85, wherein the first gene is CXCL2 and the second gene is CXCL7.

100. The method, human cell, site-specific disrupting agent, or system of any of claims 1-85, wherein the first gene is CXCL3 and the second gene is IL-8.

101. The method, human cell, site-specific disrupting agent, or system of any of claims 1-85, wherein the first gene is CXCL3 and the second gene is CXCL4.

102. The method, human cell, site-specific disrupting agent, or system of any of claims 1-85, wherein the first gene is CXCL3 and the second gene is CXCL5.

103. The method, human cell, site-specific disrupting agent, or system of any of claims 1-85, wherein the first gene is CXCL3 and the second gene is CXCL6.

104. The method, human cell, site-specific disrupting agent, or system of any of claims 1-85, wherein the first gene is CXCL3 and the second gene is CXCL7.

105. The method, human cell, site-specific disrupting agent, or system of any of claims 1-85, wherein the first gene is CXCL4 and the second gene is CXCL5.

106. The method, human cell, site-specific disrupting agent, or system of any of claims 1-85, wherein the first gene is CXCL4 and the second gene is CXCL6.

107. The method, human cell, site-specific disrupting agent, or system of any of claims 1-85, wherein the first gene is CXCL4 and the second gene is CXCL7.

108. The method, human cell, site-specific disrupting agent, or system of any of claims 1-85, wherein the first gene is CXCL4 and the second gene is IL-8.

109. The method, human cell, site-specific disrupting agent, or system of any of claims 1-85, wherein the first gene is CXCL5 and the second gene is CXCL6.

110. The method, human cell, site-specific disrupting agent, or system of any of claims 1-85, wherein the first gene is CXCL5 and the second gene is CXCL7.

111. The method, human cell, site-specific disrupting agent, or system of any of claims 1-85, wherein the first gene is CXCL5 and the second gene is IL-8.

112. The method, human cell, site-specific disrupting agent, or system of any of claims 1-85, wherein the first gene is CXCL6 and the second gene is CXCL7.

113. The method, human cell, site-specific disrupting agent, or system of any of claims 1-85, wherein the first gene is CXCL6 and the second gene is IL-8.

114. The method, human cell, site-specific disrupting agent, or system of any of claims 1-85, wherein the first gene is CXCL7 and the second gene is IL-8.

115. The method, human cell, site-specific disrupting agent, or system of any of claims 36-85, wherein the first gene is CXCL1, the second gene is CXCL2, and the third gene is CXCL3.

116. The method, human cell, site-specific disrupting agent, or system of any of claims 36-85, wherein the first, second, and third genes are chosen from CXCL1, CXCL2, CXCL3, CXCL4, CXCL5, CXCL6, CXCL7, or IL-8.

117. The method, human cell, site-specific disrupting agent, or system of any of claims 36-85, wherein the first, second, third, and fourth genes are chosen from CXCL1, CXCL2, CXCL3, CXCL4, CXCL5, CXCL6, CXCL7, or IL-8.

118. The method, human cell, site-specific disrupting agent, or system of any of claims 36-85, wherein the first, second, third, fourth, and fifth genes are chosen from CXCL1, CXCL2, CXCL3, CXCL4, CXCL5, CXCL6, CXCL7, or IL-8.

119. The method, human cell, site-specific disrupting agent, or system of any of claims 36-85, wherein the first, second, third, fourth, fifth, and sixth genes are chosen from CXCL1, CXCL2, CXCL3, CXCL4, CXCL5, CXCL6, CXCL7, or IL-8.

120. The method, human cell, site-specific disrupting agent, or system of any of claims 36-85, wherein the first, second, third, fourth, fifth, sixth, and seventh genes are chosen from CXCL1, CXCL2, CXCL3, CXCL4, CXCL5, CXCL6, CXCL7, or IL-8.

121. The method, human cell, site-specific disrupting agent, or system of any of claims 36-85, wherein the first, second, third, fourth, fifth, sixth, seventh, and eighth genes are CXCL1, CXCL2, CXCL3, CXCL4, CXCL5, CXCL6, CXCL7, or IL-8.

122. The method, human cell, site-specific disrupting agent, or system of any of the preceding claims, wherein the first gene is a cytokine.

123. The method, human cell, site-specific disrupting agent, or system of any of the preceding claims, wherein the second gene is a cytokine.

124. The method, human cell, site-specific disrupting agent, or system of any of the preceding claims, wherein the third gene is a cytokine.

125. The method, human cell, site-specific disrupting agent, or system of any of the preceding claims, wherein the fourth gene is a cytokine.

126. The method, human cell, site-specific disrupting agent, or system of any of the preceding claims, wherein the fifth gene is a cytokine.

127. The method, human cell, site-specific disrupting agent, or system of any of the preceding claims, wherein the sixth gene is a cytokine.

128. The method, human cell, site-specific disrupting agent, or system of any of the preceding claims, wherein the seventh gene is a cytokine.

129. The method, human cell, site-specific disrupting agent, or system of any of the preceding claims, wherein the eighth gene is a cytokine.

130. The method, human cell, site-specific disrupting agent, or system of any of the preceding claims, wherein the anchor sequence-mediated conjunction comprises 3, 4, or 5 proinflammatory genes.

131. The method, site-specific disrupting agent, or system of any of the preceding claims, wherein the site-specific disrupting agent comprises a nucleic acid (e.g., DNA or RNA) comprising a nucleotide sequence chosen from SEQ ID NOs: 20-62, or a sequence having at least 90%, 95%, 98%, or 99% identity thereto, or differing at no more than 1, 2, 3, 4, or 5 positions relative thereto.

132. The method, site-specific disrupting agent of any of the preceding claims, wherein the site-specific disrupting agent comprises a nucleic acid (e.g., DNA or RNA) comprising a nucleotide sequence chosen from SEQ ID NOs: 21, 22, 24, 40, or a sequence having at least 90%, 95%, 98%, or 99% identity thereto, or differing at no more than 1, 2, 3, 4, or 5 positions relative thereto.

133. The method or site-specific disrupting agent of any of the preceding claims, wherein the site-specific disrupting agent binds to a sequence at least partially overlapping with the region having genomic coordinates chosen from Table 4 5, 6, 7, or a sequence that is within 5, 10, 15, 20, 30, 40, 50, 100, 200, 300, 400, 500, 600, 700, 800, 900, or 1000 nucleotides of said region.

134. The method of any of the preceding claims, which results in a decrease in a level of a cytokine, e.g., a chemokine, e.g., upon stimulation of the cell with TNF-alpha, e.g., measured as described in Examples 2-11.

135. The method or human cell of any of the preceding claims, wherein a level of a cytokine (e.g., a chemokine) is decreased, e.g., upon stimulation of the cell with TNF-alpha, e.g., measured as described in Examples 2-11.

136. The method or human cell of any of the preceding claims, wherein the transcript level of one or more of (e.g., 2, 3, or all of) CXCL1, CXCL2, CXCL3, and IL8 is decreased, e.g., upon stimulation of the cell with TNF-alpha, e.g., measured as described in Examples 2 or 4-11.

137. The method or human cell of any of the preceding claims, wherein the transcript level of one or more of (e.g., 2, 3, or all of) CXCL1, CXCL2, CXCL3, CXCL4, CXCL5, CXCL6, CXCL7, and IL8 is decreased, e.g., upon stimulation of the cell with TNF-alpha.

138. The method or human cell of any of claims 132-137, wherein the decrease is a decrease of at least 10%, 20%, 30%, 40%, 50%, 60%, 70%, 80%, or 90% compared to pre-treatment levels or to a human cell with an undisrupted ASMC.

139. The method or human cell of any of the preceding claims, wherein the protein level (e.g., secreted protein level) of one or more of (e.g., 2, 3, or all of) of CXCL1, CXCL2, CXCL3, and IL8 is decreased, e.g., upon stimulation of the cell with TNF-alpha, e.g., measured as described Example 3.

140. The method or human cell of any of the preceding claims, wherein the protein level (e.g., secreted protein level) of one or more of (e.g., 2, 3, or all of) CXCL1, CXCL2, CXCL3, CXCL4, CXCL5, CXCL6, CXCL7, and IL8 is decreased, e.g., upon stimulation of the cell with TNF-alpha.

141. The method or human cell of claim 140, wherein the decrease is a decrease of at least 10%, 20%, 30%, 40%, 50%, 60%, 70%, 80%, or 90% compared to pre-treatment levels or to a human cell with an undisrupted ASMC.

142. The method of any of the preceding claims, which results in decrease in binding of CTCF to the first anchor sequence, e.g., a complete loss of binding or a loss of at least 20, 30, 40, 50, 60, 70, 80, 90, or 100% compared to a human cell with an undisrupted ASMC, e.g., as measured by ChIP and quantitative PCR.

143. The method of any of the preceding claims, which results in disruption of the anchor sequence-mediated conjunction.

144. The method of any of the preceding claims, wherein a population of the cells is contacted with the site-specific disrupting agent, and wherein the first anchor sequence is edited in at least 50%, 60%, 70%, 80%, 90%, or 95% of cells in the population.

145. The method of any of the preceding claims, wherein the effect (e.g., the decrease in cytokine levels) is additive or synergistic compared to the effect of inhibiting the first gene or the second gene individually.

146. The method of any of the preceding claims, wherein expression is decreased for at least 1, 2, 3, 4, 5, 6, 7, 10, or 14 days, or at least 1, 2, 3, 4, or 5 weeks.

147. The method, human cell, site-specific disrupting agent, or system of any of the preceding claims, wherein the cell is a cell of a subject having an inflammatory disease, e.g., an immune mediated inflammatory disease.

148. The method, human cell, site-specific disrupting agent, or system of any of the preceding claims, wherein the inflammatory disease is an autoimmune disorder, e.g., rheumatoid arthritis.

149. The method, human cell, site-specific disrupting agent, or system of any of the preceding claims, wherein the inflammatory disease is associated with a pathogenic infection, e.g., viral infection, e.g., SARS-CoV2 infection.

150. The method, human cell, site-specific disrupting agent, or system of any of the preceding claims, wherein the inflammatory disease is associated with a superinfection, e.g., infection caused by two or more pathogenic agents, e.g., by a virus and a bacterium, (e.g., by SARS-CoV2 and Streptococcus pneumoniae), e.g., by a virus and a fungus, (e.g., by SARS-CoV2 and mucormycosis).

151. The method, human cell, site-specific disrupting agent, or system of any of the preceding claims, wherein the cell is a cell of a subject having rheumatoid arthritis, inflammatory, arthritis, gout, asthma, neutrophilic asthma, neutrophilic dermatosis, paw edema, acute respiratory disease syndrome (ARDS), COVID-19, psoriasis, inflammatory bowel disease, infection (e.g., by a pathogen, e.g., a bacteria, a viruses, or a fungus), external injury (e.g., scrapes or foreign objects), effects of radiation or chemical injury, osteoarthritis, osteoarthritic joint pain, joint pain, inflammatory pain, acute pain, chronic pain, cystisis, bronchitis, dermatitis, dermatosis, cardiovascular disease, neurodegenerative disease, liver disease, lung disease, kidney disease, pain, swelling, stiffness, tenderness, redness, warmth, or elevated biomarkers related to disease states (e.g., cytokines, chemokines, growth factors, immune receptors, infection markers, or inflammatory markers).

152. The method, human cell, site-specific disrupting agent, or system of any of the preceding claims, wherein the cell is a cell of a subject having rheumatoid arthritis, psoriasis, or inflammatory bowel disease.

153. The method, human cell, site-specific disrupting agent, or system of any of the preceding claims, wherein the cell is a cell of a subject having rheumatoid arthritis, gout, neutrophilic asthma, neutrophilic dermatosis, acute respiratory disease syndrome (ARDS), or COVID-19.

154. The method, site-specific disrupting agent, or system of any of the preceding claims, wherein the anchor sequence-mediated conjunction comprises an internal enhancing sequence.

155. The method, site-specific disrupting agent, or system of any of the preceding claims, wherein the second gene (and optionally the third, fourth, fifth, sixth, seventh, or eighth genes) is transcribed in the same direction as the first gene.

156. The method, site-specific disrupting agent, or system of any of the preceding claims, wherein the first anchor sequence comprises a binding motif selected from a CTCF binding motif, USF1 binding motif, YY1 binding motif, TAF3 binding motif, or ZNF143 binding motif.

157. The method, site-specific disrupting agent, or system of any of the preceding claims, wherein the first anchor sequence comprises a CTCF binding motif.

158. The method, site-specific disrupting agent, or system of any of the preceding claims, wherein the site-specific disrupting agent binds specifically to or proximal to the first anchor sequence with sufficient affinity that it competes with binding of an endogenous nucleating polypeptide (e.g., CTCF, USF1, YY1, TAF3, or ZNF143) within the cell.

159. The method, site-specific disrupting agent, or system of any of the preceding claims, wherein the site-specific disrupting agent adds, deletes, or substitutes one or more nucleotides within or proximal to the first anchor sequence.

160. The method or site-specific disrupting agent of any of the preceding claims, wherein the site-specific disrupting agent comprises a targeting moiety or effector moiety comprising a first CRISPR/Cas molecule comprising a first CRISPR/Cas protein and first guide RNA.

161. The method or system of any of the preceding claims, wherein the first site-specific disrupting agent comprises a first targeting moiety or first effector moiety comprising a first CRISPR/Cas molecule comprising a first CRISPR/Cas protein and first guide RNA and the second site-specific disrupting agent comprises a second targeting moiety or second effector moiety comprising a second CRISPR/Cas molecule comprising a second CRISPR/Cas protein and second guide RNA.

162. The method or site-specific disrupting agent of any of the preceding claims, wherein the site-specific disrupting agent comprises a targeting moiety or effector moiety comprising TAL effector molecule, a CRISPR/Cas molecule, a zinc finger domain, a tetR domain, a meganuclease, or an oligonucleotide.

163. The method or system of any of the preceding claims, wherein the first site-specific disrupting agent comprises a first targeting moiety or first effector moiety comprising TAL effector molecule, a CRISPR/Cas molecule, a zinc finger domain, a tetR domain, a meganuclease, or an oligonucleotide and the second site-specific disrupting agent comprises a second targeting moiety or second effector moiety comprising TAL effector molecule, a CRISPR/Cas molecule, a zinc finger domain, a tetR domain, a meganuclease, or an oligonucleotide.

164. The method or site-specific disrupting agent of any of the preceding claims, wherein the site-specific disrupting agent comprises an effector moiety comprising a histone modifying functionality, e.g., a histone methyltransferase, histone demethylase, or histone deacetylase activity.

165. The method or system of any of the preceding claims, wherein the first and/or the second site-specific disrupting agent comprises an effector moiety comprising a histone modifying functionality, e.g., a histone methyltransferase, histone demethylase, or histone deacetylase activity.

166. The method, site-specific disrupting agent, or system of claim 164 or 165, wherein the effector moiety comprises a protein chosen from SETDB1, SETDB2, EHMT2 (i.e., G9A), EHMT1 (i.e., GLP), SUV39H1, EZH2, EZH1, SUV39H2, SETD8, SUV420H1, SUV420H2, or a functional variant or fragment of any thereof.

167. The method, site-specific disrupting agent, or system of claim 164 or 165, wherein the effector moiety comprises a protein chosen from HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, SIRT8, SIRT9, or a functional variant or fragment of any thereof.

168. The method, site-specific disrupting agent, or system of claim 164 or 165, wherein the effector moiety comprises EZH2 or a functional variant or fragment of any thereof.

169. The method, site-specific disrupting agent, or system of claim 164 or 165, wherein the effector moiety comprises HDAC8 or a functional variant or fragment of any thereof.

170. The method or site-specific disrupting agent of any of the preceding claims, wherein the site-specific disrupting agent comprises an effector moiety comprising a DNA modifying functionality, e.g., a DNA methyltransferase.

171. The method or system of any of the preceding claims, wherein the first and/or the second site-specific disrupting agent comprises an effector moiety comprising a DNA modifying functionality, e.g., a DNA methyltransferase.

172. The method, site-specific disrupting agent, or system of claim 170 or 171, wherein the effector moiety comprises a protein chosen from MQ1, DNMT1, DNMT3A1, DNMT3A2, DNMT3B1, DNMT3B2, DNMT3B3, DNMT3B4, DNMT3B5, DNMT3B6, DNMT3L, DNMT3a/31, or a functional variant or fragment of any thereof.

173. The method, site-specific disrupting agent, or system of claim 170 or 171, wherein the effector moiety comprises MQ1 or a functional variant or fragment of any thereof.

174. The method, site-specific disrupting agent, or system of claim 170 or 171, wherein the effector moiety comprises DNMT3 (e.g., DNMT3a, DNMT3L, DNMT3a/31, DNMT3B1, DNMT3B2, DNMT3B3, DNMT3B4, DNMT3B5, or DNMT3B6) or a functional variant or fragment of any thereof.

175. The method or site-specific disrupting agent of any of the preceding claims, wherein the site-specific disrupting agent comprises an effector moiety comprising a transcriptional repressor.

176. The method or site-specific disrupting agent of any of the preceding claims, wherein the first and/or the second site-specific disrupting agent comprises an effector moiety comprising a transcriptional repressor.

177. The method, site-specific disrupting agent, or system of claim 175 or 176, wherein the effector moiety comprises a protein chosen from KRAB, MeCP2, HP1, RBBP4, REST, FOG1, SUZ12, or a functional variant or fragment of any thereof.

178. The method, site-specific disrupting agent, or system of claim 177, wherein the effector moiety comprises KRAB or a functional variant or fragment of any thereof.

179. The method or site-specific disrupting agent of any of the preceding claims, wherein the site-specific disrupting agent comprises a polymer.

180. The method or system of any of the preceding claims, wherein the first and/or the second site-specific disrupting agent comprises a polymer.

181. The method, site-specific disrupting agent, or system of claim 179 or 180, wherein the polymer comprises a polyamide.

182. The method, site-specific disrupting agent, or system of claim 179 or 180, wherein the polymer is an oligonucleotide.

183. The method, site-specific disrupting agent, or system of claim 182, wherein the oligonucleotide has a sequence that comprises a complement of the first anchor sequence or to a sequence proximal to the first anchor sequence.

184. The method, site-specific disrupting agent, or system of claim 182, wherein the oligonucleotide has a sequence that comprises a complement of the second anchor sequence or to a sequence proximal to the second anchor sequence.

185. The method, site-specific disrupting agent, or system of any of claims 182-184, wherein the oligonucleotide comprises a chemical modification.

186. The method or site-specific disrupting agent, or system of claim 179 or 180, wherein the polymer is a peptide nucleic acid.

187. The method, site-specific disrupting agent, or system of any preceding claim, wherein the site-specific disrupting agent comprises a peptide-nucleic acid mixmer.

188. The method, site-specific disrupting agent, or system of any preceding claim wherein the site-specific disrupting agent (e.g., a targeting moiety or effector moiety of the site-specific disrupting agent) comprises a peptide or polypeptide.

189. The method, site-specific disrupting agent, or system of claim 188, wherein the polypeptide is a zinc finger polypeptide.

190. The method, site-specific disrupting agent, or system of claim 188, wherein the polypeptide is or comprises a Transcription activator-like effector nuclease (TALEN) polypeptide.

191. The method or site-specific disrupting agent of any preceding claim, wherein the site-specific disrupting agent comprises a small molecule.

192. The method or system of any preceding claim, wherein the first and/or the second site-specific disrupting agent comprises a small molecule.

193. The method or site-specific disrupting agent of any of the preceding claims, wherein the site-specific disrupting agent further comprises an effector moiety, e.g., an epigenetic modifying agent, e.g., a DNA methyltransferase, histone deacetylase, or a histone methyltransferase.

194. The method or system of any preceding claim, wherein the first and/or the second site-specific disrupting agent further comprises an effector moiety, e.g., an epigenetic modifying agent, e.g., a DNA methyltransferase, histone deacetylase, or a histone methyltransferase.

195. The method or site-specific disrupting agent of any of the preceding claims, wherein the site-specific disrupting agent comprises a fusion molecule.

196. The method or system of any preceding claims, wherein the first and/or the second site-specific disrupting agent comprises a fusion molecule.

197. The method or site-specific disrupting agent of any preceding claims wherein the site-specific disrupting agent comprises a targeting moiety comprising a CRISPR/Cas molecule and an effector moiety comprising a transcriptional repressor, e.g., as a fusion molecule.

198. The method or system of any preceding claim, wherein the first and/or the second site-specific disrupting agent comprises a targeting moiety comprising a CRISPR/Cas molecule and an effector moiety comprising a transcriptional repressor, e.g., as a fusion molecule.

199. The method or site-specific disrupting agent of claim 198, wherein the targeting moiety comprises dCas9 and the effector moiety KRAB or a functional variant or portion thereof.

200. The method or system of any preceding claim, wherein the first and/or the second targeting moiety comprises dCas9 and the effector moiety KRAB or a functional variant or portion thereof.

201. The method or site-specific disrupting agent of any claims 1-177, wherein the site-specific disrupting agent comprises a targeting moiety comprising a CRISPR/Cas molecule and an effector moiety comprising a histone methyltransferase, e.g., as a fusion molecule.

202. The method or system of any preceding claim, wherein the first and/or the second site-specific disrupting agent comprises a targeting moiety comprising a CRISPR/Cas molecule and an effector moiety comprising a histone methyltransferase, e.g., as a fusion molecule.

203. The method, site-specific disrupting agent, or system of claim 201, wherein the targeting moiety comprises dCas9 and the effector moiety comprises EZH2 or a functional variant or portion thereof.

204. The method, site-specific disrupting agent, or system of any of claims 1-196, wherein the site-specific disrupting agent comprises a targeting moiety comprising a CRISPR/Cas molecule and an effector moiety comprising a DNA methyltransferase, e.g., as a fusion molecule.

205. The method, site-specific disrupting agent, or system of claim 204, wherein the targeting moiety comprises dCas9 and the effector moiety comprises MQ1 or a functional variant or portion thereof.

206. The method, site-specific disrupting agent, or system of claim 203, wherein the targeting moiety comprises dCas9 and the effector moiety comprises DNMT3, e.g., DNMT3a/31 or a functional variant or portion thereof.

207. The method, site-specific disrupting agent, or system of any of the preceding claims, wherein the site-specific disrupting agent comprises a targeting moiety comprising a CRISPR/Cas molecule, a first effector moiety comprising a histone methyltransferase, and a second effector moiety comprising a transcriptional repressor, e.g., as a fusion molecule.

208. The method, site-specific disrupting agent, or system of claim 207, wherein the targeting moiety comprises dCas9, the first effector moiety comprises EZH2 or a functional variant or portion thereof, and the second effector moiety comprises KRAB or a functional variant or portion thereof.

209. The method, site-specific disrupting agent, or system of any of the preceding claims, wherein the site-specific disrupting agent comprises a targeting moiety comprising a CRISPR/Cas molecule, and an effector moiety comprising a histone deacetylase, e.g., as a fusion molecule.

210. The method, site-specific disrupting agent, or system of claim 209, wherein the targeting moiety comprises dCas9 and the effector moiety comprises HDAC8 or a functional variant or portion thereof.

211. The method, site-specific disrupting agent, or system of any of the preceding claims, wherein the site-specific disrupting agent comprises a targeting moiety comprising a CRISPR/Cas molecule, a first effector moiety comprising a histone methyltransferase, and a second effector moiety comprising a histone deacetylase, e.g., as a fusion molecule.

212. The method, site-specific disrupting agent, or system of claim 211, wherein the targeting moiety comprises dCas9, the first effector moiety comprises EZH2 or a functional variant or portion thereof, and the second effector moiety comprises HDAC8 or a functional variant or portion thereof.

213. The method, site-specific disrupting agent, or system of any of claims 195-212, wherein the site-specific disrupting agent comprises an amino acid sequence encoded by a nucleic acid sequence chosen from SEQ ID NOs: 69, 71, 85, 201, 202, 204, 205, 207, 209, 211, 213, 215, 217, or 219-242, a complementary or reverse complementary sequence of any thereof, or comprises a sequence with at least 80, 85, 90, 91, 92, 93, 94, 95, 96, 97, 98, or 99% identity to any thereof.

214. The method, site-specific disrupting agent, or system of any of claims 195-213, wherein the site-specific disrupting agent comprises an amino acid sequence chosen from any one of SEQ ID NOs:70, 72, 82, 84, 86, 203, 206, 208, 210, 212, 214, 216, or 218, or encoded by a sequence chosen from any one of SEQ ID NOs: 219-242, or comprises a sequence with at least 80, 85, 90, 91, 92, 93, 94, 95, 96, 97, 98, or 99% identity to any thereof.

215. The method, site-specific disrupting agent, or system of any of the preceding claims, wherein the cell is situated in a subject.

216. The method, site-specific disrupting agent of any of claims 1-215, wherein the cell is ex vivo.

217. The method or site-specific disrupting agent, or system of any of the preceding claims, wherein the cell is a mammalian cell, e.g., a human cell.

218. The method, site-specific disrupting agent, or system of any of the preceding claims, wherein the cell is a somatic cell.

219. The method, site-specific disrupting agent, or system of any of the preceding claims, wherein the cell is a primary cell.

220. The method of any of the preceding claims, wherein the step of contacting is performed ex vivo.

221. The method of claim 220, further comprising, prior to the step of contacting, a step of removing the cell (e.g., mammalian cell) from a subject.

222. The method of either of claim 220 or 221, wherein further comprising, after the step of contacting, a step of (b) administering the cells (e.g., mammalian cells) to a subject.

223. The method of any of claims 1-222, wherein the step of contacting comprises administering a composition comprising the site-specific disrupting agent to a subject.

224. The method of claim 223, wherein the site-specific disrupting agent is administered as a monotherapy.

225. The method of claim 223, wherein the site-specific disrupting agent is administered in combination with a second therapeutic agent.

226. A reaction mixture comprising a cell (e.g., a human cell, e.g., a primary human cell) and a site-specific disrupting agent, or system of any of preceding claims.

227. A method of treating a subject having an inflammatory disorder, comprising:
administering to the subject a site-specific disrupting agent, system or reaction mixture of any preceding claims in an amount sufficient to treat the inflammatory disorder,
thereby treating the inflammatory disorder.

228. The method of claim 227, wherein the inflammatory disorder is rheumatoid arthritis, psoriasis, or inflammatory bowel disease.

229. The method of claim 227 or 228, wherein the inflammatory disorder is rheumatoid arthritis, gout, neutrophilic asthma, neutrophilic dermatosis, acute respiratory disease syndrome (ARDS), or COVID-19.

230. The method of any of claims 227-229, wherein the inflammatory disorder is an autoimmune disorder, e.g., rheumatoid arthritis.

231. The method of any of claims 227-229, wherein the inflammatory disease is associated with a pathogenic infection, e.g., viral infection, e.g., SARS-CoV2 infection.

232. The method of any of claims 227-229, wherein the inflammatory disease is associated with a superinfection, e.g., infection caused by two or more pathogenic agents, e.g., by a virus and a bacterium, (e.g., by SARS-CoV2 and Streptococcus pneumoniae), e.g., by a virus and a fungus, (e.g., by SARS-CoV2 and mucormycosis).
